(19) United States
(12) Patent Application Publication
(10) Pub. No.: US 2025/0101107 A1
(43) Pub. Date: Mar. 27, 2025

(54) CD8-TARGETED IL-12 FUSION PROTEINS

(71) Applicant: Repertoire Immune Medicines, Inc., Cambridge, MA (US)

(72) Inventors: Jonathan D. NARDOZZI, Watertown, MA (US); Katharine L. SACKTON, Cambridge, MA (US); Charles Christopher STUTZ, Cambridge, MA (US); Douglas S. JONES, Bellevue, WA (US); Stefano Vincenzo GULLA, Sudbury, MA (US); Alvin PRATAMA, Quincy, MA (US)

(21) Appl. No.: 18/730,728

(22) PCT Filed: Jan. 20, 2023
(86) PCT No.: PCT/US2023/061037

§ 371 Date: Jul. 19, 2024

Related U.S. Application Data
(60) Provisional application No. 63/301,379, filed on Jan. 20, 2022.
(60) Provisional application No. 63/323,622, filed on Mar. 25, 2022.
(60) Provisional application No. 63/348,446, filed on Jun. 2, 2022.
(60) Provisional application No. 63/392,001, filed on Jul. 25, 2022.
(60) Provisional application No. 63/417,960, filed on Oct. 20, 2022.

(51) Int. Cl. C07K 16/28; A61K 38/00; C07K 14/54

(52) U.S. Cl. CPC C07K 16/2815; C07K 14/5434; A61K 38/00; C07K 2319/74

(57) ABSTRACT

Provided herein are fusion proteins comprising a CD8-binding site (e.g., a silent CD8-binding site) and an IL-12 protein. Also provided are pharmaceutical compositions comprising such fusion proteins, expression vectors and host cells for making such fusion proteins, and methods of using such fusion proteins in treating cancers.

### CROSS-REFERENCE TO RELATED APPLICATIONS

This application claims the benefit of and priority to U.S. Provisional Patent Application No. 63/301,379, filed Jan. 20,2022; U.S. Provisional Patent Application No. 63/323,622, filed Mar. 25, 2022; U.S. Provisional Patent Application No. 63/348,446, filed Jun. 2, 2022; U.S. Provisional Patent Application No. 63/392,001, filed Jul. 25, 2022; and U.S. Provisional Patent Application No. 63/417,960, filed Oct. 20, 2022, the entire disclosures of each which are incorporated herein by reference for all purposes.

### FIELD OF THE INVENTION

The invention relates to fusion proteins comprising a CD8-binding site (e.g., a silent CD8-binding site) and an IL-12 protein. In addition, the invention relates to pharmaceutical compositions comprising such fusion proteins, expression vectors and host cells for making such fusion proteins, and methods of using such fusion proteins in treating cancers.

### BACKGROUND

Immunocytokines are antibody-cytokine fusion proteins, with the potential to preferentially target specific sites, e.g., at tumor lesions, and to modulate an immune response, e.g., activate anticancer immunity, at such a site. Most immunocytokines developed to date include a cytokine moiety and either a tumor targeting moiety or an antibody Fc. For example, tumor targeting moieties fused with IL-2, IL-12, or TNF have been evaluated clinically (see, e.g., Neri and Sondel, (2016) Curr. Opin. Immunol. 40: 96-102).

CD8 is a co-receptor expressed on the surface of certain T cells. It can be present as a CD8a/CD8a homodimer or a CD8a/CD8P heterodimer. CD8 binds and stabilizes a complex comprising a class I major histocompatibility complex (MHC), a peptide presented by the class I MHC, and a T cell receptor (TCR) that binds to the peptide-MHC complex. CD8 is required for functional class I MHC-TCR signaling, which is mediated by, among other pathways, the Ras-ERK1/2 pathway that activates AP-1 transcription factor and the inositol triphosphate-Ca²⁺ pathway that activates NFAT transcription factor (see, e.g., Hwang et al., (2020) Exp. Mol. Med. (2020) 52(5):750-61). These signaling pathways then lead to T cell proliferation, differentiation, and effector functions.

Although significant developments have been made in constructing anti-CD8 antibodies and immunocytokines, there remains a need for new and useful fusion proteins for treating cancer that have adequate therapeutic efficacy and low toxicity.

### SUMMARY OF THE INVENTION

The present invention is based, in part, upon the development of fusion proteins (e.g., soluble fusion proteins) comprising an antigen-binding site that binds CD8 (e.g., human CD8 and/or cynomolgus CD8), an IL-12 protein, and optionally an antibody Fc domain. In certain embodiments, the antigen-binding site is silent, not substantially increasing or decreasing an activity of CD8 upon binding to a CD8 protein. The components of the fusion protein are coupled together in one or more configurations such that the resulting fusion proteins achieve favorable therapeutic efficacy and reduced toxicity relative to fusion proteins or unconjugated IL-12 that lacks an antigen-binding site disclosed herein. The fusion proteins described herein can be used to treat a variety of diseases and disorders, such as cancer, with improved safety and tolerability for higher doses.

Accordingly, in one aspect, the present disclosure provides a fusion protein (e.g., a soluble fusion protein) comprising an antigen-binding site that binds CD8 and an IL-12 protein, wherein the antigen-binding site does not substantially increase or decrease an activity of CD8, and the fusion protein lacks an antibody Fc domain having ADCC effector function.

In certain embodiments, the fusion protein comprises an antibody Fc domain in which an ADCC effector function is reduced by at least 60% relative to a wild-type human IgG1 Fc domain having the amino acid sequence of SEQ ID NO: 84. In other embodiments, the fusion protein lacks an antibody Fc domain.

In another aspect, the present disclosure provides a fusion protein (e.g., a soluble fusion protein) comprising: (a) two Fab fragments each comprising an identical antigen-binding site that binds CD8; (b) a single IL-12 protein; and (c) an antibody Fc domain, wherein a heavy chain polypeptide of each of the two Fab fragments is linked to an N-terminal amino acid residue of the antibody Fc domain, and the IL-12 protein is linked to a C-terminal amino acid residue of the antibody Fc domain. This format is referred to herein as “IgG-immunomodulator (1×HC),” with an example illustrated by FIG. 1E.

In another aspect, the present disclosure provides a fusion protein (e.g., a soluble fusion protein) comprising: (a) two Fab fragments each comprising an identical antigen-binding site that binds CD8; (b) two IL-12 proteins; and (c) an antibody Fc domain, wherein a heavy chain polypeptide of each of the two Fab fragments is linked to a N-terminal amino acid residue of the antibody Fc domain, and each of the two IL-12 proteins is linked to a C-terminal amino acid residue of a light chain polypeptide of each of the two Fab fragments. This format is referred to herein as “IgG-immunomodulator (LC),” with an example illustrated by FIG. 1B.

In certain embodiments of either of the two aspects above, an ADCC effector function of the antibody Fc domain is reduced by at least 60% relative to a wild-type human IgG1 Fc domain having the amino acid sequence of SEQ ID NO: 84. In certain embodiments, each of the antigen-binding sites does not substantially increase or decrease an activity of CD8.

In certain embodiments of any of the foregoing aspects, the IL-12 protein comprises an IL-12A subunit comprising an amino acid sequence at least 90% identical to SEQ ID NO: 89 and an IL-12B subunit comprising an amino acid sequence at least 90% identical to SEQ ID NO: 90. In certain embodiments, the IL-12A subunit and the IL-12B subunit are present in the same polypeptide chain. In certain embodiments, the IL-12A subunit is linked to the C-terminus of the IL-12B subunit by a linker comprising the amino acid sequence of SEQ ID NO: 85. In certain embodiments, the IL-12 protein comprises one or more mutations that reduce affinity for an IL-12 receptor, relative to wild-type human IL-12. In certain embodiments, the fusion protein is an immunostimulatory fusion protein.

In certain embodiments of the fusion proteins taking the IgG-immunomodulator (1×HC) format, the IL-12 protein is linked to the C-terminal amino acid residue of the antibody Fc domain by a linker comprising the amino acid sequence of SEQ ID NO: 87. In certain embodiments, the IL-12B subunit is linked to the C-terminal amino acid residue of the antibody Fc domain by a linker comprising the amino acid sequence of SEQ ID NO: 87. In certain embodiments, the antibody Fc domain comprises substitutions that promote Fc heterodimerization. In certain embodiments, the substitutions comprise T366W in a first polypeptide chain of the antibody Fc domain and T366S, L368A, and Y407V in a second polypeptide chain of the antibody Fc domain, the positions numbered under the EU numbering scheme.

In certain embodiments of the fusion proteins taking the IgG-immunomodulator (LC) format, each of the two IL-12 proteins is linked to the C-terminal amino acid residue of the light chain polypeptide of one of the two Fab fragments by a linker comprising the amino acid sequence of SEQ ID NO: 86. In certain embodiments, the antibody Fc domain comprises amino acids A at position 234, A at position 235, A at position 297, and optionally G at position 329, the positions numbered under the EU numbering.

The following embodiments, relating to the antigen-binding site that binds CD8 in the fusion protein, apply to any one of the foregoing aspects.

In certain embodiments, the antigen binding site comprises a heavy chain variable domain (VH) comprising complementarity determining regions HCDR1, HCDR2, and HCDR3 and a light chain variable domain (VL) comprising complementarity determining regions LCDR1, LCDR2, and LCDR3, wherein the HCDR1, HCDR2, HCDR3, LCDR1, LCDR2, and LCDR3 comprise the amino acid sequences of SEQ ID NOs: 112, 113, 4, 109, 12, and 8, respectively. In certain embodiments, the LCDR2 comprises the amino acid sequence of SEQ ID NO: 84. Specific embodiments of these consensus sequences are set forth below.

In certain embodiments, the HCDR1, HCDR2, HCDR3, LCDR1, LCDR2, and LCDR3 comprise the amino acid sequences of SEQ ID NOs: 112, 113, 4, 106, 7, and 8, respectively. In certain embodiments, the VH and the VL comprise amino acid sequences at least 95% identical to SEQ ID NOs: 21 and 105; 14 and 105; 15 and 105; 16 and 105; or 18 and 105, respectively.

In certain embodiments, the HCDR1, HCDR2, HCDR3, LCDR1, LCDR2, and LCDR3 comprise the amino acid sequences of SEQ ID NOs: 112, 113, 4, 6, 7, and 8, respectively. In certain embodiments, the VH and the VL comprise amino acid sequences at least 95% identical to SEQ ID NOs: 14 and 22; 15 and 22; 16 and 22; 18 and 22; 21 and 22; 14 and 23; 15 and 23; 16 and 23; 18 and 23; 21 and 23; 14 and 24; 15 and 24; 16 and 24; 18 and 24; or 21 and 24, respectively. In certain embodiments, the VH and the VL comprise amino acid sequences at least 95% identical to SEQ ID NOs: 1 and 5, respectively, or 9 and 5, respectively.

In certain embodiments, the HCDR1, HCDR2, HCDR3, LCDR1, LCDR2, and LCDR3 comprise the amino acid sequences of SEQ ID NOs: 112, 113, 4, 6, 26, and 8, respectively. In certain embodiments, the VH and the VL comprise amino acid sequences at least 95% identical to SEQ ID NOs: 14 and 25; 15 and 25; 16 and 25; 18 and 25; or 21 and 25, respectively.

In certain embodiments, the antigen-binding site comprises a heavy chain variable domain (VH) comprising complementarity determining regions HCDR1, HCDR2, and HCDR3 and a light chain variable domain (VL) comprising complementarity determining regions LCDR1, LCDR2, and LCDR3, wherein the HCDR1, HCDR2, HCDR3, LCDR1, LCDR2, and LCDR3 comprise the amino acid sequences of SEQ ID NOs: 88, 11, 4, 109, 12, and 8, respectively. In certain embodiments, the HCDR1, HCDR2, and LCDR2 comprise the amino acid sequences of SEQ ID NOs: 2, 13, and 84, respectively. Specific embodiments of these consensus sequences are set forth below.

In certain embodiments, the HCDR1, HCDR2, HCDR3, LCDR1, LCDR2, and LCDR3 comprise the amino acid sequences of SEQ ID NOs: 2, 17, 4, 106, 7, and 8, respectively. In certain embodiments, the VH and the VL comprise amino acid sequences at least 95% identical to SEQ ID NOs: 21 and 105; 16 and 105; or 18 and 105, respectively.

In certain embodiments, the HCDR1, HCDR2, HCDR3, LCDR1, LCDR2, and LCDR3 comprise the amino acid sequences of SEQ ID NOs: 2, 17, 4, 6, 7, and 8, respectively. In certain embodiments, the VH and the VL comprise amino acid sequences at least 95% identical to SEQ ID NOs: 21 and 24; 16 and 24; 18 and 24; 21 and 22; 16 and 22; 18 and 22; 21 and 23; 16 and 23; or 18 and 23, respectively.

In certain embodiments, the HCDR1, HCDR2, HCDR3, LCDR1, LCDR2, and LCDR3 comprise the amino acid sequences of SEQ ID NOs: 2, 3, 4, 6, 7, and 8, respectively. In certain embodiments, the VH and the VL comprise amino acid sequences at least 95% identical to SEQ ID NOs: 14 and 24; 15 and 24; 14 and 22; 15 and 22; 14 and 23; or 15 and 23, respectively. In certain embodiments, the VH and the VL comprise amino acid sequences at least 95% identical to SEQ ID NOs: 1 and 5, respectively.

In certain embodiments, the HCDR1, HCDR2, HCDR3, LCDR1, LCDR2, and LCDR3 comprise the amino acid sequences of SEQ ID NOs: 2, 17, 4, 6, 26, and 8, respectively. In certain embodiments, the VH and the VL comprise amino acid sequences at least 95% identical to SEQ ID NOs: 21 and 25; 16 and 25; or 18 and 25, respectively.

In certain embodiments, the HCDR1, HCDR2, HCDR3, LCDR1, LCDR2, and LCDR3 comprise the amino acid sequences of SEQ ID NOs: 2, 3, 4, 6, 26, and 8, respectively. In certain embodiments, the VH and the VL comprise amino acid sequences at least 95% identical to SEQ ID NOs: 14 and 25; or 15 and 25, respectively.

In certain embodiments, the HCDR1, HCDR2, HCDR3, LCDR1, LCDR2, and LCDR3 comprise the amino acid sequences of SEQ ID NOs: 10, 3, 4, 6, 7, and 8, respectively. In certain embodiments, the VH and the VL comprise amino acid sequences at least 95% identical to SEQ ID NOs: 9 and 5, respectively.

In certain embodiments, the antigen-binding site comprises a VH comprising complementarity determining regions HCDR1, HCDR2, and HCDR3 and a VL comprising complementarity determining regions LCDR1, LCDR2, and LCDR3, wherein the HCDR1, HCDR2, HCDR3, LCDR1, LCDR2, and LCDR3 comprise the amino acid sequences of SEQ ID NOs: 115, 118, 30, 32, 33, and 34, respectively. Specific embodiments of these consensus sequences are set forth below.

In certain embodiments, the HCDR1, HCDR2, HCDR3, LCDR1, LCDR2, and LCDR3 comprise the amino acid sequences of SEQ ID NOs: 115, 116, 30, 32, 33, and 34, respectively. In certain embodiments, the VH and the VL comprise amino acid sequences at least 95% identical to SEQ ID NOs: 39 and 45; 39 and 46; 39 and 47; 40 and 45; 40 and 46; 40 and 47; 41 and 45; 41 and 46; 41 and 47; 43 and 45; 43 and 46; or 43 and 47, respectively. In certain embodiments, the VH and the VL comprise amino acid sequences at least 95% identical to SEQ ID NOs: 27 and 31, respectively.

In certain embodiments, the HCDR1, HCDR2, HCDR3, LCDR1, LCDR2, and LCDR3 comprise the amino acid sequences of SEQ ID NOs: 115, 117, 30, 32, 33, and 34, respectively. In certain embodiments, the VH and the VL comprise amino acid sequences at least 95% identical to SEQ ID NOs: 35 and 31, respectively.

In certain embodiments, the antigen-binding site comprises a VH comprising complementarity determining regions HCDR1, HCDR2, and HCDR3 and a VL comprising complementarity determining regions LCDR1, LCDR2, and LCDR3, wherein the HCDR1, HCDR2, HCDR3, LCDR1, LCDR2, and LCDR3 comprise the amino acid sequences of SEQ ID NOs: 28, 37, 30, 32, 33, and 34, respectively. In certain embodiments, the HCDR2 comprises the amino acid sequence of SEQ ID NO: 38. Specific embodiments of these consensus sequences are set forth below.

In certain embodiments, the HCDR1, HCDR2, HCDR3, LCDR1, LCDR2, and LCDR3 comprise the amino acid sequences of SEQ ID NOs: 28, 29, 30, 32, 33, and 34, respectively. In certain embodiments, the VH and the VL comprise amino acid sequences at least 95% identical to SEQ ID NOs: 39 and 45; 39 and 46; 39 and 47; 40 and 45; 40 and 46; or 40 and 47, respectively. In certain embodiments, the VH and the VL comprise amino acid sequences at least 95% identical to SEQ ID NOs: 27 and 31, respectively.

In certain embodiments, the HCDR1, HCDR2, HCDR3, LCDR1, LCDR2, and LCDR3 comprise the amino acid sequences of SEQ ID NOs: 28, 42, 30, 32, 33, and 34, respectively. In certain embodiments, the VH and the VL comprise amino acid sequences at least 95% identical to SEQ ID NOs: 41 and 45; 41 and 46; or 41 and 47, respectively.

In certain embodiments, the HCDR1, HCDR2, HCDR3, LCDR1, LCDR2, and LCDR3 comprise the amino acid sequences of SEQ ID NOs: 28, 44, 30, 32, 33, and 34, respectively. In certain embodiments, the VH and the VL comprise amino acid sequences at least 95% identical to SEQ ID NOs: 43 and 45; 43 and 46; or 43 and 47, respectively.

In certain embodiments, the HCDR1, HCDR2, HCDR3, LCDR1, LCDR2, and LCDR3 comprise the amino acid sequences of SEQ ID NOs: 28, 36, 30, 32, 33, and 34, respectively. In certain embodiments, the VH and the VL comprise amino acid sequences at least 95% identical to SEQ ID NOs: 35 and 31, respectively.

In other embodiments, the antigen-binding site comprises a VH comprising complementarity determining regions HCDR1, HCDR2, and HCDR3 and a VL comprising complementarity determining regions LCDR1, LCDR2, and LCDR3, wherein the HCDR1, HCDR2, HCDR3, LCDR1, LCDR2, and LCDR3 comprise the amino acid sequences of SEQ ID NOs: 119, 120, 53, 55, 56, and 57, respectively. In certain embodiments, the VH and the VL comprise amino acid sequences at least 95% identical to SEQ ID NOs: 50 and 54, respectively.

In other embodiments, the antigen-binding site comprises a VH comprising complementarity determining regions HCDR1, HCDR2, and HCDR3 and a VL comprising complementarity determining regions LCDR1, LCDR2, and LCDR3, wherein the HCDR1, HCDR2, HCDR3, LCDR1, LCDR2, and LCDR3 comprise the amino acid sequences of SEQ ID NOs: 51, 52, 53, 55, 56, and 57, respectively. In certain embodiments, the VH and the VL comprise amino acid sequences at least 95% identical to SEQ ID NOs: 50 and 54, respectively.

In other embodiments, the antigen-binding site comprises a VH comprising complementarity determining regions HCDR1, HCDR2, and HCDR3 and a VL comprising complementarity determining regions LCDR1, LCDR2, and LCDR3, wherein the HCDR1, HCDR2, HCDR3, LCDR1, LCDR2, and LCDR3 comprise the amino acid sequences of SEQ ID NOs: 121, 122, 61, 32, 63, and 64, respectively. In certain embodiments, the VH and the VL comprise amino acid sequences at least 95% identical to SEQ ID NOs: 58 and 62, respectively.

In other embodiments, the antigen-binding site comprises a VH comprising complementarity determining regions HCDR1, HCDR2, and HCDR3 and a VL comprising complementarity determining regions LCDR1, LCDR2, and LCDR3, wherein the HCDR1, HCDR2, HCDR3, LCDR1, LCDR2, and LCDR3 comprise the amino acid sequences of SEQ ID NOs: 59, 60, 61, 32, 63, and 64, respectively. In certain embodiments, the VH and the VL comprise amino acid sequences at least 95% identical to SEQ ID NOs: 58 and 62, respectively.

In other embodiments, the antigen-binding site comprises a VH comprising complementarity determining regions HCDR1, HCDR2, and HCDR3 and a VL comprising complementarity determining regions LCDR1, LCDR2, and LCDR3, wherein the HCDR1, HCDR2, HCDR3, LCDR1, LCDR2, and LCDR3 comprise the amino acid sequences of SEQ ID NOs: 123, 124, 68, 70, 71, and 72, respectively. In certain embodiments, the VH and the VL comprise amino acid sequences at least 95% identical to SEQ ID NOs: 65 and 69, respectively.

In other embodiments, the antigen-binding site comprises a VH comprising complementarity determining regions HCDR1, HCDR2, and HCDR3 and a VL comprising complementarity determining regions LCDR1, LCDR2, and LCDR3, wherein the HCDR1, HCDR2, HCDR3, LCDR1, LCDR2, and LCDR3 comprise the amino acid sequences of SEQ ID NOs: 66, 67, 68, 70, 71, and 72, respectively. In certain embodiments, the VH and the VL comprise amino acid sequences at least 95% identical to SEQ ID NOs: 65 and 69, respectively.

In certain embodiments, the antigen binding-site cross-competes for binding to CD8 with an antigen-binding site comprising a VH and a VL comprising the amino acid sequences of SEQ ID NOs: 1 and 5; or 27 and 31, respectively.

In certain embodiments, the antigen-binding site binds (e.g., specifically binds) amino acids 28-32, 48, and 51 within a CD8 extracellular region defined by SEQ ID NO: 110. In certain embodiments, the antigen-binding site binds (e.g., specifically binds) amino acids 28-32, 34, 48, and 51 within a CD8 extracellular region defined by SEQ ID NO: 110. In certain embodiments, the antigen-binding site binds (e.g., specifically binds) amino acids 26, 28-32, 48, 51, 53, 97, and 100 within a CD8 extracellular region defined by SEQ ID NO: 110.

In certain embodiments of any one of the foregoing aspects, the antigen-binding site does not increase or decrease the stability of a complex by more than 20%, the complex comprising CD8, a T cell epitope presented by a human class I major histocompatibility complex (MHC) tetramer, and a cognate T cell receptor (TCR), as measured by the amount of the tetramer bound to a cell expressing the TCR. In certain embodiments of any one of the foregoing aspects, the antigen-binding site does not increase or decrease T cell activation by more than 50% as measured by cytotoxicity of cancer cells caused by T cells, wherein the T cells express a TCR that recognize a T cell epitope presented by a human class I MHC on the surface of the cancer cells.

In certain embodiments, a fusion protein taking the IgG-immunomodulator (lx HC) format comprises: (a) a polypeptide comprising the amino acid sequence of SEQ ID NO: 82; (b) a polypeptide comprising the amino acid sequence of SEQ ID NO: 83; and (c) two polypeptides each comprising the amino acid sequence of SEQ ID NO: 108 or 78.

In certain embodiments, a fusion protein taking the IgG-immunomodulator (LC) format comprises: (a) two polypeptides each comprising the amino acid sequence of SEQ ID NO: 76; and (b) two polypeptides each comprising the amino acid sequence of SEQ ID NO: 74 or 107.

In another aspect, the present disclosure provides a pharmaceutical composition comprising a fusion protein disclosed herein and a pharmaceutically acceptable carrier or excipient.

In another aspect, the present disclosure provides a method of killing a cancer cell, the method comprising exposing the cancer cell and a CD8+ T lymphocyte to a fusion protein disclosed herein.

In another aspect, the present disclosure provides a method of treating cancer, the method comprising administering to a subject in need thereof an effective amount of a fusion protein disclosed herein.

In another aspect, the present disclosure provides one or more nucleic acids encoding a fusion protein disclosed herein. In another aspect, the present disclosure provides a vector comprising the one or more nucleic acids.

In another aspect, the present disclosure provides a recombinant cell comprising the one or more nucleic acids disclosed above. In another aspect, the present disclosure provides a method of producing a protein, the method comprising culturing this recombinant cell under suitable conditions that allow expression of the fusion protein. In certain embodiments, the method further comprises isolating the fusion protein. In certain embodiments, the method further comprises formulating the fusion protein with a pharmaceutically acceptable carrier or excipient.

### BRIEF DESCRIPTION OF THE DRAWINGS

FIG. 1A, FIG. 1B, FIG. 1C, FIG. 1D, and FIG. 1E are schematic representations of fusion proteins comprising a CD8-binding agent and an immunomodulator, e.g., a single chain IL-12.

FIG. 2A is a bar graph depicting the ability of the indicated hybridoma supernatant to bind CD8+ T cells. Black bars indicate supernatant sample was not diluted (“neat”), and grey bars indicate supernatant sample was diluted 1:8. FIG. 2B is a bar graph depicting the ability of the indicated hybridoma supernatant to interfere with binding of MART-1-MHC tetramers to MART-1-specific T cells. Antibody SK1 was used as a representative anti-CD8 antibody that interferes with CD8 function. n=2; mean±SD.

FIG. 3 is a network plot which depicts the results of an epitope binning experiment with anti-CD8 antibodies. Anti-CD8 antibodies were immobilized on a surface as ligands, CD8a/P was bound to the immobilized antibodies as an antigen, and other anti-CD8 antibodies were tested for binding to the surface as analytes. Each pair of antibodies was tested in both orientations, i.e., one antibody used as ligand and the other antibody used as analyte and vice versa, with the exception of OKT8 which was only tested as an analyte. Solid connecting lines represent competitive binding in both orientations, except for OKT8, wherein the solid line indicates that binding was competitive in the one orientation tested. Dashed connecting lines represent competitive binding in only one orientation. Grey enclosures indicate that antibodies were grouped into the same epitope bin.

FIG. 4A is a bar graph depicting the ability of the indicated antibody to abrogate T-cell mediated killing of tumor cells in vitro. MART-1 T cells and SK-MEL-5 tumor cells were combined at a 2:1 ratio and co-cultured in the presence of a saturating concentration of the indicated anti-CD8 antibody, or in the absence of added antibody (“untreated”). Results are reported as the percentage of surviving tumor cells relative to a no-T-cell control. n=2; mean±SD. FIG. 4B is a bar graph depicting the ability of the indicated antibody to interfere with binding of MART-1I-MHC tetramers to MART-1 T cells. MART-1 T cells were incubated with fluorescently-labeled MART-1 tetramer alone (“tetramer”) or in combination with a saturating concentration of the indicated anti-CD8 antibody, and results were analyzed by flow cytometry. Results represent the percentage of CD3+ cells that are tetramer-positive. n=2; mean±SD. FIG. 4C is a bar graph depicting the ability of the indicated antibody to abrogate TCR-induced ERK phosphorylation. MART-1 T cells were incubated with MART-1 tetramers alone (“untreated”) or in combination with a saturating concentration of the indicated antibody. Results represent the percentage of lymphocytes that are phospho-ERK-positive, as assessed by flow cytometry. n=3; mean±SD.

FIGS. 5A-5B depict the results of in vivo experiments to assess tumor regression and toxicity following treatment with anti-CD8:IL12 fusion proteins. C57BL6 mice inoculated with MC38 tumor cells were treated weekly with the indicated fusion protein, and tumor volumes (FIG. 5A) and mouse body weight (FIG. 5B) were monitored post-treatment. Arrows indicate timing of fusion protein injection. n=9; mean±SEM.

FIGS. 6A-6C depict tumor regression results in a MC38 tumor cell inoculation mouse model, following weekly treatment with CD8IgG-IL12 (LC) Clone 2 (“anti-mCD8-IgG-IL12 (LC)”) or CD8IgG-IL12 (1×HC) (“anti-mCD8-IgG-IL12 (HC-1)”). “CR” indicates the fraction of mice per treatment that experienced a complete response. FIG. 6A depicts the results of all treatments. Arrows below the X-axis indicate timing of fusion protein injection. n=8; mean±SEM. FIG. 6B depicts the results from the individual mice in the CD8IgG-IL12 (LC) Clone 2 treatment. FIG. 6C depicts the results from the individual mice in the CD8IgG-IL12 (1×HC) treatment.

FIGS. 7A-7C depict tumor regression results in a B16-F10 tumor cell inoculation mouse model, following weekly treatment with CD8IgG-IL12 (LC) Clone 2 (“anti-mCD8-IgG-IL12 (LC)”) or CD8IgG-IL12 (1×HC) (“anti-mCD8-IgG-IL12 (HC-1)”). FIG. 7A depicts the results of all treatments. Arrows below the X-axis indicate timing of fusion protein injection. n=8; mean±SEM. FIG. 7B depicts the results from the individual mice in the CD8IgG-IL12 (LC) Clone 2 treatment. FIG. 7C depicts the results from the individual mice in the CD8IgG-IL12 (1×HC) treatment.

FIGS. 8A-8B depict biodistribution and retention of IL-12 on the surface of CD4+ T cells, CD8+ T cells, and NK cells, in a MC38 (FIG. 8A) or B16-F10 (FIG. 8B) tumor cell inoculation mouse model, following weekly treatment with CD8IgG-IL12 (LC) Clone 2 (“anti-mCD8 LC”) or CD8IgG-IL12 (1×HC) (“anti-mCD8 HC-1”). Control constructs included nontargeted recombinant mouse IL-12 (“rIL-12”) and constructs in which the CD8-binding site was replaced by an antigen-binding site that binds respiratory syncytial virus (RSV). Results are reported as the percentage of cells with detected surface-bound IL-12. n =8.

FIGS. 9A-9D depict production of pro-inflammatory cytokines IFN-7 (FIG. 9A), TNF-α (FIG. 9B), IL-6 (FIG. 9C), and IL-18 (FIG. 9D) in the serum, in a MC38 tumor cell inoculation mouse model, following treatment with CD8IgG-IL12 (LC) Clone 2 (“anti-mCD8-IgG-IL12 (LC)”) or CD8IgG-IL12 (1×HC) (“anti-mCD8-IgG-IL12 (HC-1)”). n=8; mean±SD.

FIGS. 10A-10C depict tumor regression in a MC38 tumor cell inoculation mouse model following weekly treatment with different doses of CD8IgG-IL12 (LC) Clone 2 (“anti-mCD8-IgG-IL12 (LC)”) or CD8IgG-IL12 (1×HC) (“anti-mCD8-IgG-1×IL12 (HC)”). Arrows indicate timing of fusion protein injection. n=8; mean±SEM. FIG. 10A compares 22 μg of CD8IgG-IL12 (LC) Clone 2 with 17 μg of CD8IgG-IL12 (1×HC), construct doses equivalent to 12 μg of the CD8-binding site. FIG. 10B compares 44 μg of CD8IgG-IL12 (LC) Clone 2 with 34 μg of CD8IgG-IL12 (1×HC), construct doses equivalent to 24 μg of the CD8-binding site. FIG. 10C compares 22 μg of CD8IgG-IL12 (LC) Clone 2 with 34 μg of CD8IgG-IL12 (1×HC), construct doses equivalent to 10 μg of IL-12.

FIGS. 11A-11B depict binding and retention of IL-12 to CD8+ T cells, in a MC38 tumor cell inoculation mouse model, during 72 hours following treatment with CD8IgG-IL12 (LC) Clone 2 (“anti-mCD8-IgG-IL12 (LC)”) or CD8IgG-IL12 (1×HC) (“anti-mCD8-IgG-1×IL12 (HC)”). FIG. 11A depicts the amount (mean fluorescence intensity, “MFI”) of cell surface-bound IL-12 on CD8+ T cells at the indicated timepoints. n=5; mean±SEM. FIG. 11B is a series of histograms depicting the frequency of IL-12 on CD8+ T cells at the indicated timepoints.

FIG. 12 depicts binding curves which summarize the abilities of two anti-human-CD8:IL12 fusion constructs, 02A01-IgG-IL12 (LC) and 03A01-IgG-IL12 (LC), to bind to CD8+ T cells versus NK cells from human PBMCs.

FIG. 13 is a bar graph depicting the ability of the indicated antibody, antibody fragment, protein, or fusion protein construct to reduce or enhance T cell-mediated killing of tumor cells in vitro. SK-MEL-5 tumor cells and MART-1 T cells were combined at a 1:1 ratio and co-cultured in the presence of recombinant human IL-12 (“huIL12”), 02A01-Fab-IL12 (LC) (“02A01 Fab-IL12p70”), 02A01-Fab-Fc-IL12 (LC) (“02A01-Fab-Fc k/h.-IL12p70”), 02A01-IgG-IL12 (LC) (“02A01 IgG IL12p70 (LC)”), 02A01-IgG-IL12 (LC) (“02A01 IgG IL12p70 (LC)”), 02A01 Fab alone (“02A01 Fab”), 02A01 IgG, 03A01-Fab-IL12 (LC) (“03A01 Fab-IL12p70”), 03A01-Fab-Fc-IL12 (LC) (“03A01-Fab-Fc k/h.-IL12p70”), 03A01-IgG-IL12 (LC) (“03A01 IgG IL12p70 (LC)”), 03A01-IgG-IL12 (LC) (“03A01 IgG IL12p70 (LC)”), 03A01 Fab alone (“03A01 Fab”), 03A01 IgG, or in the absence of added protein (“untreated”). Results are reported as the percentage of surviving tumor cells relative to a no-T-cell control. n=3; mean±SD.

FIGS. 14A-14C depict the ability of single chain IL-12 or an anti-human-CD8:IL-12 fusion protein to induce STAT4 phosphorylation in cynomolgus monkey resting PBMCs (FIG. 14A) or pre-activated PBMCs (FIGS. 14B and 14C). FIGS. 14A and 14B depict STAT4 phosphorylation in the indicated cell types following treatment with single chain IL-12. FIG. 14C depicts STAT4 phosphorylation in CD8+ T cells following treatment with single chain IL-12, an 02A01 IgG, or the indicated fusion protein. For each experiment, n=2; mean±SD.

FIGS. 15A-15B depict the ability of single chain IL-12 (FIG. 15A) or 02A01-IgG-IL12 (LC) (FIG. 15B) to induce STAT4 phosphorylation in in CD8+ T cells, NK cells, and CD4+ T cells from human PBMCs. n=2; mean±SD.

FIGS. 16A-16B depict binding curves showing the abilities of two anti-human-CD8:IL-12 constructs, H6K3-IgG-IL12 (LC) (“hCD8IgG-IL12 (LC)”) and H6K3-IgG-IL12 (1×HC) (“hCD8IgG-1×IL12 (HC)”), to bind CD8+ T cells (FIG. 16A) or NK cells (FIG. 16B) in human PBMCs. n=2; mean±SD.

FIGS. 17A-17D depict binding curves showing the ability of two anti-human-CD8:IL-12 constructs, H6K3-IgG-IL12 (LC) (“hCD8IgG-IL12 (LC)”) and H6K3-IgG-IL12 (1×HC) (“hCD8IgG-1×IL12 (HC)”), to bind CD8+ T cells and NK cells in human or cynomolgus monkey PBMCs at various concentrations, as analyzed by FACS. FIG. 17A and FIG. 17B show the ability of H6K3-IgG-IL12 (LC) and H6K3-IgG-IL12 (1×HC), respectively, to bind the indicated cell type in human PBMCs. X-axis represents fusion protein concentration (log₁₀(M)). n=2; mean±SD. FIG. 17C and FIG. 17D show the ability of H6K3-IgG-IL12 (LC) and H6K3-IgG-IL12 (1×HC), respectively, to bind the indicated cell type in cynomolgus monkey PBMCs. X-axis represents fusion protein concentration (log₁₀(M)). n=2; mean±SD. FIG. 17E is a series of histograms depicting the numbers of human and cynomolgus monkey CD8+ T cells and NK cells detected as having cell surface-bound IL12 following incubation with the indicated concentration of H6K3-IgG-IL12 (LC). FIG. 17F is a series of dot plots depicting the percentage of CD45+ cells staining for CD8 and/or IL-12, following the 30 minute incubation with 0.128 nM of the indicated IL-12 fusion protein. Cells incubated without a fusion protein were used as a control (“−”). The percentages of human and cynomolgus monkey CD45+ cells co-staining for IL-12 and CD8a are shown in the bar graphs of FIG. 17G and FIG. 17H, respectively.

FIG. 18 depicts the ability of H6K3-IgG-IL12 (LC) (“hCD8IgG-IL12 (LC)”), H6K3-IgG-IL12 (1×HC) (“hCD8IgG-1×IL12 (HC)”), or rhIL-12 to induce dose-dependent IL-12-stimulated signaling in HEK-Blue™ IL-12 reporter cells (Invivogen), as quantified by expression of an IL-12/pSTAT4-responsive reporter gene. Untreated cells were used as a control (“mock”). n=2; mean±SD.

FIGS. 19A-19D depict the levels of STAT4 phosphorylation in response to single-chain human IL-12 (rhIL-12), H6K3-IgG-IL12 (LC) (“hCD8IgG-IL12 (LC)”), or H6K3-IgG-IL12 (1×HC) (“hCD8IgG-1×IL12 (HC)”), in pre-activated human and cynomolgus monkey PBMCs. The results in human CD8+ T cells, human NK cells, cynomolgus monkey CD8+ T cells, and cynomolgus monkey NK cells are shown in FIGS. 19A, 19B, 19C, and 19D, respectively. For all experiments, n=2; mean±SD.

FIGS. 20A-20D depict the levels of IFN-7 released from PBMCs in response to single-chain human IL-12 (rhIL-12), H6K3-IgG-IL12 (LC) (“hCD8IgG-IL12 (LC)”), or H6K3-IgG-IL12 (1×HC) (“hCD8IgG-1×IL12 (HC)”), in the absence (“resting”) or presence (“activated”) of PMA (2 ng/mL) and PHA-L (200 ng/mL). The results obtained from rested human PBMCs, activated human PBMCs, rested cynomolgus monkey PBMCs, and activated cynomolgus monkey PBMCs are shown in FIGS. 20A, 20B, 20C and 20D, respectively. For all experiments, n=2; mean±SD.

FIG. 21 depicts percentage of SK-MEL-5 tumor cells killed by MART-1 T cells in vitro in the presence of single-chain human IL-12 (rhIL12), H6K3-IgG-IL12 (LC) (“hCD8IgG-IL12 (LC)”), or H6K3-IgG-IL12 (1×HC) (“hCD8IgG-1×IL12 (HC)”). Results are reported as the percentage of surviving tumor cells relative to a no-T-cell control. n=3; mean±SD.

FIG. 22 is a bar graph depicting the ability of one of two humanized anti-human-CD8:IL-12 fusion proteins, H6K3EQ-IgG-IL12 (LC) (“hCD8IgG-IL12 (LC)”) or H6K3EQ-IgG-IL12 (1×HC) (“hCD8IgG-1×IL12 (HC)”), to increase serum levels of IFN-7 following intravenous or subcutaneous administration to cynomolgus monkeys. n=1.

FIGS. 23A-23B depict the amounts of IL-12 bound to CD8-expressing immune cells following intravenous administration of H6K3EQ-IgG-IL12 (LC) (“hCD8IgG-IL12 (LC)”) or H6K3EQ-IgG-IL12 (1×HC) (“hCD8IgG-1×IL12 (HC)”) to cynomolgus monkeys at a dose of 10 μg/kg, 20 μg/kg, 40 μg/kg, or 100 μg/kg. FIG. 23A is a bar graph showing IL-12 binding to CD8+ T cells 24 hours post-administration. n=1 or 2; mean±SD. FIG. 23B is a series of histograms depicting the frequency of CD4+ T cells, CD8+ T cells, and NK cells with surface-bound IL-12 24 hours post-administration of 100 μg/kg of H6K3EQ-IgG-IL12 (LC) (left) or H6K3EQ-IgG-IL12 (1×HC) (right).

FIG. 24 is a bar graph showing binding of humanized 02A01-derived antibody variants to bind human CD8, as measured by binding to CD8+ T cells at an antibody concentration of 0.2 nM. n=2; mean±SD.

FIG. 25 is a bar graph showing binding of humanized 03A01-derived antibody variants to bind human CD8, as measured by binding to CD8+ T cells at an antibody concentration of 5 nM. n=2; mean±SD.

FIG. 26A-26G are bar graphs showing changes in expression of cell-surface markers on immune cells from tumors and tumor-draining lymph nodes (tdLN) following administration of CD8IgG-IL12 (1×HC) (“CD8-IL12”, triangles), untargeted IL-12 (asterisks), or a vehicle control (HBSS, circles) in a mouse MC38 tumor cell inoculation model. FIG. 26A-26C are bar graphs showing the CD8:CD4 T cell ratio at 4 days post-administration (FIG. 26A), the frequency of CD25hi CD69hi cells among CD8+ T cells in the tumor at 4 days post-administration (FIG. 26B), and the frequency of CD107a+ cells among CD8+ T cells in the tumor at 11 days post-administration (FIG. 26C). FIG. 26D is a bar graph showing the percentage of CD8+ XCR1+ cells among CD11c+ cells in the tdLN 4 days post-administration. FIG. 26E is a bar graph summarizing the level of expression (mean fluorescence intensity) of CD86 on cDC1 cells (CD8+ CD11c+ dendritic cells) in the tumor at 4 days post-administration. FIG. 26F-26G are bar graphs showing the percentage of MHCII+ CD86+ cells among monocytic myeloid-derived suppressor cells (mo-MDSCs) in the tdLNs (FIG. 26F) and in the tumor (FIG. 26G) at 4 days post-administration. For all experiments, n=5; mean±SD.

FIG. 27 is a pie chart created based on data obtained from a Crown Bioscience database, showing the type of cells in the tumor microenvironment of Pan02, a murine model of pancreatic ductal cancer.

FIG. 28A-28D depict tumor growth based on data obtained from a Crown Bioscience database, showing in vivo efficacy of various checkpoint inhibitors in a syngeneic murine Pan02 model of pancreatic ductal cancer (isotype control (LTF-2) in FIG. 28A, anti-mCTLA-4 (9D9) in FIG. 28B, anti-mPD-1 (RMP1-14) in FIG. 28C, and anti-mPD-L1 (10F.9G2) in FIG. 28D).

FIG. 29 is a bar graph showing tumor growth inhibition in mice following weekly administration of CD8IgG-IL12 (1×HC) (“CD8-IL12”) or PBS vehicle control on days 0, 7, and 14 in a syngeneic murine Pan02 model of pancreatic ductal cancer. n=10; mean±SEM.

FIGS. 30A-30E depict tumor regression in a variety of syngeneic murine tumor models following weekly administration of CD8IgG-IL12 (1×HC) (“CD8-IL12”). Results are depicted for an EMT6 model of breast cancer (FIG. 30A), an H22 model of liver cancer (FIG. 30B), an A20 model of lymphoma (FIG. 30C), a B16BL6 model of melanoma (FIG. 30D), and for a Renca model of kidney cancer (FIG. 30E).

FIG. 31 is a bar graph comparing tumor growth inhibition data from studies of CD8IgG-IL12 (1×HC) (“CD8-IL12”) with historical data from studies of an anti-murine-PD-1 antibody in the same cancer models. Black bars correspond to data depicted in FIG. 29 and FIGS. 30A-30E, and from additional murine syngeneic models of colon cancer (“MC38” and “CT26.WT”), melanoma (“B16F10”), lung cancer (“LL/2”), prostate cancer (“RM1”), and hepatoma (“Hepa1-6”). Striped bars correspond to historical data using an anti-murine-PD-1 antibody. Results are quantified as mean tumor growth inhibition (% TGI), calculated as ((mean volume_{control group}−mean volume_{treatment group})/mean volume_{control group}))*100%.

FIG. 32 depicts tumor regression results in a B16F10 syngeneic mouse tumor model following weekly administration of CD8IgG-IL12 (1×HC), administration of a dose of PMEL-specific T cells, both treatments in combination, or weekly administration of a vehicle control (HBSS). n=8, mean±SEM.

FIG. 33A-33E depict the results of in vivo cynomolgus monkey studies investigating both the amount of free and circulating IL-12 and changes in CD8+ T cell surface expression following administration of various doses of 02(VH6/VK3-D30E/N35Q)-IgG-IL12 (1×HC) (“hCD8IgG-IL12”). Where present, dotted lines represent the measurement taken prior to administration of the fusion construct. “Comp. MTD” indicates the known maximum tolerated dose of two known targeted IL-12 constructs, NHS-IL12 and AS1409. FIG. 33A is a bar graph depicting the percentage of CD8+ T cells with surface-bound IL-12 at 24 hours post-administration. FIG. 33B is a bar graph depicting the percentage of CD8+ T cells expressing Ki67 at 7 days post-administration. FIG. 33C is a bar graph depicting the percentage of CD8+ T cells expressing Perforin at 3 days post-administration. For each experiment summarized in FIGS. 33A-33C, n=1-2; data represent mean±SD. FIG. 33D depicts the percentage of CD8+ T cells with surface-bound IL-12 at the indicated timepoints following administration of 100 μg/kg 02(VH6/VK3-D30E/N35Q)-IgG-IL12 (1×HC). FIG. 33E depicts the serum concentration of IL-12 at the indicated timepoints as a percentage of the IL-12 concentration recorded 1 hour post-administration of the fusion construct (“Cmax”).

FIG. 34A-34B depict the changes in serum concentration of IFNγ in cynomolgus monkeys at various timepoints after intravenous (“IV”) or subcutaneous (“SC”) administration of the indicated dose of 02(VH6/VK3-D30E/N35Q)-IgG-IL12 (1×HC) (“hCD8IgG-IL12”). FIG. 34A depicts the results for each dose over time. FIG. 34B is a bar graph showing the serum concentration of IFNγ at 3 days post-administration of the indicated dose. n=2; mean±SD.

FIG. 35 is a stacked bar graph showing the percentage of Ki67-expressing CD8+ T cells in cynomolgus monkeys before and at various timepoints after administration of a 100 μg/kg dose of 02(VH6/VK3-D30E/N35Q)-IgG-IL12 (1×HC). Black area of bars corresponds to the percentage of naïve (CCR7+) Ki67+CD8+ T cells, and grey area of bars corresponds to the percentage of antigen-experienced (CCR7−) Ki67+CD8+ T cells. For the Day 7 timepoint, the percentage of antigen-experienced versus naïve cells is summarized as a pie chart. n=2; mean±SD.

FIG. 36A-36C depict the changes in body weight (FIG. 36A) and in serum levels of liver enzymes ALT (FIG. 36B) and AST (FIG. 36C) in cynomolgus monkeys following intravenous (“IV”) or subcutaneous (“SC”) administration of the indicated dose of 02(VH6/VK3-D30E/N35Q)-IgG-IL12 (1×HC).

FIG. 37 depicts tumor regression results in a MC38 syngeneic mouse tumor model following weekly administration of CD8IgG-IL12 (1×HC), twice weekly administration of an anti-PD-L1 antibody, both treatments in combination, or weekly administration of a vehicle control (HBSS). n=8, mean±SEM.

FIGS. 38A-38B shows the epitopes bound by anti-CD8 antibodies 02A01, 03A01, 05F10, 14E06, ch51-1, and MCD8.

### DETAILED DESCRIPTION

The present invention is based, in part, upon the development of fusion proteins (e.g., soluble fusion proteins) comprising an antigen-binding site that binds CD8 (e.g., human CD8 and/or cynomolgus CD8), an IL-12 protein, and optionally an antibody Fc domain. In certain embodiments, the antigen-binding site is silent, not substantially increasing or decreasing an activity of CD8 upon binding to a CD8 protein. The components of the fusion protein are coupled together in one or more configurations such that the resulting fusion proteins achieve favorable therapeutic efficacy and reduced toxicity relative to fusion proteins or unconjugated cytokines that lack an antigen-binding site disclosed herein. The fusion proteins described herein can be used to treat a variety of diseases and disorders, such as cancer, with improved safety and tolerability for higher doses.

### I. Definitions

To facilitate an understanding of the present invention, a number of terms and phrases are defined below.

The terms “a” and “an” as used herein mean “one or more” and include the plural unless the context is inappropriate.

As used herein, the term “antigen-binding site” refers to an antigen-binding fragment of an immunoglobulin or a derivative or variant thereof that participates in antigen binding. For example, in human antibodies, an antigen-binding site is formed by the N-terminal variable domains of the heavy chain and light chain, which are also called “heavy chain variable domain (VH)” and “light chain variable domain (VL),” respectively. In each of the variable domains, three highly divergent stretches called “hypervariable regions” are interposed between more conserved flanking stretches known as “framework regions.” The three hypervariable regions of a VH and the three hypervariable regions of a VL are disposed relative to each other in three-dimensional space to form an antigen-binding surface complementary to the three-dimensional surface of a bound antigen. The hypervariable regions are also referred to as “complementarity-determining regions” or “CDRs.” Examples of antigen-binding fragments of an immunoglobulin, include, for example, Fab, Fab′, and F(ab′)₂ fragments. Examples of variants of antigen-binding fragments of immunoglobulins include, for example, single chain antibodies or scFvs. Certain animals have different forms of antibodies. For example, camelids have antibodies comprising V_{H}H fragments and cartilaginous fishes have antibodies called “new antigen receptor immunoglobulins” or “IgNARs” comprising V_{NAR} fragments, where such fragments, which are single, monomeric antibody variable domains that are able to bind selectively to a specific antigen independently of another variable domain, are called “single domain antibody,” “sdAb,” or “nanobody.” An antigen-binding site can comprise either a pair of VH and VL or an sdAb. The antigen-binding site disclosed herein can be recombinant, chimeric, deimmunized, humanized, and/or affinity matured (see, e.g., U.S. Pat. No. 4,816,567; Morrison et al. (1984) Proc. Natl. Acad. Sci. U.S.A., 81: 6851-55; Morrison et al. (1985) Proc. Natl. Acad. Sci. U.S.A., 81:6851; Takeda et al. (1985) Nature, 314: 452).

As used herein, the term “CD8-binding site” refers to an antigen-binding site, as defined herein, that binds CD8 (e.g., human CD8).

The term “silent,” as used herein in the context of a CD8-binding site, refers to a CD8-binding site that does not substantially change the function or activity of CD8 upon binding to the CD8. In certain embodiments, the CD8-binding site lacks substantial agonistic activity or substantial antagonistic activity upon binding CD8. In certain embodiments, the antigen-binding site does not increase an activity of CD8 by more than 10%, 20%, 30%, 40%, 50%, or 60% and/or does not decrease an activity of CD8 by more than 10%, 20%, 30%, 40%, 50%, or 60% In certain embodiments, the antigen-binding site does not increase an activity of CD8 by more than 1.1 fold, 1.2 fold, 1.3 fold, 1.4 fold, 1.5 fold, 1.6 fold, 1.7 fold, 1.8 fold, 1.9 fold, or 2 fold and/or the does not decrease an activity of CD8 by more than 1.1 fold, 1.2 fold, 1.3 fold, 1.4 fold, 1.5 fold, 1.6 fold, 1.7 fold, 1.8 fold, 1.9 fold, or 2 fold. The activity of CD8 can be stabilization of a peptide-MHC-TCR complex, activation of a TCR signaling pathway, and/or activation of a transcription factor downstream of TCR signaling, as measured by the assays disclosed herein (see, e.g., the Examples section below).

The term “cross-compete,” as used herein in the context of a subject antibody and a reference antibody, indicates that the subject antibody competes for binding to an antigen (e.g., CD8) with the reference antibody and vice versa. In an exemplary assay, a first anti-CD8 antibody is immobilized on a solid surface, CD8αβ is bound to the first antibody, and binding of a second anti-CD8 antibody is assessed. If the second antibody does not generate a significant binding signal (e.g., resonance unit does not increase by at least 25%, 50%, or 2-fold), the second antibody competes with the first antibody for binding CD8 (see, e.g., Example 1 below). A subject antibody cross-competes with a reference antibody if competition is observed whether the reference antibody is used as the first antibody and the subject antibody is used as the second antibody, or the subject antibody is used as the first antibody and the reference antibody is used as the second antibody in this assay. A skilled artisan can select the concentrations of the antibodies used in the competition assays based on the affinities of the antibodies for the antigen and the valency of the antibodies. Exemplary assays are described in Cox et al., “Immunoassay Methods,” in ASSAY GUIDANCE MANUAL [INTERNET], Updated Dec. 24, 2014 (www.ncbi.nlm.nih.gov/books/NBK92434/; accessed Sep. 29, 2015); Silman et al. (2001) CYTOMETRY, 44: 30-37; and Finco et al. (2011) J. PHARM. BIOMED. ANAL., 54: 351-358.

As used herein, the term “tumor-associated antigen” refers to any antigen, including but not limited to a protein, glycoprotein, ganglioside, carbohydrate, or lipid, that is expressed or otherwise present on the surface of a malignant cell, or present in the tumor microenvironment (e.g., on the surface of tumor-associated blood vessels, mesenchymal stroma, or immune infiltrates, or in an extracellular matrix).

As used herein, the terms “subject” and “patient” are used interchangeably and refer to an organism to be treated by the methods and compositions described herein. Such organisms preferably include, but are not limited to, mammals (e.g., murines, simians, equines, bovines, porcines, canines, felines, and the like), and more preferably include humans.

As used herein, the term “effective amount” refers to the amount of a compound (e.g., a compound of the present disclosure) sufficient to effect beneficial or desired results. An effective amount can be administered in one or more administrations, applications or dosages and is not intended to be limited to a particular formulation or administration route.

As used herein, the term “treating” includes any effect, e.g., lessening, reducing, modulating, ameliorating or eliminating, that results in the improvement of the condition, disease, disorder, and the like, or ameliorating a symptom thereof.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see e.g., Adeboye Adejare, Remington: The Science and Practice of Pharmacy (23d ed. 2020).

As used herein, percent “identity” between a polypeptide sequence and a reference sequence is defined as the percentage of amino acid residues in the polypeptide sequence that are identical to the amino acid residues in the reference sequence, after aligning the sequences and introducing gaps, if necessary, to achieve the maximum percent sequence identity. Alignment for purposes of determining percent amino acid sequence identity can be achieved in various ways that are within the skill in the art, for instance, using publicly available computer software such as BLAST, BLAST-2, ALIGN, MEGALIGN (DNASTAR), CLUSTALW, CLUSTAL OMEGA, or MUSCLE software. Those skilled in the art can determine appropriate parameters for aligning sequences, including any algorithms needed to achieve maximal alignment over the full length of the sequences being compared.

As used herein, the expression “and/or” in connection with two or more recited objects includes individually each of the recited objects and the various combinations of two or more of the recited objects, unless otherwise understood from the context and use.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a ±10% variation from the nominal value unless otherwise indicated or inferred.

As a general matter, compositions specifying a percentage are by weight unless otherwise specified. Further, if a variable is not accompanied by a definition, then the previous definition of the variable controls.

### II. Fusion Proteins

In one aspect, the present disclosure provides a fusion protein (e.g., immunostimulatory fusion protein) comprising a CD8-binding site and an IL-12 protein. The CD8-binding site is believed to specifically deliver and/or increase the concentration of the IL-12 protein to the surface of a CD8⁺ immune cell (e.g., CD8⁺ T cell), thereby increasing CD8-targeted localization or distribution and/or enhancing the cell surface availability of the IL-12 protein. As a result, the fusion protein can provide localized and prolonged immunostimulation of CD8⁺ T cells, thereby to induce controlled expansion and/or activation of an immune response. It has been known that IL-12 also stimulate CD4⁺ T cells, regulatory T (Treg) cells, and/or natural killer (NK) cells. The selective targeting of the fusion protein to CD8⁺ T cells may improve the overall immune response, as activation of Treg cells could lead to immunosuppression and activation of NK cells could reduce tolerability of the immunostimulator. In certain embodiments, the fusion with the CD8-binding site does not substantially interfere with the signaling function of the IL-12 protein.

IL-12 includes two subunits called IL-12A (p35) and IL-12B (p40). These two subunits can be linked by a peptide linker to form a single chain IL-12 (see, e.g., U.S. Pat. No. 7,226,998). In certain embodiments, IL-12A is linked to the N-terminus of IL-12B. In other embodiments, IL-12B is linked to the N-terminus of IL-12A. Peptide linkers having proper length and flexibility can be selected such that the single-chain IL-12 has at least 10%, at least 20%, at least 30%, at least 40%, at least 50%, at least 60%, at least 70%, at least 80%, at least 90%, or at least 100% of the activity of a corresponding wild-type IL-12. In certain embodiments, the peptide linker comprises an amino acid sequence from Table 6 below. In certain embodiments, the peptide linker comprises the amino acid sequence of SEQ ID NO: 85.

Exemplary IL-12 amino acid sequences are provided in Table 1 below.

TABLE 1
Exemplary IL-12 Proteins

| Protein Name | Amino Acid Sequence |
|---|---|
| Human IL-12A | RNLPVATPDPGMFPCLHHSQNLLRAVSNMLQKARQTLEFYPCTSEEIDHE |
| (p35) | DITKDKTSTVEACLPLELTKNESCLNSRETSFITNGSCLASRKTSFMMALC |
| | LSSIYEDLKMYQVEFKTMNAKLLMDPKRQIFLDQNMLAVIDELMQALNF |
| | NSETVPQKSSLEEPDFYKTKIKLCILLHAFRIRAVTIDRVMSYLNAS |
| | (SEQ ID NO: 89) |
| Human IL-12B | IWELKKDVYVVELDWYPDAPGEMVVLTCDTPEEDGITWTLDQSSEVLGS |
| (p40) | GKTLTIQVKEFGDAGQYTCHKGGEVLSHSLLLLHKKEDGIWSTDILKDQ |
| | KEPKNKTFLRCEAKNYSGRFTCWWLTTISTDLTFSVKSSRGSSDPQGVTC |
| | GAATLSAERVRGDNKEYEYSVECQEDSACPAAEESLPIEVMVDAVHKLK |
| | YENYTSSFFIRDIIKPDPPKNLQLKPLKNSRQVEVSWEYPDTWSTPHSYFS |
| | LTFCVQVQGKSKREKKDRVFTDKTSATVICRKNASISVRAQDRYYSSSW |
| | SEWASVPCS (SEQ ID NO: 90) |
| Human single- | IWELKKDVYVVELDWYPDAPGEMVVLTCDTPEEDGITWTLDQSSEVLGS |
| chain IL-12 | GKTLTIQVKEFGDAGQYTCHKGGEVLSHSLLLLHKKEDGIWSTDILKDQ |
| (B-A) | KEPKNKTFLRCEAKNYSGRFTCWWLTTISTDLTFSVKSSRGSSDPQGVTC |
| | GAATLSAERVRGDNKEYEYSVECQEDSACPAAEESLPIEVMVDAVHKLK |
| | YENYTSSFFIRDIIKPDPPKNLQLKPLKNSRQVEVSWEYPDTWSTPHSYFS |
| | LTFCVQVQGKSKREKKDRVFTDKTSATVICRKNASISVRAQDRYYSSSW |
| | SEWASVPCS |
| | GGGSGGGSGGGSGGGS |
| | RNLPVATPDPGMFPCLHHSQNLLRAVSNMLQKARQTLEFYPCTSEEIDHE |
| | DITKDKTSTVEACLPLELTKNESCLNSRETSFITNGSCLASRKTSFMMALC |
| | LSSIYEDLKMYQVEFKTMNAKLLMDPKRQIFLDQNMLAVIDELMQALNF |
| | NSETVPQKSSLEEPDFYKTKIKLCILLHAFRIRAVTIDRVMSYLNAS |
| | (SEQ ID NO: 91) |
| Human single- | RNLPVATPDPGMFPCLHHSQNLLRAVSNMLQKARQTLEFYPCTSEEIDHE |
| chain IL-12 | DITKDKTSTVEACLPLELTKNESCLNSRETSFITNGSCLASRKTSFMMALC |
| (A-B) | LSSIYEDLKMYQVEFKTMNAKLLMDPKRQIFLDQNMLAVIDELMQALNF |
| | NSETVPQKSSLEEPDFYKTKIKLCILLHAFRIRAVTIDRVMSYLNAS |
| | GGGSGGGSGGGSGGGS |
| | IWELKKDVYVVELDWYPDAPGEMVVLTCDTPEEDGITWTLDQSSEVLGS |
| | GKTLTIQVKEFGDAGQYTCHKGGEVLSHSLLLLHKKEDGIWSTDILKDQ |
| | KEPKNKTFLRCEAKNYSGRFTCWWLTTISTDLTFSVKSSRGSSDPQGVTC |
| | GAATLSAERVRGDNKEYEYSVECQEDSACPAAEESLPIEVMVDAVHKLK |
| | YENYTSSFFIRDIIKPDPPKNLQLKPLKNSRQVEVSWEYPDTWSTPHSYFS |
| | LTFCVQVQGKSKREKKDRVFTDKTSATVICRKNASISVRAQDRYYSSSW |
| | SEWASVPCS |
| | (SEQ ID NO: 92) |

In certain embodiments, the fusion protein comprises an IL-12 protein comprising an amino acid sequence at least 80%, at least 85%, at least 90%, at least 91%, at least 92%, at least 93%, at least 94%, at least 95%, at least 96%, at least 97%, at least 98%, or at least 99% identical to SEQ ID NO: 89 and an amino acid sequence at least 80%, at least 85%, at least 90%, at least 91%, at least 92%, at least 93%, at least 94%, at least 95%, at least 96%, at least 97%, at least 98%, or at least 99% identical to SEQ ID NO: 90. In certain embodiments, the IL-12 protein comprises the amino acid sequences of SEQ ID NOs: 89 and 90. In certain embodiments, the immunostimulatory cytokine is a single chain IL-12 comprising an amino acid sequence at least 80%, at least 85%, at least 90%, at least 91%, at least 92%, at least 93%, at least 94%, at least 95%, at least 96%, at least 97%, at least 98%, or at least 99% identical to SEQ ID NO: 91 or 92. In certain embodiments, the immunostimulatory cytokine is a single chain IL-12 comprising the amino acid sequence of SEQ ID NO: 91 or 92.

In certain embodiments, the fusion protein comprises an IL-12 protein that has one or more mutations relative to the corresponding wild-type IL-12 (e.g., human IL-12). For example, in certain embodiments, the IL-12 protein comprises a mutation that reduces affinity for an IL-12 receptor. In certain embodiments, the IL-12 protein comprises an IL-12B (p40) subunit comprising one more mutations that reduce affinity for IL-12Rβ1 (see, e.g., Glassman et al. (2021) Cell 184:983-99). Without wishing to be bound by theory, it has been contemplated that CD8⁺ T cells, after stimulation (e.g., via TCR), upregulates IL-12Rβ1 expression to increase sensitivity to IL-12. An IL-12 variant with reduced affinity to IL-12Rβ1 may still be adequate to induce signaling in stimulated CD8⁺ T cells, but its ability to induce signaling in other cells, such as NK cells, would be more significantly impaired. As a result, such IL-12 variant preferentially targets stimulated CD8⁺ T cells over NK cells. Moreover, when fused with a CD8-binding domain, it is contemplated that an IL-12 variant having reduced affinity to one or more IL-12 receptors, or one or more subunits thereof, may still be adequate to induce signaling in CD8⁺ T cells given the enrichment of the fusion protein on the cell surface, but its ability to induce signaling in CD8-negative cells would be more significantly impaired. Therefore, such fusion protein may exhibit a reduced level of NK cell targeting via binding to an IL-12 receptor on NK cells, thereby to reduce NK cell-mediated toxicity. In certain embodiments, an IL-12 variant (e.g., murine IL-12 variant) disclosed herein comprises a p40 subunit comprising one or more mutations selected from E81A, F82A, K106A, and K217A, wherein the aforementioned amino acid positions correspond to the full-length sequence of murine IL-12 p40 subunit including its signal peptide. In certain embodiments, an IL-12 variant (e.g., murine IL-12 variant) disclosed herein comprises a p40 subunit comprising a set of mutations selected from the group consisting of. E81A and F82A; E81A, F82A, and K106A; and E81A, F82A, K106A, and K217A. In certain embodiments, an IL-12 variant (e.g., human IL-12 variant) disclosed herein comprises a p40 subunit comprising one or more mutations selected from E81A, F82A, K106A, and K219A, wherein the aforementioned amino acid positions correspond to the full-length sequence of human IL-12 p40 subunit including its signal peptide. In certain embodiments, an IL-12 variant (e.g., human IL-12 variant) disclosed herein comprises a p40 subunit comprising a set of mutations selected from the group consisting of. E81A and F82A; E81A, F82A, and K106A; and E81A, F82A, K106A, and K219A.

In certain embodiments, the fusion protein further comprises one or more immunostimulators, for example, immunostimulatory cytokines, agonists of costimulatory molecules, and/or inhibitors of immune checkpoint proteins. In certain embodiments, the immunostimulator comprises an immunostimulatory cytokine, for example, IL-15, IL-2, IL-6, IL-7, IL-18, IL-21, IL-23, IL-27, IL-4, IL-9, IFN-α, IFN-β, IFN-γ, or IL-12. In certain embodiments, the immunostimulator comprises an agonist (e.g., agonistic antibody or agonistic ligand) of a costimulatory molecule, for example, CD137, OX40, CD28, GITR, VISTA, anti-CD40, or CD3. In certain embodiments, the immunostimulator comprises an inhibitor (e.g., antagonistic antibody or neutralizing antibody) of an immune checkpoint protein, for example, PD-1, PD-L1, LAG-3, TIM-3, or CTLA-4.

It is understood that in an immunostimulatory fusion protein, it is undesirable to use an antagonistic CD8-binding site, as the antigen-binding site would inhibit a CD8 activity. On the other hand, it is also undesirable to use an agonistic CD8-binding site, as the antigen-binding site may increase a CD8 activity at a basal level, thereby increasing toxicity of the IL-12 protein. Accordingly, in certain embodiments, the fusion protein comprises a silent antigen-binding site that binds CD8 (e.g., a CD8-binding site disclosed herein) and an IL-12 protein. It is contemplated that silent CD8-binding sites can have one or more of the features described in Section III below.

It is also understood that the immunostimulatory fusion proteins disclosed herein are designed to maintain or enhance CD8⁺ T cells. As a result, it is undesirable to include a moiety that may engage immune effector cells (e.g., another T cell or an NK cell) which could have a cytotoxic effect on the CD8⁺ T cells by antibody-dependent cellular cytotoxicity (ADCC) and/or complement-dependent cytotoxicity (CDC). Accordingly, in certain embodiments, an immunostimulatory fusion protein lacks an antibody Fc domain having ADCC effector function. In certain embodiments, an immunostimulatory fusion protein lacks an antibody Fc domain having CDC effector function. In certain embodiments, the immunostimulatory fusion protein lacks an antibody Fc domain. In certain embodiments, the immunostimulatory fusion protein comprises an antibody Fc domain or a fragment thereof in which an ADCC effector function is reduced by at least 50%, at least 60%, at least 70%, at least 80%, at least 90%, at least 91%, at least 92%, at least 93%, at least 94%, at least 95%, at least 96%, at least 97%, at least 98%, or at least 99% relative to a wild-type human IgG1 Fc domain having the amino acid sequence of DKTHTCPPCPAPELLGGPSVFLFPPKPKDTLMISRTPEVTCVVVDVSHEDPEVKFNWY VDGVEVHNAKTKPREEQYNSTYRVVSVLTVLHQDWLNGKEYKCKVSNKALPAPIE KTISKAKGQPREPQVYTLPPSRDELTKNQVSLTCLVKGFYPSDIAVEWESNGQPENN YKTTPPVLDSDGSFFLYSKLTVDKSRWQQGNVFSCSVMHEALHNHYTQKSLSLSPG (SEQ ID NO: 84). In certain embodiments, the immunostimulatory fusion protein comprises an antibody Fc domain or a fragment thereof in which a CDC effector function is reduced by at least 50%, at least 60%, at least 70%, at least 80%, at least 90%, at least 91%, at least 92%, at least 93%, at least 94%, at least 95%, at least 96%, at least 97%, at least 98%, or at least 99% relative to a wild-type human IgG1 Fc domain having the amino acid sequence of SEQ ID NO: 84.

In certain embodiments, the present disclosure provides an immunostimulatory fusion protein comprising an antigen-binding site that binds CD8 and an IL-12 protein, wherein the antigen-binding site does not substantially increase or decrease an activity of CD8, and the immunostimulatory fusion protein lacks an antibody Fc domain or comprises an antibody Fc domain in which an ADCC effector function is reduced by at least 60%, at least 70%, at least 80%, at least 90%, at least 91%, at least 92%, at least 93%, at least 94%, at least 95%, at least 96%, at least 97%, at least 98%, or at least 99%, relative to a wild-type human IgG1 Fc domain having the amino acid sequence of SEQ ID NO: 84.

In certain embodiments, the immunostimulatory fusion protein promotes survival, proliferation, activation, and/or memory formation, and/or inhibits exhaustion of CD8⁺ T cells in vivo or ex vivo. The activity of a fusion protein disclosed herein can be assessed by a marker responsive to IL-12, such as STAT4 phosphorylation. In certain embodiments, the activity of the fusion protein is represented by the EC₅₀ value (half maximal effective concentration) of STAT4 phosphorylation. In certain embodiments, the EC₅₀ value of the fusion protein on CD8⁺ T cells is at least 5-fold, at least 6-fold, at least 7-fold, at least 8-fold, at least 9-fold, at least 10-fold, at least 11-fold, at least 12-fold, at least 13-fold, at least 14-fold, or at least 15-fold lower than the EC₅₀ value of the corresponding IL-12 protein without a CD8-binding site. In certain embodiments, the EC₅₀ value of the fusion protein on CD8⁺ T cells is at least 2-fold, at least 3-fold, at least 4-fold, at least 5-fold, at least 6-fold, at least 7-fold, at least 8-fold, at least 9-fold, or at least 10-fold lower than the EC₅₀ value of the same fusion protein on NK cells or CD4⁺ T cells.

The activity of a fusion protein can also be assessed in an in vitro cytotoxicity assay. Stimulated enriched CD8⁺ T cells or unstimulated peripheral blood mononuclear cells (PBMC) can be used as effector cells. The effector cells are incubated with target cells that express or otherwise display an antigen (e.g., a T cell epitope presented by a MHC) that is recognized by the effector cells. The effector to target cell (E:T) ratio is usually about 1:1 to 10:1, for example, about 1:1 to 5:1 in a long-duration (24 hours to 4 days) assay and about 5:1 to 10:1 in a short-duration (4-6 hours) assay, but can vary. Killing of the target cells can be measured in a ⁵¹Cr-release assay or in a FACS-based cytotoxicity assay. Other methods of measuring cell death are well-known to the skilled person, such as MTT or MTS assays, ATP-based assays including bioluminescent assays, the sulforhodamine B (SRB) assay, WST assay, clonogenic assay and the ECIS technology. In certain embodiments, in a cytotoxicity assay described above, the percentage of target cells killed in the presence of a fusion protein disclosed herein is at least 10%, at least 20%, at least 30%, at least 40%, or at least 50% greater (absolute difference) than that in the presence of the corresponding IL-12 protein at the same molar concentration.

According to the present disclosure, the fusion proteins and its constituent binding domains are in the form of one or more polypeptides. Such polypeptides may also include non-proteinaceous parts (e.g., chemical linkers or chemical cross-linking agents).

### III. CD8-Binding Sites

In certain embodiments, the fusion protein disclosed herein comprises a silent antigen-binding site that binds CD8 (e.g., human CD8 and/or cynomolgus CD8). Tables 2A-4B below list VH, VL, and CDR sequences of the antigen-binding sites. The CDR sequences are identified under the Kabat numbering scheme (Tables 2A, 3A, and 4A) or under the Chothia numbering scheme (Tables 2B, 3B, and 4B). In Tables 2A-2B and Tables 3A-3B, any of the humanized VH sequences can pair with any of the humanized VL sequences in the same table to form a C1D8-binding site.

TABLE 2A
Exemplary silent CD8 antigen-binding sites derived from 02A01 (Kabat)

| | Heavy Chain Variable Domain | Light Chain Variable Domain |
|---|---|---|
| Clone | (VH) | (VL) |
| Murine | EVQLQQSGPELVKPGASLKISC | DIVLTQSPAFLAVSLGQRATISCRA |
| 02A01 | KASGYTFTDFTMHWVKQSHG | SESVDSYDINSMHWYQQKPGQPP |
| | KSLEWIGLVNPNNGGNIYNQN | KLLIYRASHLQSGIPARFSGSGSRT |
| | FKGKATLTVDTSSSTAYMELRS | DFTLTINFVEADDVATYYCQQSNE |
| | LTSEDSSVYYCGRVPLYTSHG | DPLTFGAGTKLELK (SEQ ID NO: 5) |
| | MDYWGQGTSVTVSS (SEQ ID | LCDR1-RASESVDSYDINSMH |
| | NO: 1) | (SEQ ID NO: 6) |
| | HCDR1-DFTMH (SEQ ID NO: 2) | LCDR2-RASHLQS (SEQ ID NO: 7) |
| | HCDR2- | LCDR3-QQSNEDPLT (SEQ ID |
| | LVNPNNGGNIYNQNFKG (SEQ | NO: 8) |
| | ID NO: 3) | |
| | HCDR3-VPLYTSHGMDY (SEQ | |
| | ID NO: 4) | |
| Murine | EVQLQQSGPELVKPGASLKISC | DIVLTQSPAFLAVSLGQRATISCRA |
| 02A01 | KASGYTFTDFTLHWVKQSHGK | SESVDSYDINSMHWYQQKPGQPP |
| VH-M39L | SLEWIGLVNPNNGGNIYNQNF | KLLIYRASHLQSGIPARFSGSGSRT |
| | KGKATLTVDTSSSTAYMELRS | DFTLTINFVEADDVATYYCQQSNE |
| | LTSEDSSVYYCGRVPLYTSHG | DPLTFGAGTKLELK (SEQ ID NO: 5) |
| | MDYWGQGTSVTVSS (SEQ ID | LCDR1-RASESVDSYDINSMH |
| | NO: 9) | (SEQ ID NO: 6) |
| | HCDR1-DFTLH (SEQ ID | LCDR2-RASHLQS (SEQ ID NO: 7) |
| | NO: 10) | LCDR3-QQSNEDPLT (SEQ ID |
| | HCDR2- | NO: 8) |
| | LVNPNNGGNIYNQNFKG (SEQ | |
| | ID NO: 3) | |
| | HCDR3-VPLYTSHGMDY (SEQ | |
| | ID NO: 4) | |
| 02A01 | HCDR1-DFTXH, wherein X is M | LCDR1-RASESVX₁SYDIX₂SMH, |
| Consensus | or L (SEQ ID NO: 88) | wherein X₁ is D or E; and X₂ is N or Q |
| 1¹ | HCDR2- | (SEQ ID NO: 109) |
| | LVNPNNGGNIYX₁X₂X₃FX₄G, | LCDR2-X₁ASX₂X₃X₄X₅, wherein |
| | wherein X₁ is N or A; X₂ is Q or E; | X₁ is R or D; X₂ is H or N; X₃ is L or |
| | X₃ is N or K; and X₄ is K or Q | R; X₄ is Q or A; and X₅ is S or T (SEQ |
| | (SEQ ID NO: 11) | ID NO: 12) |
| | HCDR3-VPLYTSHGMDY (SEQ | LCDR3-QQSNEDPLT (SEQ ID |
| | ID NO: 4) | NO: 8) |
| 02A01 | HCDR1-DFTMH (SEQ ID NO: 2) | LCDR1-RASESVX₁SYDIX₂SMH, |
| Consensus | HCDR2- | wherein X₁ is D or E; and X₂ is N or Q |
| 2² | LVNPNNGGNIYX₁X₂X₃FX₄G, | (SEQ ID NO: 109) |
| | wherein X₁, X₂, X₃, and X₄ are A, | LCDR2-X₁ASX₂X₃X₄X₅, wherein |
| | E, K, and Q, respectively; or X₁, | X₁, X₂, X₃, X₄, and X₅ are R, H, L, Q, |
| | X₂, X₃, and X₄ are N, Q, N, and K, | and S, respectively; or X₁, X₂, X₃, X₄, |
| | respectively (SEQ ID NO: 13) | and X₅ are D, N, R, A, and T, |
| | HCDR3-VPLYTSHGMDY (SEQ | respectively (SEQ ID NO: 84) |
| | ID NO: 4) | LCDR3-QQSNEDPLT (SEQ ID |
| | | NO: 8) |
| Humanized | EVQLVQSGPEVVKPGATLKISC | |
| 02A01- | KASGYTFTDFTMHWVKQAPG | |
| VH1 | KSLEWIGLVNPNNGGNIYNQN | |
| | FKGRATLTVDTSTSTAYMELSS | |
| | LRSEDTSVYYCGRVPLYTSHG | |
| | MDYWGQGTLVTVSS (SEQ ID | |
| | NO: 14) | |
| | HCDR1-DFTMH (SEQ ID NO: 2) | |
| | HCDR2- | |
| | LVNPNNGGNIYNQNFKG (SEQ | |
| | ID NO: 3) | |
| | HCDR3-VPLYTSHGMDY (SEQ | |
| | ID NO: 4) | |
| Humanized | EVQLVQSGAEVKKPGATLKISC | |
| 02A01- | KASGYTFTDFTMHWVQQAPG | |
| VH2 | KGLEWIGLVNPNNGGNIYNQN | |
| | FKGRATLTVDTSTSTAYMELSS | |
| | LRSEDTSVYYCGRVPLYTSHG | |
| | MDYWGQGTLVTVSS (SEQ ID | |
| | NO: 15) | |
| | HCDR1-DFTMH (SEQ ID NO: 2) | |
| | HCDR2- | |
| | LVNPNNGGNIYNQNFKG (SEQ | |
| | ID NO: 3) | |
| | HCDR3-VPLYTSHGMDY (SEQ | |
| | ID NO: 4) | |
| Humanized | EVQLVQSGAEVKKPGATLKISC | |
| 02A01- | KASGYTFTDFTMHWVQQAPG | |
| VH3 | KGLEWIGLVNPNNGGNIYAEK | |
| | FQGRATLTVDTSTSTAYMELSS | |
| | LRSEDTSVYYCGRVPLYTSHG | |
| | MDYWGQGTLVTVSS (SEQ ID | |
| | NO: 16) | |
| | HCDR1-DFTMH (SEQ ID NO: 2) | |
| | HCDR2- | |
| | LVNPNNGGNIYAEKFQG (SEQ | |
| | ID NO: 17) | |
| | HCDR3-VPLYTSHGMDY (SEQ | |
| | ID NO: 4) | |
| Humanized | EVQLVQSGAEVKKPGATVKIS | |
| 02A01- | CKASGYTFTDFTMHWVQQAP | |
| VH4 | GKGLEWIGLVNPNNGGNIYAE | |
| | KFQGRATLTVDTSTSTAYMEL | |
| | SSLRSEDTAVYYCGRVPLYTSH | |
| | GMDYWGQGTLVTVSS (SEQ ID | |
| | NO: 18) | |
| | HCDR1-DFTMH (SEQ ID NO: 2) | |
| | HCDR2- | |
| | LVNPNNGGNIYAEKFQG (SEQ | |
| | ID NO: 17) | |
| | HCDR3-VPLYTSHGMDY (SEQ | |
| | ID NO: 4) | |
| Humanized | EVQLVQSGAEVKKPGATLKISC | |
| 02A01- | KASGYTFTDYYMHWVQQAPG | |
| VH5 | KGLEWIGLVNPNNGGNIYNQN | |
| | FKGRATLTVDTSTSTAYMELSS | |
| | LRSEDTSVYYCGRVPLYTSHG | |
| | MDYWGQGTLVTVSS (SEQ ID | |
| | NO: 19) | |
| | HCDR1-DYYMH (SEQ ID | |
| | NO: 20) | |
| | HCDR2- | |
| | LVNPNNGGNIYNQNFKG (SEQ | |
| | ID NO: 3) | |
| | HCDR3-VPLYTSHGMDY (SEQ | |
| | ID NO: 4) | |
| Humanized | EVQLVQSGAEVKKPGATVKIS | |
| 02A01- | CKASGYTFTDFTMHWVQQAP | |
| VH6 | GKGLEWIGLVNPNNGGNIYAE | |
| | KFQGRVTLTVDTSTSTAYMEL | |
| | SSLRSEDTAVYYCGRVPLYTSH | |
| | GMDYWGQGTLVTVSS (SEQ ID | |
| | NO: 21) | |
| | HCDR1-DFTMH (SEQ ID NO: 2) | |
| | HCDR2- | |
| | LVNPNNGGNIYAEKFQG (SEQ | |
| | ID NO: 17) | |
| | HCDR3-VPLYTSHGMDY (SEQ | |
| | ID NO: 4) | |
| Humanized | | EIVLTQSPATLSVSPGERATISCRA |
| 02A01- | | SESVDSYDINSMHWYQQKPGQPP |
| VK1 | | RLLIYRASHLQSGIPARFSGSGSRT |
| | | DFTLTISSVEPEDFATYYCQQSNE |
| | | DPLTFGQGTKLEIK (SEQ ID |
| | | NO: 22) |
| | | LCDR1-RASESVDSYDINSMH |
| | | (SEQ ID NO: 6) |
| | | LCDR2-RASHLQS (SEQ ID NO: 7) |
| | | LCDR3-QQSNEDPLT (SEQ ID |
| | | NO: 8) |
| Humanized | | EIVLTQSPATLSVSPGERATISCRA |
| 02A01- | | SESVDSYDINSMHWYQQKPGQAP |
| VK2 | | RLLIYRASHLQSGIPARFSGSGSRT |
| | | DFTLTISSVEPEDFATYYCQQSNE |
| | | DPLTFGQGTKLEIK (SEQ ID |
| | | NO: 23) |
| | | LCDR1-RASESVDSYDINSMH |
| | | (SEQ ID NO: 6) |
| | | LCDR2-RASHLQS (SEQ ID NO: 7) |
| | | LCDR3-QQSNEDPLT (SEQ ID |
| | | NO: 8) |
| Humanized | | EIVLTQSPATLSLSPGERATISCRA |
| 02A01- | | SESVDSYDINSMHWYQQKPGQAP |
| VK3 | | RLLIYRASHLQSGIPARFSGSGSRT |
| | | DFTLTISSLEPEDFATYYCQQSNED |
| | | PLTFGQGTKLEIK (SEQ ID NO: 24) |
| | | LCDR1-RASESVDSYDINSMH |
| | | (SEQ ID NO: 6) |
| | | LCDR2-RASHLQS (SEQ ID NO: 7) |
| | | LCDR3-QQSNEDPLT (SEQ ID |
| | | NO: 8) |
| Humanized | | EIVLTQSPATLSLSPGERATISCRA |
| 02A01- | | SESVESYDIQSMHWYQQKPGQAP |
| VK3 | | RLLIYRASHLQSGIPARFSGSGSRT |
| (D30E, | | DFTLTISSLEPEDFATYYCQQSNED |
| N35Q) | | PLTFGQGTKLEIK (SEQ ID NO: 105) |
| | | LCDR1-RASESVESYDIQSMH |
| | | (SEQ ID NO: 106) |
| | | LCDR2-RASHLQS (SEQ ID NO: 7) |
| | | LCDR3-QQSNEDPLT (SEQ ID |
| | | NO: 8) |
| Humanized | | EIVLTQSPATLSVSPGERATISCRA |
| 02A01- | | SESVDSYDINSMHWYQQKPGQAP |
| VK4 | | RLLIYDASNRATGIPARFSGSGSRT |
| | | DFTLTISSVEPEDFATYYCQQSNE |
| | | DPLTFGQGTKLEIK (SEQ ID |
| | | NO: 25) |
| | | LCDR1-RASESVDSYDINSMH |
| | | (SEQ ID NO: 6) |
| | | LCDR2-DASNRAT (SEQ ID |
| | | NO: 26) |
| | | LCDR3-QQSNEDPLT (SEQ ID |
| | | NO: 8) |

¹02A01 consensus 1 is based on the Kabat CDR sequences of: (a) the heavy chain CDRs of murine 02A01, murine 02A01 VH-M39L, humanized 02A01-VH1, humanized 02A01-VH2, humanized 02A01-VH3, humanized 02A01-VH4, and humanized 02A01-VH6; and (b) the light chain CDRs of murine 02A01, murine 02A01 VH-M39L, humanized 02A01-VK1, humanized 02A01-VK2, humanized 02A01-VK3, humanized 02A01-VK3(D30E/N35Q), and humanized 02A01-VK4.
²02A01 consensus 2 is based on the Kabat CDR sequences of: (a) the heavy chain CDRs of humanized 02A01-VH1, humanized 02A01-VH2, humanized 02A01-VH3, humanized 02A01-VH4, and humanized 02A01-VH6; and (b) the light chain CDRs of humanized 02A01-VK1, humanized 02A01-VK2, humanized 02A01-VK3, humanized 02A01-VK3(D30E/N35Q), and humanized 02A01-VK4.

TABLE 2B
Exemplary silent CD8 antigen-binding sites derived from 02A01 (Chothia)

| | Heavy Chain Variable Domain | Light Chain Variable Domain |
|---|---|---|
| Clone | (VH) | (VL) |
| Murine | EVQLQQSGPELVKPGASLKISC | DIVLTQSPAFLAVSLGQRATISCRA |
| 02A01 | KASGYTFTDFTMHWVKQSHG | SESVDSYDINSMHWYQQKPGQPP |
| | KSLEWIGLVNPNNGGNIYNQN | KLLIYRASHLQSGIPARFSGSGSRT |
| | FKGKATLTVDTSSSTAYMELRS | DFTLTINFVEADDVATYYCQQSNE |
| | LTSEDSSVYYCGRVPLYTSHG | DPLTFGAGTKLELK (SEQ ID NO: 5) |
| | MDYWGQGTSVTVSS (SEQ ID | LCDR1-RASESVDSYDINSMH |
| | NO: 1) | (SEQ ID NO: 6) |
| | HCDR1-GYTFTDF (SEQ ID | LCDR2-RASHLQS (SEQ ID NO: 7) |
| | NO: 112) | LCDR3-QQSNEDPLT (SEQ ID |
| | HCDR2-NPNNGG (SEQ ID | NO: 8) |
| | NO: 113) | |
| | HCDR3-VPLYTSHGMDY (SEQ | |
| | ID NO: 4) | |
| Murine | EVQLQQSGPELVKPGASLKISC | DIVLTQSPAFLAVSLGQRATISCRA |
| 02A01 | KASGYTFTDFTLHWVKQSHGK | SESVDSYDINSMHWYQQKPGQPP |
| VH-M39L | SLEWIGLVNPNNGGNIYNQNF | KLLIYRASHLQSGIPARFSGSGSRT |
| | KGKATLTVDTSSSTAYMELRS | DFTLTINFVEADDVATYYCQQSNE |
| | LTSEDSSVYYCGRVPLYTSHG | DPLTFGAGTKLELK (SEQ ID NO: 5) |
| | MDYWGQGTSVTVSS (SEQ ID | LCDR1-RASESVDSYDINSMH |
| | NO: 9) | (SEQ ID NO: 6) |
| | HCDR1-GYTFTDF (SEQ ID | LCDR2-RASHLQS (SEQ ID NO: 7) |
| | NO: 112) | LCDR3-QQSNEDPLT (SEQ ID |
| | HCDR2-NPNNGG (SEQ ID | NO: 8) |
| | NO: 113) | |
| | HCDR3-VPLYTSHGMDY (SEQ | |
| | ID NO: 4) | |
| 02A01 | HCDR1-GYTFTDF (SEQ ID | LCDR1-RASESVX₁SYDIX₂SMH, |
| Consensus | NO: 112) | wherein X₁ is D or E; and X₂ is N or Q |
| 3³ | HCDR2-NPNNGG (SEQ ID | (SEQ ID NO: 109) |
| | NO: 113) | LCDR2-X₁ASX₂X₃X₄X₅, wherein |
| | HCDR3-VPLYTSHGMDY (SEQ | X₁ is R or D; X₂ is H or N; X₃ is L or |
| | ID NO: 4) | R; X₄ is Q or A; and X₅ is S or T (SEQ |
| | | ID NO: 12) |
| | | LCDR3-QQSNEDPLT (SEQ ID |
| | | NO: 8) |
| 02A01 | HCDR1-GYTFTDF (SEQ ID | LCDR1-RASESVX₁SYDIX₂SMH, |
| Consensus | NO: 112) | wherein X₁ is D or E; and X₂ is N or Q |
| 4⁴ | HCDR2-NPNNGG (SEQ ID | (SEQ ID NO: 109) |
| | NO: 113) | LCDR2-X₁ASX₂X₃X₄X₅, wherein |
| | HCDR3-VPLYTSHGMDY (SEQ | X₁, X₂, X₃, X₄, and X₅ are R, H, L, Q, |
| | ID NO: 4) | and S, respectively; or X₁, X₂, X₃, X₄, |
| | | and X₅ are D, N, R, A, and T, |
| | | respectively (SEQ ID NO: 84) |
| | | LCDR3-QQSNEDPLT (SEQ ID |
| | | NO: 8) |
| Humanized | EVQLVQSGPEVVKPGATLKISC | |
| 02A01- | KASGYTFTDFTMHWVKQAPG | |
| VH1 | KSLEWIGLVNPNNGGNIYNQN | |
| | FKGRATLTVDTSTSTAYMELSS | |
| | LRSEDTSVYYCGRVPLYTSHG | |
| | MDYWGQGTLVTVSS (SEQ ID | |
| | NO: 14) | |
| | HCDR1-GYTFTDF (SEQ ID | |
| | NO: 112) | |
| | HCDR2-NPNNGG (SEQ ID | |
| | NO: 113) | |
| | HCDR3-VPLYTSHGMDY (SEQ | |
| | ID NO: 4) | |
| Humanized | EVQLVQSGAEVKKPGATLKISC | |
| 02A01- | KASGYTFTDFTMHWVQQAPG | |
| VH2 | KGLEWIGLVNPNNGGNIYNQN | |
| | FKGRATLTVDTSTSTAYMELSS | |
| | LRSEDTSVYYCGRVPLYTSHG | |
| | MDYWGQGTLVTVSS (SEQ ID | |
| | NO: 15) | |
| | HCDR1-GYTFTDF (SEQ ID | |
| | NO: 112) | |
| | HCDR2-NPNNGG (SEQ ID | |
| | NO: 113) | |
| | HCDR3-VPLYTSHGMDY (SEQ | |
| | ID NO: 4) | |
| Humanized | EVQLVQSGAEVKKPGATLKISC | |
| 02A01- | KASGYTFTDFTMHWVQQAPG | |
| VH3 | KGLEWIGLVNPNNGGNIYAEK | |
| | FQGRATLTVDTSTSTAYMELSS | |
| | LRSEDTSVYYCGRVPLYTSHG | |
| | MDYWGQGTLVTVSS (SEQ ID | |
| | NO: 16) | |
| | HCDR1-GYTFTDF (SEQ ID | |
| | NO: 112) | |
| | HCDR2-NPNNGG (SEQ ID | |
| | NO: 113) | |
| | HCDR3-VPLYTSHGMDY (SEQ | |
| | ID NO: 4) | |
| Humanized | EVQLVQSGAEVKKPGATVKIS | |
| 02A01- | CKASGYTFTDFTMHWVQQAP | |
| VH4 | GKGLEWIGLVNPNNGGNIYAE | |
| | KFQGRATLTVDTSTSTAYMEL | |
| | SSLRSEDTAVYYCGRVPLYTSH | |
| | GMDYWGQGTLVTVSS (SEQ ID | |
| | NO: 18) | |
| | HCDR1-GYTFTDF (SEQ ID | |
| | NO: 112) | |
| | HCDR2-NPNNGG (SEQ ID | |
| | NO: 113) | |
| | HCDR3-VPLYTSHGMDY (SEQ | |
| | ID NO: 4) | |
| Humanized | EVQLVQSGAEVKKPGATLKISC | |
| 02A01- | KASGYTFTDYYMHWVQQAPG | |
| VH5 | KGLEWIGLVNPNNGGNIYNQN | |
| | FKGRATLTVDTSTSTAYMELSS | |
| | LRSEDTSVYYCGRVPLYTSHG | |
| | MDYWGQGTLVTVSS (SEQ ID | |
| | NO: 19) | |
| | HCDR1-GYTFTDY (SEQ ID | |
| | NO: 114) | |
| | HCDR2-NPNNGG (SEQ ID | |
| | NO: 113) | |
| | HCDR3-VPLYTSHGMDY (SEQ | |
| | ID NO: 4) | |
| Humanized | EVQLVQSGAEVKKPGATVKIS | |
| 02A01- | CKASGYTFTDFTMHWVQQAP | |
| VH6 | GKGLEWIGLVNPNNGGNIYAE | |
| | KFQGRVTLTVDTSTSTAYMEL | |
| | SSLRSEDTAVYYCGRVPLYTSH | |
| | GMDYWGQGTLVTVSS (SEQ ID | |
| | NO: 21) | |
| | HCDR1-GYTFTDF (SEQ ID | |
| | NO: 112) | |
| | HCDR2-NPNNGG (SEQ ID | |
| | NO: 113) | |
| | HCDR3-VPLYTSHGMDY (SEQ | |
| | ID NO: 4) | |
| Humanized | | EIVLTQSPATLSVSPGERATISCRA |
| 02A01- | | SESVDSYDINSMHWYQQKPGQPP |
| VK1 | | RLLIYRASHLQSGIPARFSGSGSRT |
| | | DFTLTISSVEPEDFATYYCQQSNE |
| | | DPLTFGQGTKLEIK (SEQ ID |
| | | NO: 22) |
| | | LCDR1-RASESVDSYDINSMH |
| | | (SEQ ID NO: 6) |
| | | LCDR2-RASHLQS (SEQ ID NO: 7) |
| | | LCDR3-QQSNEDPLT (SEQ ID |
| | | NO: 8) |
| Humanized | | EIVLTQSPATLSVSPGERATISCRA |
| 02A01- | | SESVDSYDINSMHWYQQKPGQAP |
| VK2 | | RLLIYRASHLQSGIPARFSGSGSRT |
| | | DFTLTISSVEPEDFATYYCQQSNE |
| | | DPLTFGQGTKLEIK (SEQ ID |
| | | NO: 23) |
| | | LCDR1-RASESVDSYDINSMH |
| | | (SEQ ID NO: 6) |
| | | LCDR2-RASHLQS (SEQ ID NO: 7) |
| | | LCDR3-QQSNEDPLT (SEQ ID |
| | | NO: 8) |
| Humanized | | EIVLTQSPATLSLSPGERATISCRA |
| 02A01- | | SESVDSYDINSMHWYQQKPGQAP |
| VK3 | | RLLIYRASHLQSGIPARFSGSGSRT |
| | | DFTLTISSLEPEDFATYYCQQSNED |
| | | PLTFGQGTKLEIK (SEQ ID NO: 24) |
| | | LCDR1-RASESVDSYDINSMH |
| | | (SEQ ID NO: 6) |
| | | LCDR2-RASHLQS (SEQ ID NO: 7) |
| | | LCDR3-QQSNEDPLT (SEQ ID |
| | | NO: 8) |
| Humanized | | EIVLTQSPATLSLSPGERATISCRA |
| 02A01- | | SESVESYDIQSMHWYQQKPGQAP |
| VK3 | | RLLIYRASHLQSGIPARFSGSGSRT |
| (D30E, | | DFTLTISSLEPEDFATYYCQQSNED |
| N35Q) | | PLTFGQGTKLEIK (SEQ ID NO: 105) |
| | | LCDR1-RASESVESYDIQSMH |
| | | (SEQ ID NO: 106) |
| | | LCDR2-RASHLQS (SEQ ID NO: 7) |
| | | LCDR3-QQSNEDPLT (SEQ ID |
| | | NO: 8) |
| Humanized | | EIVLTQSPATLSVSPGERATISCRA |
| 02A01- | | SESVDSYDINSMHWYQQKPGQAP |
| VK4 | | RLLIYDASNRATGIPARFSGSGSRT |
| | | DFTLTISSVEPEDFATYYCQQSNE |
| | | DPLTFGQGTKLEIK (SEQ ID |
| | | NO: 25) |
| | | LCDR1-RASESVDSYDINSMH |
| | | (SEQ ID NO: 6) |
| | | LCDR2-DASNRAT (SEQ ID |
| | | NO: 26) |
| | | LCDR3-QQSNEDPLT (SEQ ID |
| | | NO: 8) |

³02A01 consensus 3 is based on the Chothia CDR sequences of: (a) the heavy chain CDRs of murine 02A01, murine 02A01 VH-M39L, humanized 02A01-VH1, humanized 02A01-VH2, humanized 02A01-VH3, humanized 02A01-VH4, and humanized 02A01-VH6; and (b) the light chain CDRs of murine 02A01, murine 02A01 VH-M39L, humanized 02A01-VK1, humanized 02A01-VK2, humanized 02A01-VK3, humanized 02A01-VK3(D30E/N35Q), and humanized 02A01-VK4.
⁴02A01 consensus 4 is based on the Chothia CDR sequences of: (a) the heavy chain CDRs of humanized 02A01-VH1, humanized 02A01-VH2, humanized 02A01-VH3, humanized 02A01-VH4, and humanized 02A01-VH6; and (b) the light chain CDRs of humanized 02A01-VK1, humanized 02A01-VK2, humanized 02A01-VK3, humanized 02A01-VK3(D30E/N35Q), and humanized 02A01-VK4.

TABLE 3A
Exemplary silent CD8 antigen-binding sites derived from 03A01 (Kabat)

| | Heavy Chain Variable Domain | Light Chain Variable Domain |
|---|---|---|
| Clone | (VH) | (VL) |
| 03A01 | DVQFQESGPGLVKPSQSLSLTC | DIQMTQSSSYLSVSLGGRVTITCK |
| | TVTGYSITSDFAWNWIRQFPGN | ASDHINNWLAWYQQKPGNAPRL |
| | ELEWMGYISYSGDTNYNPSLR | LISGATSLETGVPSRFTGSGSGLDY |
| | SRISITRDTSKNQFFLQLDSVTT | TLSITSLQTEDVATYYCQQYWNSP |
| | EDTATYYCTIRGYYTDSAFVF | FTFGSGTKLEIK (SEQ ID NO: 31) |
| | WGQGTLVTVSA (SEQ ID | LCDR1-KASDHINNWLA (SEQ ID |
| | NO: 27) | NO: 32) |
| | HCDR1-SDFAWN (SEQ ID | LCDR2-GATSLET (SEQ ID NO: 33) |
| | NO: 28) | LCDR3-QQYWNSPFT (SEQ ID |
| | HCDR2-YISYSGDTNYNPSLRS | NO: 34) |
| | (SEQ ID NO: 29) | |
| | HCDR3-RGYYTDSAFVF (SEQ | |
| | ID NO: 30) | |
| 03A01 | DVQFQESGPGLVKPSQSLSLTC | DIQMTQSSSYLSVSLGGRVTITCK |
| VH-D64E | TVTGYSITSDFAWNWIRQFPGN | ASDHINNWLAWYQQKPGNAPRL |
| | ELEWMGYISYSGETNYNPSLRS | LISGATSLETGVPSRFTGSGSGLDY |
| | RISITRDTSKNQFFLQLDSVTTE | TLSITSLQTEDVATYYCQQYWNSP |
| | DTATYYCTIRGYYTDSAFVFW | FTFGSGTKLEIK (SEQ ID NO: 31) |
| | GQGTLVTVSA (SEQ ID NO: 35) | LCDR1-KASDHINNWLA (SEQ ID |
| | HCDR1-SDFAWN (SEQ ID | NO: 32) |
| | NO: 28) | LCDR2-GATSLET (SEQ ID NO: 33) |
| | HCDR2-YISYSGETNYNPSLRS | LCDR3-QQYWNSPFT (SEQ ID |
| | (SEQ ID NO: 36) | NO: 34) |
| | HCDR3-RGYYTDSAFVF (SEQ | |
| | ID NO: 30) | |
| 03A01 | HCDR1-SDFAWN (SEQ ID | LCDR1-KASDHINNWLA (SEQ ID |
| Consensus | NO: 28) | NO: 32) |
| 1⁵ | HCDR2- | LCDR2-GATSLET (SEQ ID NO: 33) |
| | YISYSGX₁TX₂YNPSLX₃S, | LCDR3-QQYWNSPFT (SEQ ID |
| | wherein X₁ is D or E; X₂ is N or Y; | NO: 34) |
| | and X₃ is R or K (SEQ ID NO: 37) | |
| | HCDR3-RGYYTDSAFVF (SEQ | |
| | ID NO: 30) | |
| 03A01 | HCDR1-SDFAWN (SEQ ID | LCDR1-KASDHINNWLA (SEQ ID |
| Consensus | NO: 28) | NO: 32) |
| 2⁶ | HCDR2- | LCDR2-GATSLET (SEQ ID NO: 33) |
| | YISYSGDTX₁YNPSLX₂S, | LCDR3-QQYWNSPFT (SEQ ID |
| | wherein X₁ and X₂ are N and R, | NO: 34) |
| | respectively; X₁ and X₂ are N and | |
| | K, respectively, or X₁ and X₂ are Y | |
| | and K, respectively (SEQ ID | |
| | NO: 38) | |
| | HCDR3-RGYYTDSAFVF (SEQ | |
| | ID NO: 30) | |
| Humanized | EVQFQESGPGLVKPSQTLSLTC | |
| 03A01- | TVTGYSITSDFAWNWIRQPPGK | |
| VH1 | ELEWMGYISYSGDTNYNPSLR | |
| | SRITISRDTSKNQFSLKLSSVTA | |
| | ADTATYYCTIRGYYTDSAFVF | |
| | WGQGTLVTVSS (SEQ ID | |
| | NO: 39) | |
| | HCDR1-SDFAWN (SEQ ID | |
| | NO: 28) | |
| | HCDR2-YISYSGDTNYNPSLRS | |
| | (SEQ ID NO: 29) | |
| | HCDR3-RGYYTDSAFVF (SEQ | |
| | ID NO: 30) | |
| Humanized | EVQFQESGPGLVKPSQTLSLTC | |
| 03A01- | TVSGYSITSDFAWNWIRQPPGK | |
| VH2 | GLEWMGYISYSGDTNYNPSLR | |
| | SRITISRDTSKNQFSLKLSSVTA | |
| | ADTAVYYCTIRGYYTDSAFVF | |
| | WGQGTLVTVSS (SEQ ID | |
| | NO: 40) | |
| | HCDR1-SDFAWN (SEQ ID | |
| | NO: 28) | |
| | HCDR2-YISYSGDTNYNPSLRS | |
| | (SEQ ID NO: 29) | |
| | HCDR3-RGYYTDSAFVF (SEQ | |
| | ID NO: 30) | |
| Humanized | EVQFQESGPGLVKPSQTLSLTC | |
| 03A01- | TVSGYSITSDFAWNWIRQPPGK | |
| VH3 | GLEWMGYISYSGDTNYNPSLK | |
| | SRVTISRDTSKNQFSLKLSSVTA | |
| | ADTAVYYCTIRGYYTDSAFVF | |
| | WGQGTLVTVSS (SEQ ID | |
| | NO: 41) | |
| | HCDR1-SDFAWN (SEQ ID | |
| | NO: 28) | |
| | HCDR2-YISYSGDTNYNPSLKS | |
| | (SEQ ID NO: 42) | |
| | HCDR3-RGYYTDSAFVF (SEQ | |
| | ID NO: 30) | |
| Humanized | EVQFQESGPGLVKPSQTLSLTC | |
| 03A01- | TVSGYSITSDFAWNWIRQPPGK | |
| VH4 | GLEWIGYISYSGDTYYNPSLKS | |
| | RVTISRDTSKNQFSLKLSSVTA | |
| | ADTAVYYCTIRGYYTDSAFVF | |
| | WGQGTLVTVSS (SEQ ID | |
| | NO: 43) | |
| | HCDR1-SDFAWN (SEQ ID | |
| | NO: 28) | |
| | HCDR2-YISYSGDTYYNPSLKS | |
| | (SEQ ID NO: 44) | |
| | HCDR3-RGYYTDSAFVF (SEQ | |
| | ID NO: 30) | |
| Humanized | | DIQMTQSSSTLSVSLGDRVTITCK |
| 03A01- | | ASDHINNWLAWYQQKPGKAPRL |
| VK1 | | LISGATSLETGVPSRFSGSGSGLEY |
| | | TLTISSLQPDDFATYYCQQYWNSP |
| | | FTFGQGTKLEIK (SEQ ID NO: 45) |
| | | LCDR1-KASDHINNWLA (SEQ ID |
| | | NO: 32) |
| | | LCDR2-GATSLET (SEQ ID NO: 33) |
| | | LCDR3-QQYWNSPFT (SEQ ID |
| | | NO: 34) |
| Humanized | | DIQMTQSPSTLSVSVGDRVTITCK |
| 03A01- | | ASDHINNWLAWYQQKPGKAPKL |
| VK2 | | LISGATSLETGVPSRFSGSGSGLEY |
| | | TLTISSLQPDDFATYYCQQYWNSP |
| | | FTFGQGTKLEIK (SEQ ID NO: 46) |
| | | LCDR1-KASDHINNWLA (SEQ ID |
| | | NO: 32) |
| | | LCDR2-GATSLET (SEQ ID NO: 33) |
| | | LCDR3-QQYWNSPFT (SEQ ID |
| | | NO: 34) |
| Humanized | | DIQMTQSPSTLSASVGDRVTITCK |
| 03A01- | | ASDHINNWLAWYQQKPGKAPKL |
| VK3 | | LISGATSLETGVPSRFSGSGSGLEY |
| | | TLTISSLQPDDFATYYCQQYWNSP |
| | | FTFGQGTKLEIK (SEQ ID NO: 47) |
| | | LCDR1-KASDHINNWLA (SEQ ID |
| | | NO: 32) |
| | | LCDR2-GATSLET (SEQ ID NO: 33) |
| | | LCDR3-QQYWNSPFT (SEQ ID |
| | | NO: 34) |
| Humanized | | DIQMTQSPSTLSVSVGDRVTITCR |
| 03A01- | | ASQSISSWLAWYQQKPGKAPKLLI |
| VK4 | | SGATSLETGVPSRFSGSGSGLEYT |
| | | LTISSLQPDDFATYYCQQYWNSPF |
| | | TFGQGTKLEIK (SEQ ID NO: 48) |
| | | LCDR1-RASQSISSWLA (SEQ ID |
| | | NO: 49) |
| | | LCDR2-GATSLET (SEQ ID NO: 33) |
| | | LCDR3-QQYWNSPFT (SEQ ID |
| | | NO: 34) |

⁵03A01 consensus 1 is based on the Kabat CDR sequences of: (a) the heavy chain CDRs of murine 03A01, murine 03A01 VH-D64E, humanized 03A01-VH1, humanized 03A01-VH2, humanized 03A01-VH3, and humanized 03A01-VH4; and (b) the light chain CDRs of murine 03A01, murine 03A01 VH-D64E, humanized 03A01-VK1, humanized 03A01-VK2, and humanized 03A01-VK3.
⁶03A01 consensus 2 is based on the Kabat CDR sequences of: (a) the heavy chain CDRs of humanized 03A01-VH1, humanized 03A01-VH2, humanized 03A01-VH3, and humanized 03A01-VH4; and (b) the light chain CDRs of humanized 03A01-VK1, humanized 03A01-VK2, and humanized 03A01-VK3.

TABLE 3B
Exemplary silent CD8 antigen-binding sites derived from 03A01 (Chothia)

| | Heavy Chain Variable Domain | Light Chain Variable Domain |
|---|---|---|
| Clone | (VH) | (VL) |
| 03A01 | DVQFQESGPGLVKPSQSLSLTC | DIQMTQSSSYLSVSLGGRVTITCK |
| | TVTGYSITSDFAWNWIRQFPGN | ASDHINNWLAWYQQKPGNAPRL |
| | ELEWMGYISYSGDTNYNPSLR | LISGATSLETGVPSRFTGSGSGLDY |
| | SRISITRDTSKNQFFLQLDSVTT | TLSITSLQTEDVATYYCQQYWNSP |
| | EDTATYYCTIRGYYTDSAFVF | FTFGSGTKLEIK (SEQ ID NO: 31) |
| | WGQGTLVTVSA (SEQ ID | LCDR1-KASDHINNWLA (SEQ ID |
| | NO: 27) | NO: 32) |
| | HCDR1-GYSITSDF (SEQ ID | LCDR2-GATSLET (SEQ ID NO: 33) |
| | NO: 115) | LCDR3-QQYWNSPFT (SEQ ID |
| | HCDR2-SYSGD (SEQ ID | NO: 34) |
| | NO: 116) | |
| | HCDR3-RGYYTDSAFVF (SEQ | |
| | ID NO: 30) | |
| 03A01 | DVQFQESGPGLVKPSQSLSLTC | DIQMTQSSSYLSVSLGGRVTITCK |
| VH-D64E | TVTGYSITSDFAWNWIRQFPGN | ASDHINNWLAWYQQKPGNAPRL |
| | ELEWMGYISYSGETNYNPSLRS | LISGATSLETGVPSRFTGSGSGLDY |
| | RISITRDTSKNQFFLQLDSVTTE | TLSITSLQTEDVATYYCQQYWNSP |
| | DTATYYCTIRGYYTDSAFVFW | FTFGSGTKLEIK (SEQ ID NO: 31) |
| | GQGTLVTVSA (SEQ ID NO: 35) | LCDR1-KASDHINNWLA (SEQ ID |
| | HCDR1-GYSITSDF (SEQ ID | NO: 32) |
| | NO: 115) | LCDR2-GATSLET (SEQ ID NO: 33) |
| | HCDR2-SYSGE (SEQ ID | LCDR3-QQYWNSPFT (SEQ ID |
| | NO: 117) | NO: 34) |
| | HCDR3-RGYYTDSAFVF (SEQ | |
| | ID NO: 30) | |
| 03A01 | HCDR1-GYSITSDF (SEQ ID | LCDR1-KASDHINNWLA (SEQ ID |
| Consensus | NO: 115) | NO: 32) |
| 3⁷ | HCDR2-SYSGX₁, wherein X₁ is | LCDR2-GATSLET (SEQ ID NO: 33) |
| | D or E; (SEQ ID NO: 118) | LCDR3-QQYWNSPFT (SEQ ID |
| | HCDR3-RGYYTDSAFVF (SEQ | NO: 34) |
| | ID NO: 30) | |
| Humanized | EVQFQESGPGLVKPSQTLSLTC | |
| 03A01- | TVTGYSITSDFAWNWIRQPPGK | |
| VH1 | ELEWMGYISYSGDTNYNPSLR | |
| | SRITISRDTSKNQFSLKLSSVTA | |
| | ADTATYYCTIRGYYTDSAFVF | |
| | WGQGTLVTVSS (SEQ ID | |
| | NO: 39) | |
| | HCDR1-GYSITSDF (SEQ ID | |
| | NO: 115) | |
| | HCDR2-SYSGD (SEQ ID | |
| | NO: 116) | |
| | HCDR3-RGYYTDSAFVF (SEQ | |
| | ID NO: 30) | |
| Humanized | EVQFQESGPGLVKPSQTLSLTC | |
| 03A01- | TVSGYSITSDFAWNWIRQPPGK | |
| VH2 | GLEWMGYISYSGDTNYNPSLR | |
| | SRITISRDTSKNQFSLKLSSVTA | |
| | ADTAVYYCTIRGYYTDSAFVF | |
| | WGQGTLVTVSS (SEQ ID | |
| | NO: 40) | |
| | HCDR1-GYSITSDF (SEQ ID | |
| | NO: 115) | |
| | HCDR2-SYSGD (SEQ ID | |
| | NO: 116) | |
| | HCDR3-RGYYTDSAFVF (SEQ | |
| | ID NO: 30) | |
| Humanized | EVQFQESGPGLVKPSQTLSLTC | |
| 03A01- | TVSGYSITSDFAWNWIRQPPGK | |
| VH3 | GLEWMGYISYSGDTNYNPSLK | |
| | SRVTISRDTSKNQFSLKLSSVTA | |
| | ADTAVYYCTIRGYYTDSAFVF | |
| | WGQGTLVTVSS (SEQ ID | |
| | NO: 41) | |
| | HCDR1-GYSITSDF (SEQ ID | |
| | NO: 115) | |
| | HCDR2-SYSGD (SEQ ID | |
| | NO: 116) | |
| | HCDR3-RGYYTDSAFVF (SEQ | |
| | ID NO: 30) | |
| Humanized | EVQFQESGPGLVKPSQTLSLTC | |
| 03A01- | TVSGYSITSDFAWNWIRQPPGK | |
| VH4 | GLEWIGYISYSGDTYYNPSLKS | |
| | RVTISRDTSKNQFSLKLSSVTA | |
| | ADTAVYYCTIRGYYTDSAFVF | |
| | WGQGTLVTVSS (SEQ ID | |
| | NO: 43) | |
| | HCDR1-GYSITSDF (SEQ ID | |
| | NO: 115) | |
| | HCDR2-SYSGD (SEQ ID | |
| | NO: 116) | |
| | HCDR3-RGYYTDSAFVF (SEQ | |
| | ID NO: 30) | |
| Humanized | | DIQMTQSSSTLSVSLGDRVTITCK |
| 03A01- | | ASDHINNWLAWYQQKPGKAPRL |
| VK1 | | LISGATSLETGVPSRFSGSGSGLEY |
| | | TLTISSLQPDDFATYYCQQYWNSP |
| | | FTFGQGTKLEIK (SEQ ID NO: 45) |
| | | LCDR1-KASDHINNWLA (SEQ ID |
| | | NO: 32) |
| | | LCDR2-GATSLET (SEQ ID NO: 33) |
| | | LCDR3-QQYWNSPFT (SEQ ID |
| | | NO: 34) |
| Humanized | | DIQMTQSPSTLSVSVGDRVTITCK |
| 03A01- | | ASDHINNWLAWYQQKPGKAPKL |
| VK2 | | LISGATSLETGVPSRFSGSGSGLEY |
| | | TLTISSLQPDDFATYYCQQYWNSP |
| | | FTFGQGTKLEIK (SEQ ID NO: 46) |
| | | LCDR1-KASDHINNWLA (SEQ ID |
| | | NO: 32) |
| | | LCDR2-GATSLET (SEQ ID NO: 33) |
| | | LCDR3-QQYWNSPFT (SEQ ID |
| | | NO: 34) |
| Humanized | | DIQMTQSPSTLSASVGDRVTITCK |
| 03A01- | | ASDHINNWLAWYQQKPGKAPKL |
| VK3 | | LISGATSLETGVPSRFSGSGSGLEY |
| | | TLTISSLQPDDFATYYCQQYWNSP |
| | | FTFGQGTKLEIK (SEQ ID NO: 47) |
| | | LCDR1-KASDHINNWLA (SEQ ID |
| | | NO: 32) |
| | | LCDR2-GATSLET (SEQ ID NO: 33) |
| | | LCDR3-QQYWNSPFT (SEQ ID |
| | | NO: 34) |
| Humanized | | DIQMTQSPSTLSVSVGDRVTITCR |
| 03A01- | | ASQSISSWLAWYQQKPGKAPKLLI |
| VK4 | | SGATSLETGVPSRFSGSGSGLEYT |
| | | LTISSLQPDDFATYYCQQYWNSPF |
| | | TFGQGTKLEIK (SEQ ID NO: 48) |
| | | LCDR1-RASQSISSWLA (SEQ ID |
| | | NO: 49) |
| | | LCDR2-GATSLET (SEQ ID NO: 33) |
| | | LCDR3-QQYWNSPFT (SEQ ID |
| | | NO: 34) |

⁷03A01 consensus 1 is based on the Chothia CDR sequences of: (a) the heavy chain CDRs of murine 03A01, murine 03A01 VH-D64E, humanized 03A01-VH1, humanized 03A01-VH2, humanized 03A01-VH3, and humanized 03A01-VH4; and (b) the light chain CDRs of murine 03A01, murine 03A01 VH-D64E, humanized 03A01-VK1, humanized 03A01-VK2, and humanized 03A01-VK3.

TABLE 4A
Additional exemplary silent CD8 antigen-binding sites (Kabat)

| | Heavy Chain Variable Domain | Light Chain Variable Domain |
|---|---|---|
| Clone | (VH) | (VL) |
| 05F10 | EVQLVESGGGLVKPGGSLKLSC | DVVMTQTPLSLPVSLEDQASISCR |
| | AASGFTFSSFAMSWVRQTPAKR | SSQSLEHSNGNTYLHWYLQKPGQ |
| | LEWVATISSGDLSANYPDTVKG | SPRLLIYKVSNRFSGVPDRFSGSGS |
| | RFTISRDNAKNTLYLQMSSLRSE | GTDFTLKISRVEAEDLGVYFCSQS |
| | DTAMYYCTRPPFYFSNDWFAY | THVPFTFGSGTKLEIK (SEQ ID |
| | WGQGTLVTVSA (SEQ ID NO: 50) | NO: 54) |
| | HCDR1-SFAMS (SEQ ID NO: 51) | LCDR1-RSSQSLEHSNGNTYLH |
| | HCDR2- | (SEQ ID NO: 55) |
| | TISSGDLSANYPDTVKG (SEQ ID | LCDR2-KVSNRFS (SEQ ID |
| | NO: 52) | NO: 56) |
| | HCDR3-PPFYFSNDWFAY (SEQ | LCDR3-SQSTHVPFT (SEQ ID |
| | ID NO: 53) | NO: 57) |
| 11F05 | DVQLQESGPGLVKPSQSLSLTC | DIQMTQSSSYLSVSLGGRVTITCK |
| | TVTGYSITSDYAWNWIRQFPGN | ASDHINNWLAWYQQKPGNAPRL |
| | KLEWMGYISYSGSTSYNPSLKS | LISDATSLETGVPSRFSGSVSGMD |
| | RISITRDTSKNQFFLHLNSVTTE | YTLSITSLQTEDVATYYCQQYWST |
| | DTATYYCARRGYYSHSAFTYW | PFTFGSGTKLEIE (SEQ ID NO: 62) |
| | GQGTLVTVSA (SEQ ID NO: 58) | LCDR1-KASDHINNWLA (SEQ ID |
| | HCDR1-SDYAWN (SEQ ID | NO: 32) |
| | NO: 59) | LCDR2-DATSLET (SEQ ID NO: 63) |
| | HCDR2-YISYSGSTSYNPSLKS | LCDR3-QQYWSTPFT (SEQ ID |
| | (SEQ ID NO: 60) | NO: 64) |
| | HCDR3-RGYYSHSAFTY (SEQ | |
| | ID NO: 61) | |
| 04D08 | EVQLQQSGPELEKPGASVKISC | DIVMTQSPSSLTVTAGEKVTMSCK |
| | KASGYSFTGYSMNWVKQSNGK | SSQSLLNIGNQNNYLTWYQQKPG |
| | SLEWIGNIDPYYGGTSYNQNFK | QPPKLLIYWASTRESGVPDRFTGS |
| | GKATLTVDKSSSTAYMQLKSLT | GSGTDFTLTISSVQAEDLAVYYCQ |
| | SEDSAVYYCARSYFGNYEDTW | NDYSYPLTFGAGTKLELK (SEQ ID |
| | FAYWGQGTLVTVSA (SEQ ID | NO: 69) |
| | NO: 65) | LCDR1-KSSQSLLNIGNQNNYLT |
| | HCDR1-GYSMN (SEQ ID | (SEQ ID NO: 70) |
| | NO: 66) | LCDR2-WASTRES (SEQ ID |
| | HCDR2- | NO: 71) |
| | NIDPYYGGTSYNQNFKG (SEQ | LCDR3-QNDYSYPLT (SEQ ID |
| | ID NO: 67) | NO: 72) |
| | HCDR3-SYFGNYEDTWFAY | |
| | (SEQ ID NO: 68) | |

TABLE 4B
Additional exemplary silent CD8 antigen-binding sites (Chothia)

| | Heavy Chain Variable Domain | Light Chain Variable Domain |
|---|---|---|
| Clone | (VH) | (VL) |
| 05F10 | EVQLVESGGGLVKPGGSLKLSC | DVVMTQTPLSLPVSLEDQASISCR |
| | AASGFTFSSFAMSWVRQTPAKR | SSQSLEHSNGNTYLHWYLQKPGQ |
| | LEWVATISSGDLSANYPDTVKG | SPRLLIYKVSNRFSGVPDRFSGSGS |
| | RFTISRDNAKNTLYLQMSSLRSE | GTDFTLKISRVEAEDLGVYFCSQS |
| | DTAMYYCTRPPFYFSNDWFAY | THVPFTFGSGTKLEIK (SEQ ID |
| | WGQGTLVTVSA (SEQ ID NO: 50) | NO: 54) |
| | HCDR1-GFTFSSF (SEQ ID | LCDR1-RSSQSLEHSNGNTYLH |
| | NO: 119) | (SEQ ID NO: 55) |
| | HCDR2-SSGDLS (SEQ ID | LCDR2-KVSNRFS (SEQ ID |
| | NO: 120) | NO: 56) |
| | HCDR3-PPFYFSNDWFAY (SEQ | LCDR3-SQSTHVPFT (SEQ ID |
| | ID NO: 53) | NO: 57) |
| 11F05 | DVQLQESGPGLVKPSQSLSLTC | DIQMTQSSSYLSVSLGGRVTITCK |
| | TVTGYSITSDYAWNWIRQFPGN | ASDHINNWLAWYQQKPGNAPRL |
| | KLEWMGYISYSGSTSYNPSLKS | LISDATSLETGVPSRFSGSVSGMD |
| | RISITRDTSKNQFFLHLNSVTTE | YTLSITSLQTEDVATYYCQQYWST |
| | DTATYYCARRGYYSHSAFTYW | PFTFGSGTKLEIE (SEQ ID NO: 62) |
| | GQGTLVTVSA (SEQ ID NO: 58) | LCDR1-KASDHINNWLA (SEQ ID |
| | HCDR1-GYSITSDY (SEQ ID | NO: 32) |
| | NO: 121) | LCDR2-DATSLET (SEQ ID NO: 63) |
| | HCDR2-SYSGS (SEQ ID | LCDR3-QQYWSTPFT (SEQ ID |
| | NO: 122) | NO: 64) |
| | HCDR3-RGYYSHSAFTY (SEQ | |
| | ID NO: 61) | |
| 04D08 | EVQLQQSGPELEKPGASVKISC | DIVMTQSPSSLTVTAGEKVTMSCK |
| | KASGYSFTGYSMNWVKQSNGK | SSQSLLNIGNQNNYLTWYQQKPG |
| | SLEWIGNIDPYYGGTSYNQNFK | QPPKLLIYWASTRESGVPDRFTGS |
| | GKATLTVDKSSSTAYMQLKSLT | GSGTDFTLTISSVQAEDLAVYYCQ |
| | SEDSAVYYCARSYFGNYEDTW | NDYSYPLTFGAGTKLELK (SEQ ID |
| | FAYWGQGTLVTVSA (SEQ ID | NO: 69) |
| | NO: 65) | LCDR1-KSSQSLLNIGNQNNYLT |
| | HCDR1-GYSFTGY (SEQ ID | (SEQ ID NO: 70) |
| | NO: 123) | LCDR2-WASTRES (SEQ ID |
| | HCDR2-DPYYGG (SEQ ID | NO: 71) |
| | NO: 124) | LCDR3-QNDYSYPLT (SEQ ID |
| | HCDR3-SYFGNYEDTWFAY | NO: 72) |
| | (SEQ ID NO: 68) | |

CD8-Binding Sites Derived from 02A01

In certain embodiments, the antigen-binding site that binds CD8 disclosed herein comprises an antibody heavy chain variable domain (VH) that comprises an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to the VH of an antibody disclosed in Table 2A or 2B, and an antibody light chain variable domain (VL) that comprises an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to the VL of the same antibody disclosed in Table 2A or 2B. In certain embodiments, the antigen-binding site comprises the HCDR1, HCDR2, HCDR3, LCDR1, LCDR2, and LCDR3, determined under Kabat (e.g., as disclosed in Table 2A; see Kabat et al., (1991) Sequences of Proteins of Immunological Interest, NIH Publication No. 91-3242, Bethesda), Chothia (e.g., as disclosed in Table 2B; see, e.g., Chothia C & Lesk A M, (1987), J. Mol. Biol. 196: 901-917), MacCallum (see MacCallum R M et al., (1996) J Mol Biol 262: 732-745), IMGT (see Lefranc, (1999) The Immunologist, 7, 132-136), or any other CDR determination method known in the art, of the VH and VL sequences of an antibody disclosed in Table 2A or 2B. In certain embodiments, the antigen-binding site comprises the HCDR1, HCDR2, HCDR3, LCDR1, LCDR2, and LCDR3 sequences of an antibody disclosed in Table 2A or 2B. In certain embodiments, the antigen-binding site comprises the VH and VL sequences of an antibody disclosed in Table 2A or 2B.

In certain embodiments, the antigen-binding site that binds CD8 comprises a VH comprising HCDR1, HCDR2, and HCDR3 sequences set forth in SEQ ID NOs: 112, 113, and 4, respectively, and a VL comprising LCDR1, LCDR2, and LCDR3 sequences set forth in SEQ ID NOs: 109, 12, and 8, respectively. In certain embodiments, the LCDR1 sequence is selected from the group consisting of SEQ ID NOs: 6 or 106; the LCDR2 sequence is selected from the group consisting of SEQ ID NOs: 7 and 26; and/or the LCDR3 sequence is SEQ ID NO: 8.

In certain embodiments, the antigen-binding site that binds CD8 comprises a VH comprising HCDR1, HCDR2, and HCDR3 sequences set forth in SEQ ID NOs: 112, 113, and 4, respectively, and a VL comprising LCDR1, LCDR2, and LCDR3 sequences set forth in SEQ ID NOs: 109, 84, and 8, respectively. In certain embodiments, the LCDR1 sequence is selected from the group consisting of SEQ ID NO: 6 or 106; the LCDR2 sequence is selected from the group consisting of SEQ ID NOs: 7 and 26; and/or the LCDR3 sequence is SEQ ID NO: 8.

In certain embodiments, the antigen-binding site that binds CD8 is derived from a humanized 02A01. In certain embodiments, the antigen-binding site comprises a VH comprising HCDR1, HCDR2, and HCDR3 sequences set forth in SEQ ID NOs: 112, 113, and 4, respectively, and a VL comprising LCDR1, LCDR2, and LCDR3 sequences set forth in SEQ ID NOs: 6, 7, and 8; 106, 7, and 8; 6, 26, and 8; or 106, 26, and 8, respectively.

In certain embodiments, the antigen-binding site that binds CD8 is derived from a humanized 02A01-VH6-VK3(D30E/N35Q). In certain embodiments, the antigen-binding site comprises a VH comprising HCDR1, HCDR2, and HCDR3 sequences set forth in SEQ ID NOs: 112, 113, and 4, respectively, and a VL comprising LCDR1, LCDR2, and LCDR3 sequences set forth in SEQ ID NOs: 106, 7, and 8, respectively. In certain embodiments, the antigen-binding site comprises a VH comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 21, and a VL that comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 105. In certain embodiments, the VH and the VL comprise the amino acid sequences of SEQ ID NOs: 21 and 105, respectively.

In certain embodiments, the antigen-binding site that binds CD8 comprises a VH comprising HCDR1, HCDR2, and HCDR3 sequences set forth in SEQ ID NOs: 88, 11, and 4, respectively, and a VL comprising LCDR1, LCDR2, and LCDR3 sequences set forth in SEQ ID NOs: 109, 12, and 8, respectively. In certain embodiments, the HCDR1 sequence is selected from the group consisting of SEQ ID NOs: 2 and 10; the HCDR2 sequence is selected from the group consisting of SEQ ID NOs: 3 and 17; the HCDR3 sequence is SEQ ID NOs: 4; the LCDR1 sequence is SEQ ID NOs: 6 or 106; the LCDR2 sequence is selected from the group consisting of SEQ ID NOs: 7 and 26; and/or the LCDR3 sequence is SEQ ID NO: 8.

In certain embodiments, the antigen-binding site comprises a VH comprising HCDR1, HCDR2, and HCDR3 sequences set forth in SEQ ID NOs: 2, 13, and 4, respectively, and a VL comprising LCDR1, LCDR2, and LCDR3 sequences set forth in SEQ ID NOs: 109, 84, and 8, respectively. In certain embodiments, the HCDR1 sequence is SEQ ID NO: 2; the HCDR2 sequence is selected from the group consisting of SEQ ID NOs: 3 and 17; the HCDR3 sequence is SEQ ID NO: 4; the LCDR1 sequence is SEQ ID NO: 6 or 106; the LCDR2 sequence is selected from the group consisting of SEQ ID NOs: 7 and 26; and/or the LCDR3 sequence is SEQ ID NO: 8.

In certain embodiments, the antigen-binding site that binds CD8 is derived from a humanized 02A01. In certain embodiments, the antigen-binding site comprises a VH comprising HCDR1, HCDR2, and HCDR3 sequences set forth in SEQ ID NOs: 2, 17, and 4; or 2, 3, and 4, respectively, and a VL comprising LCDR1, LCDR2, and LCDR3 sequences set forth in SEQ ID NOs: 6, 7, and 8; 106, 7, and 8; 6, 26, and 8; or 106, 26, and 8, respectively.

In certain embodiments, the antigen-binding site that binds CD8 is derived from a humanized 02A01-VH1-VK1. In certain embodiments, the antigen-binding site comprises a VH comprising HCDR1, HCDR2, and HCDR3 sequences set forth in SEQ ID NOs: 2, 3, and 4, respectively, and a VL comprising LCDR1, LCDR2, and LCDR3 sequences set forth in SEQ ID NOs: 6, 7, and 8, respectively. In certain embodiments, the antigen-binding site comprises a VH comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 14, and a VL that comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 22. In certain embodiments, the VH and the VL comprise the amino acid sequences of SEQ ID NOs: 14 and 22, respectively.

In certain embodiments, the antigen-binding site that binds CD8 is derived from a humanized 02A01-VH2-VK1. In certain embodiments, the antigen-binding site comprises a VH comprising HCDR1, HCDR2, and HCDR3 sequences set forth in SEQ ID NOs: 2, 3, and 4, respectively, and a VL comprising LCDR1, LCDR2, and LCDR3 sequences set forth in SEQ ID NOs: 6, 7, and 8, respectively. In certain embodiments, the antigen-binding site comprises a VH comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 15, and a VL that comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 22. In certain embodiments, the VH and the VL comprise the amino acid sequences of SEQ ID NOs: 15 and 22, respectively.

In certain embodiments, the antigen-binding site that binds CD8 is derived from a humanized 02A01-VH1-VK2. In certain embodiments, the antigen-binding site comprises a VH comprising HCDR1, HCDR2, and HCDR3 sequences set forth in SEQ ID NOs: 2, 3, and 4, respectively, and a VL comprising LCDR1, LCDR2, and LCDR3 sequences set forth in SEQ ID NOs: 6, 7, and 8, respectively. In certain embodiments, the antigen-binding site comprises a VH comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 14, and a VL that comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 23. In certain embodiments, the VH and the VL comprise the amino acid sequences of SEQ ID NOs: 14 and 23, respectively.

In certain embodiments, the antigen-binding site that binds CD8 is derived from a humanized 02A01-VH2-VK2. In certain embodiments, the antigen-binding site comprises a VH comprising HCDR1, HCDR2, and HCDR3 sequences set forth in SEQ ID NOs: 2, 3, and 4, respectively, and a VL comprising LCDR1, LCDR2, and LCDR3 sequences set forth in SEQ ID NOs: 6, 7, and 8, respectively. In certain embodiments, the antigen-binding site comprises a VH comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 15, and a VL that comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 23. In certain embodiments, the VH and the VL comprise the amino acid sequences of SEQ ID NOs: 15 and 23, respectively.

In certain embodiments, the antigen-binding site that binds CD8 is derived from a humanized 02A01-VH1-VK3. In certain embodiments, the antigen-binding site comprises a VH comprising HCDR1, HCDR2, and HCDR3 sequences set forth in SEQ ID NOs: 2, 3, and 4, respectively, and a VL comprising LCDR1, LCDR2, and LCDR3 sequences set forth in SEQ ID NOs: 6, 7, and 8, respectively. In certain embodiments, the antigen-binding site comprises a VH comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 14, and a VL that comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 24. In certain embodiments, the VH and the VL comprise the amino acid sequences of SEQ ID NOs: 14 and 24, respectively.

In certain embodiments, the antigen-binding site that binds CD8 is derived from a humanized 02A01-VH1-VK3(D30E/N35Q). In certain embodiments, the antigen-binding site comprises a VH comprising HCDR1, HCDR2, and HCDR3 sequences set forth in SEQ ID NOs: 2, 3, and 4, respectively, and a VL comprising LCDR1, LCDR2, and LCDR3 sequences set forth in SEQ ID NOs: 106, 7, and 8, respectively. In certain embodiments, the antigen-binding site comprises a VH comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 14, and a VL that comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 105. In certain embodiments, the VH and the VL comprise the amino acid sequences of SEQ ID NOs: 14 and 105, respectively.

In certain embodiments, the antigen-binding site that binds CD8 is derived from a humanized 02A01-VH2-VK3. In certain embodiments, the antigen-binding site comprises a VH comprising HCDR1, HCDR2, and HCDR3 sequences set forth in SEQ ID NOs: 2, 3, and 4, respectively, and a VL comprising LCDR1, LCDR2, and LCDR3 sequences set forth in SEQ ID NOs: 6, 7, and 8, respectively. In certain embodiments, the antigen-binding site comprises a VH comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 15, and a VL that comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 24. In certain embodiments, the VH and the VL comprise the amino acid sequences of SEQ ID NOs: 15 and 24, respectively.

In certain embodiments, the antigen-binding site that binds CD8 is derived from a humanized 02A01-VH2-VK3(D30E/N35Q). In certain embodiments, the antigen-binding site comprises a VH comprising HCDR1, HCDR2, and HCDR3 sequences set forth in SEQ ID NOs: 2, 3, and 4, respectively, and a VL comprising LCDR1, LCDR2, and LCDR3 sequences set forth in SEQ ID NOs: 106, 7, and 8, respectively. In certain embodiments, the antigen-binding site comprises a VH comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 15, and a VL that comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 105. In certain embodiments, the VH and the VL comprise the amino acid sequences of SEQ ID NOs: 15 and 105, respectively.

In certain embodiments, the antigen-binding site that binds CD8 is derived from a humanized 02A01-VH3-VK1. In certain embodiments, the antigen-binding site comprises a VH comprising HCDR1, HCDR2, and HCDR3 sequences set forth in SEQ ID NOs: 2, 17, and 4, respectively, and a VL comprising LCDR1, LCDR2, and LCDR3 sequences set forth in SEQ ID NOs: 6, 7, and 8, respectively. In certain embodiments, the antigen-binding site comprises a VH comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 16, and a VL that comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 22. In certain embodiments, the VH and the VL comprise the amino acid sequences of SEQ ID NOs: 16 and 22, respectively.

In certain embodiments, the antigen-binding site that binds CD8 is derived from a humanized 02A01-VH4-VK1. In certain embodiments, the antigen-binding site comprises a VH comprising HCDR1, HCDR2, and HCDR3 sequences set forth in SEQ ID NOs: 2, 17, and 4, respectively, and a VL comprising LCDR1, LCDR2, and LCDR3 sequences set forth in SEQ ID NOs: 6, 7, and 8, respectively. In certain embodiments, the antigen-binding site comprises a VH comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 18, and a VL that comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 22. In certain embodiments, the VH and the VL comprise the amino acid sequences of SEQ ID NOs: 18 and 22, respectively.

In certain embodiments, the antigen-binding site that binds CD8 is derived from a humanized 02A01-VH6-VK1. In certain embodiments, the antigen-binding site comprises a VH comprising HCDR1, HCDR2, and HCDR3 sequences set forth in SEQ ID NOs: 2, 17, and 4, respectively, and a VL comprising LCDR1, LCDR2, and LCDR3 sequences set forth in SEQ ID NOs: 6, 7, and 8, respectively. In certain embodiments, the antigen-binding site comprises a VH comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 21, and a VL that comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 22. In certain embodiments, the VH and the VL comprise the amino acid sequences of SEQ ID NOs: 21 and 22, respectively.

In certain embodiments, the antigen-binding site that binds CD8 is derived from a humanized 02A01-VH3-VK2. In certain embodiments, the antigen-binding site comprises a VH comprising HCDR1, HCDR2, and HCDR3 sequences set forth in SEQ ID NOs: 2, 17, and 4, respectively, and a VL comprising LCDR1, LCDR2, and LCDR3 sequences set forth in SEQ ID NOs: 6, 7, and 8, respectively. In certain embodiments, the antigen-binding site comprises a VH comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 16, and a VL that comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 23. In certain embodiments, the VH and the VL comprise the amino acid sequences of SEQ ID NOs: 16 and 23, respectively.

In certain embodiments, the antigen-binding site that binds CD8 is derived from a humanized 02A01-VH4-VK2. In certain embodiments, the antigen-binding site comprises a VH comprising HCDR1, HCDR2, and HCDR3 sequences set forth in SEQ ID NOs: 2, 17, and 4, respectively, and a VL comprising LCDR1, LCDR2, and LCDR3 sequences set forth in SEQ ID NOs: 6, 7, and 8, respectively. In certain embodiments, the antigen-binding site comprises a VH comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 18, and a VL that comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 23. In certain embodiments, the VH and the VL comprise the amino acid sequences of SEQ ID NOs: 18 and 23, respectively.

In certain embodiments, the antigen-binding site that binds CD8 is derived from a humanized 02A01-VH6-VK2. In certain embodiments, the antigen-binding site comprises a VH comprising HCDR1, HCDR2, and HCDR3 sequences set forth in SEQ ID NOs: 2, 17, and 4, respectively, and a VL comprising LCDR1, LCDR2, and LCDR3 sequences set forth in SEQ ID NOs: 6, 7, and 8, respectively. In certain embodiments, the antigen-binding site comprises a VH comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 21, and a VL that comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 23. In certain embodiments, the VH and the VL comprise the amino acid sequences of SEQ ID NOs: 21 and 23, respectively.

In certain embodiments, the antigen-binding site that binds CD8 is derived from a humanized 02A01-VH3-VK3. In certain embodiments, the antigen-binding site comprises a VH comprising HCDR1, HCDR2, and HCDR3 sequences set forth in SEQ ID NOs: 2, 17, and 4, respectively, and a VL comprising LCDR1, LCDR2, and LCDR3 sequences set forth in SEQ ID NOs: 6, 7, and 8, respectively. In certain embodiments, the antigen-binding site comprises a VH comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 16, and a VL that comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 24. In certain embodiments, the VH and the VL comprise the amino acid sequences of SEQ ID NOs: 16 and 24, respectively.

In certain embodiments, the antigen-binding site that binds CD8 is derived from a humanized 02A01-VH3-VK3(D30E/N35Q). In certain embodiments, the antigen-binding site comprises a VH comprising HCDR1, HCDR2, and HCDR3 sequences set forth in SEQ ID NOs: 2, 17, and 4, respectively, and a VL comprising LCDR1, LCDR2, and LCDR3 sequences set forth in SEQ ID NOs: 106, 7, and 8, respectively. In certain embodiments, the antigen-binding site comprises a VH comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 16, and a VL that comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 105. In certain embodiments, the VH and the VL comprise the amino acid sequences of SEQ ID NOs: 16 and 105, respectively.

In certain embodiments, the antigen-binding site that binds CD8 is derived from a humanized 02A01-VH4-VK3. In certain embodiments, the antigen-binding site comprises a VH comprising HCDR1, HCDR2, and HCDR3 sequences set forth in SEQ ID NOs: 2, 17, and 4, respectively, and a VL comprising LCDR1, LCDR2, and LCDR3 sequences set forth in SEQ ID NOs: 6, 7, and 8, respectively. In certain embodiments, the antigen-binding site comprises a VH comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 18, and a VL that comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 24. In certain embodiments, the VH and the VL comprise the amino acid sequences of SEQ ID NOs: 18 and 24, respectively.

In certain embodiments, the antigen-binding site that binds CD8 is derived from a humanized 02A01-VH4-VK3(D30E/N35Q). In certain embodiments, the antigen-binding site comprises a VH comprising HCDR1, HCDR2, and HCDR3 sequences set forth in SEQ ID NOs: 2, 17, and 4, respectively, and a VL comprising LCDR1, LCDR2, and LCDR3 sequences set forth in SEQ ID NOs: 106, 7, and 8, respectively. In certain embodiments, the antigen-binding site comprises a VH comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 18, and a VL that comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 105. In certain embodiments, the VH and the VL comprise the amino acid sequences of SEQ ID NOs: 18 and 105, respectively.

In certain embodiments, the antigen-binding site that binds CD8 is derived from a humanized 02A01-VH6-VK3. In certain embodiments, the antigen-binding site comprises a VH comprising HCDR1, HCDR2, and HCDR3 sequences set forth in SEQ ID NOs: 2, 17, and 4, respectively, and a VL comprising LCDR1, LCDR2, and LCDR3 sequences set forth in SEQ ID NOs: 6, 7, and 8, respectively. In certain embodiments, the antigen-binding site comprises a VH comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 21, and a VL that comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 24. In certain embodiments, the VH and the VL comprise the amino acid sequences of SEQ ID NOs: 21 and 24, respectively.

In certain embodiments, the antigen-binding site that binds CD8 is derived from a humanized 02A01-VH6-VK3(D30E/N35Q). In certain embodiments, the antigen-binding site comprises a VH comprising HCDR1, HCDR2, and HCDR3 sequences set forth in SEQ ID NOs: 2, 17, and 4, respectively, and a VL comprising LCDR1, LCDR2, and LCDR3 sequences set forth in SEQ ID NOs: 106, 7, and 8, respectively. In certain embodiments, the antigen-binding site comprises a VH comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 21, and a VL that comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 105. In certain embodiments, the VH and the VL comprise the amino acid sequences of SEQ ID NOs: 21 and 105, respectively.

In certain embodiments, the antigen-binding site that binds CD8 is derived from a humanized 02A01-VH1-VK4. In certain embodiments, the antigen-binding site comprises a VH comprising HCDR1, HCDR2, and HCDR3 sequences set forth in SEQ ID NOs: 2, 3, and 4, respectively, and a VL comprising LCDR1, LCDR2, and LCDR3 sequences set forth in SEQ ID NOs: 6, 26, and 8, respectively. In certain embodiments, the antigen-binding site comprises a VH comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 14, and a VL that comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 25. In certain embodiments, the VH and the VL comprise the amino acid sequences of SEQ ID NOs: 14 and 25, respectively.

In certain embodiments, the antigen-binding site that binds CD8 is derived from a humanized 02A01-VH2-VK4. In certain embodiments, the antigen-binding site comprises a VH comprising HCDR1, HCDR2, and HCDR3 sequences set forth in SEQ ID NOs: 2, 3, and 4, respectively, and a VL comprising LCDR1, LCDR2, and LCDR3 sequences set forth in SEQ ID NOs: 6, 26, and 8, respectively. In certain embodiments, the antigen-binding site comprises a VH comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 15, and a VL that comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 25. In certain embodiments, the VH and the VL comprise the amino acid sequences of SEQ ID NOs: 15 and 25, respectively.

In certain embodiments, the antigen-binding site that binds CD8 is derived from a humanized 02A01-VH3-VK4. In certain embodiments, the antigen-binding site comprises a VH comprising HCDR1, HCDR2, and HCDR3 sequences set forth in SEQ ID NOs: 2, 17, and 4, respectively, and a VL comprising LCDR1, LCDR2, and LCDR3 sequences set forth in SEQ ID NOs: 6, 26, and 8, respectively. In certain embodiments, the antigen-binding site comprises a VH comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 16, and a VL that comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 25. In certain embodiments, the VH and the VL comprise the amino acid sequences of SEQ ID NOs: 16 and 25, respectively.

In certain embodiments, the antigen-binding site that binds CD8 is derived from a humanized 02A01-VH4-VK4. In certain embodiments, the antigen-binding site comprises a VH comprising HCDR1, HCDR2, and HCDR3 sequences set forth in SEQ ID NOs: 2, 17, and 4, respectively, and a VL comprising LCDR1, LCDR2, and LCDR3 sequences set forth in SEQ ID NOs: 6, 26, and 8, respectively. In certain embodiments, the antigen-binding site comprises a VH comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 18, and a VL that comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 25. In certain embodiments, the VH and the VL comprise the amino acid sequences of SEQ ID NOs: 18 and 25, respectively.

In certain embodiments, the antigen-binding site that binds CD8 is derived from a humanized 02A01-VH6-VK4. In certain embodiments, the antigen-binding site comprises a VH comprising HCDR1, HCDR2, and HCDR3 sequences set forth in SEQ ID NOs: 2, 17, and 4, respectively, and a VL comprising LCDR1, LCDR2, and LCDR3 sequences set forth in SEQ ID NOs: 6, 26, and 8, respectively. In certain embodiments, the antigen-binding site comprises a VH comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 21, and a VL that comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 25. In certain embodiments, the VH and the VL comprise the amino acid sequences of SEQ ID NOs: 21 and 25, respectively.

In certain embodiments, the antigen-binding site that binds CD8 is derived from murine antibody 02A01. In certain embodiments, the antigen-binding site comprises a VH comprising HCDR1, HCDR2, and HCDR3 sequences set forth in SEQ ID NOs: 2, 3, and 4, respectively, and a VL comprising LCDR1, LCDR2, and LCDR3 sequences set forth in SEQ ID NOs: 6, 7, and 8, respectively. In certain embodiments, the antigen-binding site comprises a VH comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 1, and a VL that comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 5. In certain embodiments, the VH and the VL comprise the amino acid sequences of SEQ ID NOs: 1 and 5, respectively.

In certain embodiments, the antigen-binding site that binds CD8 is derived from murine antibody 02A01 VH-M39L. In certain embodiments, the antigen-binding site comprises a VH comprising HCDR1, HCDR2, and HCDR3 sequences set forth in SEQ ID NOs: 10, 3, and 4, respectively, and a VL comprising LCDR1, LCDR2, and LCDR3 sequences set forth in SEQ ID NOs: 6, 7, and 8, respectively. In certain embodiments, the antigen-binding site comprises a VH comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 9, and a VL that comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 5. In certain embodiments, the VH and the VL comprise the amino acid sequences of SEQ ID NOs: 9 and 5, respectively.

In certain embodiments, the antigen-binding site disclosed herein derived from a murine or humanized 02A01 antibody binds human CD8 in a surface plasmon resonance (SPR) assay with a dissociation constant (K_{D}) value smaller than or equal to (binding affinity greater than or equal to) 10 nM, 15 nM, 20 nM, 25 nM, 30 nM, 40 nM or 50 nM. An exemplary method of SPR assay is described in Example 2 below. In certain embodiments, a bivalent construct of the antigen-binding site disclosed herein derived from a murine or humanized 02A01 antibody, for example, an anti-CD8 IgG antibody, binds cells expressing human CD8 (e.g., human CD8⁺ T cells or human T cells comprising a CD8⁺ population) with a K_{D} value smaller than or equal to (binding affinity greater than or equal to) 60 pM, 70 pM, 80 pM, 90 pM, 0.1 nM, 0.2 nM, 0.3 nM, 0.4 nM, 0.5 nM, 0.6 nM, 0.7 nM, 0.8 nM, 0.9 nM, 1 nM, 2 nM, 3 nM, 4 nM, or 5 nM. An exemplary method of the cell binding assay is described in Example 2 below. Without wishing to be bound by theory, it has been contemplated that the apparent K_{D} value of a bivalent construct measured by a cell binding assay is lower than the K_{D} value of the same construct measured by SPR, as a result of increased avidity over affinity.

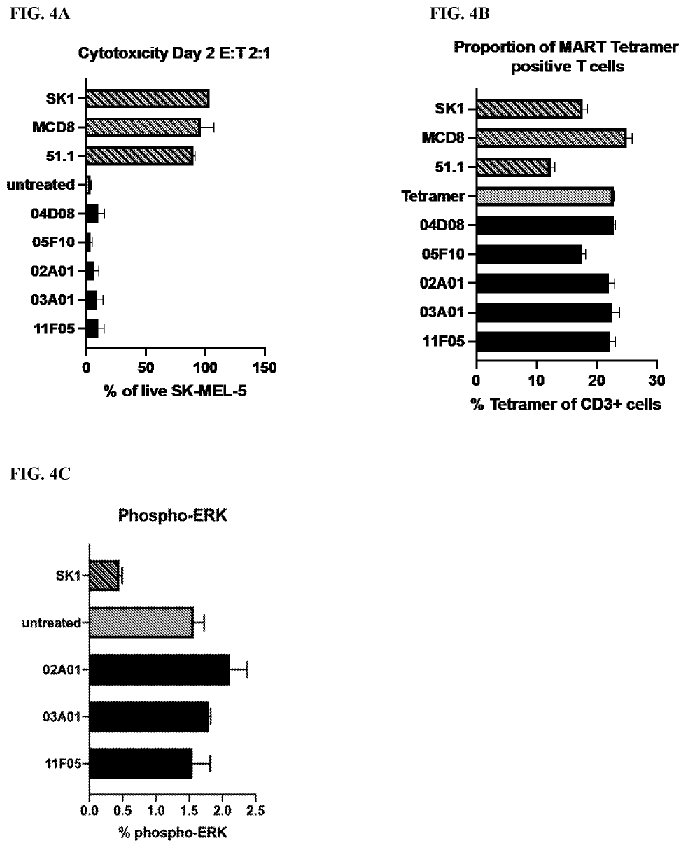

In certain embodiments, the antigen-binding site disclosed herein derived from a murine or humanized 02A01 antibody does not cross-compete with OKT8, SK1, 51.1, or MCD8. OKT8 has been found to enhance the on-rates of TCR binding to peptide-MHC class I (pMHC-I), improve pMHC-I tetramer staining on CD8⁺ T cells, and induce effector function in CD8⁺ T cells (see, e.g., Clement et al., J. Immunol. (2011) 187(2):654-63). SK1 and MCD8 have been found to inhibit pMHC-I tetramer staining on CD8⁺ T cells (see, e.g., Wooldridge et al., J. Immunol. (2003) 171:6650-60; Denkberg et al., J. Immunol. (2001) 167:270-76). Data presented herein indicate that 51.1 inhibits pMHC-I tetramer binding to CD8+ T cells and abrogates T cell-mediated killing of SK-MEL-5 tumor cells (see Example 1, FIGS. 4A-4B). Thus, it is understood that OKT8 is an agonistic antibody and SK1, 51.1, and MCD8 are antagonistic antibodies. Without wishing to be bound by theory, it is contemplated that an antigen-binding site disclosed herein derived from a murine or humanized 02A01 antibody binds a different epitope on CD8 from OKT8, SK1, 51.1, or MCD8.

In certain embodiments, an antigen-binding site disclosed herein cross-competes for binding CD8 (e.g., human CD8) with an antibody or antigen-binding site derived from a murine or humanized 02A01 antibody, e.g., an antibody or antigen-binding site comprising the VH and VL sequences provided in Table 2A or 2B.

In certain embodiments, an antigen-binding site disclosed herein (e.g., one derived from a murine or humanized 02A01 antibody) binds (e.g., specifically binds) to an epitope corresponding to amino acids 28-32, 34, 48, and 51 of a protein having the amino acid sequence of

(SEQ ID NO: 110)
SQFRVSPLDRTWNLGETVELKCQVLLSNPTSGCSWLFQPRGAAASPTFL
LYLSQNKPKAAEGLDTQRFSGKRLGDTFVLTLSDFRRENEGYYFCSALS
NSIMYFSHFVPVFLPAKPTTTPAPRPPTPAPTIASQPLSLRPEACRPAA
GGAVHTRGLDFACD.

In certain embodiments, an antigen-binding site disclosed herein (e.g., one derived from a murine or humanized 02A01 antibody) binds (e.g., specifically binds) to an epitope corresponding to amino acids 49-53, 55, 69, and 72 of a protein having the amino acid sequence of

(SEQ ID NO: 111)
MALPVTALLLPLALLLHAARPSQFRVSPLDRTWNLGETVELKCQVLLSN
PTSGCSWLFQPRGAAASPTFLLYLSQNKPKAAEGLDTQRFSGKRLGDTF
VLTLSDFRRENEGYYFCSALSNSIMYFSHFVPVFLPAKPTTTPAPRPPT
PAPTIASQPLSLRPEACRPAAGGAVHTRGLDFACDIYIWAPLAGTCGVL
LLSLVITLYCNHRNRRRVCKCPRPVVKSGDKPSLSARYV.

CD8-Binding Sites Derived from 03A01

In certain embodiments, the antigen-binding site that binds CD8 disclosed herein comprises a VH that comprises an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to the VH of an antibody disclosed in Table 3A or 3B, and a VL that comprises an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to the VL of the same antibody disclosed in Table 3A or 3B. In certain embodiments, the antigen-binding site comprises the HCDR1, HCDR2, HCDR3, LCDR1, LCDR2, and LCDR3, determined under Kabat (e.g., as disclosed in Table 3A; see Kabat et al., (1991) Sequences of Proteins of Immunological Interest, NIH Publication No. 91-3242, Bethesda), Chothia (e.g., as disclosed in Table 3B; see, e.g., Chothia C & Lesk A M, (1987), J. Mol. Biol. 196: 901-917), MacCallum (see MacCallum R M et al., (1996) J. Mol. Biol. 262: 732-745), IMGT (see Lefranc, (1999) The Immunologist, 7, 132-136), or any other CDR determination method known in the art, of the VH and VL sequences of an antibody disclosed in Table 3A or 3B. In certain embodiments, the antigen-binding site comprises the HCDR1, HCDR2, HCDR3, LCDR1, LCDR2, and LCDR3 sequences of an antibody disclosed in Table 3A or 3B. In certain embodiments, the antigen-binding site comprises the VH and VL sequences of an antibody disclosed in Table 3A or 3B.

In certain embodiments, the antigen-binding site that binds CD8 comprises a VH comprising HCDR1, HCDR2, and HCDR3 sequences set forth in SEQ ID NOs: 115, 118, and 30, respectively, and a VL comprising LCDR1, LCDR2, and LCDR3 sequences set forth in SEQ ID NOs: 32, 33, and 34, respectively. In certain embodiments, the HCDR1 sequence is SEQ ID NO: 115; the HCDR2 sequence is selected from the group consisting of SEQ ID NOs: 116 and 117; the HCDR3 sequence is SEQ ID NO: 30; the LCDR1 sequence is SEQ ID NO: 32; the LCDR2 sequence is SEQ ID NO: 33; and/or the LCDR3 sequence is SEQ ID NO: 34.

In certain embodiments, the antigen-binding site that binds CD8 is derived from a humanized 03A01. In certain embodiments, the antigen-binding site comprises a VH comprising HCDR1, HCDR2, and HCDR3 sequences set forth in SEQ ID NOs: 115, 116, and 30; or 115, 117, and 30, respectively, and a VL comprising LCDR1, LCDR2, and LCDR3 sequences set forth in SEQ ID NOs: 32, 33, and 34, respectively.

In certain embodiments, the antigen-binding site that binds CD8 comprises a VH comprising HCDR1, HCDR2, and HCDR3 sequences set forth in SEQ ID NOs: 28, 37, and 30, respectively, and a VL comprising LCDR1, LCDR2, and LCDR3 sequences set forth in SEQ ID NOs: 32, 33, and 34, respectively. In certain embodiments, the HCDR1 sequence is SEQ ID NO: 28; the HCDR2 sequence is selected from the group consisting of SEQ ID NOs: 29, 36, 42, and 44; the HCDR3 sequence is SEQ ID NO: 30; the LCDR1 sequence is SEQ ID NO: 32; the LCDR2 sequence is SEQ ID NO: 33; and/or the LCDR3 sequence is SEQ ID NO: 34.

In certain embodiments, the antigen-binding site that binds CD8 comprises a VH comprising HCDR1, HCDR2, and HCDR3 sequences set forth in SEQ ID NOs: 28, 38, and 30, respectively, and a VL comprising LCDR1, LCDR2, and LCDR3 sequences set forth in SEQ ID NOs: 32, 33, and 34, respectively. In certain embodiments, the HCDR1 sequence is SEQ ID NO: 28; the HCDR2 sequence is selected from the group consisting of SEQ ID NOs: 29, 42, and 44; the HCDR3 sequence is SEQ ID NO: 30; the LCDR1 sequence is SEQ ID NO: 32; the LCDR2 sequence is SEQ ID NO: 33; and/or the LCDR3 sequence is SEQ ID NO: 34.

In certain embodiments, the antigen-binding site that binds CD8 is derived from a humanized 03A01. In certain embodiments, the antigen-binding site comprises a VH comprising HCDR1, HCDR2, and HCDR3 sequences set forth in SEQ ID NOs: 28, 29, and 30; 28, 42, and 30; or 28, 44, and 30, respectively, and a VL comprising LCDR1, LCDR2, and LCDR3 sequences set forth in SEQ ID NOs: 32, 33, and 34, respectively.

In certain embodiments, the antigen-binding site that binds CD8 is derived from a humanized 03A01-VH1-VK1. In certain embodiments, the antigen-binding site comprises a VH comprising HCDR1, HCDR2, and HCDR3 sequences set forth in SEQ ID NOs: 28, 29, and 30, respectively, and a VL comprising LCDR1, LCDR2, and LCDR3 sequences set forth in SEQ ID NOs: 32, 33, and 34, respectively. In certain embodiments, the antigen-binding site comprises a VH comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 39, and a VL that comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 45. In certain embodiments, the VH and the VL comprise the amino acid sequences of SEQ ID NOs: 39 and 45, respectively.

In certain embodiments, the antigen-binding site that binds CD8 is derived from a humanized 03A01-VH2-VK1. In certain embodiments, the antigen-binding site comprises a VH comprising HCDR1, HCDR2, and HCDR3 sequences set forth in SEQ ID NOs: 28, 29, and 30, respectively, and a VL comprising LCDR1, LCDR2, and LCDR3 sequences set forth in SEQ ID NOs: 32, 33, and 34, respectively. In certain embodiments, the antigen-binding site comprises a VH comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 40, and a VL that comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 45. In certain embodiments, the VH and the VL comprise the amino acid sequences of SEQ ID NOs: 40 and 45, respectively.

In certain embodiments, the antigen-binding site that binds CD8 is derived from a humanized 03A01-VH1-VK2. In certain embodiments, the antigen-binding site comprises a VH comprising HCDR1, HCDR2, and HCDR3 sequences set forth in SEQ ID NOs: 28, 29, and 30, respectively, and a VL comprising LCDR1, LCDR2, and LCDR3 sequences set forth in SEQ ID NOs: 32, 33, and 34, respectively. In certain embodiments, the antigen-binding site comprises a VH comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 39, and a VL that comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 46. In certain embodiments, the VH and the VL comprise the amino acid sequences of SEQ ID NOs: 39 and 46, respectively.

In certain embodiments, the antigen-binding site that binds CD8 is derived from a humanized 03A01-VH2-VK2. In certain embodiments, the antigen-binding site comprises a VH comprising HCDR1, HCDR2, and HCDR3 sequences set forth in SEQ ID NOs: 28, 29, and 30, respectively, and a VL comprising LCDR1, LCDR2, and LCDR3 sequences set forth in SEQ ID NOs: 32, 33, and 34, respectively. In certain embodiments, the antigen-binding site comprises a VH comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 40, and a VL that comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 46. In certain embodiments, the VH and the VL comprise the amino acid sequences of SEQ ID NOs: 40 and 46, respectively.

In certain embodiments, the antigen-binding site that binds CD8 is derived from a humanized 03A01-VH1-VK3. In certain embodiments, the antigen-binding site comprises a VH comprising HCDR1, HCDR2, and HCDR3 sequences set forth in SEQ ID NOs: 28, 29, and 30, respectively, and a VL comprising LCDR1, LCDR2, and LCDR3 sequences set forth in SEQ ID NOs: 32, 33, and 34, respectively. In certain embodiments, the antigen-binding site comprises a VH comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 39, and a VL that comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 47. In certain embodiments, the VH and the VL comprise the amino acid sequences of SEQ ID NOs: 39 and 47, respectively.

In certain embodiments, the antigen-binding site that binds CD8 is derived from a humanized 03A01-VH2-VK3. In certain embodiments, the antigen-binding site comprises a VH comprising HCDR1, HCDR2, and HCDR3 sequences set forth in SEQ ID NOs: 28, 29, and 30, respectively, and a VL comprising LCDR1, LCDR2, and LCDR3 sequences set forth in SEQ ID NOs: 32, 33, and 34, respectively. In certain embodiments, the antigen-binding site comprises a VH comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 40, and a VL that comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 47. In certain embodiments, the VH and the VL comprise the amino acid sequences of SEQ ID NOs: 40 and 47, respectively.

In certain embodiments, the antigen-binding site that binds CD8 is derived from a humanized 03A01-VH3-VK1. In certain embodiments, the antigen-binding site comprises a VH comprising HCDR1, HCDR2, and HCDR3 sequences set forth in SEQ ID NOs: 28, 42, and 30, respectively, and a VL comprising LCDR1, LCDR2, and LCDR3 sequences set forth in SEQ ID NOs: 32, 33, and 34, respectively. In certain embodiments, the antigen-binding site comprises a VH comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 41, and a VL that comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 45. In certain embodiments, the VH and the VL comprise the amino acid sequences of SEQ ID NOs: 41 and 45, respectively.

In certain embodiments, the antigen-binding site that binds CD8 is derived from a humanized 03A01-VH3-VK2. In certain embodiments, the antigen-binding site comprises a VH comprising HCDR1, HCDR2, and HCDR3 sequences set forth in SEQ ID NOs: 28, 42, and 30, respectively, and a VL comprising LCDR1, LCDR2, and LCDR3 sequences set forth in SEQ ID NOs: 32, 33, and 34, respectively. In certain embodiments, the antigen-binding site comprises a VH comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 41, and a VL that comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 46. In certain embodiments, the VH and the VL comprise the amino acid sequences of SEQ ID NOs: 41 and 46, respectively.

In certain embodiments, the antigen-binding site that binds CD8 is derived from a humanized 03A01-VH3-VK3. In certain embodiments, the antigen-binding site comprises a VH comprising HCDR1, HCDR2, and HCDR3 sequences set forth in SEQ ID NOs: 28, 42, and 30, respectively, and a VL comprising LCDR1, LCDR2, and LCDR3 sequences set forth in SEQ ID NOs: 32, 33, and 34, respectively. In certain embodiments, the antigen-binding site comprises a VH comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 41, and a VL that comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 47. In certain embodiments, the VH and the VL comprise the amino acid sequences of SEQ ID NOs: 41 and 47, respectively.

In certain embodiments, the antigen-binding site that binds CD8 is derived from a humanized 03A01-VH4-VK1. In certain embodiments, the antigen-binding site comprises a VH comprising HCDR1, HCDR2, and HCDR3 sequences set forth in SEQ ID NOs: 28, 44, and 30, respectively, and a VL comprising LCDR1, LCDR2, and LCDR3 sequences set forth in SEQ ID NOs: 32, 33, and 34, respectively. In certain embodiments, the antigen-binding site comprises a VH comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 43, and a VL that comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 45. In certain embodiments, the VH and the VL comprise the amino acid sequences of SEQ ID NOs: 43 and 45, respectively.

In certain embodiments, the antigen-binding site that binds CD8 is derived from a humanized 03A01-VH4-VK2. In certain embodiments, the antigen-binding site comprises a VH comprising HCDR1, HCDR2, and HCDR3 sequences set forth in SEQ ID NOs: 28, 44, and 30, respectively, and a VL comprising LCDR1, LCDR2, and LCDR3 sequences set forth in SEQ ID NOs: 32, 33, and 34, respectively. In certain embodiments, the antigen-binding site comprises a VH comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 43, and a VL that comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 46. In certain embodiments, the VH and the VL comprise the amino acid sequences of SEQ ID NOs: 43 and 46, respectively.

In certain embodiments, the antigen-binding site that binds CD8 is derived from a humanized 03A01-VH4-VK3. In certain embodiments, the antigen-binding site comprises a VH comprising HCDR1, HCDR2, and HCDR3 sequences set forth in SEQ ID NOs: 28, 44, and 30, respectively, and a VL comprising LCDR1, LCDR2, and LCDR3 sequences set forth in SEQ ID NOs: 32, 33, and 34, respectively. In certain embodiments, the antigen-binding site comprises a VH comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 43, and a VL that comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 47. In certain embodiments, the VH and the VL comprise the amino acid sequences of SEQ ID NOs: 43 and 47, respectively.

In certain embodiments, the antigen-binding site that binds CD8 is derived from murine antibody 03A01. In certain embodiments, the antigen-binding site comprises a VH comprising HCDR1, HCDR2, and HCDR3 sequences set forth in SEQ ID NOs: 28, 29, and 30, respectively, and a VL comprising LCDR1, LCDR2, and LCDR3 sequences set forth in SEQ ID NOs: 32, 33, and 34, respectively. In certain embodiments, the antigen-binding site comprises a VH comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 27, and a VL that comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 31. In certain embodiments, the VH and the VL comprise the amino acid sequences of SEQ ID NOs: 27 and 31, respectively.

In certain embodiments, the antigen-binding site that binds CD8 is derived from murine antibody 03A01 VH-D64E. In certain embodiments, the antigen-binding site comprises a VH comprising HCDR1, HCDR2, and HCDR3 sequences set forth in SEQ ID NOs: 28, 36, and 30, respectively, and a VL comprising LCDR1, LCDR2, and LCDR3 sequences set forth in SEQ ID NOs: 32, 33, and 34, respectively. In certain embodiments, the antigen-binding site comprises a VH comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 35, and a VL that comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 31. In certain embodiments, the VH and the VL comprise the amino acid sequences of SEQ ID NOs: 35 and 31, respectively.

In certain embodiments, a bivalent construct of the antigen-binding site disclosed herein derived from a murine or humanized 03A01 antibody, for example, an anti-CD8 IgG antibody, binds cells expressing human CD8 (e.g., human CD8⁺ T cells or human T cells comprising a CD8⁺ population) with a K_{D} value smaller than or equal to (binding affinity greater than or equal to 1 nM, 2 nM, 3 nM, 4 nM, 5 nM, 6 nM, 7 nM, 8 nM, 9 nM, or 10 nM. An exemplary method of the cell binding assay is described in Example 2 below. Without wishing to be bound by theory, it has been contemplated that the apparent K_{D} value of a bivalent construct measured by a cell binding assay is lower than the K_{D} value of the same construct measured by SPR, as a result of increased avidity over affinity.

In certain embodiments, the antigen-binding site disclosed herein derived from a murine or humanized 03A01 antibody does not cross-compete with OKT8, SK1, 51.1, or MCD8. It is understood that OKT8 is an agonistic antibody and SK1, 51.1, and MCD8 are antagonistic antibodies. Without wishing to be bound by theory, it is contemplated that an antigen-binding site disclosed herein derived from a murine or humanized 03A01 antibody binds a different epitope on CD8 from OKT8, SK1, 51.1, or MCD8.

The present disclosure also provides an antigen-binding site that cross-competes for binding CD8 (e.g., human CD8) with an antibody or antigen-binding site derived from a murine or humanized 03A01 antibody, e.g., an antibody or antigen-binding site comprising the VH and VL sequences provided in Table 3A or 3B.

In certain embodiments, an antigen-binding site disclosed herein (e.g., one derived from a murine or humanized 03A01 antibody) binds (e.g., specifically binds) to an epitope corresponding to amino acids 26, 28-32, 48, 51, 53, 97, and 100 of a protein having the amino acid sequence of SEQ ID NO: 110. In certain embodiments, an antigen-binding site disclosed herein (e.g., one derived from a murine or humanized 03A01 antibody) binds (e.g., specifically binds) to an epitope corresponding to amino acids 47, 49-53, 69, 72, 74, 118, and 121 of a protein having the amino acid sequence of SEQ ID NO: 111.

CD8-Binding Sites Derived from 05F10, 11F05, and 04D08

In certain embodiments, the antigen-binding site that binds CD8 disclosed herein comprises a VH that comprises an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to the VH of an antibody disclosed in Table 4A or 4B, and a VL that comprises an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to the VL of the same antibody disclosed in Table 4A or 4B. In certain embodiments, the antigen-binding site comprises the HCDR1, HCDR2, HCDR3, LCDR1, LCDR2, and LCDR3, determined under Kabat (e.g., as disclosed in Table 4A; see Kabat et al., (1991) Sequences of Proteins of Immunological Interest, NIH Publication No. 91-3242, Bethesda), Chothia (e.g., as disclosed in Table 4B; see, e.g., Chothia C & Lesk A M, (1987), J. Mol. Biol. 196: 901-917), MacCallum (see MacCallum R M et al., (1996) J. Mol. Biol. 262: 732-745), IMGT (see Lefranc, (1999) The Immunologist, 7, 132-136), or any other CDR determination method known in the art, of the VH and VL sequences of an antibody disclosed in Table 4A or 4B. In certain embodiments, the antigen-binding site comprises the HCDR1, HCDR2, HCDR3, LCDR1, LCDR2, and LCDR3 sequences of an antibody disclosed in Table 4A or 4B. In certain embodiments, the antigen-binding site comprises the VH and VL sequences of an antibody disclosed in Table 4A or 4B.

In certain embodiments, the antigen-binding site that binds CD8 is derived from 05F10. In certain embodiments, the antigen-binding site comprises a VH comprising HCDR1, HCDR2, and HCDR3 sequences set forth in SEQ ID NOs: 51, 52, and 53, respectively, and a VL comprising LCDR1, LCDR2, and LCDR3 sequences set forth in SEQ ID NOs: 55, 56, and 57, respectively. In certain embodiments, the antigen-binding site comprises a VH comprising HCDR1, HCDR2, and HCDR3 sequences set forth in SEQ ID NOs: 119, 120, and 53, respectively, and a VL comprising LCDR1, LCDR2, and LCDR3 sequences set forth in SEQ ID NOs: 55, 56, and 57, respectively. In certain embodiments, the antigen-binding site comprises a VH comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 50, and a VL that comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 54. In certain embodiments, the VH and the VL comprise the amino acid sequences of SEQ ID NOs: 50 and 54, respectively.

In certain embodiments, the antigen-binding site that binds CD8 is derived from 11F05. In certain embodiments, the antigen-binding site comprises a VH comprising HCDR1, HCDR2, and HCDR3 sequences set forth in SEQ ID NOs: 59, 60, and 61, respectively, and a VL comprising LCDR1, LCDR2, and LCDR3 sequences set forth in SEQ ID NOs: 32, 63, and 64, respectively. In certain embodiments, the antigen-binding site comprises a VH comprising HCDR1, HCDR2, and HCDR3 sequences set forth in SEQ ID NOs: 121, 122, and 61, respectively, and a VL comprising LCDR1, LCDR2, and LCDR3 sequences set forth in SEQ ID NOs: 32, 63, and 64, respectively. In certain embodiments, the antigen-binding site comprises a VH comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 58, and a VL that comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 62. In certain embodiments, the VH and the VL comprise the amino acid sequences of SEQ ID NOs: 58 and 62, respectively.

In certain embodiments, the antigen-binding site that binds CD8 is derived from 04D08. In certain embodiments, the antigen-binding site comprises a VH comprising HCDR1, HCDR2, and HCDR3 sequences set forth in SEQ ID NOs: 66, 67, and 68, respectively, and a VL comprising LCDR1, LCDR2, and LCDR3 sequences set forth in SEQ ID NOs: 70, 71, and 72, respectively. In certain embodiments, the antigen-binding site comprises a VH comprising HCDR1, HCDR2, and HCDR3 sequences set forth in SEQ ID NOs: 123, 124, and 68, respectively, and a VL comprising LCDR1, LCDR2, and LCDR3 sequences set forth in SEQ ID NOs: 70, 71, and 72, respectively. In certain embodiments, the antigen-binding site comprises a VH comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 65, and a VL that comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 69. In certain embodiments, the VH and the VL comprise the amino acid sequences of SEQ ID NOs: 65 and 69, respectively.

Any one of the antigen-binding sites disclosed herein may be affinity matured. In immunology, affinity maturation is the process by which B cells produce antibodies with increased affinity for antigen during the course of an immune response. With repeated exposures to the same antigen, a host will produce antibodies of successively greater affinities. Like the natural prototype, the in vitro affinity maturation is based on the principles of mutation and selection. Two or three rounds of mutation and selection using display methods such as phage display can result in antibody fragments with affinities in the low nanomolar range.

An amino acid substitution variation can be introduced into the antigen-binding site by substituting one or more hypervariable region residues of a parent antibody (e.g., a humanized or human antibody). Generally, the resulting variant(s) selected for further development will have improved biological properties relative to the parent antibody from which they are generated. A convenient way for generating such substitutional variants involves affinity maturation using phage display. Briefly, several hypervariable region sides (e.g., 6-7 sides) are mutated to generate all possible amino acid substitutions at each side. The antibody variants thus generated are displayed in a monovalent fashion from filamentous phage particles as fusions to the gene III product of M13 packaged within each particle. The phage-displayed variants are then screened for their biological activity (e.g., binding affinity) as herein disclosed. In order to identify candidate hypervariable region sides for modification, alanine scanning mutagenesis can be performed to identify hypervariable region residues contributing significantly to antigen binding. Alternatively, or additionally, it may be beneficial to analyze a crystal structure of the antigen-antibody complex to identify contact points between the binding domains. Such contact residues and neighboring residues are candidates for substitution according to the techniques elaborated herein. Once such variants are generated, the panel of variants is subjected to screening as described herein and antibodies with superior properties in one or more relevant assays may be selected for further development.

In certain embodiments, the antigen-binding site disclosed herein binds (e.g., specifically binds) to an epitope corresponding to amino acids 28-32, 48, and 51 of a protein having the amino acid sequence of SEQ ID NO: 110. In certain embodiments, the antigen-binding site disclosed herein binds (e.g., specifically binds) to an epitope corresponding to amino acids 28-32, 34, 48, and 51 of a protein having the amino acid sequence of SEQ ID NO: 110. In certain embodiments, the antigen-binding site disclosed herein binds (e.g., specifically binds) to an epitope corresponding to amino acids 26, 28-32, 48, 51, 53, 97, and 100 of a protein having the amino acid sequence of SEQ ID NO: 110. The epitope can be mapped using a method known in the art, for example, as described in Example 1 herein. In certain embodiments, the antigen-binding site disclosed herein does not cross-compete with OKT8, SK1, 51.1, or MCD8 for binding to CD8 (e.g., human CD8).

In certain embodiments, the antigen-binding site disclosed herein binds human CD8, in a surface plasmon resonance (SPR) assay, with a dissociation constant (K_{D}) value smaller than or equal to (binding affinity greater than or equal to) 10 nM, 15 nM, 20 nM, 25 nM, 30 nM, 40 nM, 50 nM, 60 nM, 70 nM, 80 nM, 90 nM, or 100 nM. An exemplary method of SPR assay is described in Example 2 below. In certain embodiments, a bivalent construct of the antigen-binding site disclosed herein, for example, an anti-CD8 IgG antibody, binds cells expressing human CD8 (e.g., human CD8⁺ T cells or human T cells comprising a CD8⁺ population) with a K_{D} value smaller than or equal to (binding affinity greater than or equal to) 50 pM, 60 pM, 70 pM, 80 pM, 90 pM, 0.1 nM, 0.2 nM, 0.3 nM, 0.4 nM, 0.5 nM, 0.6 nM, 0.7 nM, 0.8 nM, 0.9 nM, 1 nM, 2 nM, 3 nM, 4 nM, 5 nM, 6 nM, 7 nM, 8 nM, 9 nM, or 10 nM. An exemplary method of the cell binding assay is described in Example 2 below. Without wishing to be bound by theory, it has been contemplated that the apparent K_{D} value of a bivalent construct measured by a cell binding assay is lower than the K_{D} value of the same construct measured by SPR, as a result of increased avidity over affinity. In certain embodiments, the CD8-binding portion of the fusion protein binds cells expressing human CD8 (e.g., human CD8⁺ T cells or human T cells comprising a CD8⁺ population) with a K_{D} value smaller than or equal to (binding affinity greater than or equal to) 50 pM, 60 pM, 70 pM, 80 pM, 90 pM, 0.1 nM, 0.2 nM, 0.3 nM, 0.4 nM, 0.5 nM, 0.6 nM, 0.7 nM, 0.8 nM, 0.9 nM, 1 nM, 2 nM, 3 nM, 4 nM, 5 nM, 6 nM, 7 nM, 8 nM, 9 nM, 10 nM, 15 nM, 20 nM, 25 nM, 30 nM, 40 nM, 50 nM, 60 nM, 70 nM, 80 nM, 90 nM, or 100 nM. This binding affinity can be assessed, for example, by truncating the IL-12 portion from the fusion protein or abrogating its binding affinity to IL-12 receptor. In certain embodiments, the CD8-binding portion of the fusion protein binds cells expressing human CD8 (e.g., human CD8⁺ T cells or human T cells comprising a CD8⁺ population) with a K_{D} value smaller than (binding affinity greater than) the K_{D} value at which the IL-12 portion of the fusion protein binds the cells by at least 5-fold, 10-fold, 20-fold, 30-fold, 40-fold, 50-fold, 60-fold, 70-fold, 80-fold, 90-fold, or 100-fold.

The antigen-binding sites that bind CD8 disclosed herein is silent and, for example, is lacking a substantial agonistic activity and lacking a substantial antagonistic activity of the antibody or antigen-binding site. In certain embodiments, the antigen-binding site, at a concentration that is at least 10-fold, 20-fold, 30-fold, 40-fold, 50-fold, 60-fold, 70-fold, 80-fold, 90-fold, or 100-fold higher than the K_{D} value with which the antigen-binding site binds cells expressing human CD8 (e.g., human CD8⁺ T cells or human T cells comprising a CD8* population), does not increase an activity of CD8 by more than 10%, 20%, 30%, 40%, 50%, 60%, 70%, 80%, or 90% and/or does not decrease an activity of CD8 by more than 10%, 20%, 30%, 40%, 50%, 60%, 70%, 80%, or 90%. In certain embodiments, the antigen-binding site, at a concentration that is at least 10-fold, 20-fold, 30-fold, 40-fold, 50-fold, 60-fold, 70-fold, 80-fold, 90-fold, or 100-fold higher than the K_{D} value with which the antigen-binding site binds cells expressing human CD8 (e.g., human CD8⁺ T cells or human T cells comprising a CD8⁺ population), does not increase an activity of CD8 by more than 1.5 fold, 2 fold, 2.5 fold, 3 fold, 4 fold, or 5 fold and/or does not decrease an activity of CD8 by more than 1.5 fold, 2 fold, 2.5 fold, 3 fold, 4 fold, or 5 fold. The activity of CD8 can be stabilization of a peptide-MHC-TCR complex, activation of a TCR signaling pathway, and/or activation of a transcription factor downstream of TCR signaling, as measured by the assays disclosed herein (see, e.g., the Examples section below).

In certain embodiments, the antigen-binding site, at a concentration that is at least 10-fold, 20-fold, 30-fold, 40-fold, 50-fold, 60-fold, 70-fold, 80-fold, 90-fold, or 100-fold higher than the K_{D} value with which the antigen-binding site binds cells expressing human CD8 (e.g., human CD8⁺ T cells or human T cells comprising a CD8⁺ population), does not increase or decrease the stability of a complex by more than 10%, 20%, 30%, 40%, 50%, 60%, 70%, 80%, or 90%, the complex comprising CD8, a peptide or T cell epitope presented by a human class I MHC tetramer, and a cognate TCR, as measured by the amount of the tetramer bound to a cell expressing the TCR. In certain embodiments, the antigen-binding site, at a concentration that is at least 10-fold, 20-fold, 30-fold, 40-fold, 50-fold, 60-fold, 70-fold, 80-fold, 90-fold, or 100-fold higher than the K_{D} value with which the antigen-binding site binds cells expressing human CD8 (e.g., human CD8⁺ T cells or human T cells comprising a CD8⁺ population), does not increase or decrease the stability of the complex by more than 1.5 fold, 2 fold, 2.5 fold, 3 fold, 4 fold, or 5 fold. An exemplary assay measuring the stability of the complex is described in Example 1 below.

In certain embodiments, the antigen-binding site, at a concentration that is at least 10-fold, 20-fold, 30-fold, 40-fold, 50-fold, 60-fold, 70-fold, 80-fold, 90-fold, or 100-fold higher than the K_{D} value with which the antigen-binding site binds cells expressing human CD8 (e.g., human CD8⁺ T cells or human T cells comprising a CD8* population), does not increase or decrease T cell activation by more than 10%, 20%, 30%, 40%, 50%, 60%, 70%, 80%, or 90%, as measured by ERK phosphorylation in T cells stimulated by a cognate T cell epitope presented by a human class I MHC tetramer (e.g., the amount of T cells in a population that have phospho-ERK, for example, as assessed by flow cytometry). In certain embodiments, the antigen-binding site, at a concentration that is at least 10-fold, 20-fold, 30-fold, 40-fold, 50-fold, 60-fold, 70-fold, 80-fold, 90-fold, or 100-fold higher than the K_{D} value with which the antigen-binding site binds cells expressing human CD8 (e.g., human CD8⁺ T cells or human T cells comprising a CD8⁺ population), does not increase or decrease the ERK phosphorylation by more than 1.5 fold, 2 fold, 2.5 fold, 3 fold, 4 fold, or 5 fold. An exemplary assay measuring the stability of the complex is described in Example 1 below.

In certain embodiments, the antigen-binding site, at a concentration that is at least 10-fold, 20-fold, 30-fold, 40-fold, 50-fold, 60-fold, 70-fold, 80-fold, 90-fold, or 100-fold higher than the K_{D} value with which the antigen-binding site binds cells expressing human CD8 (e.g., human CD8⁺ T cells or human T cells comprising a CD8⁺ population), does not increase or decrease T cell activation by more than 10%, 20%, 30%, 40%, 50%, 60%, 70%, 80%, or 90%, as measured by cytotoxicity of cancer cells caused by T cells, wherein the T cells express a TCR that recognize a T cell epitope presented by a human class I MHC on the surface of the cancer cells. In certain embodiments, the antigen-binding site, at a concentration that is at least 10-fold, 20-fold, 30-fold, 40-fold, 50-fold, 60-fold, 70-fold, 80-fold, 90-fold, or 100-fold higher than the K_{D} value with which the antigen-binding site binds cells expressing human CD8 (e.g., human CD8⁺ T cells or human T cells comprising a CD8⁺ population), does not increase or decrease the cytotoxicity by more than 1.5 fold, 2 fold, 2.5 fold, 3 fold, 4 fold, or 5 fold. An exemplary assay measuring the stability of the complex is described in Example 1 below.

Other methods can be used to assess the stability of peptide-MHC-TCR complex, the strength of TCR signaling, and the consequent T cell activation. For example, the activation of a transcription factor downstream of TCR signaling (e.g., NFAT) can be assessed using a recombinant construct comprising a NFAT-responsive promoter operably linked to a reporter gene. Exemplary methods are described in Laugel et al. (2007) J. Biol. Chem. 282(33):23799-23810 and in Gaud et al. (2018) Nat. Rev. Immunol. 18(8):485-497, including assessment of degranulation, IFNγ production, TCR downregulation, LCK phosphorylation, and ZAP70 phosphorylation.

### IV. Fusion Protein Formats

The fusion proteins disclosed herein, comprising a CD8-binding site and an IL-12 protein, can take various formats by linking its components in various ways.

For example, the antigen-binding site that binds CD8 can be present in any one of the following antibody fragments or variants: (1) a Fab fragment, a monovalent fragment having the VL, VH, CL and CH1 domains; (2) a F(ab′)2 fragment, a bivalent fragment having two Fab fragments linked by a disulfide bridge at the hinge region; (3) an Fd fragment having two VH and CH1 domains; (4) an Fv fragment having the VL and VH domains of a single arm of an antibody; (5) a sdAb fragment; and (6) a single chain Fv (scFv). Exemplary formats of antigen-binding sites and combinations thereof are described in WO2000006605A2, WO2013026837A1, WO2013026833A1, US20140308285A1, US20140302037A1, WO2014144722A2, WO2014151910A1, and WO2015048272A1.

In certain embodiments, the fusion protein comprises an scFv comprising a CD8-binding site. As used herein, the terms “single-chain Fv” and “scFv” refer to a single-polypeptide-chain antibody fragment that comprise the variable domains from both the heavy and light chains, but lack the constant regions. Generally, a scFv further comprises a peptide linker connecting the VH and VL domains which enables it to form the desired structure to bind to antigen. scFvs are discussed in detail by Pluckthun in The Pharmacology of Monoclonal Antibodies, vol. 113, Rosenburg and Moore eds. Springer-Verlag, New York, pp. 269-315 (1994). Various methods of generating scFvs are known, including those described in U.S. Pat. Nos. 4,694,778 and 5,260,203; International Patent Application Publication No. WO 88/01649; Bird (1988) Science 242:423-442; Huston et al. (1988) Proc. Natl. Acad. Sci. USA 85:5879-5883; Ward et al. (1989) Nature 334:54454; Skerra et al. (1988) Science 242:1038-1041. In specific embodiments, a scFv can also be human, humanized, and/or synthetic.

In certain embodiments, in the scFv comprising the CD8-binding site, the VH is positioned C-terminal to the VL. In certain embodiments, in the scFv comprising the CD8-binding site, the VH is positioned N-terminal to the VL. In certain embodiments, the VH and the VL are linked by a peptide linker, for example, a linker disclosed in the subsection below titled “Linkers.” To stabilize the scFv, the amino acid residues at position 44 of the VH and at position 100 of the VL (under Kabat numbering) can be substituted by Cys, thereby facilitating the formation of a disulfide bond between the VH and the VL. Accordingly, in certain embodiments, the VH and VL comprise Cys at positions 100 and 44, respectively.

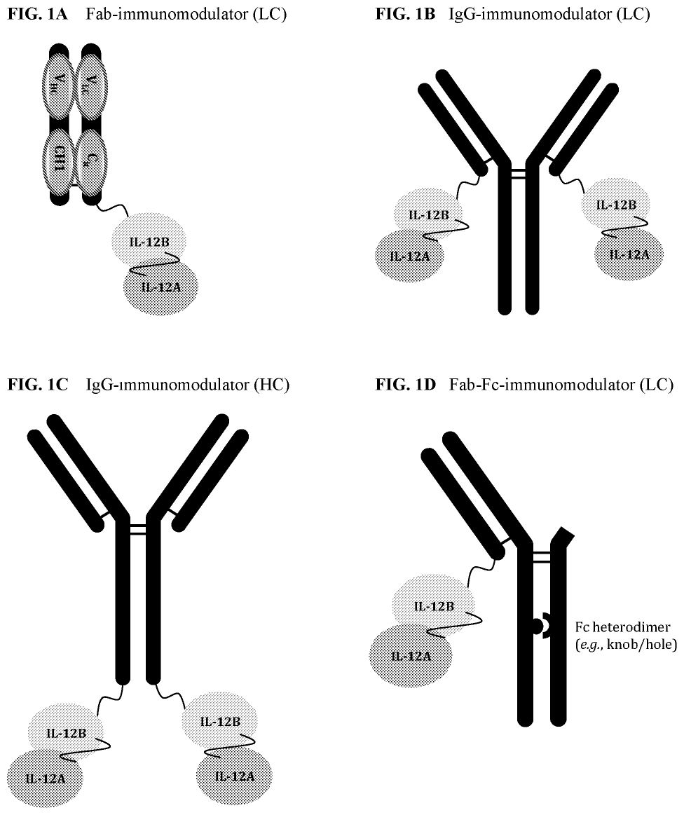

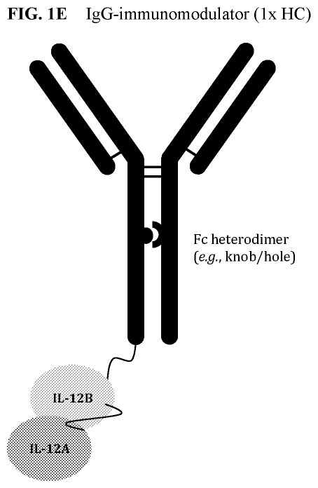

In certain embodiments, the fusion protein comprises a Fab comprising a CD8-binding site. In certain embodiments, the IL-12 protein is linked to the C-terminus of the light chain of the Fab (see, e.g., FIG. 1A, format called “Fab-immunomodulator (LC)”). In certain embodiments, the IL-12 protein is linked to the C-terminus of the heavy chain of the Fab. In certain embodiments, the IL-12 protein is linked to the N-terminus of the light chain of the Fab. In certain embodiments, the IL-12 protein is linked to the N-terminus of the heavy chain of the Fab.

In certain embodiments, the fusion protein disclosed herein further comprises an antibody Fc domain. The IL-12 protein and/or the CD8-binding site can be linked to various positions of the Fc domain.

In certain embodiments, the fusion protein comprises two Fabs each comprising a CD8-binding site (e.g., the same CD8-binding site). The Fabs can be linked to the antibody Fc domain to form a complete antibody (e.g., IgG) structure having two heavy chains and two light chains. The IL-12 protein can be linked to various positions of the antibody, for example, the C-terminus of a heavy chain, the C-terminus of a light chain, the N-terminus of a heavy chain, or the N-terminus of a light chain.

In certain embodiments, the fusion protein comprises two IL-12 proteins. In certain embodiments, the two IL-12 proteins have identical amino acid sequences. Such fusion protein can form a symmetrical structure. For example, in certain embodiments, the fusion protein comprises two Fab fragments each comprising a CD8-binding site, two IL-12 proteins, and an antibody Fc domain, wherein the heavy chain polypeptide of each of the two Fab fragments is linked to the N-terminus of the antibody Fc domain, and each of the two IL-12 proteins is linked to the C-terminus of a light chain polypeptide of each of the two Fab fragments. In certain embodiments, the fusion protein comprises a complete anti-CD8 antibody (e.g., IgG) and two IL-12 proteins linked to the C-terminus of the light chain polypeptides of the two Fab fragments (see, e.g., FIG. 1B, format called “IgG-immunomodulator (LC)”). In other embodiments, the fusion protein comprises two Fab fragments each comprising the CD8-binding site, two IL-12 proteins, and an antibody Fc domain, wherein the heavy chain polypeptide of each of the two Fab fragments is linked to the N-terminus of the antibody Fc domain, and each of the two IL-12 proteins is linked to the C-terminus of a heavy chain polypeptide of the Fc domain. In certain embodiments, the fusion protein comprises a complete anti-CD8 antibody (e.g., IgG) and two IL-12 proteins linked to the C-terminus of the two heavy chain polypeptides of the Fc domain (see, e.g., FIG. 1C, format called “IgG-immunomodulator (HC)”).

In certain embodiments, the fusion protein comprises two Fabs each comprising the same CD8-binding site and a single IL-12 protein. The Fabs can be linked to the antibody Fc domain to form a complete antibody (e.g., IgG) structure having two heavy chains and two light chains. For example, in certain embodiments, the fusion protein comprises two Fab fragments each comprising a CD8-binding site, a single IL-12 protein, and an antibody Fc domain, wherein the heavy chain polypeptide of each of the two Fab fragments is linked to the N-terminus of the antibody Fc domain, and the IL-12 protein is linked to the C-terminus of the antibody Fc domain. In certain embodiments, the fusion protein comprises a complete anti-CD8 antibody (e.g., IgG) and a single IL-12 protein linked to the C-terminus of a heavy chain polypeptide of the Fc domain (see, e.g., FIG. 1E, format called “IgG-immunomodulator (1×HC)”).

In certain embodiments, the fusion protein comprises a single Fab comprising a CD8-binding site, a single IL-12 protein, and an antibody Fc domain. In certain embodiments, the IL-12 protein and the antibody Fc domain are each linked to the C-terminus of a Fab polypeptide. In certain embodiments, the IL-12 protein is linked to the C-terminus of the Fab light chain polypeptide and the antibody Fc domain is linked to the C-terminus of the Fab heavy chain polypeptide (see, e.g., FIG. 1D, format called “Fab-Fc-immunomodulator (LC)”).

In certain embodiments of any of the formats above that includes an antibody Fc domain, the antibody Fc domain comprises one or more mutations that reduce ADCC effector function, as described in more detail in the subsection below.

### Antibody Fc Domains

In certain embodiments, the fusion protein (e.g., immunostimulatory fusion protein) disclosed herein lacks an antibody Fc domain. Without wishing to be bound by theory, it is contemplated that the absence of an antibody Fc domain reduces cytotoxicity against the target CD8⁺ T cells as a result of ADCC, improves tissue penetration, facilitates clearance of circulating protein that is not attached to an immune cell, and improves expression of the protein in vitro or in vivo as a result of the reduced size. The ADCC effector function of an antibody Fc domain is primarily mediated by the CH2 domain. Accordingly, in certain embodiments, the fusion protein lacks an antibody Fc CH2 domain.

In other embodiments, the fusion protein (e.g., immunostimulatory fusion protein) disclosed herein comprises an antibody Fc domain having low or no ADCC effector function. Without wishing to be bound by theory, it is contemplated that the presence of an Fc domain (e.g., an Fc domain that binds FcRn) may increase the serum half-life of the fusion protein and the low level or absence of ADCC effector function reduces the risk of killing the target CD8⁺ T cells. While the longer serum half-life could in theory increase both efficacy and toxicity, it is contemplated that the potential toxicity is mitigated by specific targeting of the fusion protein to CD8-expressing cells. ADCC is mediated by binding to Fcγ receptors. Accordingly, in certain embodiments, the Fc domain incorporates one or more mutations or modifications, in either or both Fc polypeptide chains, that alter the binding to an Fcγ receptor (e.g., FcγRI/CD64, FcγRIIA/CD32A, FcγRIIB/CD32B, FcγRIIIIA/CD16, or FcγRIIIB).

In certain embodiments, the antibody Fc domain is an IgG1, IgG2, IgG3, or IgG4 Fc domain from human or another mammal, such as rabbit, dog, cat, mouse, or horse. In certain embodiments, the antibody Fc domain is a human IgG1, IgG2, IgG3, or IgG4 Fc domain. Fc domains of human IgG1 and IgG3 are known to have ADCC and CDC effector functions. In certain embodiments, the fusion protein disclosed herein comprises an antibody Fc domain or a fragment thereof in which an ADCC effector function is reduced by at least 50%, at least 60%, at least 70%, at least 80%, at least 90%, at least 91%, at least 92%, at least 93%, at least 94%, at least 95%, at least 96%, at least 97%, at least 98%, or at least 99% relative to a wild-type human IgG1 Fc domain having the amino acid sequence of SEQ ID NO: 84. In certain embodiments, the fusion protein disclosed herein comprises an antibody Fc domain or a fragment thereof in which a CDC effector function is reduced by at least 50%, at least 60%, at least 70%, at least 80%, at least 90%, at least 91%, at least 92%, at least 93%, at least 94%, at least 95%, at least 96%, at least 97%, at least 98%, or at least 99% relative to a wild-type human IgG1 Fc domain having the amino acid sequence of SEQ ID NO: 84.

Within the Fc domain, CD16 binding is mediated by the hinge region and the CH2 domain. For example, within human IgG1, the interaction with CD16 is primarily focused on amino acid residues Asp 265 -Glu 269, Asn 297 -Thr 299, Ala 327 -Ile 332, Leu 234 -Ser 239, and carbohydrate residue N-acetyl-D-glucosamine in the CH2 domain (see, Sondermann et al., Nature, 406 (6793):267-273). Based on the known domains, mutations can be selected to enhance or reduce the binding affinity to CD16, such as by using phage-displayed libraries or yeast surface-displayed cDNA libraries, or can be designed based on the known three-dimensional structure of the interaction. All the amino acid positions in antibody Fc domains in this application are numbered according to the EU numbering system (see Edelman et al., (1969) Proc. Natl. Acad. Sci. USA 63:78-85; Kabat et al., Sequences of Proteins of Immunological Interest (5th ed. 1991)).

Mutations in IgG Fc domain and their impact on ADCC and CDC effector functions have been characterized (see, e.g., Wang et al., (2018) Protein Cell 9(1):63-73). In certain embodiments, the fusion protein comprising an antibody (e.g., IgG, e.g., human IgG1) Fc domain comprising one or more mutations that reduce ADCC and/or CDC effector functions. Exemplary Fc domain mutations that reduce ADCC and CDC effector functions are disclosed in U.S. Pat. No. 11,084,863. In certain embodiments, the antibody Fc domain differs from the wild-type human IgG1 Fc domain at one or more positions selected from R292, S298, L234, L235, G237, N297, S298, A327, P329, A330, P331, and K414. In certain embodiments, the antibody Fc domain differs from the human IgG1 Fc domain at one or more positions selected from L234, L235, G237, A327, P329, A330, and P331. In certain embodiments, the antibody Fc domain differs from the human IgG1 Fc domain at one or more positions selected from R292, S298, and K414. In some embodiments, the antibody Fc domain differs from the human IgG1 Fc domain at: (a) positions L234 and L235; (b) positions L234, L235, and P329; or (c) positions L234, L235, A327, A330, and P331.

In certain embodiments, the one or more mutations are selected from L234A, L235A, L235E, N297A, N297G, N297Q, S298A, A327G, P329A, P329G, A330S, and P331S. In certain embodiments, the fusion protein comprises a human IgG1 Fc comprising L234A and L235A substitutions. In certain embodiments, the fusion protein comprises a human IgG1 Fc comprising L234A, L235A, and P329G or P329A substitutions. In certain embodiments, the fusion protein comprises a human IgG1 Fc comprising N297A substitution. In certain embodiments, the fusion protein comprises a human IgG1 Fc comprising L234A, L235A, and N297A substitutions. In certain embodiments, the fusion protein comprises a human IgG1 Fc comprising L234A, L235A, N297A, and P329G or P329A substitutions. In certain embodiments, the fusion protein comprises a human IgG1 Fc comprising L234A, L235A, A327G, A330S, and P331S substitutions. In certain embodiments, the fusion protein comprises a human IgG1 Fc comprising L234A, L235A, N297A, A327G, A330S, and P331S substitutions.

Fe domains of human IgG4 and IgG2 are known to have little or no ADCC effector function. In certain embodiments, the fusion protein comprises a human IgG4 Fc domain or a fragment thereof. In certain embodiments, the IgG4 Fc domain comprises a S228P substitution. In certain embodiments, the fusion protein comprises a human IgG2 Fc domain or a fragment thereof.

Where the format of a Fc-containing fusion protein disclosed herein is asymmetric, for example, in the Fab-Fc-immunomodulator (LC) format illustrated by FIG. 1D or the IgG-immunomodulator (1×HC) format illustrated by FIG. 1E, one or more mutations can be introduced into the Fc domain to promote heterodimerization. One or more mutations can be incorporated into the constant region as compared to human IgG1 constant region, for example at Q347, Y349, L351, S354, E356, E357, K360, Q362, S364, T366, L368, K370, N390, K392, T394, D399, S400, D401, F405, Y407, K409, T411 and/or K439. Exemplary substitutions include, for example, Q347E, Q347R, Y349S, Y349K, Y349T, Y349D, Y349E, Y349C, T350V, L351K, L351D, L351Y, S354C, E356K, E357Q, E357L, E357W, K360E, K360W, Q362E, S364K, S364E, S364H, S364D, T366V, T3661, T366L, T366M, T366K, T366W, T366S, L368E, L368A, L368D, K370S, N390D, N390E, K392L, K392M, K392V, K392F, K392D, K392E, T394F, T394W, D399R, D399K, D399V, S400K, S400R, D401K, F405A, F405T, Y407A, Y4071, Y407V, K409F, K409W, K409D, T411D, T411E, K439D, and K439E.

In certain embodiments, at least one amino acid substitution can be made at K392, K370, K409, and/or K439 of the first Fc polypeptide chain, and at least one amino acid substitution can be made at D399, E356, and/or E357 of the second polypeptide chain, where the amino acid residues in the first Fc polypeptide chain is replaced by any known negatively-charged amino acid, and the amino acid residue in the second Fc polypeptide chain is replaced by any known positively-charged amino acid.

In some embodiments, the amino acid sequence of one polypeptide chain of the antibody constant region differs from the amino acid sequence of an IgG1 constant region at position T366, and wherein the amino acid sequence of the other polypeptide chain of the antibody constant region differs from the amino acid sequence of an IgG1 constant region at one or more positions selected from the group consisting of T366, L368 and Y407.

In some embodiments, the amino acid sequence of one polypeptide chain of the antibody constant region differs from the amino acid sequence of an IgG1 constant region at one or more positions selected from the group consisting of E357, K360, Q362, S364, L368, K370, T394, D401, F405, and T411 and wherein the amino acid sequence of the other polypeptide chain of the antibody constant region differs from the amino acid sequence of an IgG1 constant region at one or more positions selected from the group consisting of Y349, E357, S364, L368, K370, T394, D401, F405 and T411.

In some embodiments, the amino acid sequence of one polypeptide chain of the antibody constant region differs from the amino acid sequence of an IgG1 constant region at one or more positions selected from the group consisting of L351, D399, S400 and Y407 and wherein the amino acid sequence of the other polypeptide chain of the antibody constant region differs from the amino acid sequence of an IgG1 constant region at one or more positions selected from the group consisting of T366, N390, K392, K409 and T411.

In some embodiments, the amino acid sequence of one polypeptide chain of the antibody constant region differs from the amino acid sequence of an IgG1 constant region at one or more positions selected from the group consisting of Q347, Y349, K360, and K409, and wherein the amino acid sequence of the other polypeptide chain of the antibody constant region differs from the amino acid sequence of an IgG1 constant region at one or more positions selected from the group consisting of Q347, E357, D399 and F405.

In some embodiments, the amino acid sequence of one polypeptide chain of the antibody constant region differs from the amino acid sequence of an IgG1 constant region at one or more positions selected from the group consisting of K370, K392, K409 and K439, and wherein the amino acid sequence of the other polypeptide chain of the antibody constant region differs from the amino acid sequence of an IgG1 constant region at one or more positions selected from the group consisting of D356, E357 and D399.

In some embodiments, the amino acid sequence of one polypeptide chain of the antibody constant region differs from the amino acid sequence of an IgG1 constant region at one or more positions selected from the group consisting of L351, E356, T366 and D399, and wherein the amino acid sequence of the other polypeptide chain of the antibody constant region differs from the amino acid sequence of an IgG1 constant region at one or more positions selected from the group consisting of Y349, L351, L368, K392 and K409.

In certain embodiments, an amino acid substitution in the first polypeptide replaces the original amino acid with a larger amino acid, selected from arginine (R), phenylalanine (F), tyrosine (Y) or tryptophan (W), and at least one amino acid substitution in the second polypeptide replaces the original amino acid(s) with a smaller amino acid(s), chosen from alanine (A), serine (S), threonine (T), or valine (V), such that the larger amino acid substitution (a protuberance) fits into the surface of the smaller amino acid substitutions (a cavity). For example, one polypeptide can incorporate a T366W substitution, and the other can incorporate three substitutions including T366S, L368A, and Y407V. This set of substitutions are referred to as “knobs-in-holes,” wherein the polypeptide chain comprising a T366W substitution has a “knob” and the polypeptide chain comprising T366S, L368A, and Y407V substitutions has a “hole.” In certain embodiments, the polypeptide chain linked to a cytokine (e.g., IL-12) comprises the “knob” substitution and the polypeptide chain not linked to a cytokine (e.g., IL-12) comprises the “hole” substitutions, as depicted in FIGS. 1D and 1E.

Alternatively, amino acid substitutions could be selected from the following sets of substitutions shown in Table 5 below. Additional exemplary Fc domain substitutions that promote heterodimerization are disclosed in U.S. Pat. No. 11,084,863.

TABLE 5
Fc Heterodimerization Mutations

| | First Fc Polypeptide | Second Fc Polypeptide |
|---|---|---|
| Set 1 | S364E/F405A | Y349K/T394F |
| Set 2 | S364H/D401K | Y349T/T411E |
| Set 3 | S364H/T394F | Y349T/F405A |
| Set 4 | S364E/T394F | Y349K/F405A |
| Set 5 | S364E/T411E | Y349K/D401K |
| Set 6 | S364D/T394F | Y349K/F405A |
| Set 7 | S364H/F405A | Y349T/T394F |
| Set 8 | S364K/E357Q | L368D/K370S |
| Set 9 | L368D/K370S | S364K |
| Set 10 | L368E/K370S | S364K |
| Set 11 | K360E/Q362E | D401K |
| Set 12 | L368D/K370S | S364K/E357L |
| Set 13 | K370S | S364K/E357Q |
| Set 14 | F405L | K409R |
| Set 15 | K409R | F405L |
| Set 16 | K409W | D399V/F405T |
| Set 17 | Y349S | E357W |
| Set 18 | K360E | Q347R |
| Set 19 | K360E/K409W | Q347R/D399V/F405T |
| Set 20 | Q347E/K360E/K409W | Q347R/D399V/F405T |
| Set 21 | Y349S/K409W | E357W/D399V/F405T |
| Set 22 | T366K/L351K | L351D/L368E |
| Set 23 | T366K/L351K | L351D/Y349E |
| Set 24 | T366K/L351K | L351D/Y349D |
| Set 25 | T366K/L351K | L351D/Y349E/L368E |
| Set 26 | T366K/L351K | L351D/Y349D/L368E |
| Set 27 | E356K/D399K | K392D/K409D |
| Set 28 | L351Y, D399R, | T366V, T366I, T366L, |
| | D399K, S400K, | T366M, N390D, N390E, |
| | S400R, Y407A, | K392L, K392M, K392V, |
| | Y407I, Y407V | K392F K392D, K392E, |
| | | K409F, K409W, T411D |
| | | and T411E |
| Set 29 | T350V, L351Y, | T350V, T366L, K392L, |
| | F405A, and Y407V | and T394W |

In certain embodiments, the structural stability of a hetero-multimeric protein may be increased by introducing S354C on either of the first or second polypeptide chain, and Y349C on the opposing polypeptide chain, which forms an artificial disulfide bridge within the interface of the two polypeptides.

Unless indicated otherwise, the amino acid substitutions described above are identified in the context of human IgG1. It is understood that corresponding substitutions are also contemplated in the context of human IgG2, IgG3, and IgG4. For example, the amino acid residue at position 234 of human IgG4 is F, whereas the amino acid residue at position 234 of human IgG1 is L. Where an L234A substitution is described in the context of human IgG1, the F234A substitution in the context of human IgG4 is also contemplated.

It is understood that where the two Fc polypeptide chains, either referred to as the first polypeptide chain and the second polypeptide chain or as one polypeptide chain and the other polypeptide chain, contain different amino acid sequences or mutations, either chain can be linked to any other domain of the fusion proteins disclosed herein. Where an exemplary fusion protein is described to contain a first set of mutations in a given first Fc polypeptide chain and a second set of mutations in the second Fc polypeptide chain, the reverse construct, in which the first Fc polypeptide chain contains the second set of mutations and the second Fc polypeptide chain contains the first set of mutations, is also contemplated.

### Linkers

The fusion protein disclosed herein includes multiple components, which can be linked to each other by a peptide bond or a linker (e.g., peptide linker). In certain embodiments, the peptide linker does not comprise any multimerization or polymerization activity. A peptide linker disclosed herein can be used to link a VH and a VL in a scFv. A peptide linker disclosed herein can also be used to link between a CD8-binding site, an IL-12 protein, and/or an antibody Fc domain. Exemplary peptide linkers are described in U.S. Pat. Nos. 4,751,180 and 4,935,233 and International Application Publication No. WO198809344A1.

A peptide linker in a given fusion protein may have an optimized length and/or amino acid composition. In certain embodiments, a peptide linker is short, consisting of 1, 2, 3, 4, 5, 6, 7, 8, 9, 10, 11, 12, 13, or 14 amino acid residues. Thus, in certain embodiments, the peptide linker consists of about 12 or fewer amino acid residues. In certain embodiments, a peptide linker is longer, consisting of 15, 20, 25, or more amino acid residues. In certain embodiments, a peptide linker consists of about 3 to about 15, for example 8, 9 or 10 amino acid residues.

Regarding the amino acid composition of a peptide linker, peptides are selected with properties that confer flexibility to the fusion proteins disclosed herein, do not interfere with the CD8-binding site and the IL-12 protein, and resist cleavage from proteases. For example, glycine and serine residues generally provide protease resistance. Examples of the linkers suitable for linking the domains in the fusion proteins include but are not limited to (GS)ₙ, (GGS)ₙ, (GGGS)ₙ, (GGSG)ₙ, (GGSGG)ₙ, and (GGGGS)ₙ, wherein n is 1, 2, 3, 4, 5, 6, 7, 8, 9, 10, 11, 12, 13, 14, 15, 16, 17, 18, 19, or 20. In some embodiments, a peptide linker is selected from the peptide sequences listed in Table 6.

In certain embodiments, the fusion protein disclosed herein comprises an IL-12 protein (e.g., a single chain IL-12) linked to the C-terminus of a Fab light chain by a peptide linker comprising the amino acid sequence of SEQ ID NO: 96, 97, 98, 99, 100, 101, 86, 102, 103, 104, 85, or 87. In certain embodiments, the fusion protein disclosed herein comprises an IL-12 protein (e.g., a single chain IL-12) linked to the C-terminus of a Fab light chain by a peptide linker comprising the amino acid sequence of SEQ TD NO: 86.

In certain embodiments, the fusion protein disclosed herein comprises an IL-12 protein (e.g., a single chain IL-12) linked to the C-terminus of a Fc domain polypeptide by a peptide linker comprising the amino acid sequence of SEQ TD NO: 96, 97, 98, 99, 100, 101, 86, 102, 103, 104, 85, or 87. In certain embodiments, the fusion protein disclosed herein comprises an IL-12 protein (e.g., a single chain IL-12) linked to the C-terminus of a Fc domain polypeptide by a peptide linker comprising the amino acid sequence of SEQ TD NO: 87.

TABLE 6
Exemplary Peptide Linkers

| Linker | Amino Acid Sequence |
|---|---|
| (GS)₁₀ | GSGSGSGSGSGSGSGSGSGS (SEQ ID NO: 96) |
| (GGS)₁₀ | GGSGGSGGSGGSGGSGGSGGSGGSGGSGGS (SEQ ID NO: 97) |
| (GGGS)₁₀ | GGGSGGGSGGGSGGGSGGGSGGGSGGGSGGGSGGGSGGGS |
| | (SEQ ID NO: 98) |
| (GGSG)₁₀ | GGSGGGSGGGSGGGSGGGSGGGSGGGSGGGSGGGSGGGSG |
| | (SEQ ID NO: 99) |
| (GGSGG)₁₀ | GGSGGGGSGGGGSGGGGSGGGGGGGGSGGGGSGGGGSGG |
| | GGSGGGGSGG (SEQ ID NO: 100) |
| (GGGGS)₁₀ | GGGGSGGGGSGGGGSGGGGSGGGGSGGGGSGGGGSGGGGS |
| | GGGGSGGGGS (SEQ ID NO: 101) |
| (GGGGS)₄ | GGGGSGGGGSGGGGSGGGGS (SEQ ID NO: 86) |
| (GGGGS)₃ | GGGGSGGGGSGGGGS (SEQ ID NO: 102) |
| (GGGGS)₂₀ | GGGGSGGGGSGGGGSGGGGSGGGGSGGGGSGGGGSGGGGS |
| | GGGGSGGGGSGGGGSGGGGSGGGGSGGGGSGGGGSGGGGS |
| | GGGGSGGGGSGGGGSGGGGS (SEQ ID NO: 103) |
| (GGSGG)₂₀ | GGSGGGGSGGGGSGGGGSGGGGSGGGGSGGGGSGGGGSGG |
| | GGSGGGGSGGGGSGGGGSGGGGSGGGGSGGGGSGGGGSGG |
| | GGSGGGGSGGGGSGGGGSGG (SEQ ID NO: 104) |
| (GGGS)₄ | GGGSGGGSGGGSGGGS (SEQ ID NO: 85) |
| GGGS-(GGGGS)₃ | GGGSGGGGSGGGGSGGGGS (SEQ ID NO: 87) |

### V. Exemplary Immunostimulatory Fusion Proteins

A desirable immunostimulatory fusion protein effectively promotes survival, proliferation, activation, and/or memory formation and/or inhibits exhaustion of CD8⁺ T cells without causing significant toxicity. Proper formats of fusion proteins comprising a CD8-binding site and an IL-12 protein can be selected by assessing the efficacy and tolerability of the fusion protein after administration (e.g., systemic administration) to an animal. An exemplary method is described in Example 3 below.

It is understood that IL-12, despite stimulating an immune response towards a target (e.g., cancer), has high toxicity when administered systemically in a non-targeted form. It is contemplated that a fusion protein comprising IL-12 and a CD8-binding site would selectively deliver IL-12 to CD8⁺ T cells over other cells expressing an IL-12 receptor (e.g., NK cells), thereby reducing the minimum effective dose of IL-12 and/or reducing toxicity to the subject (e.g., NK cell-mediated toxicity). In certain embodiments, the immunostimulatory fusion protein comprises two Fab fragments each comprising a CD8-binding site, two IL-12 proteins, and an antibody Fc domain, wherein the heavy chain polypeptide of each of the two Fab fragments is linked to the N-terminus of the antibody Fc domain, and each of the two IL-12 proteins is linked to the C-terminus of a light chain polypeptide of each of the two Fab fragments. In certain embodiments, the immunostimulatory fusion protein comprises a complete anti-CD8 antibody (e.g., IgG) and two IL-12 proteins linked to the C-terminus of the light chain polypeptides of the two Fab fragments (IgG-immunomodulator (LC) format, FIG. 1B). In certain embodiments, the immunostimulatory fusion protein comprises two Fab fragments each comprising a CD8-binding site, a single IL-12 protein, and an antibody Fc domain, wherein the heavy chain polypeptide of each of the two Fab fragments is linked to the N-terminus of the antibody Fc domain, and the IL-12 protein is linked to the C-terminus of the antibody Fc domain. In certain embodiments, the immunostimulatory fusion protein comprises a complete anti-CD8 antibody (e.g., IgG) and a single IL-12 protein linked to the C-terminus of a heavy chain polypeptide of the Fc domain (IgG-immunomodulator (1×HC) format, FIG. 1E). In certain embodiments of the immunostimulatory fusion protein in any one of the formats above, the antibody Fc domain comprises one or more mutations that reduce ADCC effector function (e.g., L234A, L235A, N297A, and optionally P329G or P329A substitutions). In certain embodiments of the immunostimulatory fusion protein in the IgG-cytokine (1×HC) format, the antibody Fc domain further comprises one or more mutations that promote Fc heterodimerization.

Listed below in Table 7 are examples of immunostimulatory fusion proteins comprising a single-chain IL-12, an antigen-binding site that binds CD8, and an antibody Fc domain that lacks effector function. The constructs 02(VH13/VK3)-IgG-IL12 (LC), 02(VH4/VK3)-IgG-IL12 (LC), and 02(V6/VK3)-IgG-IL12 (LC) take the format described in FIG. GB. The constructs 02(VH3/VK3)-IgG-G12 (1×HC), 02(VH4/VK3)-IgG-G12 (1×HI, and 02(V(6/VK3)-IgG-4, 12 (1×HC take the format described in FIG. 1E.

TABLE 7
Exemplary Immunostimulatory Fusion Proteins

| Protein | Polypeptide | |
|---|---|---|
| Name | Components | Component Sequence |
| 02(VH3/VK3)- | Heavy chain: | EVQLVQSGAEVKKPGATLKISCKASGYTFTDFTMH |
| IgG- | HC02-VH3 | WVQQAPGKGLEWIGLVNPNNGGNIYAEKFQGRATL |
| IL12(LC) | (L234A, | TVDTSTSTAYMELSSLRSEDTSVYYCGRVPLYTSHG |
| | L235A, | MDYWGQGTLVTVSS |
| | N297A) | ASTKGPSVFPLAPSSKSTSGGTAALGCLVKDYFPEPV |
| | | TVSWNSGALTSGVHTFPAVLQSSGLYSLSSVVTVPS |
| | | SSLGTQTYICNVNHKPSNTKVDKKVEPKSCDKTHTC |
| | | PPCPAPEAAGGPSVFLFPPKPKDTLMISRTPEVTCVV |
| | | VDVSHEDPEVKFNWYVDGVEVHNAKTKPREEQYA |
| | | STYRVVSVLTVLHQDWLNGKEYKCKVSNKALPAPI |
| | | EKTISKAKGQPREPQVYTLPPSRDELTKNQVSLTCLV |
| | | KGFYPSDIAVEWESNGQPENNYKTTPPVLDSDGSFF |
| | | LYSKLTVDKSRWQQGNVFSCSVMHEALHNHYTQK |
| | | SLSLSPGK |
| | | (SEQ ID NO: 73) |
| | Light chain: | EIVLTQSPATLSLSPGERATISCRASESVDSYDINSMH |
| | LC02-VK3- | WYQQKPGQAPRLLIYRASHLQSGIPARFSGSGSRTD |
| | IL12 | FTLTISSLEPEDFATYYCQQSNEDPLTFGQGTKLEIK |
| | | RTVAAPSVFIFPPSDEQLKSGTASVVCLLNNFYPREA |
| | | KVQWKVDNALQSGNSQESVTEQDSKDSTYSLSSTL |
| | | TLSKADYEKHKVYACEVTHQGLSSPVTKSFNRGEC |
| | | GGGGSGGGGSGGGGSGGGGS |
| | | IWELKKDVYVVELDWYPDAPGEMVVLTCDTPEED |
| | | GITWTLDQSSEVLGSGKTLTIQVKEFGDAGQYTCHK |
| | | GGEVLSHSLLLLHKKEDGIWSTDILKDQKEPKNKTF |
| | | LRCEAKNYSGRFTCWWLTTISTDLTFSVKSSRGSSD |
| | | PQGVTCGAATLSAERVRGDNKEYEYSVECQEDSAC |
| | | PAAEESLPIEVMVDAVHKLKYENYTSSFFIRDIIKPDP |
| | | PKNLQLKPLKNSRQVEVSWEYPDTWSTPHSYFSLTF |
| | | CVQVQGKSKREKKDRVFTDKTSATVICRKNASISVR |
| | | AQDRYYSSSWSEWASVPCS |
| | | GGGSGGGSGGGSGGGS |
| | | RNLPVATPDPGMFPCLHHSQNLLRAVSNMLQKARQ |
| | | TLEFYPCTSEEIDHEDITKDKTSTVEACLPLELTKNES |
| | | CLNSRETSFITNGSCLASRKTSFMMALCLSSIYEDLK |
| | | MYQVEFKTMNAKLLMDPKRQIFLDQNMLAVIDEL |
| | | MQALNFNSETVPQKSSLEEPDFYKTKIKLCILLHAFR |
| | | IRAVTIDRVMSYLNAS |
| | | (SEQ ID NO: 74) |
| 02(VH4/VK3)- | Heavy chain: | EVQLVQSGAEVKKPGATVKISCKASGYTFTDFTMH |
| IgG- | HC02-VH4 | WVQQAPGKGLEWIGLVNPNNGGNIYAEKFQGRATL |
| IL12(LC) | (L234A, | TVDTSTSTAYMELSSLRSEDTAVYYCGRVPLYTSHG |
| | L235A, | MDYWGQGTLVTVSS |
| | N297A) | ASTKGPSVFPLAPSSKSTSGGTAALGCLVKDYFPEPV |
| | | TVSWNSGALTSGVHTFPAVLQSSGLYSLSSVVTVPS |
| | | SSLGTQTYICNVNHKPSNTKVDKKVEPKSCDKTHTC |
| | | PPCPAPEAAGGPSVFLFPPKPKDTLMISRTPEVTCVV |
| | | VDVSHEDPEVKFNWYVDGVEVHNAKTKPREEQYA |
| | | STYRVVSVLTVLHQDWLNGKEYKCKVSNKALPAPI |
| | | EKTISKAKGQPREPQVYTLPPSRDELTKNQVSLTCLV |
| | | KGFYPSDIAVEWESNGQPENNYKTTPPVLDSDGSFF |
| | | LYSKLTVDKSRWQQGNVFSCSVMHEALHNHYTQK |
| | | SLSLSPGK |
| | | (SEQ ID NO: 75) |
| | Light chain: | EIVLTQSPATLSLSPGERATISCRASESVDSYDINSMH |
| | LC02-VK3- | WYQQKPGQAPRLLIYRASHLQSGIPARFSGSGSRTD |
| | IL12 | FTLTISSLEPEDFATYYCQQSNEDPLTFGQGTKLEIK |
| | | RTVAAPSVFIFPPSDEQLKSGTASVVCLLNNFYPREA |
| | | KVQWKVDNALQSGNSQESVTEQDSKDSTYSLSSTL |
| | | TLSKADYEKHKVYACEVTHQGLSSPVTKSFNRGEC |
| | | GGGGSGGGGSGGGGSGGGGS |
| | | IWELKKDVYVVELDWYPDAPGEMVVLTCDTPEED |
| | | GITWTLDQSSEVLGSGKTLTIQVKEFGDAGQYTCHK |
| | | GGEVLSHSLLLLHKKEDGIWSTDILKDQKEPKNKTF |
| | | LRCEAKNYSGRFTCWWLTTISTDLTFSVKSSRGSSD |
| | | PQGVTCGAATLSAERVRGDNKEYEYSVECQEDSAC |
| | | PAAEESLPIEVMVDAVHKLKYENYTSSFFIRDIIKPDP |
| | | PKNLQLKPLKNSRQVEVSWEYPDTWSTPHSYFSLTF |
| | | CVQVQGKSKREKKDRVFTDKTSATVICRKNASISVR |
| | | AQDRYYSSSWSEWASVPCS |
| | | GGGSGGGSGGGSGGGS |
| | | RNLPVATPDPGMFPCLHHSQNLLRAVSNMLQKARQ |
| | | TLEFYPCTSEEIDHEDITKDKTSTVEACLPLELTKNES |
| | | CLNSRETSFITNGSCLASRKTSFMMALCLSSIYEDLK |
| | | MYQVEFKTMNAKLLMDPKRQIFLDQNMLAVIDEL |
| | | MQALNFNSETVPQKSSLEEPDFYKTKIKLCILLHAFR |
| | | IRAVTIDRVMSYLNAS |
| | | (SEQ ID NO: 74) |
| 02(VH6/VK3)- | Heavy chain: | EVQLVQSGAEVKKPGATVKISCKASGYTFTDFTMH |
| IgG- | HC02-VH6 | WVQQAPGKGLEWIGLVNPNNGGNIYAEKFQGRVTL |
| IL12(LC) | (L234A, | TVDTSTSTAYMELSSLRSEDTAVYYCGRVPLYTSHG |
| | L235A, | MDYWGQGTLVTVSS |
| | N297A) | ASTKGPSVFPLAPSSKSTSGGTAALGCLVKDYFPEPV |
| | | TVSWNSGALTSGVHTFPAVLQSSGLYSLSSVVTVPS |
| | | SSLGTQTYICNVNHKPSNTKVDKKVEPKSCDKTHTC |
| | | PPCPAPEAAGGPSVFLFPPKPKDTLMISRTPEVTCVV |
| | | VDVSHEDPEVKFNWYVDGVEVHNAKTKPREEQYA |
| | | STYRVVSVLTVLHQDWLNGKEYKCKVSNKALPAPI |
| | | EKTISKAKGQPREPQVYTLPPSRDELTKNQVSLTCLV |
| | | KGFYPSDIAVEWESNGQPENNYKTTPPVLDSDGSFF |
| | | LYSKLTVDKSRWQQGNVFSCSVMHEALHNHYTQK |
| | | SLSLSPGK |
| | | (SEQ ID NO: 76) |
| | Light chain: | EIVLTQSPATLSLSPGERATISCRASESVDSYDINSMH |
| | LC02-VK3- | WYQQKPGQAPRLLIYRASHLQSGIPARFSGSGSRTD |
| | IL12 | FTLTISSLEPEDFATYYCQQSNEDPLTFGQGTKLEIK |
| | | RTVAAPSVFIFPPSDEQLKSGTASVVCLLNNFYPREA |
| | | KVQWKVDNALQSGNSQESVTEQDSKDSTYSLSSTL |
| | | TLSKADYEKHKVYACEVTHQGLSSPVTKSFNRGEC |
| | | GGGGSGGGGSGGGGSGGGGS |
| | | IWELKKDVYVVELDWYPDAPGEMVVLTCDTPEED |
| | | GITWTLDQSSEVLGSGKTLTIQVKEFGDAGQYTCHK |
| | | GGEVLSHSLLLLHKKEDGIWSTDILKDQKEPKNKTF |
| | | LRCEAKNYSGRFTCWWLTTISTDLTFSVKSSRGSSD |
| | | PQGVTCGAATLSAERVRGDNKEYEYSVECQEDSAC |
| | | PAAEESLPIEVMVDAVHKLKYENYTSSFFIRDIIKPDP |
| | | PKNLQLKPLKNSRQVEVSWEYPDTWSTPHSYFSLTF |
| | | CVQVQGKSKREKKDRVFTDKTSATVICRKNASISVR |
| | | AQDRYYSSSWSEWASVPCS |
| | | GGGSGGGSGGGSGGGS |
| | | RNLPVATPDPGMFPCLHHSQNLLRAVSNMLQKARQ |
| | | TLEFYPCTSEEIDHEDITKDKTSTVEACLPLELTKNES |
| | | CLNSRETSFITNGSCLASRKTSFMMALCLSSIYEDLK |
| | | MYQVEFKTMNAKLLMDPKRQIFLDQNMLAVIDEL |
| | | MQALNFNSETVPQKSSLEEPDFYKTKIKLCILLHAFR |
| | | IRAVTIDRVMSYLNAS |
| | | (SEQ ID NO: 74) |
| 02(VH6/VK3- | Heavy chain: | EVQLVQSGAEVKKPGATVKISCKASGYTFTDFTMH |
| D30E/N35Q)- | HC02-VH6 | WVQQAPGKGLEWIGLVNPNNGGNIYAEKFQGRVTL |
| IgG- | (L234A, | TVDTSTSTAYMELSSLRSEDTAVYYCGRVPLYTSHG |
| IL12(LC) | L235A, | MDYWGQGTLVTVSS |
| | N297A) | ASTKGPSVFPLAPSSKSTSGGTAALGCLVKDYFPEPV |
| | | TVSWNSGALTSGVHTFPAVLQSSGLYSLSSVVTVPS |
| | | SSLGTQTYICNVNHKPSNTKVDKKVEPKSCDKTHTC |
| | | PPCPAPEAAGGPSVFLFPPKPKDTLMISRTPEVTCVV |
| | | VDVSHEDPEVKFNWYVDGVEVHNAKTKPREEQYA |
| | | STYRVVSVLTVLHQDWLNGKEYKCKVSNKALPAPI |
| | | EKTISKAKGQPREPQVYTLPPSRDELTKNQVSLTCLV |
| | | KGFYPSDIAVEWESNGQPENNYKTTPPVLDSDGSFF |
| | | LYSKLTVDKSRWQQGNVFSCSVMHEALHNHYTQK |
| | | SLSLSPGK |
| | | (SEQ ID NO: 76) |
| | Light chain: | EIVLTQSPATLSLSPGERATISCRASESVESYDIQSMH |
| | LC02-VK3 | WYQQKPGQAPRLLIYRASHLQSGIPARFSGSGSRTD |
| | (D30E/N35Q)- | FTLTISSLEPEDFATYYCQQSNEDPLTFGQGTKLEIK |
| | IL12 | RTVAAPSVFIFPPSDEQLKSGTASVVCLLNNFYPREA |
| | | KVQWKVDNALQSGNSQESVTEQDSKDSTYSLSSTL |
| | | TLSKADYEKHKVYACEVTHQGLSSPVTKSFNRGEC |
| | | GGGGSGGGGSGGGGSGGGGS |
| | | IWELKKDVYVVELDWYPDAPGEMVVLTCDTPEED |
| | | GITWTLDQSSEVLGSGKTLTIQVKEFGDAGQYTCHK |
| | | GGEVLSHSLLLLHKKEDGIWSTDILKDQKEPKNKTF |
| | | LRCEAKNYSGRFTCWWLTTISTDLTFSVKSSRGSSD |
| | | PQGVTCGAATLSAERVRGDNKEYEYSVECQEDSAC |
| | | PAAEESLPIEVMVDAVHKLKYENYTSSFFIRDIIKPDP |
| | | PKNLQLKPLKNSRQVEVSWEYPDTWSTPHSYFSLTF |
| | | CVQVQGKSKREKKDRVFTDKTSATVICRKNASISVR |
| | | AQDRYYSSSWSEWASVPCS |
| | | GGGSGGGSGGGSGGGS |
| | | RNLPVATPDPGMFPCLHHSQNLLRAVSNMLQKARQ |
| | | TLEFYPCTSEEIDHEDITKDKTSTVEACLPLELTKNES |
| | | CLNSRETSFITNGSCLASRKTSFMMALCLSSIYEDLK |
| | | MYQVEFKTMNAKLLMDPKRQIFLDQNMLAVIDEL |
| | | MQALNFNSETVPQKSSLEEPDFYKTKIKLCILLHAFR |
| | | IRAVTIDRVMSYLNAS |
| | | (SEQ ID NO: 107) |
| 02(VH3/VK3)- | Heavy chain 1: | EVQLVQSGAEVKKPGATLKISCKASGYTFTDFTMH |
| IgG- | HC02-VH3- | WVQQAPGKGLEWIGLVNPNNGGNIYAEKFQGRATL |
| IL12(1x | IL12(L234A, | TVDTSTSTAYMELSSLRSEDTSVYYCGRVPLYTSHG |
| HC) | L235A, | MDYWGQGTLVTVSS |
| | N297A, | ASTKGPSVFPLAPSSKSTSGGTAALGCLVKDYFPEPV |
| | T366W) | TVSWNSGALTSGVHTFPAVLQSSGLYSLSSVVTVPS |
| | | SSLGTQTYICNVNHKPSNTKVDKKVEPKSCDKTHTC |
| | | PPCPAPEAAGGPSVFLFPPKPKDTLMISRTPEVTCVV |
| | | VDVSHEDPEVKFNWYVDGVEVHNAKTKPREEQYA |
| | | STYRVVSVLTVLHQDWLNGKEYKCKVSNKALPAPI |
| | | EKTISKAKGQPREPQVYTLPPSRDELTKNQVSLWCL |
| | | VKGFYPSDIAVEWESNGQPENNYKTTPPVLDSDGSF |
| | | FLYSKLTVDKSRWQQGNVFSCSVMHEALHNHYTQ |
| | | KSLSLSPGK |
| | | GGGSGGGGSGGGGSGGGGS |
| | | IWELKKDVYVVELDWYPDAPGEMVVLTCDTPEED |
| | | GITWTLDQSSEVLGSGKTLTIQVKEFGDAGQYTCHK |
| | | GGEVLSHSLLLLHKKEDGIWSTDILKDQKEPKNKTF |
| | | LRCEAKNYSGRFTCWWLTTISTDLTFSVKSSRGSSD |
| | | PQGVTCGAATLSAERVRGDNKEYEYSVECQEDSAC |
| | | PAAEESLPIEVMVDAVHKLKYENYTSSFFIRDIIKPDP |
| | | PKNLQLKPLKNSRQVEVSWEYPDTWSTPHSYFSLTF |
| | | CVQVQGKSKREKKDRVFTDKTSATVICRKNASISVR |
| | | AQDRYYSSSWSEWASVPCS |
| | | GGGSGGGSGGGSGGGS |
| | | RNLPVATPDPGMFPCLHHSQNLLRAVSNMLQKARQ |
| | | TLEFYPCTSEEIDHEDITKDKTSTVEACLPLELTKNES |
| | | CLNSRETSFITNGSCLASRKTSFMMALCLSSIYEDLK |
| | | MYQVEFKTMNAKLLMDPKRQIFLDQNMLAVIDEL |
| | | MQALNFNSETVPQKSSLEEPDFYKTKIKLCILLHAFR |
| | | IRAVTIDRVMSYLNAS |
| | | (SEQ ID NO: 77) |
| | Heavy chain 2: | EVQLVQSGAEVKKPGATLKISCKASGYTFTDFTMH |
| | HC02-VH3 | WVQQAPGKGLEWIGLVNPNNGGNIYAEKFQGRATL |
| | (L234A, | TVDTSTSTAYMELSSLRSEDTSVYYCGRVPLYTSHG |
| | L235A, | MDYWGQGTLVTVSS |
| | N297A, | ASTKGPSVFPLAPSSKSTSGGTAALGCLVKDYFPEPV |
| | T366S, | TVSWNSGALTSGVHTFPAVLQSSGLYSLSSVVTVPS |
| | L368A, | SSLGTQTYICNVNHKPSNTKVDKKVEPKSCDKTHTC |
| | Y407V) | PPCPAPEAAGGPSVFLFPPKPKDTLMISRTPEVTCVV |
| | | VDVSHEDPEVKFNWYVDGVEVHNAKTKPREEQYA |
| | | STYRVVSVLTVLHQDWLNGKEYKCKVSNKALPAPI |
| | | EKTISKAKGQPREPQVYTLPPSRDELTKNQVSLSCA |
| | | VKGFYPSDIAVEWESNGQPENNYKTTPPVLDSDGSF |
| | | FLVSKLTVDKSRWQQGNVFSCSVMHEALHNHYTQ |
| | | KSLSLSPGK |
| | | (SEQ ID NO: 79) |
| | Light chain: | EIVLTQSPATLSLSPGERATISCRASESVDSYDINSMH |
| | LC02-VK3 | WYQQKPGQAPRLLIYRASHLQSGIPARFSGSGSRTD |
| | | FTLTISSLEPEDFATYYCQQSNEDPLTFGQGTKLEIK |
| | | RTVAAPSVFIFPPSDEQLKSGTASVVCLLNNFYPREA |
| | | KVQWKVDNALQSGNSQESVTEQDSKDSTYSLSSTL |
| | | TLSKADYEKHKVYACEVTHQGLSSPVTKSFNRGEC |
| | | (SEQ ID NO: 78) |
| 02(VH4/VK3)- | Heavy chain 1: | EVQLVQSGAEVKKPGATVKISCKASGYTFTDFTMH |
| IgG- | HC02-VH4- | WVQQAPGKGLEWIGLVNPNNGGNIYAEKFQGRATL |
| IL12(1x | IL12(L234A, | TVDTSTSTAYMELSSLRSEDTAVYYCGRVPLYTSHG |
| HC) | L235A, | MDYWGQGTLVTVSS |
| | N297A, | ASTKGPSVFPLAPSSKSTSGGTAALGCLVKDYFPEPV |
| | T366W) | TVSWNSGALTSGVHTFPAVLQSSGLYSLSSVVTVPS |
| | | SSLGTQTYICNVNHKPSNTKVDKKVEPKSCDKTHTC |
| | | PPCPAPEAAGGPSVFLFPPKPKDTLMISRTPEVTCVV |
| | | VDVSHEDPEVKFNWYVDGVEVHNAKTKPREEQYA |
| | | STYRVVSVLTVLHQDWLNGKEYKCKVSNKALPAPI |
| | | EKTISKAKGQPREPQVYTLPPSRDELTKNQVSLWCL |
| | | VKGFYPSDIAVEWESNGQPENNYKTTPPVLDSDGSF |
| | | FLYSKLTVDKSRWQQGNVFSCSVMHEALHNHYTQ |
| | | KSLSLSPGK |
| | | GGGSGGGGSGGGGSGGGGS |
| | | IWELKKDVYVVELDWYPDAPGEMVVLTCDTPEED |
| | | GITWTLDQSSEVLGSGKTLTIQVKEFGDAGQYTCHK |
| | | GGEVLSHSLLLLHKKEDGIWSTDILKDQKEPKNKTF |
| | | LRCEAKNYSGRFTCWWLTTISTDLTFSVKSSRGSSD |
| | | PQGVTCGAATLSAERVRGDNKEYEYSVECQEDSAC |
| | | PAAEESLPIEVMVDAVHKLKYENYTSSFFIRDIIKPDP |
| | | PKNLQLKPLKNSRQVEVSWEYPDTWSTPHSYFSLTF |
| | | CVQVQGKSKREKKDRVFTDKTSATVICRKNASISVR |
| | | AQDRYYSSSWSEWASVPCS |
| | | GGGSGGGSGGGSGGGS |
| | | RNLPVATPDPGMFPCLHHSQNLLRAVSNMLQKARQ |
| | | TLEFYPCTSEEIDHEDITKDKTSTVEACLPLELTKNES |
| | | CLNSRETSFITNGSCLASRKTSFMMALCLSSIYEDLK |
| | | MYQVEFKTMNAKLLMDPKRQIFLDQNMLAVIDEL |
| | | MQALNFNSETVPQKSSLEEPDFYKTKIKLCILLHAFR |
| | | IRAVTIDR VMSYLNAS |
| | | (SEQ ID NO: 80) |
| | Heavy chain 2: | EVQLVQSGAEVKKPGATVKISCKASGYTFTDFTMH |
| | HC02-VH4 | WVQQAPGKGLEWIGLVNPNNGGNIYAEKFQGRATL |
| | (L234A, | TVDTSTSTAYMELSSLRSEDTAVYYCGRVPLYTSHG |
| | L235A, | MDYWGQGTLVTVSS |
| | N297A, | ASTKGPSVFPLAPSSKSTSGGTAALGCLVKDYFPEPV |
| | T366S, | TVSWNSGALTSGVHTFPAVLQSSGLYSLSSVVTVPS |
| | L368A, | SSLGTQTYICNVNHKPSNTKVDKKVEPKSCDKTHTC |
| | Y407V) | PPCPAPEAAGGPSVFLFPPKPKDTLMISRTPEVTCVV |
| | | VDVSHEDPEVKFNWYVDGVEVHNAKTKPREEQYA |
| | | STYRVVSVLTVLHQDWLNGKEYKCKVSNKALPAPI |
| | | EKTISKAKGQPREPQVYTLPPSRDELTKNQVSLSCA |
| | | VKGFYPSDIAVEWESNGQPENNYKTTPPVLDSDGSF |
| | | FLVSKLTVDKSRWQQGNVFSCSVMHEALHNHYTQ |
| | | KSLSLSPGK |
| | | (SEQ ID NO: 81) |
| | Light chain: | EIVLTQSPATLSLSPGERATISCRASESVDSYDINSMH |
| | LC02-VK3 | WYQQKPGQAPRLLIYRASHLQSGIPARFSGSGSRTD |
| | | FTLTISSLEPEDFATYYCQQSNEDPLTFGQGTKLEIK |
| | | RTVAAPSVFIFPPSDEQLKSGTASVVCLLNNFYPREA |
| | | KVQWKVDNALQSGNSQESVTEQDSKDSTYSLSSTL |
| | | TLSKADYEKHKVYACEVTHQGLSSPVTKSFNRGEC |
| | | (SEQ ID NO: 78) |
| 02(VH6/VK3)- | Heavy chain 1: | EVQLVQSGAEVKKPGATVKISCKASGYTFTDFTMH |
| IgG- | HC02-VH6- | WVQQAPGKGLEWIGLVNPNNGGNIYAEKFQGRVTL |
| IL12(1x | IL12(L234A, | TVDTSTSTAYMELSSLRSEDTAVYYCGRVPLYTSHG |
| HC) | L235A, | MDYWGQGTLVTVSS |
| | N297A, | ASTKGPSVFPLAPSSKSTSGGTAALGCLVKDYFPEPV |
| | T366W) | TVSWNSGALTSGVHTFPAVLQSSGLYSLSSVVTVPS |
| | | SSLGTQTYICNVNHKPSNTKVDKKVEPKSCDKTHTC |
| | | PPCPAPEAAGGPSVFLFPPKPKDTLMISRTPEVTCVV |
| | | VDVSHEDPEVKFNWYVDGVEVHNAKTKPREEQYA |
| | | STYRVVSVLTVLHQDWLNGKEYKCKVSNKALPAPI |
| | | EKTISKAKGQPREPQVYTLPPSRDELTKNQVSLWCL |
| | | VKGFYPSDIAVEWESNGQPENNYKTTPPVLDSDGSF |
| | | FLYSKLTVDKSRWQQGNVFSCSVMHEALHNHYTQ |
| | | KSLSLSPGK |
| | | GGGSGGGGSGGGGSGGGGS |
| | | IWELKKDVYVVELDWYPDAPGEMVVLTCDTPEED |
| | | GITWTLDQSSEVLGSGKTLTIQVKEFGDAGQYTCHK |
| | | GGEVLSHSLLLLHKKEDGIWSTDILKDQKEPKNKTF |
| | | LRCEAKNYSGRFTCWWLTTISTDLTFSVKSSRGSSD |
| | | PQGVTCGAATLSAERVRGDNKEYEYSVECQEDSAC |
| | | PAAEESLPIEVMVDAVHKLKYENYTSSFFIRDIIKPDP |
| | | PKNLQLKPLKNSRQVEVSWEYPDTWSTPHSYFSLTF |
| | | CVQVQGKSKREKKDRVFTDKTSATVICRKNASISVR |
| | | AQDRYYSSSWSEWASVPCS |
| | | GGGSGGGSGGGSGGGS |
| | | RNLPVATPDPGMFPCLHHSQNLLRAVSNMLQKARQ |
| | | TLEFYPCTSEEIDHEDITKDKTSTVEACLPLELTKNES |
| | | CLNSRETSFITNGSCLASRKTSFMMALCLSSIYEDLK |
| | | MYQVEFKTMNAKLLMDPKRQIFLDQNMLAVIDEL |
| | | MQALNFNSETVPQKSSLEEPDFYKTKIKLCILLHAFR |
| | | IRAVTIDRVMSYLNAS |
| | | (SEQ ID NO: 82) |
| | Heavy chain 2: | EVQLVQSGAEVKKPGATVKISCKASGYTFTDFTMH |
| | HC02-VH6 | WVQQAPGKGLEWIGLVNPNNGGNIYAEKFQGRVTL |
| | (L234A, | TVDTSTSTAYMELSSLRSEDTAVYYCGRVPLYTSHG |
| | L235A, | MDYWGQGTLVTVSS |
| | N297A, | ASTKGPSVFPLAPSSKSTSGGTAALGCLVKDYFPEPV |
| | T366S, | TVSWNSGALTSGVHTFPAVLQSSGLYSLSSVVTVPS |
| | L368A, | SSLGTQTYICNVNHKPSNTKVDKKVEPKSCDKTHTC |
| | Y407V) | PPCPAPEAAGGPSVFLFPPKPKDTLMISRTPEVTCVV |
| | | VDVSHEDPEVKFNWYVDGVEVHNAKTKPREEQYA |
| | | STYRVVSVLTVLHQDWLNGKEYKCKVSNKALPAPI |
| | | EKTISKAKGQPREPQVYTLPPSRDELTKNQVSLSCA |
| | | VKGFYPSDIAVEWESNGQPENNYKTTPPVLDSDGSF |
| | | FLVSKLTVDKSRWQQGNVFSCSVMHEALHNHYTQ |
| | | KSLSLSPGK |
| | | (SEQ ID NO: 83) |
| | Light chain: | EIVLTQSPATLSLSPGERATISCRASESVDSYDINSMH |
| | LC02-VK3 | WYQQKPGQAPRLLIYRASHLQSGIPARFSGSGSRTD |
| | | FTLTISSLEPEDFATYYCQQSNEDPLTFGQGTKLEIK |
| | | RTVAAPSVFIFPPSDEQLKSGTASVVCLLNNFYPREA |
| | | KVQWKVDNALQSGNSQESVTEQDSKDSTYSLSSTL |
| | | TLSKADYEKHKVYACEVTHQGLSSPVTKSFNRGEC |
| | | (SEQ ID NO: 78) |
| 02(VH6/VK3- | Heavy chain 1: | EVQLVQSGAEVKKPGATVKISCKASGYTFTDFTMH |
| D30E/N35Q)- | HC02-VH6- | WVQQAPGKGLEWIGLVNPNNGGNIYAEKFQGRVTL |
| IgG- | IL12(L234A, | TVDTSTSTAYMELSSLRSEDTAVYYCGRVPLYTSHG |
| IL12(1x | L235A, | MDYWGQGTLVTVSS |
| HC) | N297A, | ASTKGPSVFPLAPSSKSTSGGTAALGCLVKDYFPEPV |
| | T366W) | TVSWNSGALTSGVHTFPAVLQSSGLYSLSSVVTVPS |
| | | SSLGTQTYICNVNHKPSNTKVDKKVEPKSCDKTHTC |
| | | PPCPAPEAAGGPSVFLFPPKPKDTLMISRTPEVTCVV |
| | | VDVSHEDPEVKFNWYVDGVEVHNAKTKPREEQYA |
| | | STYRVVSVLTVLHQDWLNGKEYKCKVSNKALPAPI |
| | | EKTISKAKGQPREPQVYTLPPSRDELTKNQVSLWCL |
| | | VKGFYPSDIAVEWESNGQPENNYKTTPPVLDSDGSF |
| | | FLYSKLTVDKSRWQQGNVFSCSVMHEALHNHYTQ |
| | | KSLSLSPGK |
| | | GGGSGGGGSGGGGSGGGGS |
| | | IWELKKDVYVVELDWYPDAPGEMVVLTCDTPEED |
| | | GITWTLDQSSEVLGSGKTLTIQVKEFGDAGQYTCHK |
| | | GGEVLSHSLLLLHKKEDGIWSTDILKDQKEPKNKTF |
| | | LRCEAKNYSGRFTCWWLTTISTDLTFSVKSSRGSSD |
| | | PQGVTCGAATLSAERVRGDNKEYEYSVECQEDSAC |
| | | PAAEESLPIEVMVDAVHKLKYENYTSSFFIRDIIKPDP |
| | | PKNLQLKPLKNSRQVEVSWEYPDTWSTPHSYFSLTF |
| | | CVQVQGKSKREKKDRVFTDKTSATVICRKNASISVR |
| | | AQDRYYSSSWSEWASVPCS |
| | | GGGSGGGSGGGSGGGS |
| | | RNLPVATPDPGMFPCLHHSQNLLRAVSNMLQKARQ |
| | | TLEFYPCTSEEIDHEDITKDKTSTVEACLPLELTKNES |
| | | CLNSRETSFITNGSCLASRKTSFMMALCLSSIYEDLK |
| | | MYQVEFKTMNAKLLMDPKRQIFLDQNMLAVIDEL |
| | | MQALNFNSETVPQKSSLEEPDFYKTKIKLCILLHAFR |
| | | IRAVTIDR VMSYLNAS |
| | | (SEQ ID NO: 82) |
| | Heavy chain 2: | EVQLVQSGAEVKKPGATVKISCKASGYTFTDFTMH |
| | HC02-VH6 | WVQQAPGKGLEWIGLVNPNNGGNIYAEKFQGRVTL |
| | (L234A, | TVDTSTSTAYMELSSLRSEDTAVYYCGRVPLYTSHG |
| | L235A, | MDYWGQGTLVTVSS |
| | N297A, | ASTKGPSVFPLAPSSKSTSGGTAALGCLVKDYFPEPV |
| | T366S, | TVSWNSGALTSGVHTFPAVLQSSGLYSLSSVVTVPS |
| | L368A, | SSLGTQTYICNVNHKPSNTKVDKKVEPKSCDKTHTC |
| | Y407V) | PPCPAPEAAGGPSVFLFPPKPKDTLMISRTPEVTCVV |
| | | VDVSHEDPEVKFNWYVDGVEVHNAKTKPREEQYA |
| | | STYRVVSVLTVLHQDWLNGKEYKCKVSNKALPAPI |
| | | EKTISKAKGQPREPQVYTLPPSRDELTKNQVSLSCA |
| | | VKGFYPSDIAVEWESNGQPENNYKTTPPVLDSDGSF |
| | | FLVSKLTVDKSRWQQGNVFSCSVMHEALHNHYTQ |
| | | KSLSLSPGK |
| | | (SEQ ID NO: 83) |
| | Light chain: | EIVLTQSPATLSLSPGERATISCRASESVESYDIQSMH |
| | LC02- | WYQQKPGQAPRLLIYRASHLQSGIPARFSGSGSRTD |
| | VK3(D30E/N35Q) | FTLTISSLEPEDFATYYCQQSNEDPLTFGQGTKLEIK |
| | | RTVAAPSVFIFPPSDEQLKSGTASVVCLLNNFYPREA |
| | | KVQWKVDNALQSGNSQESVTEQDSKDSTYSLSSTL |
| | | TLSKADYEKHKVYACEVTHQGLSSPVTKSFNRGEC |
| | | (SEQ ID NO: 108) |

In certain embodiments, an immunostimulatory fusion protein disclosed herein comprises the amino acid sequences of SEQ ID NOs: 73 and 74; 75 and 74; 76 and 74, or 107 and 74. In certain embodiments, the immunostimulatory fusion protein comprises two polypeptides each comprising the amino acid sequence of SEQ ID NO: 73 and two polypeptides each comprising the amino acid sequence of SEQ ID NO: 74. In certain embodiments, the immunostimulatory fusion protein comprises two polypeptides each comprising the amino acid sequence of SEQ ID NO: 75 and two polypeptides each comprising the amino acid sequence of SEQ ID NO: 74. In certain embodiments, the immunostimulatory fusion protein comprises two polypeptides each comprising the amino acid sequence of SEQ ID NO: 76 and two polypeptides each comprising the amino acid sequence of SEQ ID NO: 74. In certain embodiments, the immunostimulatory fusion protein comprises two polypeptides each comprising the amino acid sequence of SEQ ID NO: 107 and two polypeptides each comprising the amino acid sequence of SEQ ID NO: 74.

In certain embodiments, an immunostimulatory fusion protein disclosed herein comprises the amino acid sequences of SEQ ID NOs: 77, 79, and 78. In certain embodiments, an immunostimulatory fusion protein disclosed herein comprises the amino acid sequences of SEQ ID NOs: 80, 81, and 78. In certain embodiments, an immunostimulatory fusion protein disclosed herein comprises the amino acid sequences of SEQ ID NOs: 82, 83, and 78. In certain embodiments, an immunostimulatory fusion protein disclosed herein comprises the amino acid sequences of SEQ ID NOs: 77, 79, and 108. In certain embodiments, an immunostimulatory fusion protein disclosed herein comprises the amino acid sequences of SEQ ID NOs: 80, 81, and 108. In certain embodiments, an immunostimulatory fusion protein disclosed herein comprises the amino acid sequences of SEQ ID NOs: 82, 83, and 108.

### VI. Methods of Preparation

The fusion proteins described above can be made using recombinant DNA technology well known to a skilled person in the art. For example, one or more isolated polynucleotides encoding a fusion protein disclosed herein can be ligated to other appropriate nucleotide sequences, including, for example, constant region coding sequences, and expression control sequences, to produce conventional gene expression constructs (i.e., expression vectors) encoding the desired fusion protein. Production of defined gene constructs is within routine skill in the art.

Nucleic acids encoding desired fusion proteins can be incorporated (ligated) into expression vectors, which can be introduced into host cells through conventional transfection or transformation techniques. Exemplary host cells are E. coli cells, Chinese hamster ovary (CHO) cells, human embryonic kidney 293 (HEK 293) cells, HeLa cells, baby hamster kidney (BHK) cells, monkey kidney cells (COS), human hepatocellular carcinoma cells (e.g., Hep G2), and myeloma cells that do not otherwise produce IgG protein. Transformed host cells can be grown under conditions that permit the host cells to express the genes that encode the fusion proteins.

Specific expression and purification conditions will vary depending upon the expression system employed. For example, if a gene is to be expressed in E. coli, it is first cloned into an expression vector by positioning the engineered gene downstream from a suitable bacterial promoter, e.g., Trp or Tac, and a prokaryotic signal sequence. The expressed protein may be secreted. The expressed protein may accumulate in refractile or inclusion bodies, which can be harvested after disruption of the cells by French press or sonication. The refractile bodies then are solubilized, and the protein may be refolded and/or cleaved by methods known in the art.

If the engineered gene is to be expressed in eukaryotic host cells, e.g., CHO cells, it is first inserted into an expression vector containing a suitable eukaryotic promoter, a secretion signal, a poly A sequence, and a stop codon. Optionally, the vector or gene construct may contain enhancers and introns. In embodiments involving fusion proteins comprising an antibody or portion thereof, the expression vector optionally contains sequences encoding all or part of a constant region, enabling an entire, or a part of, a heavy or light chain to be expressed. The gene construct can be introduced into eukaryotic host cells using conventional techniques.

The fusion proteins disclosed herein may comprise a single polypeptide chain. In this instance, a host cell can be transfected with a single vector expressing the polypeptide (e.g., containing an expression control sequence operably linked to a nucleotide sequence encoding the polypeptide). Alternatively, the fusion proteins disclosed herein may comprise two or more polypeptides. In this instance, a host cell can be co-transfected with more than one expression vector, for example, one expression vector expressing each polypeptide. A host cell can also be transfected with a single expression vector that expresses the two or more polypeptides. For example, the coding sequences of the two or more polypeptides can be operably linked to different expression control sequences (e.g., promoter, enhancer, and/or internal ribosome entry site (IRES)). The coding sequences of the two or more polypeptides can also be separated by a ribosomal skipping sequence or self-cleaving sequence, such as a 2A peptide.

In certain embodiments, in order to express a fusion protein, an N-terminal signal sequence is included in the protein construct. Exemplary N-terminal signal sequences include signal sequences from interleukin-2, CD-5, IgG kappa light chain, trypsinogen, serum albumin, and prolactin.

After transfection, single clones can be isolated for cell bank generation using methods known in the art, such as limited dilution, ELISA, FACS, microscopy, or Clonepix. Clones can be cultured under conditions suitable for bio-reactor scale-up and maintained expression of the fusion proteins.

The fusion proteins can be isolated and purified using methods known in the art including centrifugation, depth filtration, cell lysis, homogenization, freeze-thawing, affinity purification, gel filtration, ion exchange chromatography, hydrophobic interaction exchange chromatography, and mixed-mode chromatography.

### VII. PHARMACEUTICAL COMPOSITIONS

The present disclosure also features pharmaceutical compositions that contain therapeutically effective amounts of the fusion proteins described herein. The composition can be formulated for use in a variety of drug delivery systems. One or more physiologically acceptable excipients or carriers can also be included in the composition for proper formulation. Suitable formulations for use in the present disclosure are found in Adeboye Adejare, Remington: The Science and Practice of Pharmacy (23d ed. 2020). For a brief review of methods for drug delivery, see, e.g., Langer, Science (1990) 249:1527-1533, 1990.

In certain embodiments, a pharmaceutical composition may contain formulation materials for modifying, maintaining or preserving, for example, the pH, osmolarity, viscosity, clarity, color, isotonicity, odor, sterility, stability, rate of dissolution or release, adsorption or penetration of the composition. In such embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants (see, Adeboye Adejare, Remington: The Science and Practice of Pharmacy (23d ed. 2020)).

In certain embodiments, a pharmaceutical composition may contain nanoparticles, e.g., polymeric nanoparticles, liposomes, or micelles (See Anselmo et al. (2016) BIOENG. TRANSL. MED. 1: 10-29).

In certain embodiments, a pharmaceutical composition may contain a sustained-or controlled-delivery formulation. Techniques for formulating sustained- or controlled-delivery means, such as liposome carriers, bio-erodible microparticles or porous beads and depot injections, are also known to those skilled in the art. Sustained-release preparations may include, e.g., porous polymeric microparticles or semipermeable polymer matrices in the form of shaped articles, e.g., films, or microcapsules. Sustained release matrices may include polyesters, hydrogels, polylactides, copolymers of L-glutamic acid and gamma ethyl-L-glutamate, poly (2-hydroxyethyl-inethacrylate), ethylene vinyl acetate, or poly-D(−)-3-hydroxybutyric acid. Sustained release compositions may also include liposomes that can be prepared by any of several methods known in the art.

Pharmaceutical compositions containing a fusion protein disclosed herein can be presented in a dosage unit form and can be prepared by any suitable method. A pharmaceutical composition should be formulated to be compatible with its intended route of administration. Examples of routes of administration are intravenous (IV), intradermal, inhalation, transdermal, topical, transmucosal, intrathecal and rectal administration. In certain embodiments, a fusion protein disclosed herein is administered by IV infusion. In certain embodiments, a fusion protein disclosed herein is administered by intratumoral injection. Useful formulations can be prepared by methods known in the pharmaceutical art. For example, see Adeboye Adejare, Remington: The Science and Practice of Pharmacy (23d ed. 2020). Formulation components suitable for parenteral administration include a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as EDTA; buffers such as acetates, citrates or phosphates; and agents for the adjustment of tonicity such as sodium chloride or dextrose.

For intravenous administration, suitable carriers include physiological saline, bacteriostatic water, Cremophor ELTM (BASF, Parsippany, NJ) or phosphate buffered saline (PBS). The carrier should be stable under the conditions of manufacture and storage, and should be preserved against microorganisms. The carrier can be a solvent or dispersion medium containing, for example, water, ethanol, polyol (for example, glycerol, propylene glycol, and liquid polyetheylene glycol), and suitable mixtures thereof.

An intravenous drug delivery formulation may be contained in a syringe, pen, or bag. In certain embodiments, the bag may be connected to a channel comprising a tube and/or a needle. In certain embodiments, the formulation may be a lyophilized formulation or a liquid formulation. In certain embodiments, the formulation may freeze-dried (lyophilized) and contained in about 12-60 vials. In certain embodiments, the formulation may be freeze-dried and 45 mg of the freeze-dried formulation may be contained in one vial. In certain embodiments, the about 40 mg-about 100 mg of freeze-dried formulation may be contained in one vial. In certain embodiments, freeze dried formulation from 12, 27, or 45 vials are combined to obtain a therapeutic dose of the protein in the intravenous drug formulation. In certain embodiments, the formulation may be a liquid formulation and stored as about 250 mg/vial to about 1,000 mg/vial. In certain embodiments, the formulation may be a liquid formulation and stored as about 600 mg/vial. In certain embodiments, the formulation may be a liquid formulation and stored as about 250 mg/vial.

These compositions may be sterilized by conventional sterilization techniques, or may be sterile filtered. The resulting aqueous solutions may be packaged for use as-is, or lyophilized, the lyophilized preparation being combined with a sterile aqueous carrier prior to administration. The pH of the preparations typically will be between 3 and 11, more preferably between 5 and 9 or between 6 and 8, and most preferably between 7 and 8, such as 7 to 7.5. The resulting compositions in solid form may be packaged in multiple single dose units, each containing a fixed amount of the above-mentioned agent or agents. The composition in solid form can also be packaged in a container for a flexible quantity.

In certain embodiments, the present disclosure provides a formulation with an extended shelf life including the protein of the present disclosure, in combination with mannitol, citric acid monohydrate, sodium citrate, disodium phosphate dihydrate, sodium dihydrogen phosphate dihydrate, sodium chloride, polysorbate 80, water, and sodium hydroxide.

In certain embodiments, an aqueous formulation is prepared including the protein of the present disclosure in a pH-buffered solution. The buffer of this invention may have a pH ranging from about 4 to about 8, e.g., from about 4.5 to about 6.0, or from about 4.8 to about 5.5, or may have a pH of about 5.0 to about 5.2. Ranges intermediate to the above recited pH's are also intended to be part of this disclosure. For example, ranges of values using a combination of any of the above recited values as upper and/or lower limits are intended to be included. Examples of buffers that will control the pH within this range include acetate (e.g. sodium acetate), succinate (such as sodium succinate), gluconate, histidine, citrate and other organic acid buffers.

In certain embodiments, the formulation includes a buffer system which contains citrate and phosphate to maintain the pH in a range of about 4 to about 8. In certain embodiments the pH range may be from about 4.5 to about 6.0, or from about pH 4.8 to about 5.5, or in a pH range of about 5.0 to about 5.2. In certain embodiments, the buffer system includes citric acid monohydrate, sodium citrate, disodium phosphate dihydrate, and/or sodium dihydrogen phosphate dihydrate. In certain embodiments, the buffer system includes about 1.3 mg/ml of citric acid (e.g., 1.305 mg/ml), about 0.3 mg/ml of sodium citrate (e.g., 0.305 mg/ml), about 1.5 mg/ml of disodium phosphate dihydrate (e.g., 1.53 mg/ml), about 0.9 mg/ml of sodium dihydrogen phosphate dihydrate (e.g., 0.86), and about 6.2 mg/ml of sodium chloride (e.g., 6.165 mg/ml). In certain embodiments, the buffer system includes 1-1.5 mg/ml of citric acid, 0.25 to 0.5 mg/ml of sodium citrate, 1.25 to 1.75 mg/ml of disodium phosphate dihydrate, 0.7 to 1.1 mg/ml of sodium dihydrogen phosphate dihydrate, and 6.0 to 6.4 mg/ml of sodium chloride. In certain embodiments, the pH of the formulation is adjusted with sodium hydroxide.

A polyol, which acts as a tonicifier and may stabilize a fusion protein disclosed herein, may also be included in the formulation. The polyol is added to the formulation in an amount which may vary with respect to the desired isotonicity of the formulation. In certain embodiments, the aqueous formulation may be isotonic. The amount of polyol added may also be altered with respect to the molecular weight of the polyol. For example, a lower amount of a monosaccharide (e.g., mannitol) may be added, compared to a disaccharide (such as trehalose). In certain embodiments, the polyol which may be used in the formulation as a tonicity agent is mannitol. In certain embodiments, the mannitol concentration may be about 5 to about 20 mg/ml. In certain embodiments, the concentration of mannitol may be about 7.5 to 15 mg/ml. In certain embodiments, the concentration of mannitol may be about 10-14 mg/ml. In certain embodiments, the concentration of mannitol may be about 12 mg/ml. In certain embodiments, the polyol sorbitol may be included in the formulation.

A detergent or surfactant may also be added to the formulation. Exemplary detergents include nonionic detergents such as polysorbates (e.g., polysorbates 20, 80 etc.) or poloxamers (e.g., poloxamer 188). The amount of detergent added is such that it reduces aggregation of the formulated antibody and/or minimizes the formation of particulates in the formulation and/or reduces adsorption. In certain embodiments, the formulation may include a surfactant which is a polysorbate. In certain embodiments, the formulation may contain the detergent polysorbate 80 or Tween 80. Tween 80 is a term used to describe polyoxyethylene (20) sorbitanmonooleate (see Fiedler, Lexikon der Hifsstoffe, Editio Cantor Verlag Aulendorf, 4th ed., 1996). In certain embodiments, the formulation may contain between about 0.1 mg/mL and about 10 mg/mL of polysorbate 80, or between about 0.5 mg/mL and about 5 mg/mL. In certain embodiments, about 0.1% polysorbate 80 may be added in the formulation.

In embodiments, the protein product of the present disclosure is formulated as a liquid formulation. The liquid formulation may be presented at a 10 mg/mL concentration in either a USP/Ph Eur type I 50R vial closed with a rubber stopper and sealed with an aluminum crimp seal closure. The stopper may be made of elastomer complying with USP and Ph Eur. In certain embodiments, the liquid formulation may be diluted with 0.9% saline solution. In certain embodiments, the liquid formulation of the disclosure may be prepared as a 10 mg/mL concentration solution in combination with a sugar at a stabilizing level. In certain embodiments the liquid formulation may be prepared in an aqueous carrier. In certain embodiments, a stabilizer may be added in an amount no greater than that which may result in a viscosity undesirable or unsuitable for intravenous administration. In certain embodiments, the sugar may be disaccharides, e.g., sucrose. In certain embodiments, the liquid formulation may also include one or more of a buffering agent, a surfactant, and a preservative.

In certain embodiments, the pH of the liquid formulation may be set by addition of a pharmaceutically acceptable acid and/or base. In certain embodiments, the pharmaceutically acceptable acid may be hydrochloric acid. In certain embodiments, the base may be sodium hydroxide.

The aqueous carrier of interest herein is one which is pharmaceutically acceptable (safe and non-toxic for administration to a human) and is useful for the preparation of a liquid formulation. Illustrative carriers include sterile water for injection (SWFI), bacteriostatic water for injection (BWFI), a pH buffered solution (e.g., phosphate-buffered saline), sterile saline solution, Ringer's solution or dextrose solution.

A preservative may be optionally added to the formulations herein to reduce bacterial action. The addition of a preservative may, for example, facilitate the production of a multi-use (multiple-dose) formulation.

The fusion protein may be lyophilized to produce a lyophilized formulation including the proteins and a lyoprotectant. The lyoprotectant may be sugar, e.g., disaccharides. In certain embodiments, the lyoprotectant may be sucrose or maltose. The lyophilized formulation may also include one or more of a buffering agent, a surfactant, a bulking agent, and/or a preservative.

The amount of sucrose or maltose useful for stabilization of the lyophilized formulation may be in a weight ratio of at least 1:2 protein to sucrose or maltose. In certain embodiments, the protein to sucrose or maltose weight ratio may be of from 1:2 to 1:5. In certain embodiments, the pH of the formulation, prior to lyophilization, may be set by addition of a pharmaceutically acceptable acid and/or base. In certain embodiments the pharmaceutically acceptable acid may be hydrochloric acid. In certain embodiments, the pharmaceutically acceptable base may be sodium hydroxide. Before lyophilization, the pH of the solution containing the protein of the present disclosure may be adjusted between 6 to 8. In certain embodiments, the pH range for the lyophilized drug product may be from 7 to 8.

Actual dosage levels of the active ingredients in the pharmaceutical compositions described herein may be varied so as to obtain an amount of the active ingredient which is effective to achieve the desired therapeutic response for a particular patient, composition, and mode of administration, without being toxic to a subject.

The specific dose can be a uniform dose for each patient, for example, 50-5,000 mg of protein. Alternatively, a patient's dose can be tailored to the approximate body weight or surface area of the patient. Other factors in determining the appropriate dosage can include the disease or condition to be treated or prevented, the severity of the disease, the route of administration, and the age, sex and medical condition of the patient. Further refinement of the calculations necessary to determine the appropriate dosage for treatment is routinely made by those skilled in the art, especially in light of the dosage information and assays disclosed herein. The dosage can also be determined through the use of known assays for determining dosages used in conjunction with appropriate dose-response data. An individual patient's dosage can be adjusted as the progress of the disease is monitored. Blood levels of the targetable construct or complex in a patient can be measured to see if the dosage needs to be adjusted to reach or maintain an effective concentration. Pharmacogenomics may be used to determine which targetable constructs and/or complexes, and dosages thereof, are most likely to be effective for a given individual (Schmitz et al., Clinica Chimica Acta 308: 43-53, 2001; Steimer et al., Clinica Chimica Acta 308: 33-41, 2001).

In general, dosages based on body weight are from about 0.01 μg to about 100 mg per kg of body weight, such as about 0.01 μg to about 100 mg/kg of body weight, about 0.01 g to about 50 mg/kg of body weight, about 0.01 μg to about 10 mg/kg of body weight, about 0.01 μg to about 1 mg/kg of body weight, about 0.01 pg to about 500 μg/kg of body weight, about 0.01 pg to about 250 μg/kg of body weight, about 0.01 μg to about 100 μg/kg of body weight, about 0.01 μg to about 50 μg/kg of body weight, about 0.01 pg to about 40 μg/kg of body weight, about 0.01 μg to about 10 μg/kg of body weight, about 0.01 μg to about 1 μg/kg of body weight, about 0.01 μg to about 0.1 μg/kg of body weight, about 0.1 μg to about 100 mg/kg of body weight, about 0.1 μg to about 50 mg/kg of body weight, about 0.1 μg to about 10 mg/kg of body weight, about 0.1 μg to about 1 mg/kg of body weight, about 0.1 pg to about 500 μg/kg of body weight, about 0.1 pg to about 250 μg/kg of body weight, about 0.1 g to about 100 μg/kg of body weight, about 0.1 pg to about 40 μg/kg of body weight, about 0.1 μg to about 10 μg/kg of body weight, about 0.1 μg to about 1 μg/kg of body weight, about 1 μg to about 100 mg/kg of body weight, about 1 μg to about 50 mg/kg of body weight, about 1 μg to about 10 mg/kg of body weight, about 1 μg to about 1 mg/kg of body weight, about 1 μg to about 500 μg/kg of body weight, about 1 μg to about 250 μg/kg of body weight, about 1 μg to about 100 μg/kg of body weight, about 1 μg to about 50 μg/kg of body weight, about 1 μg to about 40 μg/kg of body weight, about 1 μg to about 10 μg/kg of body weight, about g to about 100 mg/kg of body weight, about 10 μg to about 50 mg/kg of body weight, about 10 μg to about 10 mg/kg of body weight, about 10 μg to about 1 mg/kg of body weight, about 10 μg to about 500 μg/kg of body weight, about 10 μg to about 250 μg/kg of body weight, about 10 μg to about 100 μg/kg of body weight, about 10 μg to about 50 μg/kg of body weight, about 10 μg to about 40 μg/kg of body weight, about 40 μg to about 100 mg/kg of body weight, about 40 μg to about 50 mg/kg of body weight, about 40 μg to about 10 mg/kg of body weight, about 40 μg to about 1 mg/kg of body weight, about 40 μg to about 500 μg/kg of body weight, about 40 μg to about 250 μg/kg of body weight, about 40 μg to about 100 μg/kg of body weight, about 40 μg to about 50 μg/kg of body weight, about 50 μg to about 100 mg/kg of body weight, about 50 μg to about 50 mg/kg of body weight, about 50 g to about 10 mg/kg of body weight, about 50 μg to about 1 mg/kg of body weight, about 50 μg to about 500 μg/kg of body weight, about 50 μg to about 250 μg/kg of body weight, about 50 μg to about 100 μg/kg of body weight, about 100 μg to about 100 mg/kg of body weight, about 100 μg to about 50 mg/kg of body weight, about 100 μg to about 10 mg/kg of body weight, about 100 μg to about 1 mg/kg of body weight, about 100 μg to about 500 μg/kg of body weight, about 100 μg to about 250 μg/kg of body weight, about 250 μg to about 100 mg/kg of body weight, about 250 μg to about 50 mg/kg of body weight, about 250 μg to about 10 mg/kg of body weight, about 250 μg to about 1 mg/kg of body weight, about 250 μg to about 500 μg/kg of body weight, about 500 μg to about 100 mg/kg of body weight, about 500 μg to about 50 mg/kg of body weight, about 500 μg to about 10 mg/kg of body weight, about 500 μg to about 1 mg/kg of body weight, about 1 mg to about 100 mg/kg of body weight, about 1 mg to about 50 mg/kg of body weight, about 1 mg to about 10 mg/kg of body weight, about 10 mg to about 100 mg/kg of body weight, about 10 mg to about 50 mg/kg of body weight, about 50 mg to about 100 mg/kg of body weight.

Doses may be given once or more times daily, weekly, monthly or yearly, or even once every 2 to 20 years. Persons of ordinary skill in the art can estimate repetition rates for dosing based on measured residence times and concentrations of the targetable construct or complex in bodily fluids or tissues. The compositions disclosed herein can be administered intravenously, intraarterially, intraperitoneally, intramuscularly, subcutaneously, or intrathecally by perfusion through a catheter or by direct intralesional injection. In certain embodiments, the composition is administered intravenously. In certain embodiments, the composition is administered subcutaneously. This may be administered once or more times daily, once or more times weekly, once or more times monthly, and once or more times annually.

### VIII. Therapeutic Methods

It is contemplated that the fusion proteins disclosed herein can be used either alone or in combination with other therapeutic agents.

The present disclosure provides a method of treating a disease or condition associated with insufficient CD8⁺ T cell activity, such as a proliferative disorder, cancer, an infectious disease, or a viral disease. In certain embodiments, the method comprises administering a fusion protein (e.g., immunostimulatory fusion protein) disclosed herein to a subject in need thereof.

In certain embodiments, the present disclosure provides a method of treating cancer. In certain embodiments, the cancer to be treated is a solid cancer, such as brain cancer, bladder cancer, breast cancer, cervical cancer, colon cancer, colorectal cancer, endometrial cancer, esophageal cancer, leukemia, lung cancer, liver cancer, melanoma, ovarian cancer, pancreatic cancer, prostate cancer, rectal cancer, renal cancer, stomach cancer, testicular cancer, or uterine cancer. In certain embodiments, the cancer is a vascularized tumor, squamous cell carcinoma, adenocarcinoma, small cell carcinoma, melanoma, glioma, neuroblastoma, sarcoma (e.g., an angiosarcoma or chondrosarcoma), larynx cancer, parotid cancer, biliary tract cancer, thyroid cancer, acral lentiginous melanoma, actinic keratoses, acute lymphocytic leukemia, acute myeloid leukemia, adenoid cystic carcinoma, adenomas, adenosarcoma, adenosquamous carcinoma, anal canal cancer, anal cancer, anorectum cancer, astrocytic tumor, bartholin gland carcinoma, basal cell carcinoma, biliary cancer, bone cancer, bone marrow cancer, bronchial cancer, bronchial gland carcinoma, carcinoid, cholangiocarcinoma, chondosarcoma, choriod plexus papilloma/carcinoma, chronic lymphocytic leukemia, chronic myeloid leukemia, clear cell carcinoma, connective tissue cancer, cystadenoma, digestive system cancer, duodenum cancer, endocrine system cancer, endodermal sinus tumor, endometrial hyperplasia, endometrial stromal sarcoma, endometrioid adenocarcinoma, endothelial cell cancer, ependymal cancer, epithelial cell cancer, Ewing's sarcoma, eye and orbit cancer, female genital cancer, focal nodular hyperplasia, gallbladder cancer, gastric antrum cancer, gastric fundus cancer, gastrinoma, glioblastoma, glucagonoma, heart cancer, hemangiblastomas, hemangioendothelioma, hemangiomas, hepatic adenoma, hepatic adenomatosis, hepatobiliary cancer, hepatocellular carcinoma, Hodgkin's disease, ileum cancer, insulinoma, intaepithelial neoplasia, interepithelial squamous cell neoplasia, intrahepatic bile duct cancer, invasive squamous cell carcinoma, jejunum cancer, joint cancer, Kaposi's sarcoma, pelvic cancer, large cell carcinoma, large intestine cancer, leiomyosarcoma, lentigo maligna melanomas, lymphoma, male genital cancer, malignant melanoma, malignant mesothelial tumors, medulloblastoma, medulloepithelioma, meningeal cancer, mesothelial cancer, metastatic carcinoma, mouth cancer, mucoepidermoid carcinoma, multiple myeloma, muscle cancer, nasal tract cancer, nervous system cancer, neuroepithelial adenocarcinoma nodular melanoma, non-epithelial skin cancer, non-Hodgkin's lymphoma, oat cell carcinoma, oligodendroglial cancer, oral cavity cancer, osteosarcoma, papillary serous adenocarcinoma, penile cancer, pharynx cancer, pituitary tumors, plasmacytoma, pseudosarcoma, pulmonary blastoma, rectal cancer, renal cell carcinoma, respiratory system cancer, retinoblastoma, rhabdomyosarcoma, sarcoma, serous carcinoma, sinus cancer, skin cancer, small cell carcinoma, small intestine cancer, smooth muscle cancer, soft tissue cancer, somatostatin-secreting tumor, spine cancer, squamous cell carcinoma, striated muscle cancer, submesothelial cancer, superficial spreading melanoma, T cell leukemia, tongue cancer, undifferentiated carcinoma, ureter cancer, urethra cancer, urinary bladder cancer, urinary system cancer, uterine cervix cancer, uterine corpus cancer, uveal melanoma, vaginal cancer, verrucous carcinoma, VIPoma, vulva cancer, well differentiated carcinoma, or Wilms tumor. In certain embodiments, the cancer is non-Hodgkin's lymphoma, such as a B-cell lymphoma or a T-cell lymphoma. In certain embodiments, the non-Hodgkin's lymphoma is a B-cell lymphoma, such as a diffuse large B-cell lymphoma, primary mediastinal B-cell lymphoma, follicular lymphoma, small lymphocytic lymphoma, mantle cell lymphoma, marginal zone B-cell lymphoma, extranodal marginal zone B-cell lymphoma, nodal marginal zone B-cell lymphoma, splenic marginal zone B-cell lymphoma, Burkitt lymphoma, lymphoplasmacytic lymphoma, hairy cell leukemia, or primary central nervous system (CNS) lymphoma. In certain other embodiments, the non-Hodgkin's lymphoma is a T-cell lymphoma, such as a precursor T-lymphoblastic lymphoma, peripheral T-cell lymphoma, cutaneous T-cell lymphoma, angioimmunoblastic T-cell lymphoma, extranodal natural killer/T-cell lymphoma, enteropathy type T-cell lymphoma, subcutaneous panniculitis-like T-cell lymphoma, anaplastic large cell lymphoma, or peripheral T-cell lymphoma.

The methods and compositions described herein can be used alone or in combination with other therapeutic agents and/or modalities. The term administered “in combination,” as used herein, is understood to mean that two (or more) different treatments are delivered to the subject during the course of the subject's affliction with the disorder, such that the effects of the treatments on the patient overlap at a point in time. In certain embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In certain embodiments of either case, the treatment is more effective because of combined administration. In certain embodiments, delivery is such that the reduction of a symptom, or other parameter related to the disorder is greater than what would be observed with one treatment delivered in the absence of the other. The effect of the two treatments can be partially additive, wholly additive, or greater than additive.

The disclosure provides a method of treating a subject by the administration of a second therapeutic agent in combination with one or more of the fusion proteins (e.g., immunostimulatory fusion proteins) disclosed herein.

### Combination Therapies

Exemplary therapeutic agents that may be used as part of a combination therapy in treating cancer, include, for example, radiation, mitomycin, tretinoin, ribomustin, gemcitabine, vincristine, etoposide, cladribine, mitobronitol, methotrexate, doxorubicin, carboquone, pentostatin, nitracrine, zinostatin, cetrorelix, letrozole, raltitrexed, daunorubicin, fadrozole, fotemustine, thymalfasin, sobuzoxane, nedaplatin, cytarabine, bicalutamide, vinorelbine, vesnarinone, aminoglutethimide, amsacrine, proglumide, elliptinium acetate, ketanserin, doxifluridine, etretinate, isotretinoin, streptozocin, nimustine, vindesine, flutamide, drogenil, butocin, carmofur, razoxane, sizofilan, carboplatin, mitolactol, tegafur, ifosfamide, prednimustine, picibanil, levamisole, teniposide, improsulfan, enocitabine, lisuride, oxymetholone, tamoxifen, progesterone, mepitiostane, epitiostanol, formestane, interferon-alpha, interferon-2 alpha, interferon-beta, interferon-gamma, colony stimulating factor-1, colony stimulating factor-2, denileukin diftitox, interleukin-2, luteinizing hormone releasing factor and variations of the aforementioned agents that may exhibit differential binding to its cognate receptor, and increased or decreased serum half-life.

An additional class of agents that may be used as part of a combination therapy in treating cancer is immune checkpoint inhibitors. The checkpoint inhibitor may, for example, be selected from a PD-1 antagonist, PD-L1 antagonist, CTLA-4 antagonist, adenosine A2A receptor antagonist, B7-H3 antagonist, B7-H4 antagonist, BTLA antagonist, KIR antagonist, LAG3 antagonist, TIM-3 antagonist, VISTA antagonist or TIGIT antagonist.

In certain embodiments, the checkpoint inhibitor is a PD-1 or PD-L1 inhibitor. PD-1 is a receptor present on the surface of T-cells that serves as an immune system checkpoint that inhibits or otherwise modulates T-cell activity at the appropriate time to prevent an overactive immune response. Cancer cells, however, can take advantage of this checkpoint by expressing ligands, for example, PD-L1, that interact with PD-1 on the surface of T-cells to shut down or modulate T-cell activity. Exemplary PD-1/PD-L1 based immune checkpoint inhibitors include antibody based therapeutics. Exemplary treatment methods that employ PD-1/PD-L1 based immune checkpoint inhibition are described, e.g., in U.S. Pat. Nos. 8,728,474 and 9,073,994, and EP Patent No. 1537878B1, and can include, e.g., the use of anti-PD-1 antibodies. Exemplary anti-PD-1 antibodies are described, for example, in U.S. Pat. Nos. 8,952,136, 8,779,105, 8,008,449, 8,741,295, 9,205,148, 9,181,342, 9,102,728, 9,102,727, 8,952,136, 8,927,697, 8,900,587, 8,735,553, and 7,488,802. Exemplary anti-PD-1 antibodies include, for example, nivolumab (Opdivo®, Bristol-Myers Squibb Co.), pembrolizumab (Keytruda®, Merck Sharp & Dohme Corp.), PDR001 (Novartis Pharmaceuticals), and pidilizumab (CT-011, Cure Tech). Exemplary anti-PD-L1 antibodies are described, for example, in U.S. Pat. Nos. 9,273,135, 7,943,743, 9,175,082, 8,741,295, 8,552,154, and 8,217,149. Exemplary anti-PD-L1 antibodies include, for example, atezolizumab (Tecentriq®, Genentech), duvalumab (AstraZeneca), MEDI4736, avelumab, and BMS 936559 (Bristol Myers Squibb Co.).

In certain embodiments, a composition described herein is administered in combination with a CTLA-4 inhibitor. In the CTLA-4 pathway, the interaction of CTLA-4 on a T-cell with its ligands (e.g., CD80, also known as B7-1, and CD86) on the surface of an antigen presenting cells (rather than cancer cells) leads to T-cell inhibition. Exemplary CTLA-4 based immune checkpoint inhibition methods are described in U.S. Pat. Nos. 5,811,097, 5,855,887, 6,051,227. Exemplary anti-CTLA-4 antibodies are described in U.S. Pat. Nos. 6,984,720, 6,682,736, 7,311,910; 7,307,064, 7,109,003, 7,132,281, 6,207,156, 7,807,797, 7,824,679, 8,143,379, 8,263,073, 8,318,916, 8,017,114, 8,784,815, and 8,883,984, International (PCT) Publication Nos. WO1998042752, WO2000037504, and WO2001014424, and European Patent No. EP 1212422 B1. Exemplary CTLA-4 antibodies include ipilimumab or tremelimumab.

In certain embodiments, a composition described herein is administered in combination with an IDO inhibitor. Exemplary IDO inhibitors include 1-methyl-D-tryptophan (known as indoximod), epacadostat (INCB24360), navoximod (GDC-0919), and BMS-986205.

Yet other agents that may be used as part of a combination therapy in treating cancer are monoclonal antibody agents that target non-checkpoint targets (e.g., herceptin) and non-cytotoxic agents (e.g., tyrosine-kinase inhibitors).

Yet other categories of anti-cancer agents include, for example: (i) an agent selected from an ALK inhibitor, an ATR inhibitor, an A2A Antagonist, a Base Excision Repair inhibitor, a Bcr-Abl Tyrosine Kinase inhibitor, a Bruton's Tyrosine Kinase inhibitor, a CDC7 inhibitor, a CHK1 inhibitor, a Cyclin-Dependent Kinase inhibitor, a DNA-PK inhibitor, an inhibitor of both DNA-PK and mTOR, a DNMT1 inhibitor, a DNMT1 inhibitor plus 2-chloro-deoxyadenosine, an HDAC inhibitor, a Hedgehog Signaling Pathway inhibitor, an IDO inhibitor, a JAK inhibitor, a mTOR inhibitor, a MEK inhibitor, a MELK inhibitor, a MTH1 inhibitor, a PARP inhibitor, a Phosphoinositide 3-Kinase inhibitor, an inhibitor of both PARP1 and DHODH, a Proteasome inhibitor, a Topoisomerase-II inhibitor, a Tyrosine Kinase inhibitor, a VEGFR inhibitor, and a WEE1 inhibitor; (ii) an agonist of OX40, CD137, CD40, GITR, CD27, HVEM, TNFRSF25, or ICOS; and (iii) a cytokine selected from IL-12, IL-15, GM-CSF, and G-CSF.

The fusion proteins (e.g., immunostimulatory fusion proteins) disclosed herein may also be used in combination with an immune cell therapy (e.g., adoptive cell therapy). In certain embodiments, a method of treatment disclosed herein further comprises administering to the subject an immune cell composition comprising immune cells (e.g., T cells, B cells, NK cells, tumor-infiltrating lymphocytes, and/or dendritic cells). In certain embodiments, the immune cell composition comprises CD8⁺ T cells. In certain embodiments, at least 40%, at least 50%, at least 60%, at least 70%, at least 80%, at least 90%, at least 95%, or at least 99% of the cells in the immune cell composition are CD8⁺ T cells. In certain embodiments, the immune cell composition further comprises CD4⁺ T cells, optionally wherein at least 20%, at least 30%, at least 40%, or at least 50% of the cells in the immune cell composition are CD4⁺ T cells.

Immune cells can be obtained from a blood sample of a patient by apheresis. In some embodiments, a lymphocyte-rich fraction and a monocyte-rich fraction can be acquired by elutriating peripheral blood mononuclear cells (PBMCs) of the patient. The monocyte-rich fraction can then be used to prepare antigen-presenting cells (APCs) for priming T cells, which can be obtained from the lymphocyte-rich fraction. Exemplary methods are described in WO2020055931 and U.S. Pat. Nos. 9,963,677 and 9,642,906. In certain embodiments, the immune cell therapy is autologous, i.e., immune cells obtained from a patient, after in vitro culture, are administered to the same patient. In certain embodiments, the immune cell therapy is allogeneic, optionally wherein the immune cells are genetically engineered to inactivate a component of class I MHC (e.g., 02M).

In certain embodiments, the combination therapy of the fusion protein (e.g., immunostimulatory fusion protein) and the immune cell composition has a partially additive effect, wholly additive effect, or greater than additive effect (e.g., synergistic effect). In certain embodiments, the fusion protein improves the efficacy of the immune cell composition (e.g., leads to greater cancer remission, greater reduction in relapse, and/or greater reduction in symptoms). In certain embodiments, the combination therapy permits use of a lower dose of the immune cell composition compared to the dose of normally required to achieve similar effects when administered as a monotherapy.

In certain embodiments, the fusion protein and the immune cell composition are administered simultaneously. For example, in certain embodiments, administration of the fusion protein is continuous or intermittent during the course of the immune cell therapy. In certain embodiments, the fusion protein and the immune cell composition are administered in a single composition, optionally wherein the fusion protein binds CD8⁺ T cells in the immune cell composition through interaction with CD8. Also provided herein is a composition comprising the fusion protein and the immune cell composition.

In certain embodiments, the fusion protein and the immune cell composition are administered sequentially (with or without overlap). In certain embodiments, the fusion protein is administered after the administration of a dose of the immune cell composition has completed. In certain embodiments, the fusion protein is administered at least one week, at least two weeks, at least three weeks, at least four weeks, at least one month, at least two months, at least three months, at least four months, at least five months, at least six months, at least seven months, at least eight months, at least nine months, at least ten months, at least 11 months, at least 12 months, at least one year, or at least two years after the administration of the immune cell composition.

In certain embodiments, the fusion protein is administered in multiple doses. In certain embodiments, at least one dose of the fusion protein is administered at least one week, at least two weeks, at least three weeks, at least four weeks, at least one month, at least two months, at least three months, at least four months, at least five months, at least six months, at least seven months, at least eight months, at least nine months, at least ten months, at least 11 months, at least 12 months, at least one year, or at least two years after the administration of the immune cell composition. In certain embodiments, at least one dose of the fusion protein is administered simultaneously (e.g., in a single composition) with the immune cell therapy, and at least one other dose of the fusion protein is administered at least one week, at least two weeks, at least three weeks, at least four weeks, at least one month, at least two months, at least three months, at least four months, at least five months, at least six months, at least seven months, at least eight months, at least nine months, at least ten months, at least 11 months, at least 12 months, at least one year, or at least two years after the administration of the immune cell composition. Without wishing to be bound by theory, it is contemplated that the later dose can function as a booster dose to augment the survival, proliferation, memory formation, and/or function of the earlier-administered immune cell composition or cells derived therefrom in vivo.

Immune cells (e.g., T cells, e.g., CD8⁺ T cells) can have antigen specificity via a receptor expressed on the cell surface. T cells can naturally express T cell receptors (TCRs), such as αβTCRs or γδTCRs, and can be “trained” to express TCRs that target a given antigen or epitope. Immune cells can also be genetically engineered to express a recombinant TCR or a chimeric antigen receptor (CAR). Where the combination therapy disclosed herein is used to treat cancer, the TCR or CAR can target one or more tumor-associated antigens or epitopes thereof.

In certain embodiments, the T cells have been trained to recognize one or more T cell epitopes of an antigen (e.g., tumor-associated antigen). In certain embodiments, the T cells have been primed by APCs, for example, by co-culturing a T cell composition (e.g., the lymphocyte-rich fraction of PBMCs) with APCs that present one or more T cell epitopes of the antigen. Such co-culture, optionally carried out at a ratio of about 1:1 to about 40:1 (T cells to APCs), can result in expansion of T cells that are reactive to the one or more T cell epitopes. Cytokines such as IL-2, IL-6, IL-7, IL-12, IL-15, and/or IL-21 can be added to the cell culture to increase survival, proliferation, and/or memory formation of the T cells. In certain embodiments, the APCs present a plurality of T cell epitopes, optionally from a plurality of antigens (e.g., tumor-associated antigens), thereby to produce multi-targeted T cells (MTCs).

In certain embodiments, the immune cell composition comprises immune cells (e.g., T cells, e.g., CD8⁺ T cells) engineered to express an antigen receptor. Exemplary antigen receptors include TCRs and CARs. In certain embodiments, the immune cells are genetically engineered to inactivate their endogenous TCRs, for example, by knocking out the TRAC or TRBC gene, to reduce ligand-independent tonic T cell signaling and enhances T cell potency.

In certain embodiments, the immune cells express a CAR comprising an extracellular antigen-binding domain, a transmembrane domain, and a primary signaling domain comprising a functional intracellular signaling domain derived from a stimulatory molecule. In certain embodiments, the CAR further comprises one or more costimulatory signaling domains comprising functional signaling domains derived from one or more costimulatory molecules. Further examples of CAR are provided in U.S. Pat. Nos. 7,446,190 and 9,181,527, U.S. Patent Application Publication Nos. 2016/0340406 and 2017/0049819, and International Patent Application Publication No. WO2018/140725.

In certain embodiments, the extracellular antigen-binding domain of the CAR comprises an antigen-binding site, as defined herein, that binds a target antigen. In certain embodiments, the extracellular antigen-binding domain comprises a Fab fragment or an scFv. In certain embodiments, the extracellular antigen-binding domain comprises an scFv comprising a heavy chain variable domain and a light chain variable domain linked by a peptide linker (e.g., a peptide linker described in the “Linkers” subsection above. In certain embodiments, the extracellular antigen-binding domain comprises an antigen (e.g., autoimmune antigen), wherein cells reactive to this antigen (e.g., expressing antibodies that bind the antigen) are target cells of the immune cell therapy disclosed herein.

The extracellular antigen-binding domain can be fused to the transmembrane domain of the CAR. In certain embodiments, the transmembrane domain of the CAR is derived from a naturally occurring transmembrane protein. In certain embodiments, the transmembrane domain is capable of signaling to the intracellular domain(s) whenever the CAR has bound to a target. In some embodiments, the transmembrane domain comprises the transmembrane region(s) of one or more proteins selected from the group consisting of TCR α chain, TCR β chain, TCR ζ chain, CD28, CD3ε, CD45, CD4, CD5, CD8, CD9, CD16, CD22, EGFR, CD37, CD64, CD80, CD86, CD134, CD137, CD154, KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), NKp44, NKp30, NKp46, CD160, CD19, IL2Rβ, IL2Rγ, IL7Rα, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAMI, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKG2D, and NKG2C. In certain embodiments, the transmembrane domain is one that naturally is associated with one of the domains (e.g., primary signaling domain or co-stimulatory signaling domain) in the CAR. In one embodiment, the transmembrane domain can be selected or modified by amino acid substitution to avoid multimerization with a transmembrane domain of the same or a different surface membrane protein, thereby to minimize interactions with other members of a receptor complex (e.g., the CAR complex). In another embodiment, the transmembrane domain is capable of homodimerization with another CAR on the immune cell (e.g., T cell) surface. In another embodiment, the amino acid sequence of the transmembrane domain may be modified or substituted so as to minimize interactions with the binding domains of the native binding partner present in the same immune cell.

The extracellular antigen-binding domain of the CAR can be connected to the transmembrane domain by a hinge region. A variety of hinges can be employed, including but not limited to the human Ig (immunoglobulin) hinge (e.g., an IgG4 hinge, an IgD hinge), a Gly-Ser linker, a (G4S)4 linker, a KIR2DS2 hinge, and a CD8a hinge.

The intracellular signaling domain of the CAR comprises a primary signaling domain (i.e., a functional signaling domain derived from a stimulatory molecule) and, optionally, one or more costimulatory signaling domains (i.e., functional signaling domains derived from at least one costimulatory molecule). These intracellular signaling domains are responsible for an immune cell response, including but not limited to proliferation, differentiation, and activation of a specialized function of the immune cell (e.g., cytotoxic activity or secretion of cytokines of a T cell) in which the CAR has been placed in. The intracellular signaling domain is thus meant to include any truncated portion of the intracellular signaling domain sufficient to transduce the effector function signal.

Primary signaling domains that act in a stimulatory manner may contain signaling motifs which are known as immunoreceptor tyrosine-based activation motifs or ITAMs. Examples of ITAM containing cytoplasmic signaling sequences that are of particular use in the present application include those derived from CD3 zeta, common FcR gamma (FCERIG), Fc gamma RIIa, FcR beta (Fc Epsilon Rib), CD3 gamma, CD3 delta, CD3 epsilon, CD79a, CD79b, DAP10, and DAP12. In one embodiment, the primary signaling domain comprises a functional, cytoplasmic signaling domain derived from CD3 zeta, FcR gamma, FcR beta, CD3 gamma, CD3 delta, CD3 epsilon, CD5, CD22, CD79a, CD79b, CD66d, 4-1BB, common FcR gamma (FCER1G), Fc gamma RIIa, FcR beta (Fc Epsilon R1b), DAP10, and/or DAP12.

A costimulatory signaling domains comprises a functional signaling domain derived from a costimulatory molecule, a cell surface molecule other than an antigen receptor or its ligands that is required for an efficient response of lymphocytes to an antigen. Examples of costimulatory molecules include CD27, CD28, 4-1BB (CD137), OX40, CD30, CD40, PD-1, ICOS, lymphocyte function-associated antigen-1 (LFA-1, CD 11a/CD18), CD2, CD7, CD258 (LIGHT), NKG2C, B7-H3, CD83 ligands, CD5, ICAM-1, GITR, BAFFR, HVEM (LIGHTR), SLAMF7, NKp80 (KLRF1), NKp44, NKp30, NKp46, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD 11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAMI, CRTAM, Ly9 (CD229), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, and PAG/Cbp. In some embodiments, a costimulatory signaling domain of the CAR comprises a functional signaling domain of a costimulatory molecule described herein, e.g., OX40, CD27, CD28, CD30, CD40, PD-1, CD2, CD7, CD258, NKG2C, B7-H3, a CD83 ligand, ICAM-1, LFA-1 (CD1 la/CD18), ICOS and 4-1BB (CD137), or any combination thereof.

The signaling domains within the cytoplasmic portion of the CAR may be linked to each other in a random or specified order. In certain embodiments, a costimulatory signaling domain is deployed N-terminal to the primary signaling domain. Optionally, the signaling domains are linked by a short oligo- or polypeptide linker, for example, between 2 and 10 amino acids in length.

In certain embodiments, the immune cells of the immune cell composition express a recombinant TCR. Generally, an αβTCR is capable of binding a peptide presented by a major histocompatibility complex (MHC) molecule, whereas a γδTCR does not require MHC-mediated antigen presentation. In some embodiments, the TCR comprises variable a and β chains (also known as TCRα and TCRβ, respectively) or variable γ and δ chains (also known as TCRγ and TCRδ, respectively), or a portion thereof that binds the antigen (e.g., peptide-MHC complex) comprising the α and β chain variable domains or the 7 and S chain variable domains. Generally, the variable domains of a TCR comprise complementarity determining regions involved in recognition of the antigen (e.g., peptide-MHC complex). In certain embodiments, the TCR expressed by the immune cells is cloned from a naturally occurring T cell.

In some embodiments, the TCR comprises a constant domain, a transmembrane domain, and/or a short cytoplasmic tail. In some embodiments, each chain of the TCR can possess one N-terminal immunoglobulin variable domain, one immunoglobulin constant domain, a transmembrane region, and a short cytoplasmic tail at the C-terminal end. In some embodiments, the TCR is associated with invariant proteins of the CD3 complex involved in mediating signal transduction.

The immune cells disclosed herein can be genetically engineered to express a CAR or TCR by introducing a nucleic acid encoding the CAR or TCR. In certain embodiments, the nucleic acid is a DNA molecule (e.g., a cDNA molecule). In certain embodiments, the nucleic acid further comprises an expression control sequence (e.g., promoter and/or enhancer) operably linked to the CAR or TCR coding sequence. In certain embodiments, the immune cells are transduced by a vector, such as a viral vector (e.g., AAV vector, lentiviral vector, or adenoviral vector) or a non-viral vector (e.g., plasmid), comprising a nucleic acid encoding the CAR or TCR. In certain embodiments, the nucleic acid is an RNA molecule (e.g., an mRNA molecule). Methods for generating and modifying mRNA for use in transfection are disclosed in U.S. Pat. Nos. 8,278,036; 8,883,506, and 8,716,465. In certain embodiments, the nucleic acid encodes an amino acid sequence comprising a signal peptide at the N-terminus of the CAR or TCR. Such signal peptide can facilitate cell surface localization of the CAR or TCR when it is expressed in an effector cell, and is cleaved from the CAR during cellular processing.

In certain embodiments, the immune cell composition comprises a protein cluster, also known as a protein nanogel, comprising a plurality of therapeutic protein monomers reversibly crossed-linked to one another by biodegradable linkers. The size of the protein nanogel can range from 30 nm to 1,000 nm in diameter, as measured by dynamic light scattering. In certain embodiments, the protein nanogel can be prepared by reacting the plurality of therapeutic protein monomers with a plurality of cross-linkers. Examples of therapeutic protein monomers comprised in a protein nanogel include, without limitation, cytokines, chemokines, enzymes, co-factors, ligands, receptors and soluble fragments thereof, and other regulatory factors, antibodies and fragments thereof (e.g., IgG, Fab, scFv, single domain antibodies), and engineered proteins such as antibody Fc domain or human serum albumin fused with a regulatory factor or antibody or fragment thereof described above. Additional methods of reversibly cross-linking protein monomers to form a protein nanogel are disclosed in WO2015048498, WO2017218533, WO2019050978, WO2019050977, and WO2020205808.

It is understood that the immunostimulatory fusion proteins disclosed herein, which are designed to activate T lymphocytes, may, under certain circumstances, cause side effects such as neurotoxicity under certain conditions. Accordingly, in certain embodiments, the second therapeutic agent that can be used in combination with the immunostimulatory fusion protein comprises an agent that mitigates a side effect of the immunostimulatory fusion protein, e.g., reduces neurotoxicity. In certain embodiments, the second therapeutic agent inhibits T cell trafficking, for example, reduces or inhibits immune cells from crossing the blood-brain barrier. Non-limiting examples of such therapeutic agents include antagonists (e.g., antagonistic antibodies) of adhesion molecules on immune cells (e.g., α4 integrin), such as natalizumab. In certain embodiments, the second therapeutic agent increases the internalization of a sphingosine-1-phosphate (SIP) receptor (e.g., S1PR1 or S1PR5), such as fingolimod or ozanimod. In certain embodiments, the second therapeutic agent is a nitric oxide synthase (NOS) inhibitor, such as ronopterin, cindunistat, A- 84643, ONO-1714, L-NOARG, NCX-456, VAS-2381, GW-273629, NXN-462, CKD-712, KD-7040, or guanidinoethyldisulfide. In certain embodiments, the second therapeutic agent is an antagonist of CSF1 or CSF1R, such as pexidartinib, emactuzumab, cabiralizumab, LY-3022855, JNJ-40346527, or MCS 110. Additional non-limiting examples of the second therapeutic agents include pentosan polysulfate, minocycline, anti-ICAM-1 antibodies, anti-P-selectin antibodies, anti-CD11a antibodies, anti-CD162 antibodies, and anti-IL-6R antibodies (e.g., tocilizumab).

The amount of the fusion protein and additional therapeutic agent and the relative timing of administration may be selected in order to achieve a desired combined therapeutic effect. For example, when administering a combination therapy to a patient in need of such administration, the therapeutic agents in the combination, or a pharmaceutical composition or compositions comprising the therapeutic agents, may be administered in any order such as, for example, sequentially, concurrently, together, simultaneously and the like. Further, for example, a fusion protein may be administered during a time when the additional therapeutic agent(s) exerts its prophylactic or therapeutic effect, or vice versa.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

It should be understood that the order of steps or order for performing certain actions is immaterial so long as the present invention remain operable. Moreover, two or more steps or actions may be conducted simultaneously.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

The description above describes multiple aspects and embodiments of the invention. The patent application specifically contemplates all combinations and permutations of the aspects and embodiments.

### EXAMPLES

### Example 1. Characterization of Murine Anti-CD8 Antibodies

This example describes an antibody campaign to identify silent murine anti-CD8 antibodies referred to herein as 02A01, 03A01, 04D08, 05F10, and 11F05.

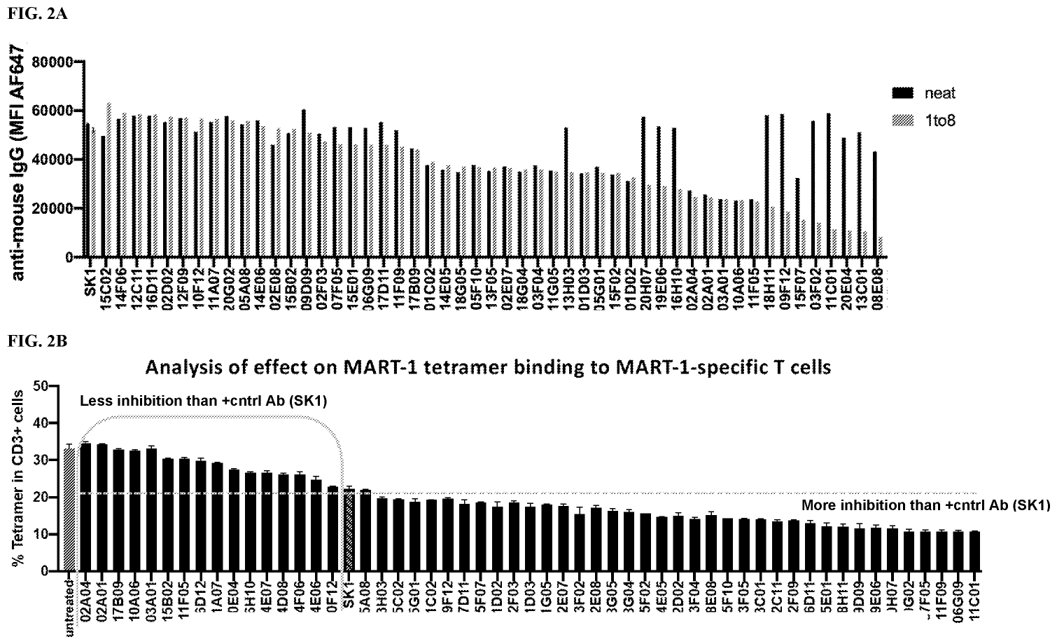

Mice were immunized with human CD8a, and hybridomas that produce antibodies specific for human CD8a were generated using standard techniques. Hybridoma supernatants were evaluated for the ability to bind CD8⁺ T cells. Hybridoma supernatants (undiluted or diluted 1:8) were mixed with human CD8⁺ T cells, incubated with Alexafluor-674-conjugated anti-mouse IgG secondary antibody, and analyzed by flow cytometry. The median fluorescence intensity (MFI) of the anti-mouse IgG bound to anti-CD8 antibodies from the hybridoma supernatants are shown in FIG. 2A. Many of the hybridoma supernatants showed comparable binding in the diluted and undiluted form (e.g. 14F06, 12F09, and 20G02). By contrast, some hybridoma supernatants did not bind comparably in the diluted and 1:8 dilution conditions (e.g. 13C01, 11C01, and 18H11), suggesting that the supernatants did not saturate CD8 binding when diluted 1:8.

Hybridoma supernatants were further evaluated for the capacity to inhibit or enhance TCR/MHC binding and downstream T cell function. Hybridoma supernatants at saturating concentrations (undiluted or diluted 1:8 based on the results shown in FIG. 2A) were analyzed for the ability to inhibit binding of MART-1-specific T cells to fluorophore-conjugated MART-1-MHC tetramers (FIG. 2B) relative to an untreated control. Purified SK1, a commercial anti-CD8 antibody that interferes with CD8 function, was used as a positive control. As shown in FIG. 2B, five of the tested hybridoma pools (02A01, 03A01, 02A04, 10A06, and 17B09) did not inhibit tetramer binding. Additional hybridoma pools (e.g., 11A07, 15B02, and 11F05) only minimally impacted tetramer binding.

Selected hybridoma pools that did not inhibit or that minimally inhibited TCR:MHC-tetramer binding were used to develop monoclonal antibodies. The amino acid sequences of the resulting murine anti-CD8 antibodies 02A01, 03A01, 04D08, 05F10, and 11F05 are provided in Tables 2-4 herein above. These antibodies were recombinantly produced. Their binding affinity to CD8 expressed on the cell surface was measured by flow cytometry, and their respective binding affinities are reported in Table 8 and Table 9. Briefly, human or cynomolgus monkey CD3⁺ T cells were incubated with 7 serial 5-fold dilutions of anti-CD8 antibodies (from 250 nM to 0.08 nM) for 30 minutes. Following the incubation, unbound antibodies were washed away, and the bound antibodies were detected with a AlexaFluor-647-labeled anti-human Fc detection antibody. The median fluorescence intensity of AlexaFluor-647 on the live cells was used to evaluate anti-CD8 antibody levels on the cell surface. Binding affinity (K_{D}) values for anti-CD8 antibody T cell binding were determined with GraphPad Prism 7.0 using 4-Parameter logistic regression.

TABLE 8
Binding of Murine Anti-CD8 Antibodies
to Human CD8-Expressing Cells

| Murine anti-human | CD8⁺ T cell binding |
|---|---|
| CD8 antibody clone | Apparent K_{D} (nM) |
| 02A01 | 0.047 ± 0.023 |
| 03A01 | 0.860 ± 0.101 |
| 05F10 | 0.242 ± 0.058 |
| 04D08 | >100 |
| 11F05 | >100 |

TABLE 9
Binding of Murine Anti-CD8 Antibodies to
Cynomolgus Monkey CD8-Expressing Cells

| Murine anti-human | CD8⁺ Cynomolgus T cell |
|---|---|
| CD8 antibody clone | binding Apparent K_{D} (nM) |
| 02A01 | 0.075 ± 0.010 |
| 03A01 | 1.77 ± 0.20 |
| 05F10 | 39.8 ± 3.73 |

### A. Antibody Competition

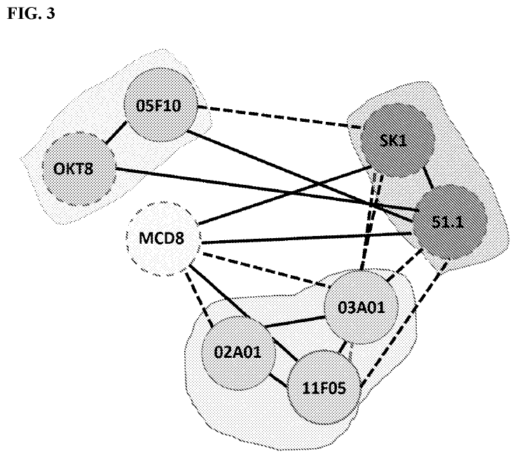

Purified anti-CD8 antibodies that were recombinantly produced herein and known commercial anti-CD8 antibodies SK1, 51.1, and OKT8 were analyzed via epitope binning for their ability to cross-compete with other anti-CD8 antibodies using a Carterra instrument. Briefly, the anti-CD8 antibodies were immobilized to a surface as ligands, CD8a/P was bound to the immobilized antibodies as an antigen, and other anti-CD8 antibodies were tested for binding to the surface as analytes. However, immobilized OKT8 did not capture CD8a/P in this assay format and thus no data was generated for this antibody in this orientation. The results of the binning analysis are summarized in FIG. 3.

Solid lines in FIG. 3 indicate competitive binding in both orientations, and dashed lines indicate competitive binding in only one orientation. Based on the binding patterns, the Carterra software algorithm grouped the antibodies into 4 epitope bins. Novel anti-CD8 antibodies 02A01 and 03A01 were binned together and did not cross-compete with any commercial anti-CD8 antibodies that were tested. Commercial anti-CD8 antibodies SK1 and 51.1 were found to cross-compete with each other and were binned together.

### B. Silent Anti-CD8 Antibodies

The ability of purified anti-CD8 antibodies to bind CD8 without interfering with TCR signaling or T cell function was analyzed by three assays, described below.

Purified recombinant murine anti-CD8 antibodies 02A01, 03A01, 05F10, 11F05, and 04D08 and commercial antibodies SK1, 51.1, and MCD8 were analyzed for their ability to bind T cells without abrogating T cell-mediated killing of a melanoma tumor cell line, SK-MEL-5. Briefly, SK-MEL-5 tumor cells were plated at a density of 5×10³ cells per well on Day −1. On Day 0, the tumor cell culture supernatant was aspirated and the tumor cells were combined with 1×10⁴ melanoma antigen recognized by T cells 1 (MART-1)-specific T cells. Cultures were further supplemented with a saturating concentration of an anti-CD8 IgG antibody (400 nM for 04D08; 25 nM for all other antibodies tested). Cultures without supplementation of an IgG antibody were used to establish a baseline of T cell cytotoxicity. On Day 2, the T cells and supernatant were removed from the cultures, the cultures were washed with PBS, and tumor cell viability was measured with Cell Titer Glo. Results were quantified as the amount of viable tumor cells in the well relative to a well that was not treated with T cells. The results are shown in FIG. 4A.

As shown in FIG. 4A, treatment with any of the three commercial anti-CD8 antibodies (SK1, 51.1, or MCD8) significantly reduced T cell-mediated killing of the tumor cells, resulting in approximately 80-100% post-treatment tumor cell viability relative to an untreated control culture. By contrast, treatment with any of the five silent anti-CD8 antibodies (04D08, 05F10, 02A01, 03A01, and 1IF05) did not substantially inhibit T cell-mediated cytotoxicity.

Purified recombinant anti-CD8 antibodies were also analyzed for their ability to interfere with TCR binding to peptide-MHC tetramers. Briefly, MART-1-specific T cells were plated in a 96-well plate and incubated for 15 minutes with either MACS buffer alone or MACS buffer supplemented with a saturating concentration of an anti-CD8 IgG (25 nM for 05F10; 400 nM for 04D08; 100 nM for all other antibodies tested). The cells were subsequently incubated with 20 nM Brilliant Violet 421-labeled MART-1-MHC tetramers for 30 minutes. Following a dilutive wash, the cells were stained with an anti-CD3 antibody and a viability stain and analyzed by flow cytometry. The results were quantified as the percentage of CD3+ which were tetramer-positive. The results are summarized in FIG. 4B.

As shown in FIG. 4B, the treatment with anti-CD8 antibodies 04D08, 02A01, 03A01, or 11F05 did not impact tetramer binding. By contrast, commercial antibodies SK1 and 51.1 competed with tetramer binding to CD8. Although commercial antibody MCD8 had been found to abrogate T cell-mediated cytotoxicity in the preceding experiment, it was not found to compete with tetramer binding for CD8.

In addition, the purified recombinant anti-CD8 antibodies were analyzed for their ability to abrogate TCR signaling-induced phosphorylation of ERK. Briefly, MART-1-specific T cells were incubated with MART-1 tetramers alone or in combination with a saturating concentration (100 nM) of an anti-CD8 antibody. The cells were then fixed, stained for phospho-ERK, and analyzed by flow cytometry. The results were quantified as the percentage of lymphocytes that were phospho-ERK-positive. The results are summarized in FIG. 4C.

As shown in FIG. 4C, the treatment with anti-CD8 antibodies 02A01, 03A01, or 11F05 did not inhibit TCR-signaling-induced phosphorylation, in contrast to the commercial antibody SK1.

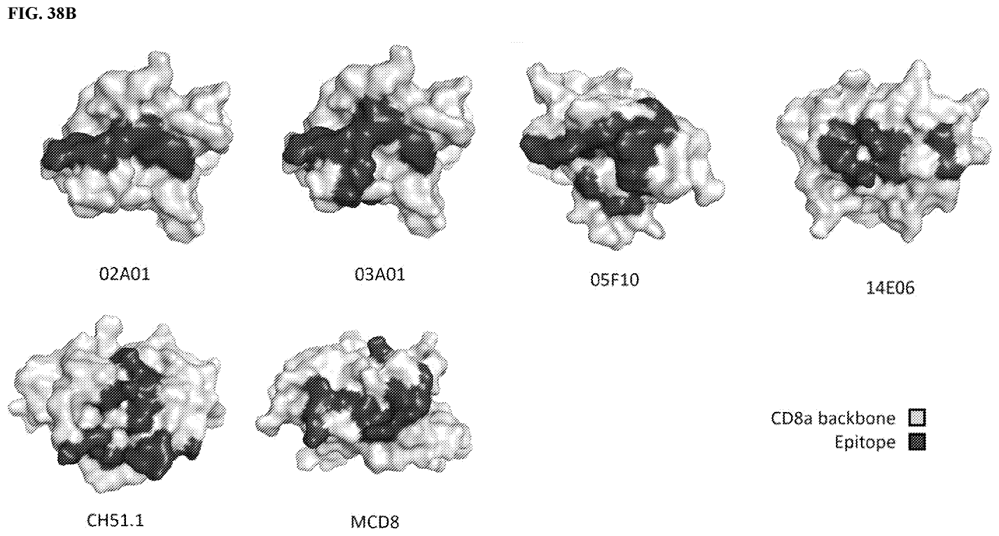

The epitope bound by the anti-CD8 antibody portions was determined by Yeast Surface Display of a scanning mutagenesis library of CD8a. Heatmaps were generated to show enrichment ratios of CD8a variants normalized by wildtype enrichment ratios. Residues that should not be part of an epitope map were denoted and filtered out (e.g., buried residues as determined by 5% and 10% cutoff in solvent-accessible surface area (SASA); paired cysteines; and residues that showed up as hotspots for all antibodies analyzed). The number of substitutions that gave an enrichment ratio greater than 4 was determined, and residues were mapped onto the crystal structure to confirm the resulting map (see FIGS. 38A-38B).

### Example 2. Humanization of Anti-CD8 Antibodies

A. Humanized Antibodies Derived from 02A01

This example describes humanized anti-CD8 antibody heavy chain variable domains 02A01-VH1, 02A01-VH2, 02A01-VH3, 02A01-VH4, 02A01-VH5, and 02A01-VH6, and light chain variable domains 02A01-VK1, 02A01-VK2, 02A01-VK3, and 02A01-VK4, which are derived from the VH and VL, respectively, of murine antibody 02A01. Another VL sequence called 02A01-VK3(D30E/N35Q) was generated by introducing D30E and N35Q substitutions into 02A01-VK3, to address a potential sequence liability issue. In 02A01-VH5, the parental murine HCDR1 was completely replaced with the HCDR1 region of the top human germline hit. The amino acid sequences of these variable domains are provided in Table 2A and Table 2B above. For each humanized VH variant and VL variant, the number of mutated amino acid residues in framework regions (under Kabat) and degree of homology relative to the top human germline hit are reported in Table 10.

TABLE 10
Humanization of anti-CD8 Antibody 02A01

| | | Percentage Homology |
|---|---|---|
| Variable Region | Number of Framework | to the Closest |
| Humanized Variant | Residue Mutations | Human Germline Hit |
| 02A01-VH (Wild Type) | 0 | 65.6 |
| 02A01-VH1 | 11 | 76.0 |
| 02A01-VH2 | 15 | 80.2 |
| 02A01-VH3 | 19 | 84.4 |
| 02A01-VH4 | 21 | 86.5 |
| 02A01-VH5 | 17 | 82.3 |
| 02A01-VH6 | 22 | 87.5 |
| 02A01-VK (Wild Type) | 0 | 65.7 |
| 02A01-VK1 | 14 | 78.7 |
| 02A01-VK2 | 15 | 79.8 |
| 02A01-VK3 | 18 | 81.9 |
| 02A01-VK4 | 21 | 85.1 |

A total of 24 antibodies (excluding 02A01-VK3(D30E/N35Q)) were recombinantly produced in a human IgG1 format, combining each of the six humanized VH variants and each of the four humanized VL variants. Proteins were produced from suspension-adapted HEK 293 cells in serum-free media. Proteins were then purified by either protein A resin (for Fc fusions and full-length (IgG) antibodies), KappaSelect resin (GE Healthcare; for fusions to antibody Fab fragments), or histidine affinity resin (e.g. nickel-nitrilotriacetic acid, Ni-NTA; for 6-His-tagged scFv fusions) as appropriate. Purified proteins were buffer exchanged into phosphate buffered saline (PBS). Approximate molecular weights were confirmed by reducing and non-reducing SDS-PAGE and aggregation and multimeric status was determined by size-exclusion chromatography (SEC). Based on aggregation or multimeric status, proteins were optionally purified by SEC using a HiLoad Superdex 200 prep-grade columns (GE Life Sciences) on an AKTA Pure chromatography system (GE Life Sciences).

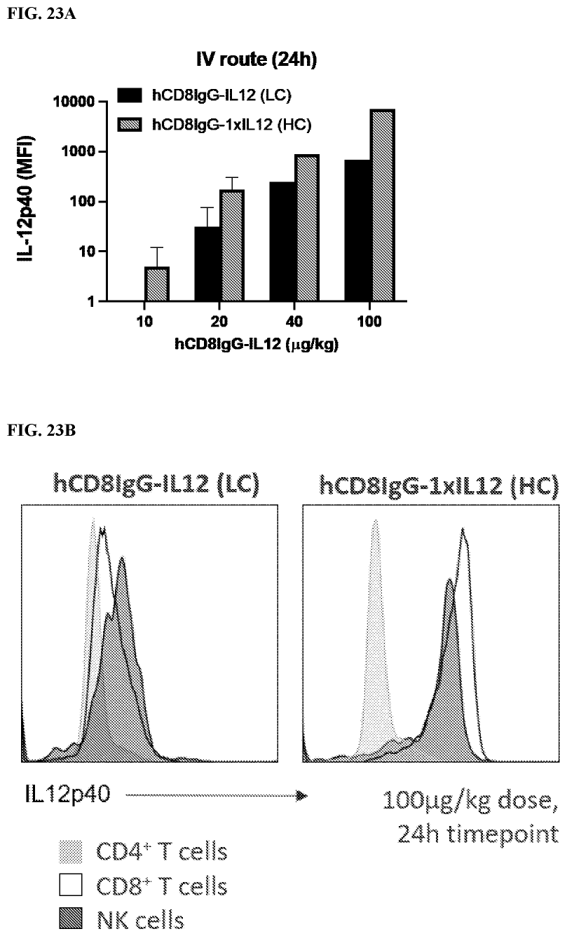

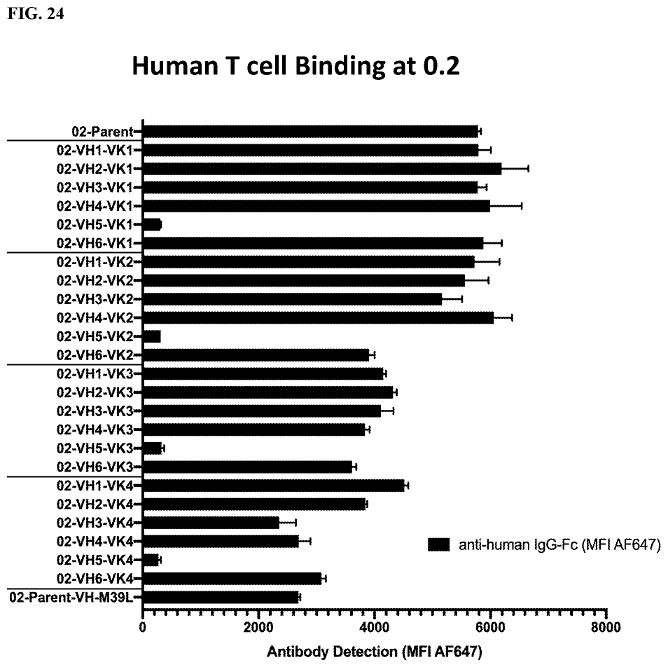

The 24 humanized 02A01-derived anti-CD8 antibodies (excluding 02A01-VK3(D30E/N35Q)) were screened for their ability to bind human CD8 by evaluating binding to CD8+ T cells. The non-humanized parent (“02-Parent”) and the non-humanized parent comprising a heavy chain M39L liability mutation (“02-Parent-VH-M39L”) were used as controls. Briefly, 30×10⁴ human CD8+ T cells (obtained from BioIVT) were incubated with a 0.2 nM concentration of a humanized 02A01 antibody variant (approximately 5× the apparent K_{D} of the parent antibody) for 30 minutes at room temperature. Cells were washed with 1×phosphate buffered saline (PBS) containing 1 mg/mL bovine serum albumin (PBS/BSA) and then stained with a 1:100 dilution of DyLight650-conjugated anti-human IgG H+L polyclonal antibody (ThermoFisher cat. no. Sa5-10129) in PBS/BSA. Following a 20-30 min incubation at 4° C., the cells were washed once with PBS/BSA, and then resuspended in PBS/BSA for analysis on a FACSCelesta using Diva software (BD Biosciences). The resulting data were analyzed using FlowJo. Data were quantified and reported as Max fluorescence intensity (MFI) of human IgG-Fc. As shown in FIG. 24, the humanized antibodies, excluding those comprising VHS, exhibited comparable or moderately decreased binding relative to the non-humanized parent antibody. Complete replacement of the HCDR1 (antibodies comprising VH5) eliminated binding to the CD8+ T cells.

The apparent binding affinities of 14 of the humanized anti-CD8 antibodies to human CD8 were determined by evaluating binding to CD8⁺ T cells using the method described above. Binding to human CD8+ T cells was tested at 7 serial 5-fold dilutions of each tested humanized 02A01 variant (from 50 nM to 3.2 pM). The resulting data were analyzed using FlowJo. The apparent K_{D} for each tested variant is reported in Table 11.

The binding affinities of six of the humanized anti-CD8 antibodies to human CD8 were also determined by surface plasmon resonance (SPR). Briefly, equilibrium dissociation constants for human CD8α/β were determined using a real-time SPR biosensor using a Protein A Sensor Chip (Cytiva) to capture purified anti-CD8 monoclonal antibodies at a concentration of 0.5 nM. SPR binding studies were performed in a buffer composed of Phosphate buffered saline, pH 7.2 containing 0.05% v/v Surfactant P20 (PBS-P20 buffer). Different concentrations of hCD8α/β prepared in PBS-P20 running buffer (ranging from 500 nM to 7.8 nM, 2-fold dilutions) were injected over the anti-CD8 monoclonal antibody captured surface. Association of hCD8α/β to the captured monoclonal antibody was monitored for 400 seconds and the dissociation of hCD8α/b in PBS-P20 running buffer was monitored for 600 sec. All of the binding kinetics experiments were performed at 25° C. Kinetic association (kₐ) and dissociation (kₐ) rate constants were determined by fitting the real-time sensorgrams to a 1:1 binding model using Scrubber 2.0c curve fitting software. The K_{D} values are also provided in Table 11.

TABLE 11
Binding of Humanized 02A01 Antibodies to CD8

| | CD8⁺ T cell | Fold Change | | | Fold Change |
|---|---|---|---|---|---|
| | Binding | to Parental | Maximum | SPR | to Parental |
| 02A01 | Apparent | Apparent | CD8⁺ T cell | Calculated | Calculated |
| Variant | K_{D} (nM) | K_{D} (nM) | Binding (MFI) | K_{D} (nM) | K_{D} (nM) |
| 02-parental | 0.057 | 1.00 | 2904 ± 120 | 9.29 | 1.00 |
| 02-VH4-VK1 | 0.049 | 0.86 | 2814 ± 5 | | |
| 02-VH6/VK1 | 0.058 | 1.02 | 2617 ± 193 | 15.90 | 1.71 |
| 02-VH4/VK2 | 0.065 | 1.14 | 2676 ± 13 | 19.48 | 2.10 |
| 02-VH6/VK2 | 0.096 | 1.68 | 2766 ± 9 | 16.64 | 1.79 |
| 02-VH2-VK3 | 0.067 | 1.18 | 2883 ± 93 | | |
| 02-VH3/VK3 | 0.090 | 1.58 | 2545 ± 17 | 22.04 | 2.37 |
| 02-VH4/VK3 | 0.089 | 1.56 | 2624 ± 77 | 19.79 | 2.13 |
| 02-VH6/VK3 | 0.089 | 1.51 | 2543 ± 70 | 19.65 | 2.12 |
| 02-VH1-VK4 | 0.051 | 0.89 | 2049 ± 66 | | |
| 02-VH2-VK4 | 0.051 | 0.89 | 2045 ± 105 | | |
| 02-VH3-VK4 | 0.066 | 1.16 | 1560 ± 15 | | |
| 02-VH4-VK4 | 0.059 | 1.04 | 1639 ± 62 | | |
| 02-VH5-VK4 | 165.1 | 2896 | 400 ± 42 | | |
| 02-VH6-VK4 | 0.064 | 1.12 | 1694 ± 16 | | |

As shown in Table 11, in the assay measuring apparent binding affinities using human CD8⁺ T cells, the apparent K_{D} of all humanized variants of 02A01 was either similar to that of the parent or was approximately 1.1- to 2-fold higher than that of the parent antibody. Complete replacement of the HCDR1 (the antibodies comprising VHS) abolished binding to the CD8⁺ T cells. There was an approximately 150-300 fold difference in the K_{D} values as measured between SPR and CD8⁺ T cell binding. Without wishing to be bound by theory, it is contemplated that this difference may reflect the ability of anti-CD8 antibodies to avidly bind multiple CD8 proteins in the context of the CD8⁺ T cell-binding assay.

B. Humanized Antibodies Derived from 03A01

This example describes humanized anti-CD8 antibody heavy chain variable domains 03A01-VH1, 03A01-VH2, 03A01-VH3, and 03A01-VH4, and light chain variable domains 03A01-VK1, 03A01-VK2, 03A01-VK3, and 03A01-VK4, which are derived from the VH and VL, respectively, of murine antibody 03A01. In humanized light chain variable domain 03A01-VK4, the parental murine LCDR1 was completely replaced with the LCDR1 region of the top human germline hit. The amino acid sequences of these antibodies are provided in Table 3A and 3B herein above.

A total of 16 antibodies were recombinantly produced, in a human IgG1 format, combining each of the four humanized VH variants and each of the four humanized VL variants. Proteins were produced and purified using the same method as described with respect to humanized 02A01 antibodies.

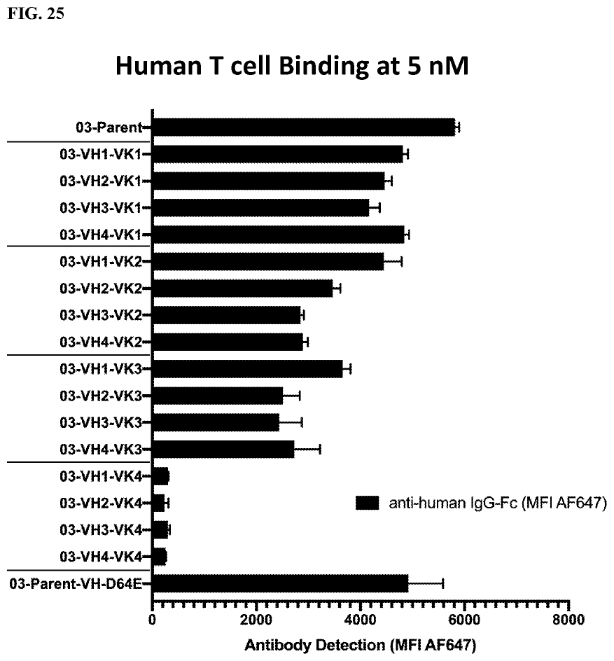

The 16 above-referenced humanized 03A01-derived anti-CD8 antibodies were screened for their ability to bind human CD8 by evaluating binding to CD8⁺ T cells. The non-humanized parent (“03-Parent”) and the non-humanized parent comprising a heavy chain D64E liability mutation (“03-Parent-VH-D64E”) were used as controls. Briefly, 30×10⁴ human CD8+ T cells (obtained from BioIVT) were incubated with a 5 nM concentration of a humanized 03A01 antibody variant (approximately 5× the apparent K_{D} of the parent antibody) for 30 minutes at room temperature. Cells were washed with 1 X phosphate buffered saline (PBS) containing 1 mg/mL bovine serum albumin (PBS/BSA) and then stained with a 1:100 dilution of DyLight650-conjugated anti-human IgG H+L polyclonal antibody (ThermoFisher cat. no. Sa5-10129) in PBS/BSA. Following a 20-30 min incubation at 4° C., the cells were washed once with PBS/BSA, and then resuspended in PBS/BSA for analysis on a FACSCelesta using Diva software (BD Biosciences). The resulting data were analyzed using FlowJo. Data were quantified and reported as Max fluorescence intensity (MFI) of human IgG-Fc. As shown in FIG. 25, the humanized antibodies (excluding those comprising VK4) exhibited comparable or moderately decreased binding relative to the non-humanized parent antibody. Complete replacement of the LCDR1 (antibodies comprising VK4) eliminated binding to the CD8+ T cells.

The binding affinities of five of the above-referenced humanized anti-CD8 antibodies to human CD8 were determined by evaluating binding to CD8 human T cells using the method described above. Binding to human CD8+ T cells was tested at 7 serial 5-fold dilutions of each humanized 03A01 variant (from 50 nM to 3.2 pM). The resulting data were analyzed using FlowJo. The apparent K_{D} for each tested variant is reported in Table 12.

TABLE 12
Binding of Humanized 03A01 Antibodies to CD8

| | CD8⁺ T cell | Fold Change |
|---|---|---|
| | binding | to Parental |
| 03A01 | Apparent | Apparent |
| Variant | K_{D} (nM) | K_{D} (nM) |
| 03-parental | 0.926 | 1.0 |
| 03-VH3/VK1 | 1.651 | 1.8 |
| 03-VH2/VK2 | 1.836 | 2.0 |
| 03-VH1/VK3 | 1.600 | 1.7 |
| 03-VH2/VK3 | 1.909 | 2.1 |
| 03-VH3/VK3 | 1.976 | 2.1 |

As shown in Table 12, the apparent K_{D} of the functional humanized variants of 03A01 was approximately 1.5- to 2-fold higher than that of the parent antibody.

### Example 3. Anti-Mouse CD8/IL-12 Fusion Proteins

### A. Murine CD8-Targeted IL-12 Fusion Proteins

This example describes fusion proteins comprising a mouse CD8 binder and murine IL-12 in various formats.

Six fusion proteins comprising (1) an anti-mouse CD8 antibody or antigen-binding fragment thereof and (2) a murine IL-12 protein comprising an IL-12A subunit and an IL-12B subunit on the same polypeptide chain were generated and evaluated for efficacy and safety in vivo in a syngeneic mouse model. All anti-mouse CD8 antigens and antigen-binding fragments thereof used in these constructs were derived from one of two antagonistic anti-mouse CD8 antibodies, YTS105 or YTS169. Four constructs were derived from YTS105, and were constructed in accordance with the following four scaffold formats described herein: “Fab-immunomodulator (LC)”, “IgG-immunomodulator (LC)”, “IgG-immunomodulator (HC)”, “Fab-Fc-immunomodulator (LC)” (see FIG. 1A-1D). The fifth and sixth constructs, derived from YTS169, were built according to the “IgG-immunomodulator (LC)” and “IgG-immunomodulator (1×HC) scaffold formats described herein, respectively (see FIG. 1B and FIG. 1E). These six fusion constructs are referred to herein as CD8Fab-IL12 (LC), CD8IgG-IL12 (LC), CD8IgG-IL12 (HC), CD8Fab-Fc-IL12 (LC), CD8IgG-IL12 (LC) Clone 2, and CD8IgG-IL12 (1×HC), respectively. The five fusion proteins comprising an Fc region (all except CD8Fab-IL12 (LC)) utilized a human IgG1 Fc, and further comprised the substitutions L234A, L235A, P329G, and N297A in the IgG1 Fc domain that reduce ADCC effector function. The CD8Fab-Fc-IL12 (LC) and CD8IgG-IL12 (1×HC) constructs further comprised the substitution T366W in the Fc polypeptide chain linked to IL-12 and the substitutions T366S, L368A, and Y407V in the other Fc polypeptide chain, which promote heterodimerization.

### MC38 Tumor Model

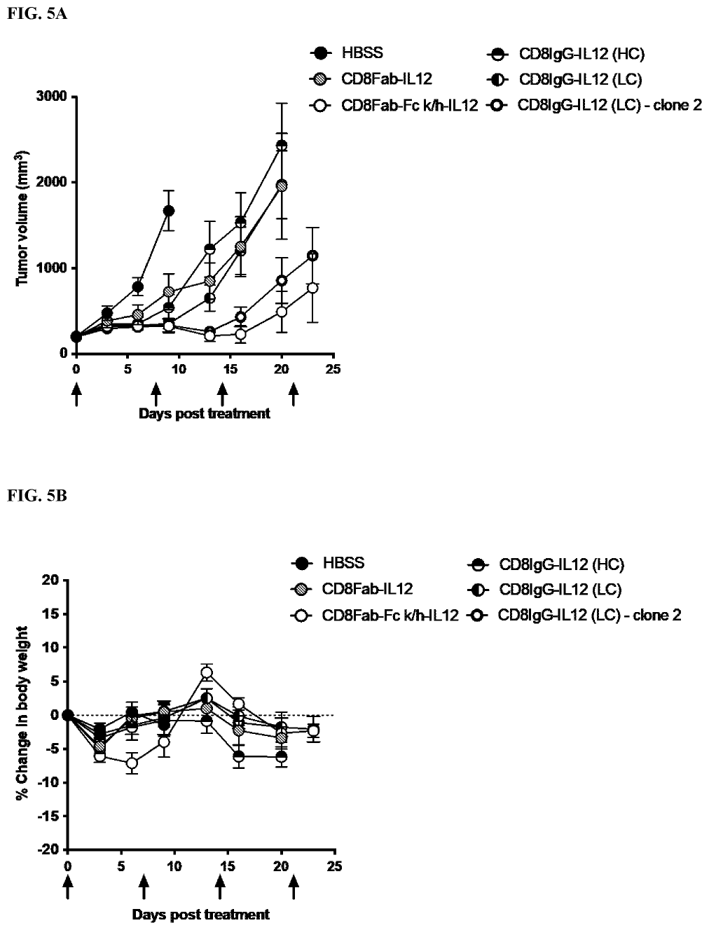

Five of the above-referenced constructs, namely, CD8Fab-IL12 (LC), CD8IgG-IL12 (LC), CD8IgG-IL12 (HC), CD8Fab-Fc-IL12 (LC), and CD8IgG-IL12 (LC) Clone 2, were tested in vivo in a syngeneic mouse tumor model. Briefly, nine C57BL/6 mice (females, 8 weeks old) were used for each study group. Mice were inoculated subcutaneously on the right flank with 2×10⁶ MC38 cells (a murine colon adenocarcinoma cell line from a C57BL/6 mouse). Once tumor volumes were measured as 20 to 250 mm³, mice were injected intravenously with one of the five aforementioned anti-mouse-CD8:IL12 fusion protein constructs. Mice injected with Hanks' Balanced Salt Solution (HBSS) were used as a control. Mice were injected weekly for a total of 4 doses, at a dose equivalent to 10 μg of recombinant IL12. Tumor volume and body weight of the mice were measured twice per week. Mice were euthanized if tumor volume exceeded 2000 mm³ or if body weight fell by more than 20%. As shown in FIG. 5A, treatment with any of the anti-CD8:IL12 fusion proteins resulted in a decrease in tumor volume. Treatment with the CD8Fab-Fc-IL12 (LC) construct (i.e. “Fab-Fc-k/h-IL-12” in FIG. 5A) resulted in the best tumor growth inhibition, but also the most severe early toxicity (measured by body weight loss). The CD8IgG-IL12 (LC) construct derived from YTS169 (“CD8IgG-IL12 (LC)—clone 2”) showed the second-best efficacy, and more modest body weight loss. At early timepoints of the study, mice treated with the CD8Fab-IL12 (LC) construct (“CD8Fab-IL12” in FIG. 5A) showed faster tumor growth than those treated with the other formats of CD8-targeted IL12. In a comparison between CD8IgG-IL12 (HC) and CD8IgG-IL12 (LC) derived from YTS105, better efficacy was observed when IL12 was fused to the light chain rather than the heavy chain. With the exception of CD8Fab-Fc-IL12 (LC) (i.e. “CD8Fab-Fc k/h-IL12 (LC)” in FIG. 5B), mice treated with the other constructs experienced small (<5%) and transient body weight loss following treatment.

### Tissue Retention of Fusion Proteins

Selected anti-CD8:IL12 fusion protein constructs described above were also evaluated for their ability to be retained in tissues following administration to a mouse. Briefly, 8 C57BL/6 mice (females, 9 weeks old) were used for each study group. On Day 0, mice were injected with either CD8IgG-IL12 (LC) (Table 13, columns 3-8) or with CD8Fab-IL12 (LC) or (Table 13, columns 9-14) at a dose equivalent to 10 μg of IL-12. 4 mice in each study group were sacrificed on Day 1, and the remaining 4 mice were sacrificed on Day 3. The mice were not injected with tumor cells. The blood, lymph nodes, and spleens of the sacrificed mice were analyzed for the presence of immune cells with bound fusion protein construct. As summarized in Table 13, both CD8Fab-IL12 (LC) and CD8IgG-IL12 (LC) could direct specific binding of IL-12 to CD8-expressing cells (i.e., CD8⁺ T cells and CD8+ dendritic cells (DCs), rather than NK cells). The CD8IgG-IL12 (LC) construct was retained in tissues for longer than the CD8Fab-IL12 (LC) construct. On Day 1, higher levels of CD8IgG-IL12 (LC) were detected in the blood and spleen, whereas CD8Fab-IL12 (LC) was more prevalent in the spleen and lymph nodes. On Day 3 post-injection, the CD8IgG-IL12 (LC) was present at higher levels in tissues than CD8Fab-IL12 (LC): IL-12 was still detectable on CD8⁺ T cells and CD8⁺ dendritic cells at Day 3 when mice were injected with CD8IgG-IL12 (LC) but not when mice were injected with CD8Fab-IL12 (LC).

TABLE 13
Frequency of cells bound with IL-12 (%)

| | | CD8IgG-IL12 (LC) | | | | | | CD8Fab-IL12 (LC) | | | | | |
|---|---|---|---|---|---|---|---|---|---|---|---|---|---|
| | | CD4⁺ | CD8⁺ | | Mono/ | | | CD4⁺ | CD8⁺ | | Mono/ | | |
| | | T | T | NK | MΦ/ | CD8⁺ | CD8⁻ | T | T | NK | MΦ/ | CD8⁺ | CD8⁻ |
| | | cells | cells | cells | NΦ | DCs | DCs | cells | cells | cells | NΦ | DCs | DCs |
| Day | Blood | 0.0 | 97.2 | 0.9 | 0.6 | n.d. | 4.4 | 0.1 | 80.7 | 1.4 | 0.2 | n.d. | 4.3 |
| 1 | LN | 0.2 | 79.1 | 1.5 | 0.4 | 33.2 | 3.1 | 0.1 | 99.7 | 5.8 | 0.9 | 38.3 | 5.9 |
| | Spleen | 0.5 | 92.0 | 1.5 | 0.3 | 65.1 | 4.5 | 0.1 | 98.7 | 0.9 | 0.1 | 33.4 | 1.6 |
| Day | Blood | 0.0 | 45.2 | 0.9 | 0.0 | n.d. | 0.0 | 0.0 | 0.6 | 0.7 | 0.0 | n.d. | 0.1 |
| 3 | LN | 0.0 | 15.7 | 1.8 | 0.3 | 5.0 | 1.0 | 0.0 | 0.3 | 0.9 | 0.2 | 1.3 | 0.3 |
| | Spleen | 0.0 | 15.6 | 0.8 | 0.1 | 13.5 | 1.1 | 0.0 | 0.0 | 0.3 | 0.0 | 0.5 | 0.2 |

### MC38 and B16-F10 Tumor Models & Tissue Retention of Fusion Proteins

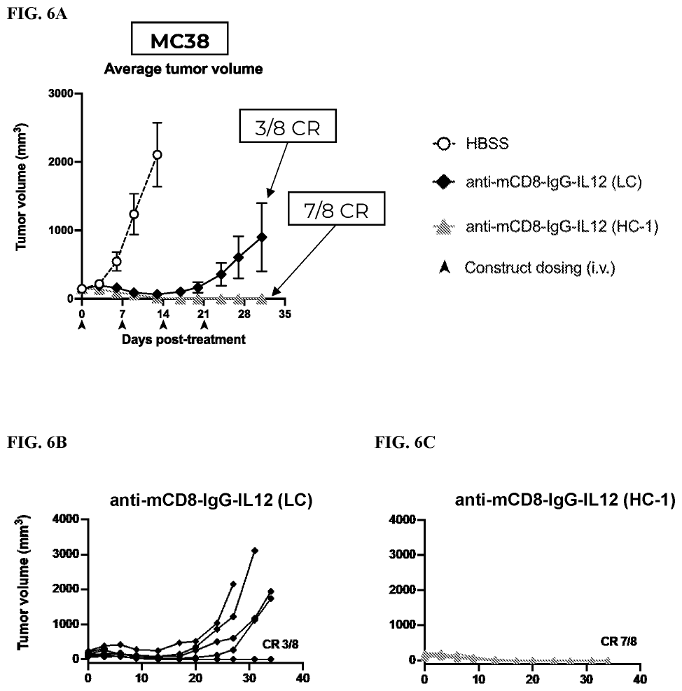

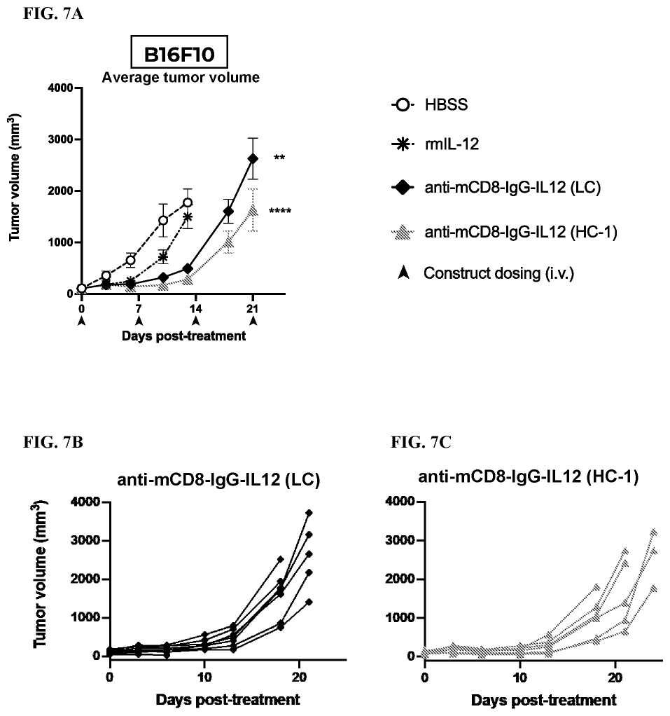

The two anti-CD8: IL-12 fusion protein constructs described above were subsequently tested in vivo in two syngeneic mouse tumor models. Briefly, eight C57BL/6J mice (females, 8 weeks old) were used for each study group. Mice were inoculated subcutaneously on the right lower flank with 2×10⁶ MC38 cells or with 1×10⁶ B16-F10 cells (a murine melanoma cell line from a C57BL/6J mouse). Once tumor volumes were measured as 100 to 150 mm³, mice were injected intravenously with CD8IgG-IL12 (LC) Clone 2, CD8IgG-IL12 (1×HC), non-targeted recombinant mouse IL-12 (rmIL-12), or with HBSS as a control. Constructs in which the CD8-binding site was replaced by an antigen-binding site that binds respiratory syncytial virus (RSV) were used as additional controls (“anti-RSV-IgG-IL12 (LC)” and “anti-RSV-IgG-1×IL12 (HC)”, corresponding to the fusion protein formats illustrated in FIG. 1B and FIG. 1E, respectively). Mice were injected weekly for a total of 4 doses, at a dose equivalent to 10 μg of recombinant IL12. Tumor volume was measured twice per week, and mice were euthanized if tumor volume exceeded 2000 mm³. As shown in FIGS. 6A-6C and FIGS. 7A-7C, both anti-CD8:IL-12 fusion protein constructs showed efficacy in the MC38 and B16-F10 tumor models, and the CD8IgG-IL12 (1×HC) construct was more efficacious than the CD8-IgG1L12 (LC) Clone 2 construct in both models. Specifically, in the MC38 model, treatment with the CD8IgG-IL12 (LC) Clone 2 construct controlled tumor growth up to day 21 and yielded 3/8 complete responders, whereas treatment with the CD8IgG-IL12 (1×HC) construct controlled tumor growth for the entire duration of the study and yielded 7/8 complete responders (FIGS. 6A-6C). In the B16-F10 model, which is typically less responsive to immunotherapy, both constructs limited tumor growth for up to 14 days after the initial dose (FIGS. 7A-7C). In contrast, animals injected with rmIL-12 showed poorer tumor control, and the majority had to be euthanized by about 14 days after the first dose, at a similar time as many animals injected with the vehicle control were also euthanized due to large tumor volumes (FIG. 7A).

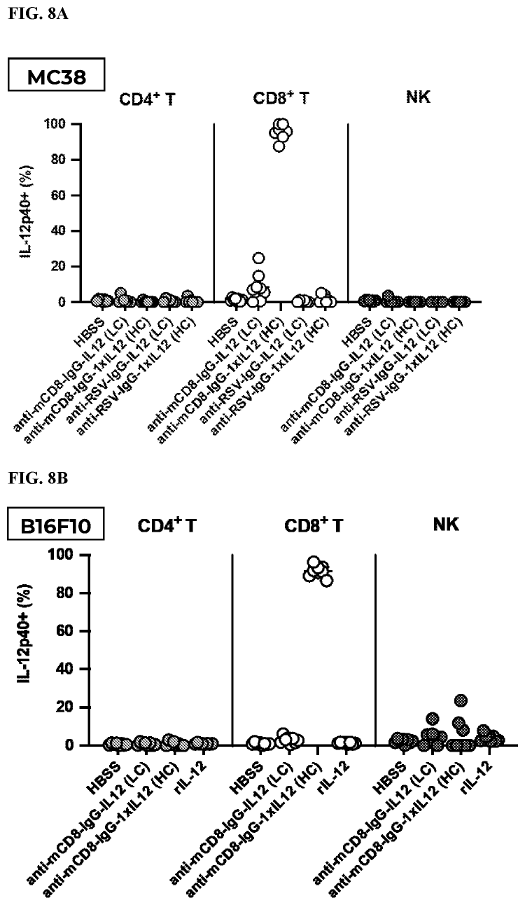

In the same study, the two anti-CD8:IL-12 fusion constructs were also evaluated for their ability to be retained in tissues. Peripheral blood cells were collected from MC38 and B16-F10 tumor-bearing mice 4 days after administration of the first dose of the IL-12 construct. Samples were analyzed by flow cytometry. In the case of the MC38 tumor-bearing mice, IL-12 was detected on the surface of CD8⁺ T cells 4 days post-treatment with either of the two anti-CD8:IL-12 fusion protein constructs, but not on the surface of CD4⁺ T cells or NK cells (FIG. 8A). Notably, IL-12 was detected on a greater amount of CD8⁺ T cells after CD8IgG-IL12 (1×HC) infusion compared to CD8IgG-IL12 (LC) Clone 2 infusion. These observations indicate that CD8-targeted IL-12 binds specifically to CD8⁺ T cells and that the CD8IgG-IL12 (1×HC) scaffold persists on the surface of CD8⁺ T cells longer than the CD8IgG-IL12 (LC) scaffold or untargeted controls. Similar results were obtained in B16-F10-bearing mice, although the CD8IgG-IL12 (LC) Clone 2 construct was found to have a shorter persistence on CD8⁺ T cells as compared to in the MC38-bearing mice (FIG. 8B).

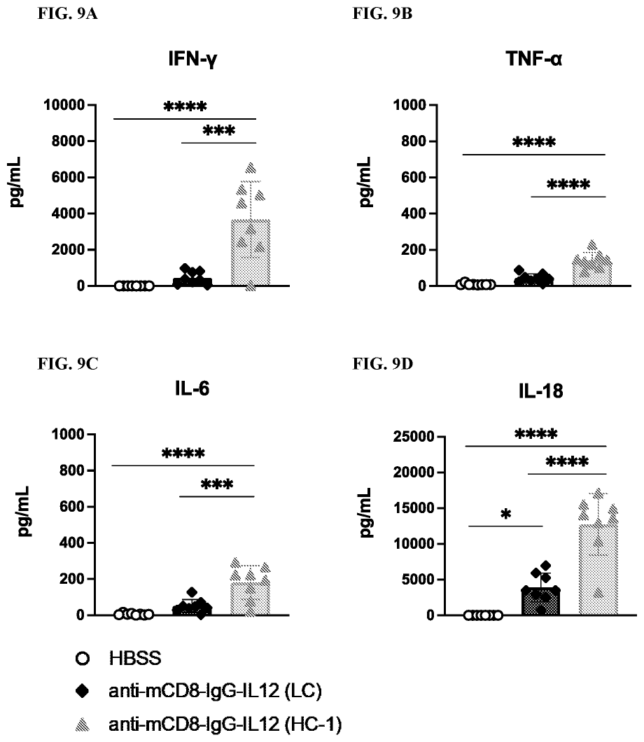

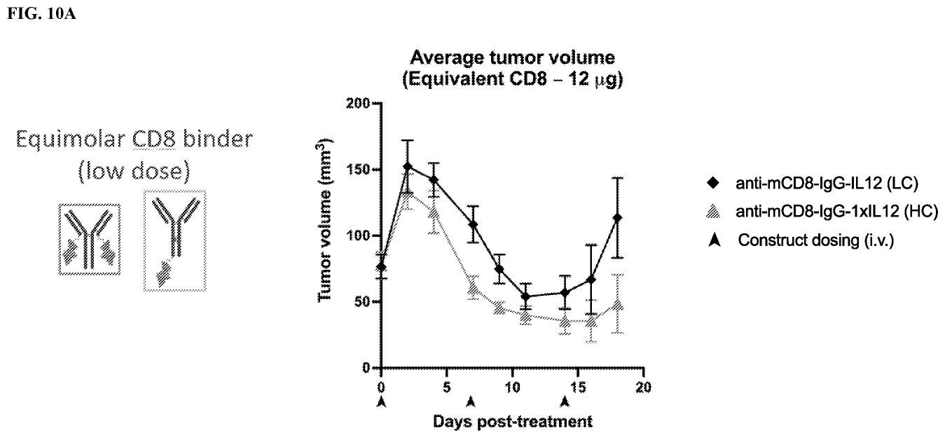

In the same study using the MC38 cell inoculation mouse model, the two anti-CD8:IL-12 fusion constructs were also evaluated for their ability to induce production of pro-inflammatory cytokines. Serum levels of cytokines were measured 4 days post-treatment with the fusion construct using Mouse Th1/Th2 Luminex assay panel (Thermo Fisher). As shown in FIGS. 9A-9D, both CD8IgG-IL12 (1×HC) infusion and CD8IgG-IL12 (LC) Clone 2 infusion increased serum levels of IFN-γ, TNF-α, IL-6, and IL-18. The CD8IgG-IL12 (lx HC) construct was a more potent inducer of the pro-inflammatory cytokines than the CD8IgG-IL12 (LC) Clone 2 construct.

Two of the anti-mouse-CD8:IL-12 fusion constructs described above were subsequently tested in vivo at different doses in a syngeneic mouse tumor model. Briefly, eight C57BL/6J mice (females, 8 weeks old) were used for each study group. Mice were inoculated subcutaneously on the right lower flank with 5×10⁵ MC38 tumor cells. Once tumor volumes were measured as 50 to 100 mm³, mice were injected intravenously with CD8IgG-IL12 (LC) Clone 2 (“anti-mCD8-IgG-IL12 (LC)”) or CD8IgG-IL12 (1×HC) (“anti-mCD8-IgG-1×IL12 (HC)”). Two doses of CD8IgG-IL12 (LC) Clone 2 were tested (22 pg and 44 pg), and two doses of CD8IgG-IL12 (1×HC) were tested (17 pg and 34 pg). Mice were injected weekly for a total of 3 doses. Tumor volume was measured twice per week.

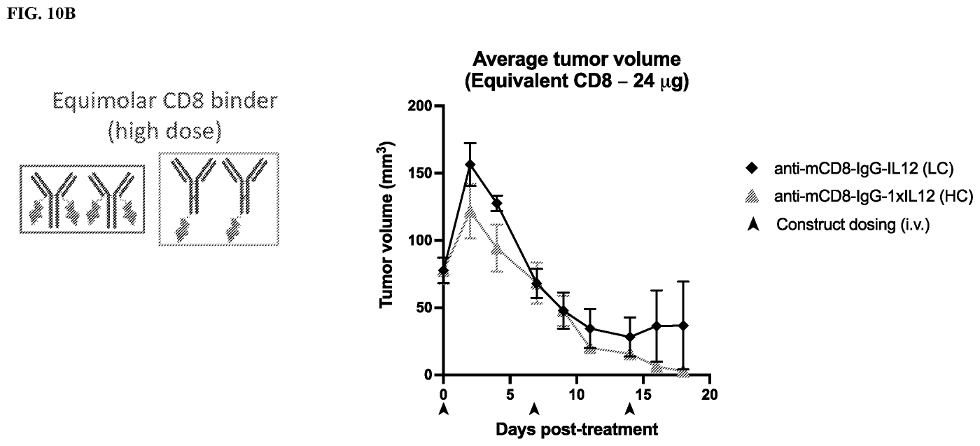

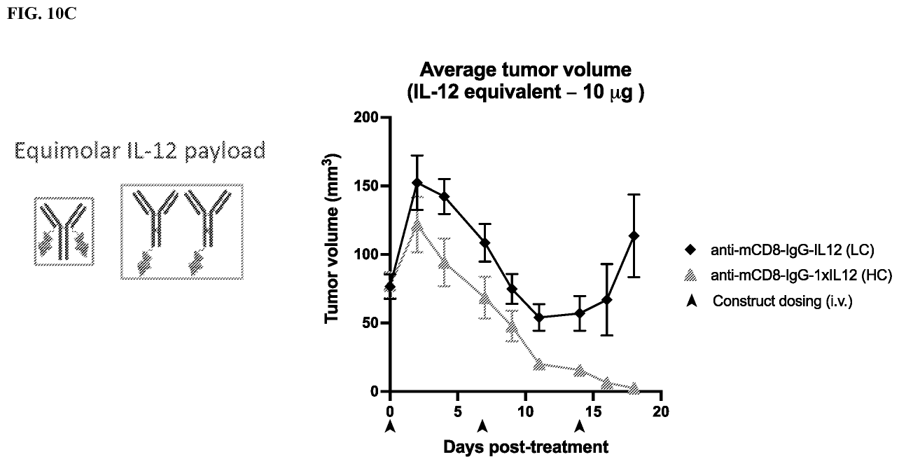

The results of this study are summarized in FIGS. 10A-10C. FIG. 10A summarizes the results of treatment with equimolar construct doses equivalent to 12 μg of the CD8-binder, corresponding to 22 μg of CD8IgG-IL12 (LC) Clone 2 and 17 μg of CD8IgG-IL12 (1×HC). FIG. 10B summarizes the results of treatment with equimolar construct doses equivalent to 24 μg of the CD8-binder, corresponding to 44 μg of CD8IgG-IL12 (LC) Clone 2 and 34 μg of CD8IgG-IL12 (1×HC). FIG. 10C summarizes the results of treatment with construct doses equivalent to 10 pg IL12, corresponding to 22 μg of CD8IgG-IL12 (LC) Clone 2 and 34 μg of CD8IgG-IL12 (1×HC). In all three comparisons, treatment with CD8IgG-IL12 (1×HC) showed better anti-tumor efficacy than CD8IgG-IL12 (LC) Clone 2. The difference in efficacy between the two constructs was less pronounced at a higher dose.

### MC38 Tumor Model & Fusion Protein Binding to T Cells

The same two anti-mouse-CD8:IL-12 fusion constructs described above were also tested for their association with CD8+ T cells for 72 hours following administration in a syngeneic mouse tumor model. Briefly, five C57BL/6J mice (females, 8 weeks old) were used for each study group. Mice were inoculated subcutaneously on the right lower flank with 5×10⁵ MC38 tumor cells. Once tumor volumes were measured as 50 to 100 mm³, mice were injected intravenously with CD8IgG-IL12 (LC) Clone 2 (“anti-mCD8-IgG-IL12 (LC)”) or with CD8IgG-IL12 (1×HC) (“anti-CD8-IgG-1×IL12 (HC)”) at a dose equivalent to 10 μg of rhIL-12. Blood for analysis was collected at 10 minutes, 30 minutes, 1.5 hours, 4 hours, 24 hours, 48 hours, and 72 hours after administration. Red blood cells were lysed using 150 μL ACK Lysing Buffer (Thermo Fisher) and washed with 100 μL MACS buffer. The lysis protocol was performed twice. Samples from each timepoint were incubated with viability dye and with fluorophore-conjugated antibodies against CD45, CD3, CD4, CD8, NK1.1, and IL-12 for 20 minutes at room temperature. Cells were then washed with MACS buffer and analyzed via flow cytometry. CD8+ T cell surface-associated IL-12 was quantified and reported as the mean fluorescence intensity values of IL-12 on CD8+ T cells (FIG. 11A) and as the frequency of IL12+CD8+ T cells (FIG. 11B).

As shown in FIG. 11A, a larger amount of IL-12 was detected on CD8⁺ T cells at every timepoint following treatment with CD8IgG-IL12 (1×HC) relative to CD8IgG-IL12 (LC) Clone 2. As shown in FIG. 11B, IL-12 was detected on the surface of CD8⁺ T cells as early as 10 minutes following administration with either fusion protein construct. In contrast to treatment with CD8IgG-IL12 (1×HC), IL-12 was only detectable on a portion of cells 10 and 30 minutes post-injection with CD8IgG-IL12 (LC) Clone 2, indicating that the (LC) construct was slower to associate with target cells. Additionally, IL-12 was still detectable on CD8⁺ T cells 72 hours post-treatment CD8IgG-IL12 (1×HC), whereas IL-12 was not detectable at the same timepoint after treatment with CD8IgG-IL12 (LC) Clone 2. This result indicates that the (1×HC) construct persists on target cells longer than the (LC) construct.

### MC38 Mouse Model & T Cell Infiltration

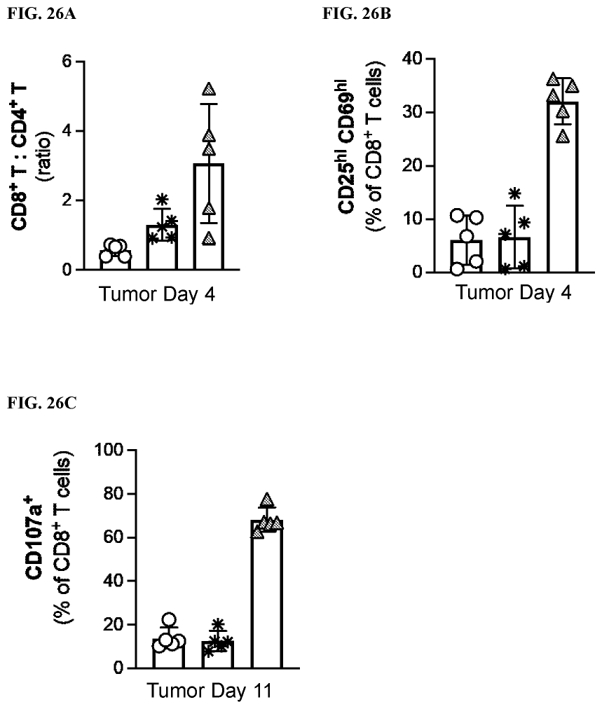

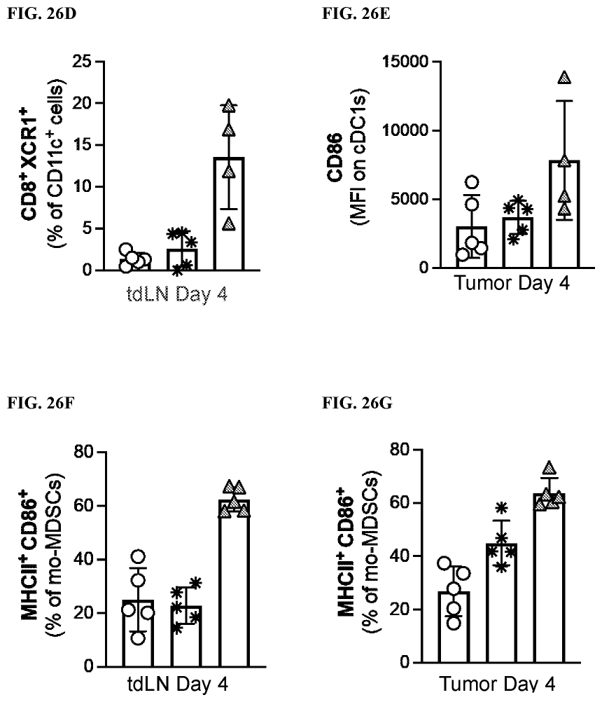

The CD8IgG-IL12 (1×HC) fusion construct was also tested in vivo in a syngeneic mouse tumor model for its ability to stimulate multiple different cell types associated with cancer immunity. Briefly, 8-week-old C57BL/6J mice were inoculated subcutaneously with 10⁶ MC38 tumor cells in the lower right flank. When mean tumor volume of the cohort was 100-150 mm³, mice were randomized according to their tumor volumes and dosed weekly with 17 μg of CD8IgG-IL12 (1×HC). Mice were administered either an equivalent dose of recombinant IL-12 or a vehicle control (HBSS). Mice were sacrificed on day 4, while others on day 11, after the first dose. Tumors and tumor-draining lymph nodes (tdLN) were harvested, homogenized, and stained with fluorophore-conjugated antibodies for flow cytometry analysis. The results are shown in FIGS. 26A-26G.

As summarized in FIGS. 26A-C, CD8IgG-IL12 (1×HC) treatment induced higher CD8⁺ T cell infiltration in the tumor (FIG. 26A), higher expression of T cell activation markers (CD25 and CD69) (FIG. 26B), higher T cell cytotoxicity/degranulation marker (CD107a) (FIG. 26C), higher activation status of dendritic cells (XCR1) (FIG. 26D), higher expression of costimulatory molecule (CD86) on cDC1s, (FIG. 26E), and repolarization of monocytic myeloid-derived suppressor cells (mo-MSDCs) into antigen-presenting cells in the tdLNs and tumor (FIG. 26F and FIG. 26G, respectively), compared to untargeted IL-12 and vehicle controls.

Collectively, FIGS. 26A-26C, show that the targeted IL-12 increases T cell function, e.g., CD8⁺ T cell infiltration into the tumor, activation, and cytotoxic activity. Collectively, FIGS. 26D-26E, show that the targeted IL-12 can activate dendritic cells, e.g., cDC1s. Collectively, FIGS. 26F-26G, show that the targeted IL-12 can overcome the immune-suppressive tumor microenvironment, e.g., the targeted IL-12 repolarized mo-MDSCs from a suppressive to an inflammatory phenotype in tdLNs and in the tumor.

### Pan02 Tumor Model

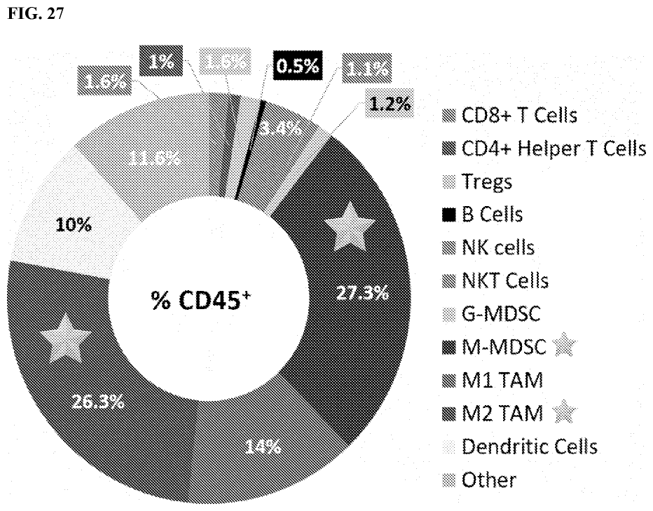

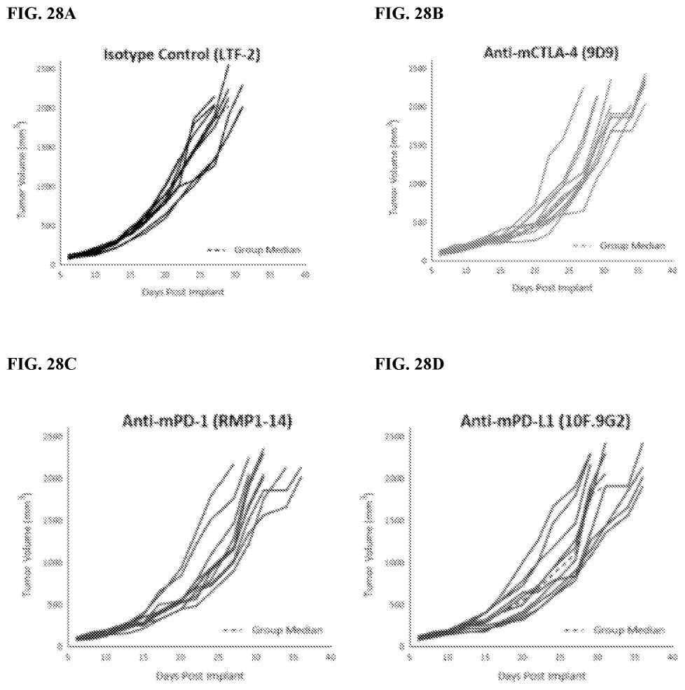

CD8IgG-IL12 (1×HC) was also tested in vivo for anti-tumor efficacy in a syngeneic mouse model of pancreatic ductal cancer (PDAC), Pan02. Human PDAC presents a highly immune-suppressed tumor microenvironment (TME) that is widely unresponsive to therapy. Similarly to human PDAC, the Pan02 model (a C57BL6-derived murine pancreatic cancer) is refractory to immune therapy and presents an immune-suppressed TME. As shown in FIG. 27 and as summarized in Table 14, the Pan02 TME is rich in monocytic myeloid-derived suppressor cells (M-MDSC) and anti-inflammatory M2 tumor-associated macrophages (TAMs), according to data obtained from a Crown Bioscience database. As shown in FIGS. 28A-28D, treatment of Pan02-inoculated mice with standard-of-care checkpoint inhibitor therapies (anti-mCTLA2, anti-mPD-1, or anti-mPD-L1) does not substantially reduce tumor volumes relative to an isotype control, according to data obtained from a Crown Bioscience database.

TABLE 14
Percentage of CD45+ cells corresponding to various
cell types in the mouse Pan02 tumor microenvironment

| | Percentage of |
|---|---|
| Cell Type | CD45+ Cells |
| CD8+ T Cells | 1.6% |
| CD4+ Helper T Cells | 1% |
| Tregs | 1.6% |
| B Cells | 0.5% |
| NK Cells | 3.4% |
| NKT Cells | 1.1% |
| G-MDSC | 1.2% |
| M-MDSC | 27.3% |
| M1 TAM | 14% |
| M2 TAM | 26.3% |
| Dendritic Cells | 10% |
| Other | 11.6% |

To test the efficacy of CD8IgG-IL12 (1×HC) in the Pan02 model, 6- to 8-week-old female C57BL/6J mice were inoculated subcutaneously with 3×10⁶ Pan02 cells in the right flank. When the mean tumor volume of the cohort was 100-120 mm³, the mice were randomized according to their tumor volumes and dosed with either 34 μg of the CD8IgG-IL12 (1×HC) construct subcutaneously or a PBS vehicle control intraperitoneally. Subsequent doses were injected 7 and 14 days after the initial dose. Tumor measurements were recorded twice weekly by caliper after treatment initiation until endpoint.

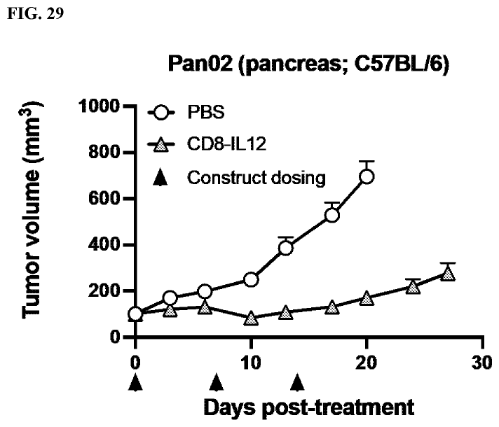

As shown in FIG. 29, CD8IgG-IL12 (1×HC) showed anti-tumor efficacy in the Pan02 PDAC model that is known to be resistant to checkpoint inhibitors (e.g., anti-PD-1, anti-PD-L1, and anti-CTLA4 therapies, see FIGS. 28A-28D). At each timepoint, treatment with CD8IgG-IL12 (1×HC) resulted in better anti-tumor efficacy than vehicle. Collectively, the CD8-targeted IL-12 showed anti-tumor efficacy against a tumor model that presents a TME that contains a high number of M-MDSCs and TAMs. It is contemplated that CD8-targeted IL12 may also be effective in human tumors having similar TMEs.

EMT6, H22, A20, B16BL6, Renca, MC38, CT26, B16F10, LL/2, RM1, and Hepa1-6 tumor models

CD8IgG-IL12 (1×HC) was tested in vivo for anti-cancer efficacy in syngeneic mouse models of breast cancer, liver cancer, lymphoma, melanoma, and kidney cancer. Briefly, 6- to 8-week old female BALB/c mice were inoculated subcutaneously with 0.5-1.0×10⁶ EMT6 tumor cells (breast cancer), H22 tumor cells (liver cancer), A20 tumor cells (lymphoma), or Renca tumor cells (kidney cancer) in the right flank. In the melanoma model, 6- to 8-week old C57BL/6 mice were inoculated with 0.2×10⁶ B16BL6 tumor cells in the right flank. When the mean tumor volume of the cohort reached 100-120 mm³, the mice were randomized according to their tumor volumes and were dosed with either with 34 μg of the fusion construct subcutaneously or a PBS vehicle control peritoneally. Subsequent doses were injected 7 and 14 days after the initial dose. Tumor measurements were recorded twice weekly by caliper until endpoint.

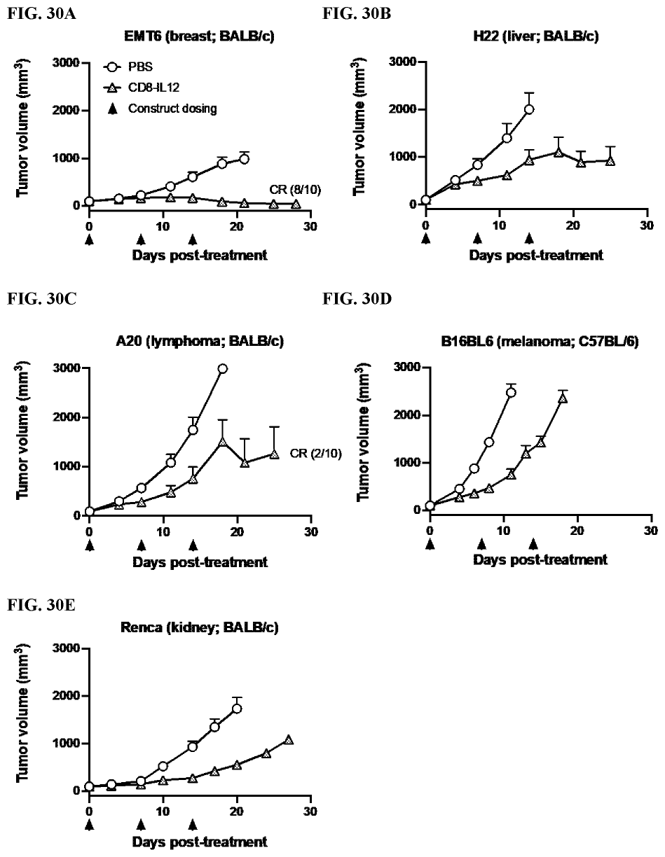

As shown in FIGS. 30A-30E, CD8IgG-IL12 (1×HC) showed anti-tumor efficacy in every cancer model tested. At each timepoint, CD8IgG-IL12 treatment resulted in better anti-tumor efficacy than vehicle.

CD8IgG-IL12 (1×HC) was also tested in vivo for anti-cancer efficacy in syngeneic mouse models of colon cancer (“MC38” and “CT26.WT”), melanoma (“B16F10”), lung cancer (“LL/2”), prostate cancer (“RM1”), and hepatoma (“Hepa1-6”). Briefly, 6- to 8-week old female C57BL/6 mice were inoculated subcutaneously with about 2.0×105 to 5.0×10⁶ B16F10 tumor cells, MC38 tumor cells, or Hepa1-6 tumor cells in the right flank. In the lung cancer model, 6- to 8-week old female C57BL/6 mice were inoculated subcutaneously with about 3×10⁵ LL/2 cells in the right flank. In the prostate cancer model, 7- to 9-week old male C57BL/6 mice were inoculated subcutaneously with about 1×10⁶ RM1 cells in the right flank. In the CT26 colon cancer model, 6- to 8-week old female BALB/c mice were inoculated subcutaneously with about 0.5-1.0×10⁶ CT26 tumor cells in the right flank. When the mean tumor volume of the cohort reached about 80-100 mm³ (in the lung cancer model), about 90-100 mm³ (in the prostate cancer model), or about 100-120 mm³ (in the B16F10, MC38, Hepa1-6, and CT26 models), the mice were dosed with either 34 μg of the fusion construct subcutaneously or a PBS vehicle control intraperitoneally. Subsequent doses were injected 7 and 14 days after the initial dose. Tumor measurements and body weights were measured twice per week until endpoint.

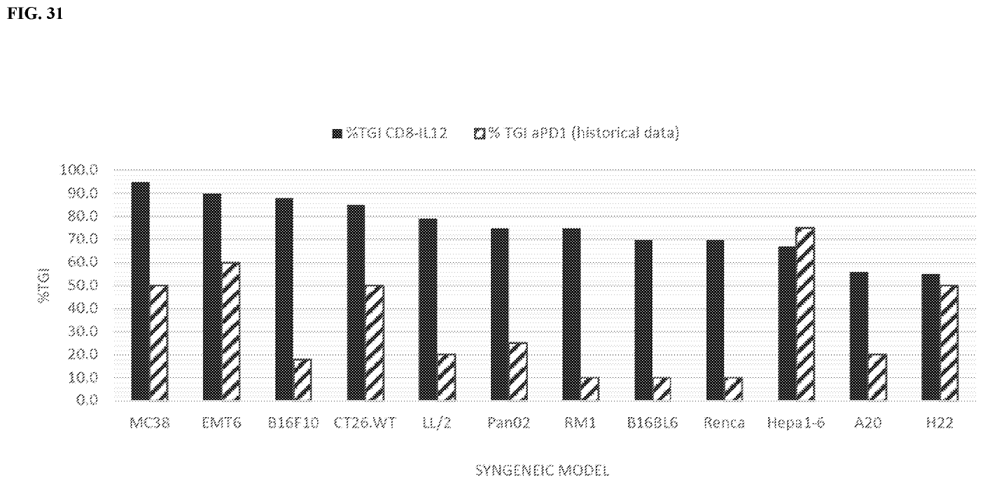

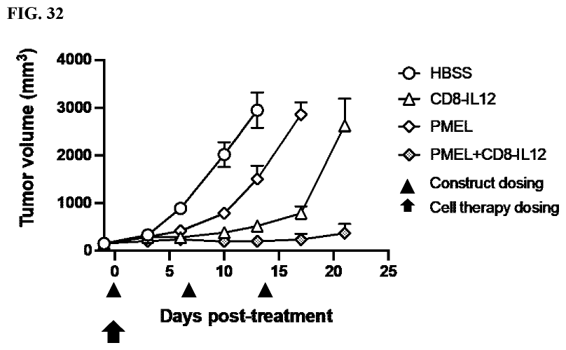

Mean tumor growth inhibition (% TGI), calculated as 100% x ((mean tumor volume of the control group)−(mean tumor volume of the treatment group))/(mean tumor volume of the control group)), by CD8IgG-IL12 (1×HC) in the eleven syngeneic tumors was compared with historical data testing the efficacy of a murine anti-PD-1 antibody (Crown Bioscience). Mean % TGI by CD8IgG-IL12 (1×HC) in the syngeneic Pan02 tumor model was also calculated using the data summarized in FIG. 29 and compared to historical data using murine anti-PD-1 antibody (Crown Bioscience). As shown in FIG. 31, treatment with CD8IgG-IL12 (1×HC) resulted in about 55%-95% tumor growth inhibition in these tumor models, which was significantly higher than treatment with the anti-PD-1 antibody in the majority of tumor models.

Combination with Adoptive Cell Therapy

CD8IgG-IL12 (1×HC) was tested for its ability to inhibit tumor growth in combination with an adoptive cell therapy. Briefly, 8-week-old female C57BL/6 mice were inoculated subcutaneously with 1×10⁶ B16F10 cells in the right lower flank. When mean tumor volume of the cohort reached 100-150 mm³, mice were randomized according to their tumor volumes. Three weekly 34 pg doses of mCD8IgG-IL12 (1×HC) were administered subcutaneously. Separately or in combination, a dose of 2.5×10⁶ PMEL-antigen-specific T cells was also administered intravenously with the first dose of the mCD8IgG-IL12 (1×HC). Another group of mice received HBSS as a vehicle control. Tumor measurements were recorded twice weekly by caliper until endpoint.

As shown in FIG. 32, the combination of the fusion protein and cell therapy showed enhanced anti-tumor efficacy as compared to the individual monotherapies. Remarkably, either monotherapy only delayed tumor progression, whereas tumor volume remained low throughout the experiment in the mice that received the combination therapy.

Combination with Immune Checkpoint Blockade

CD8IgG-IL12 (1×HC) was tested for its ability to inhibit tumor growth in combination with an immune checkpoint inhibitor. Briefly, 8- to 10-week-old C57BL6/J mice were inoculated subcutaneously with 1×10⁶ MC38 tumor cells. When mean tumor volume reached about 150 mm³, the mice were intravenously dosed with 0.5 mg/kg CD8IgG-IL12 (1×HC). Subsequent doses of CD8IgG-IL12 (1×HC) were administered at 7, 14, and 21 days after the initial dose. Separately or in combination, a 10 mg/kg dose of anti-PD-L1 antibody (clone 10F.9G2) was also administered intraperitoneally to mice twice per week for 4 weeks. Another group of mice received HBSS as a vehicle control. Tumor volume and body weight were measured twice per week until endpoint.

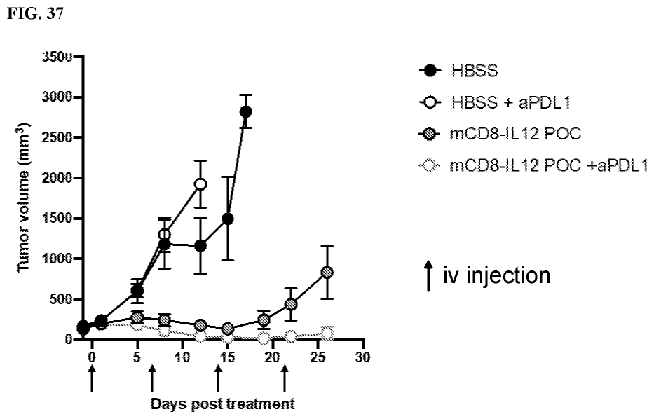

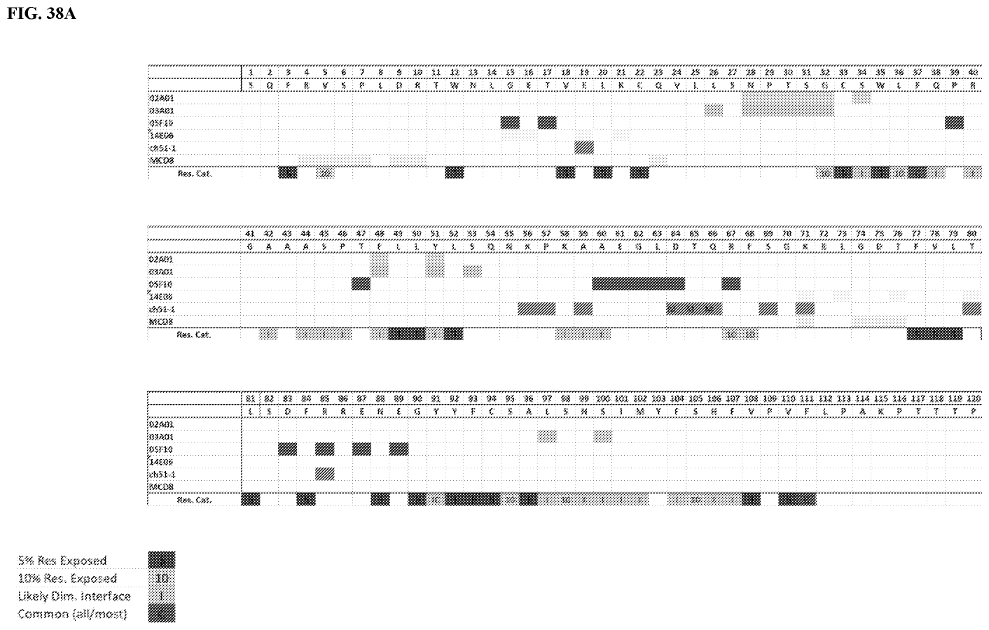

As shown in FIG. 37, the combination of the fusion protein and the anti-PD-L1 antibody showed enhanced anti-tumor efficacy as compared to the individual monotherapies. Notably, the anti-PD-L1 antibody monotherapy had no significant effect on tumor growth and the CD8IgG-IL12 (1×HC) monotherapy slowed tumor growth inhibition, whereas tumor volume remained low throughout the experiment in the mice that received the combination therapy.

### Example 4. Anti-Human CD8/Human IL-12 Fusion Proteins

### A. In Vitro Assessment of Anti-Human-CD8:Human IL-12 Fusion Proteins

Eight fusion proteins were generated that each comprised (1) a silent anti-hCD8 IgG antibody (either 02A01 or 03A01) or an antigen-binding fragment thereof, and 2) a human IL-12p70 protein. Four constructs were derived from 02A01 and four constructs were derived from 03A01. The constructs were built in accordance with one of the following four scaffold formats described herein: “Fab-immunomodulator (LC)”, “IgG-immunomodulator (LC)”, “IgG-immunomodulator (HC)”, and “Fab-Fc-immunomodulator (LC)” (see FIGS. 1A-1D). These fusion proteins are referred to herein as 02A01/03A01-Fab-IL12 (LC), 02A01/03A01-IgG-IL12 (LC), 02A01/03A01-IgG-IL12 (HC), and 02A01/03A01-Fab-Fc-IL12 (LC), respectively. The fusion proteins comprising an Fc region (all except 02A01-Fab-IL12 (LC) and 03A01-Fab-IL12 (LC)) utilized a human IgG1 Fc, and further comprised the substitutions L234A, L235A, P329G, and N297A in the IgG1 Fc domain that reduce ADCC effector function. The 02A01-Fab-Fc-IL12 (LC) and 02A01-Fab-Fc-IL12 (LC) constructs further comprised the substitution T366W in the Fc polypeptide chain linked to IL-12 and the substitutions T366S, L368A, and Y407V in the other Fc polypeptide chain, which promote heterodimerization. These eight fusion proteins were tested for their ability to target IL12 to T cells and for their ability to silently bind hCD8.

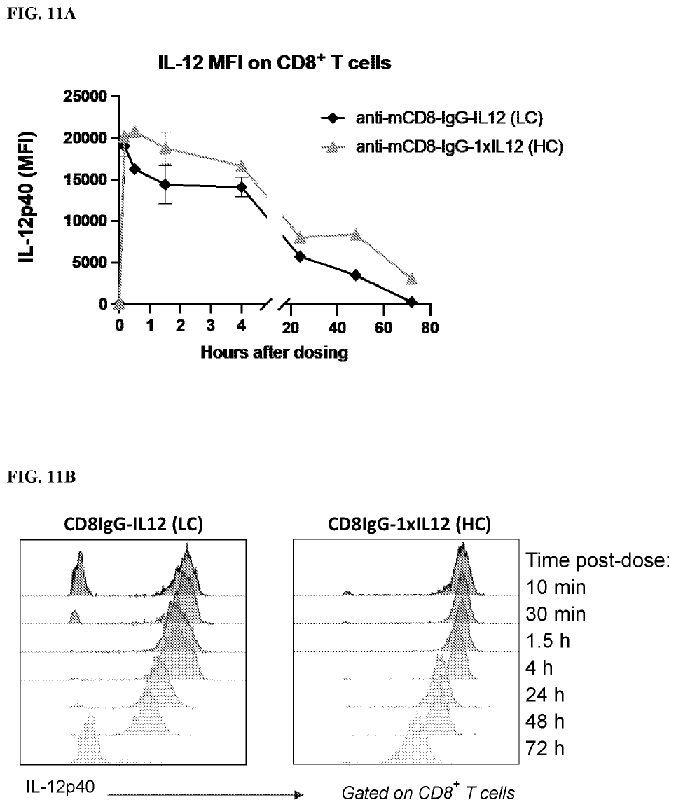

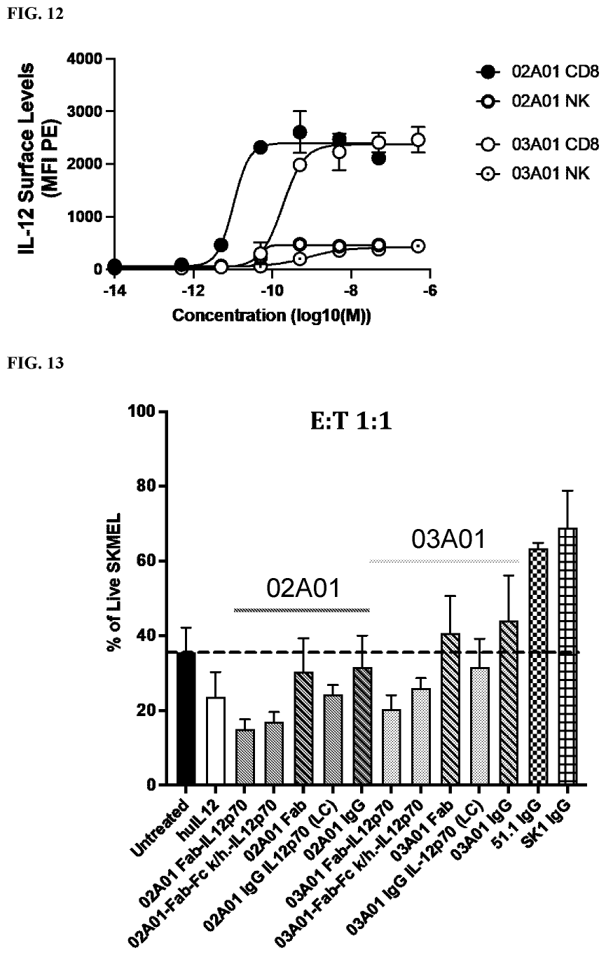

Fusion proteins 02A01-IgG-IL12 (LC) and 03A01-IgG-IL12 (LC) were evaluated for their ability to specifically target IL12 to CD8⁺ T cells. Briefly, 5×10⁴ human PBMCs were plated on 96-well plates in complete RPMI 1640 medium and incubated with either 02A01-IgG-IL12 (LC) and 03A01-IgG-IL12 (LC) at one of a series of concentrations between 500 nM and 0.5 pM. The cells were incubated with media lacking a fusion protein as a negative control. Following 1 hour of incubation with the IL12 fusion constructs, the cells were washed once with complete RPMI medium and prepared for flow cytometry analysis. Cells were washed once with phosphate-buffered saline (PBS) containing 1% bovine serum albumin (PBS/BSA) and then stained with a 1:100 dilution of PE-conjugated anti-IL12 antibody (Clone No. C11.5; BioLegend Cat. No. 501807) in PBS/BSA. Following a 20 minute incubation at room temperature, the cells were washed once with PBS/BSA and then resuspended in PBS/BSA for analysis on a FACSCelesta using Diva software (BD Biosciences). Binding activity was quantified and represented as the mean fluorescent intensity values of IL-12 on either CD8⁺ T cells or NK cells. As shown in FIG. 12, both 02A01-IgG-IL12 (LC) and 03A01-IgG-IL12 (LC) were able to selectively bind CD8⁺ T cells over NK cells at a range of concentrations. At a saturating dose, there was a 4- to 5-fold more IL-12 bound to CD8⁺ T cells than was bound to NK cells. The 02A01 construct had a higher binding affinity for CD8⁺ T cells and NK cells than 03A01.

Six of the eight aforementioned anti-hCD8/hIL12 fusion proteins were also evaluated for their ability to silently bind CD8⁺ T cells without abrogating T cell-mediated killing of a melanoma tumor cell line, SK-MEL-5. Briefly, SK-MEL-5 tumor cells were plated at a density of 5×10³ cells per well on Day −1. On Day 0, the tumor cell culture supernatant was aspirated and the tumor cells were combined with 1×10⁴ MART-1 (melanoma antigen recognized by T cells 1)-specific T cells, supplemented with a saturating concentration of one of the fusion proteins to be tested, or supplemented with a control protein (human IL-12 alone, an 02A01 or 03A01 Fab lacking IL-12, an 02A01 or 03A01 IgG lacking IL-12, or commercial IgGs SK1 or 51.1). Cultures lacking supplementation with any added protein were used to establish a baseline of T cell cytotoxicity. On Day 1, the T cells and supernatant were removed from the cultures, the cultures were washed with PBS, and tumor cell viability was quantified with Cell Titer Glo. The results were quantified as the number of viable tumor cells in the well relative to a no-T-cell control. As shown in FIG. 13, treatment with 02A01-IgG-IL12 (LC) or 03A01-IgG-IL12 (LC) resulted in enhanced cytotoxicity relative to untreated T cells, and resulted in comparable cytotoxicity to treatment with T cells supplemented with recombinant human IL12 alone. As anticipated, treatment with silent-CD8-binding antibodies and fragments thereof did not abrogate T cell-mediated toxicity (02A01 IgG, 03A01 IgG, 02A01 Fab, or 03A01 Fab; all lacking an IL-12 protein). By contrast, treatment with commercial antibodies SK1 or 51.1 (known non-silent-binding anti-CD8 antibodies) inhibited T-cell mediated cytotoxicity, and resulted in an approximately 2-fold increase in tumor cell survival relative to a control culture utilizing untreated T cells alone.

All eight anti-hCD8/IL12 fusion proteins were tested for their ability to selectively induce pSTAT4 signaling in CD8⁺ T cells. Human PBMCs (StemCell Technologies) were pre-activated by treatment with anti-human CD3/CD28 Dynabeads (Thermo Fisher Scientific) and recombinant hIL-2 (10 ng/mL) for 3 days at 37° C. 2×10⁵ pre-activated PBMCs were incubated with various concentrations of either recombinant hIL-12 or an 02A01-derived or 03A01-derived IL12 fusion protein for 20 minutes at 37° C. The cells were washed twice and stained with antibodies against surface markers for 20 minutes at room temperature. The cells were then fixed with Cytofix buffer (BD), permeabilized with Perm Buffer III (BD), stained with anti-phospho-STAT4 antibody, and analyzed via flow cytometry. Results were reported as MFI values of phospho-STAT4 on CD4⁺ T cells, CD8⁺ T cells, and NK cells, and EC₅₀ (nM) values for all tested constructs are reported in Table 15.

TABLE 15
EC₅₀ values for stimulation of pSTAT4 signaling in
human PBMCs by rhIL12 and by hIL12 fusion proteins

| | | | | CD8⁺ |
|---|---|---|---|---|
| | CD8⁺ | CD4⁺ | NK | T/NK |
| Construct | T Cells | T Cells | Cells | Ratio |
| rhIL 12 | 0.0196 | 0.00874 | 0.0271 | 1.38 |
| 02A01-IgG-IL12 (LC) | 0.00272 | 0.0229 | 0.0173 | 6.36 |
| 03A01-IgG-IL12 (LC) | 0.0147 | 0.0214 | 0.0306 | 2.08 |
| 02A01-IgG-IL12 (HC) | 0.00303 | 0.0227 | 0.0156 | 5.15 |
| 03A01-IgG-IL12 (HC) | 0.0157 | 0.0256 | 0.0300 | 1.91 |
| 02A01-Fab-IL12 (LC) | 0.00174 | 0.0203 | 0.0127 | 7.30 |
| 03A01-Fab-IL12 (LC) | 0.0111 | 0.0241 | 0.0336 | 3.03 |
| 02A01-Fab-Fc-IL12 (LC) | 0.00211 | 0.0290 | 0.0196 | 9.29 |
| 03A01-Fab-Fc-IL12 (LC) | 0.0134 | 0.0306 | 0.0477 | 3.56 |

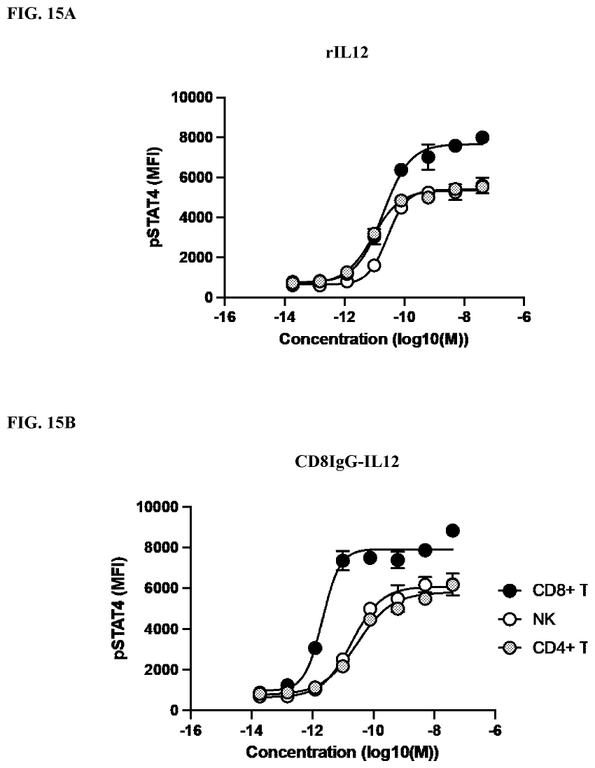

As shown in Table 15, CD4⁺ T cells, CD8⁺ T cells, and NK cells showed similar EC₅₀ values when stimulated with rhIL12. By contrast, incubation with any tested format of 02A01-conjugated IL12 (and 03A01-conjugated IL-12 to a lesser extent) resulted in pSTAT4 upregulation more selectively in CD8⁺ T cells. The complete results for recombinant hIL12 and 02A01-IgG-IL12 are illustrated in FIG. 15A and FIG. 15B, respectively.

The aforementioned 02A01-derived anti-hCD8/hIL12 fusion proteins were also evaluated for their ability to stimulate dose-dependent IL12-stimulated STAT4 signaling in CD4+ T cells, CD8⁺ T cells, and NK cells from resting or activated cynomolgus monkey PBMCs. Cynomolgus monkey PBMCs were obtained from iQ Biosciences and, for the appropriate treatments, PBMC pre-activation was performed with 5 μg/mL plate-bound anti-CD3 (clone SP34-2) for 3 days at 37° C. 2×10⁵ resting or pre-activated PBMCs were incubated with various concentrations of either recombinant hIL-12 or an 02A01-derived IL12 fusion protein for 20 minutes at 37° C. Cells were then washed twice and stained with antibodies against surface markers for 20 minutes at room temperature. Cells were then fixed with Cytofix buffer (BD), permeabilized with Perm Buffer III (BD), stained with anti-phospho-STAT4 antibody, and analyzed via flow cytometry. Results were reported as MFI values of phospho-STAT4 on CD4⁺ T cells, CD8⁺ T cells, and NK cells.

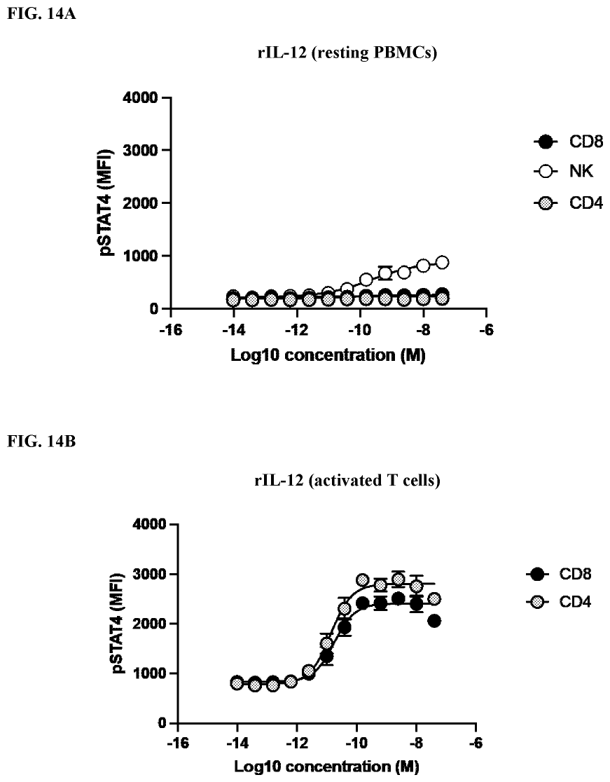

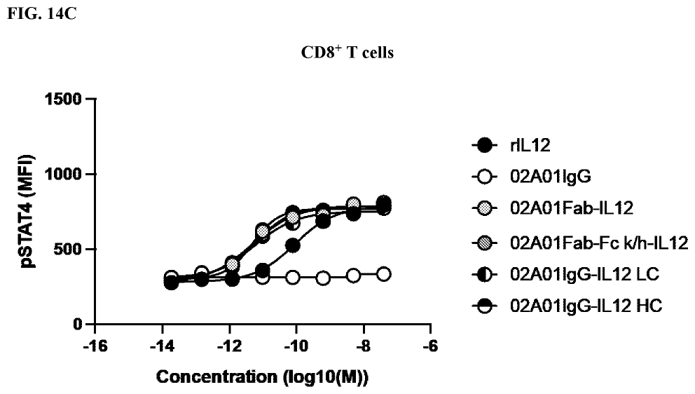

As shown in FIG. 14A, in resting PBMCs, only NK cells showed dose-dependent pSTAT4 upregulation in response to recombinant hIL12 treatment, whereas CD4⁺ and CD8⁺ T cells did not. After TCR activation by a plate-bound anti-CD3 antibody, CD4⁺ and CD8⁺ T cells showed dose-dependent upregulation pSTAT4 in response to recombinant hIL-12 (rhIL12) treatment (FIG. 14B). Treatment of pre-activated cells with any of the four tested formats of 02A01-fused IL12 was able to trigger dose-dependent pSTAT4 upregulation in CD8⁺ T cells, and all four tested formats of 02A01-conjugated IL12 showed higher affinity to CD8⁺ T cells than rhIL12 (FIG. 14C). The upregulation of pSTAT4 was a specific response to IL12 stimulation, as 02A01 IgG alone did not trigger this response (FIG. 14C). B. In vitro assessment of humanized anti-human-CD8:human IL-12 fusion protein constructs

Two fusion protein constructs were generated that each comprised (1) a humanized anti-human-CD8 IgG1 antibody comprising humanized 02A01 VH6 and humanized 02A01 VK3 (see Table 1), and 2) a human IL-12p70 protein. The constructs were built in accordance with one of the two following scaffold formats described herein: “IgG-immunomodulator (LC)” and “IgG-immunomodulator (1×HC) (see FIG. 1C and FIG. 1E). These fusion proteins are referred to herein as “H6K3-IgG-IL12 (LC)” and “H6K3-IgG-IL12 (1×HC)”, respectively. Both fusion protein constructs utilized a human IgG1 Fe, and comprised the substitutions L234A, L235A, P329G, and N297A in the IgG1 Fc domain that reduce ADCC effector function. The H6K3-IgG-IL12 (1×HC) construct further comprised the substitution T366W in the Fc polypeptide chain linked to IL-12 and the substitutions T366S, L368A, and Y407V in the other Fc polypeptide chain, which promote heterodimerization. The two fusion protein constructs were tested for their ability to selectively bind human and cynomolgus monkey CD8-expressing cells, their ability to induce IL-12-dependent signaling, and their ability to stimulate T cell anti-tumor cytotoxicity.

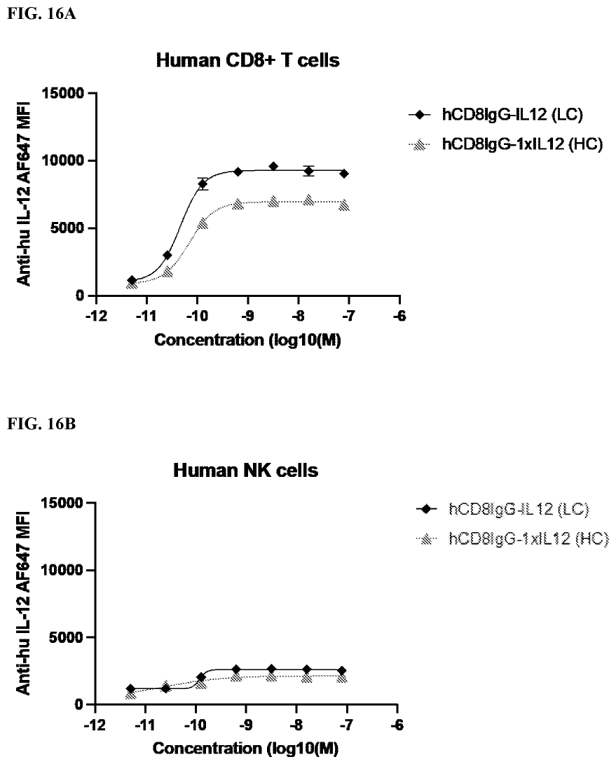

Fusion proteins H6K3-IgG-IL12 (LC) and H6K3-IgG-IL12 (1×HC) were evaluated for their ability to selectively bind human CD8+ T cells in PBMCs. Briefly, 5×10⁴ human PBMCs (StemCell Technologies) were incubated with 7 serial 5-fold dilutions of one of the two aforementioned H6K3-IgG-IL12 constructs for 30 minutes at 37° C. Cells were washed twice with MACS buffer and stained for 20 minutes at 4° C. with fluorophore-conjugated anti-CD3, anti-CD4, anti-CD8, anti-CD56, and anti-IL12 antibodies, and viability dye. Cells were analyzed on a FACSCelesta using Diva software (BD Biosciences). Binding activity was quantified and represented as the mean fluorescent intensity values of IL-12 on either CD8+ T cells or NK Cells. As shown in FIGS. 16A-16B, both H6K3-IgG-IL12 (LC) and H6K3-IgG-IL12 (1×HC) were able to selectively bind human CD8+ T cells over NK cells at a range of concentrations.

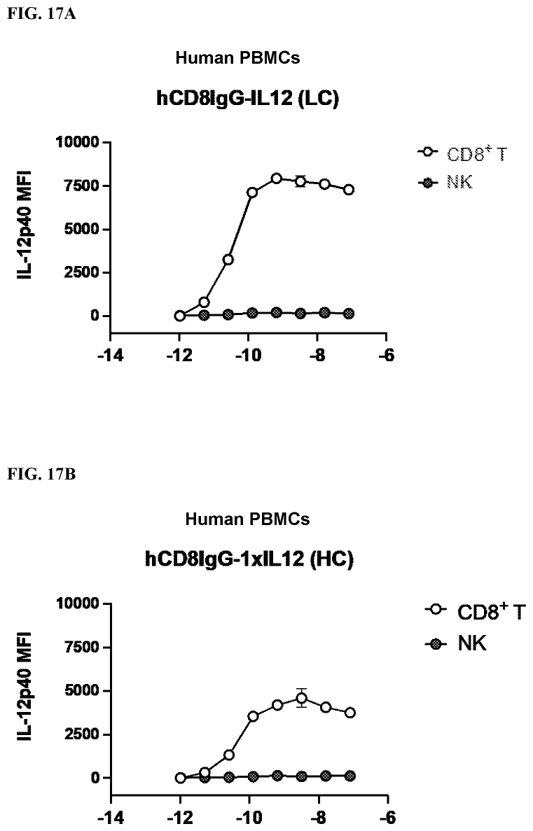

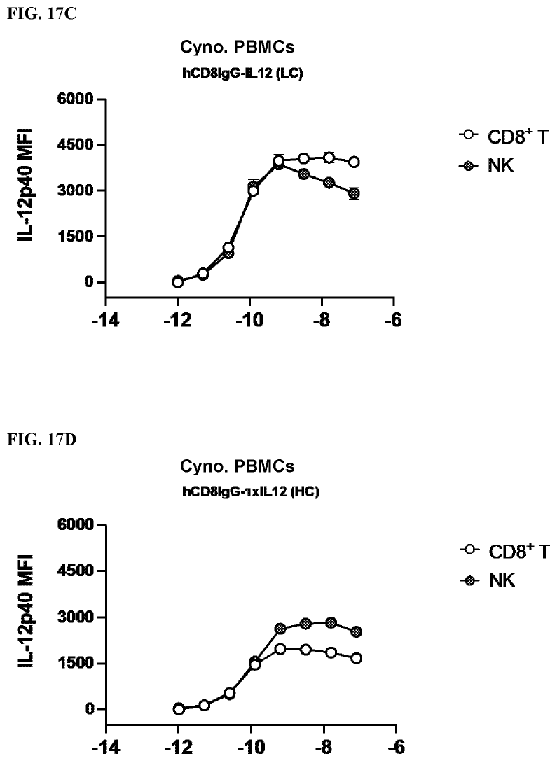

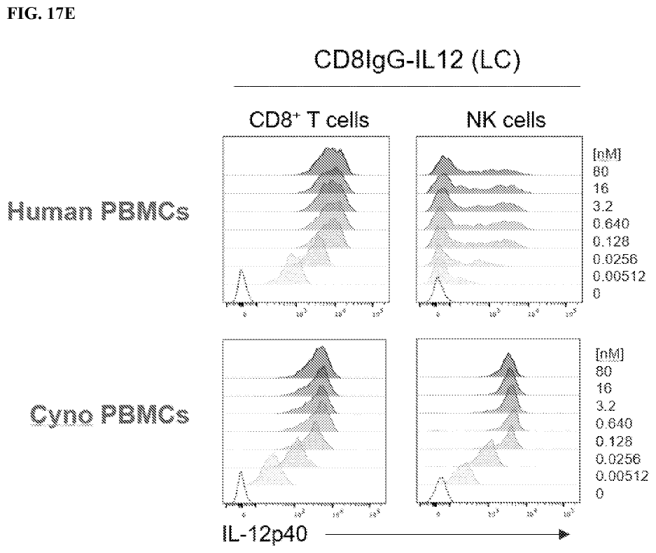

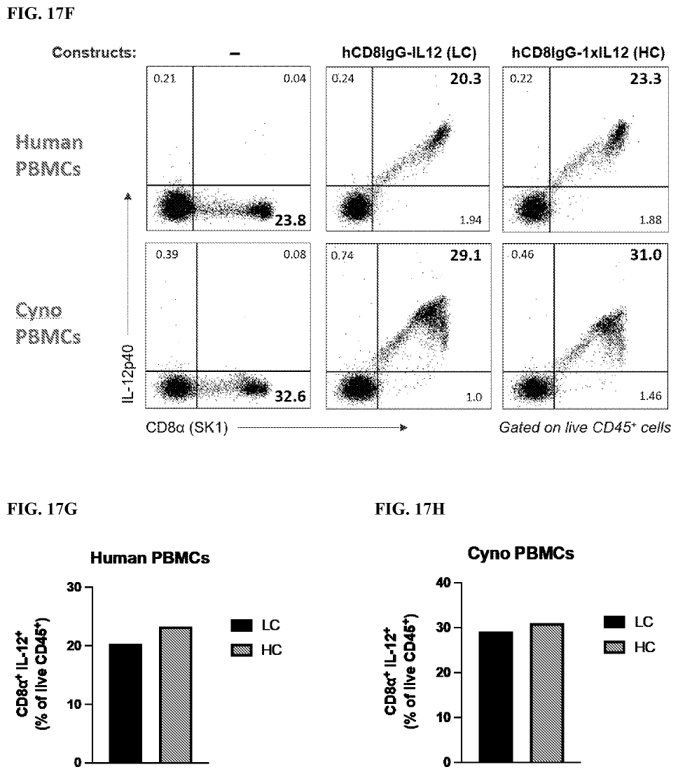

Fusion proteins H6K3-IgG-IL12 (LC) and H6K3-IgG-IL12 (1×HC) were also evaluated for their ability to bind CD8-expressing cells in human and cynomolgus monkey PBMCs. Briefly, 5×10⁴ human PBMCs (StemCell Technologies) or cynomolgus monkey PBMCs (iQ Biosciences) were incubated with 7 serial 5-fold dilutions of one of the two aforementioned H6K3-IgG-IL12 constructs for 30 minutes at 37° C. Cells were washed twice with MACS buffer and stained with viability dye and fluorophore-conjugated antibodies against IL-12 and cell-surface markers for 20 minutes at 4° C. Cells were analyzed on a FACSCelesta using Diva software (BD Biosciences). Binding activity for all treatments was quantified and represented as the mean fluorescence intensity values of IL-12 on human CD8+ T cells and NK cells (FIGS. 17A-17B) and on cynomolgus monkey CD8+ T cells and NK cells (FIGS. 17C-17D). For the H6K3-IgG-IL12 (LC) treatments, results are also reported as the frequency of IL-12+CD8+ T cells and NK cells (FIG. 17E). As shown in FIGS. 17A-17D, both H6K3-IgG-IL12 (LC) and H6K3-IgG-IL12 (1×HC) were able to selectively bind human CD8+ T cells over NK cells at a range of concentrations. Only a small proportion of human NK cells express CD8, and therefore the H6K3-IgG-IL12 (LC) construct only bound a small proportion of human NK cells (FIG. 17E upper left and upper right). By contrast, almost all cynomolgus monkey NK cells express CD8. As a result, both constructs bound cynomolgus monkey CD8+ T cells and NK cells (FIG. 17C-17D; FIG. 17E lower left and lower right). Where the cells were incubated with 0.128 nM of the IL-12 fusion constructs, the number of live CD45+ cells staining as CD8⁺ and IL-12⁺ were assessed by flow cytometry. As shown by the dot plots in FIG. 17F, both IL-12 fusion constructs specifically bound CD8-expressing cells in both human and cynomolgus monkey PBMCs. As quantified by the bar graphs in FIGS. 17G and 17H, the frequency of CD8⁺ IL-12⁺ co-stained cells was comparable between H6K3-IgG-IL12 (LC) and H6K3-IgG-IL12 (1×HC), indicating that the two constructs exhibited similar targeting and loading profiles.

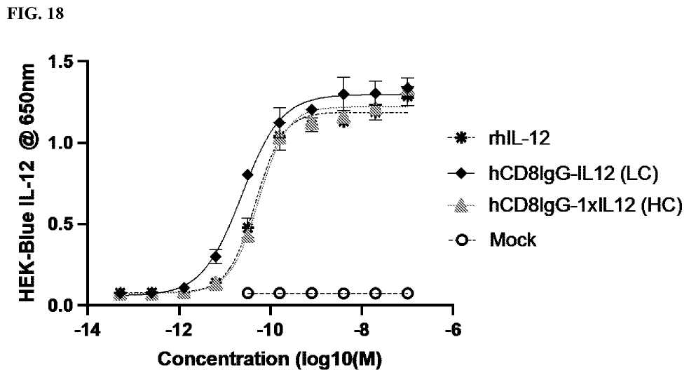

Fusion proteins H6K3-IgG-IL12 (LC) and H6K3-IgG-IL12 (1×HC) were evaluated for their ability to stimulate IL-12-mediated signaling in HEK-Blue™ IL-12 reporter cells (Invivogen). HEK-Blue™ IL-12 cells are HEK 293 embryonic kidney cells which comprise the IL-12 signaling pathway, as well as a pSTAT4-inducible SEAP genetic reporter construct which is activated in the presence of extracellular IL-12. The HEK-Blue™ IL-12 reported cells do not express CD8. Briefly, 5×10⁴ HEK-Blue™ IL-12 cells per well were plated in a 96-well plate, and cells were incubated overnight at 4° C. with 10 serial 5-fold dilutions of H6K3-IgG-IL12 (LC), H6K3-IgG-IL12 (1×HC), or with recombinant human single-chain IL12 (“rhIL-12”) as a control. The assay was continued on the following day according to the manufacturer's instructions. Briefly, 180 μL of QUANTI-Blue™ solution (Invivogen) was mixed with 20 μL of supernatant from a treatment well and incubated at 37° C. for 30 minutes. 180 μL QUANTI-Blue™ was mixed with 20 μL of culture medium as a control (“mock”). Samples were analyzed using a spectrophotometer at 650 nm. As shown in FIG. 18, H6K3-IgG-IL12 (1×HC) was able to induce IL-12 signaling in the reporter cells with similar potency as rhIL-12. The H6K3-IgG-IL12 (LC) construct was a more potent signaling inducer in the reporter cells because it contains two IL-12 moieties per construct. These results indicate that fusion with the CD8-binding site in these constructs do not interfere with the activity of IL-12.

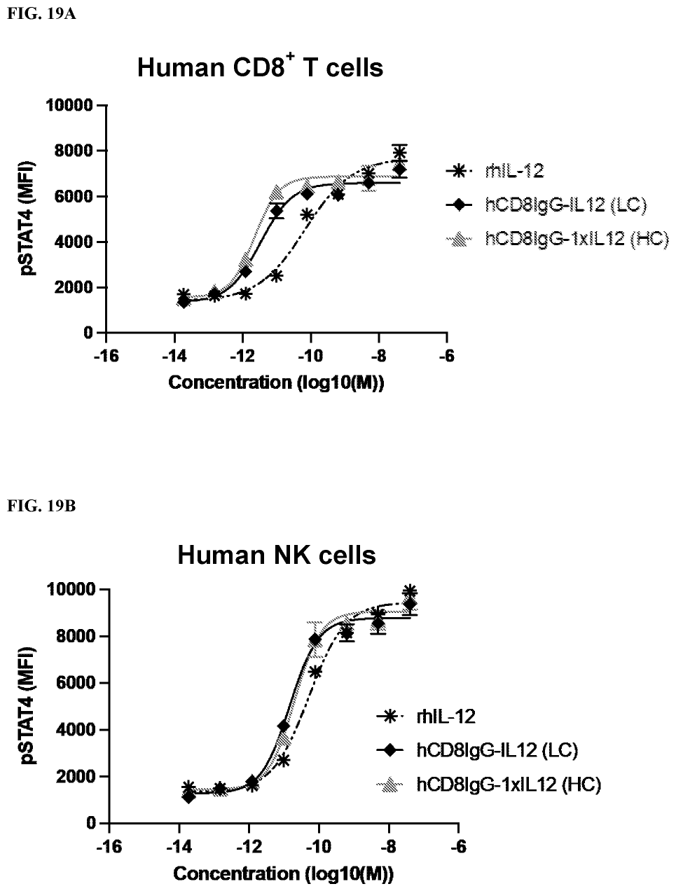

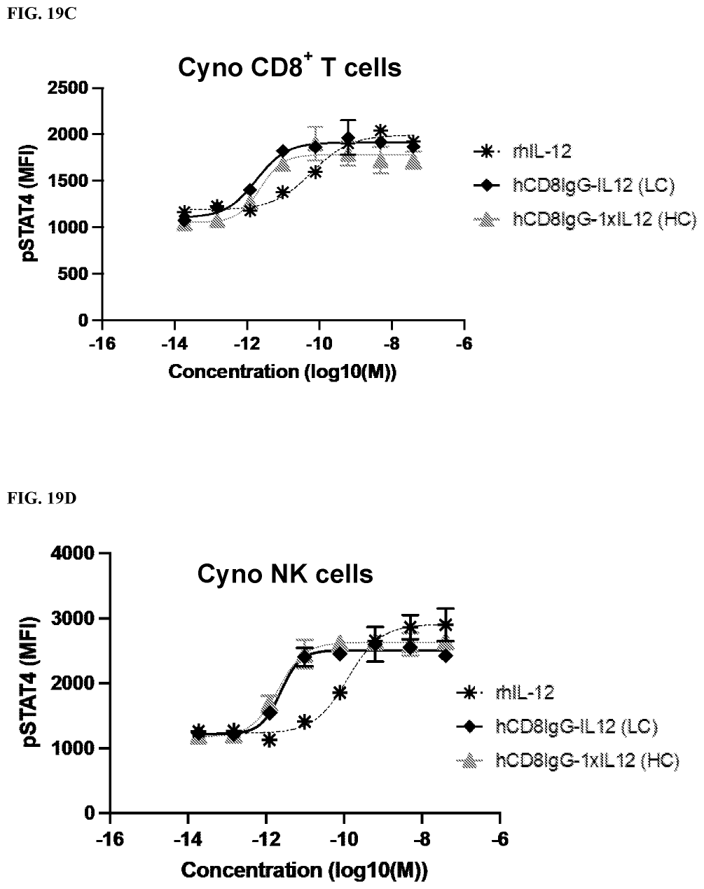

Fusion protein constructs H6K3-IgG-IL12 (LC) and H6K3-IgG-IL12 (1×HC) were also evaluated for their ability to induce dose-dependent IL-12-stimulated STAT4 signaling in human or cynomolgus monkey CD8+ T cells and NK cells. Human PBMCs (StemCell Technologies) were activated by treatment with anti-human-CD3/CD28 dynabeads (Thermo Fischer Scientific) and recombinant IL-2 (rIL-2; 10 ng/mL) for 3 days according to manufacturer instructions. Separately, cynomolgus monkey PBMCs (iQ Biosciences) were activated by incubation with 5 μg/mL of plate-bound anti-human-CD3 (BD Biosciences) and rIL-2 in a flat-bottom 96-well plate (2×10⁵ cells/well) for 3 days. After 3 days, 2×10⁵ activated human or cynomolgus monkey PBMCs were incubated with 7 serial 8-fold dilutions of H6K3-IgG-IL12 (LC), H6K3-IgG-IL12 (1×HC), or nontargeted rhIL-12 for 20 minutes at 37° C. The cells were washed twice and stained with antibodies against surface markers for 20 minutes at room temperature. The cells were then fixed with Cytofix buffer (BD), permeabilized with Perm Buffer III (BD), stained with anti-phospho-STAT4 antibody, and analyzed via flow cytometry. Results were reported as MFI values of phospho-STAT4 on CD8+ T cells, CD4+ T Cells, and NK cells. EC₅₀ values for all tested constructs are listed in Table 16 for human PBMCs and in Table 17 for cynomolgus monkey PBMCs. The complete results for human CD8+ T cells and NK cells are shown in FIG. 19A-19B, and the complete results for cynomolgus monkey CD8+ T cells and NK cells are shown in FIG. 19C-19D.

TABLE 16
EC₅₀ values (pM) for stimulation of pSTAT4 signaling in human
PBMCs by rhIL12 and by humanized anti-CD8-hIL12 fusion proteins

| | Human | Human | Human | CD8⁺ |
|---|---|---|---|---|
| | CD8⁺ | CD4⁺ | NK | T/NK |
| Construct | T Cells | T Cells | Cells | Ratio |
| rhIL12 | 67.6 | 39.8 | 51.2 | 0.8 |
| hCD8IgG-IL12 (LC) | 3.3 | 54.3 | 14.7 | 4.5 |
| hCD8IgG-IL12 (1 × HC) | 2.3 | 140 | 20.2 | 8.8 |

TABLE 17
EC₅₀ values (pM) for stimulation of pSTAT4 signaling
in cynomolgus monkey PBMCs by rhIL12 and by
humanized anti-CD8-hIL12 fusion proteins

| | Cyno. | Cyno. | Cyno. | CD8⁺ |
|---|---|---|---|---|
| | CD8⁺ | CD4⁺ | NK | T/NK |
| Construct | T Cells | T Cells | Cells | Ratio |
| rhIL12 | 61.7 | 58.5 | 12.3 | 0.2 |
| hCD8IgG-IL12 (LC) | 1.8 | 14.1 | 2.4 | 1.3 |
| hCD8IgG-IL12 (1 × HC) | 2.3 | 14.7 | 2.0 | 0.9 |

As shown in Table 16, human CD8⁺ T cells and NK cells showed similar EC₅₀ values when stimulated with rhIL12. By contrast, incubation with H6K3-IgG-IL12 (LC) or H6K3-IgG-IL12 (1×HC) resulted in pSTAT4 upregulation more selectively in human CD8⁺ T cells. Cynomolgus monkey CD8+ T cells and NK cells express CD8 at similar levels, and so incubation of cynomolgus monkey PBMCs with one of the anti-hCD8-IL12 fusion constructs did not selectively activate CD8+ T cells over NK cells (Table 17).

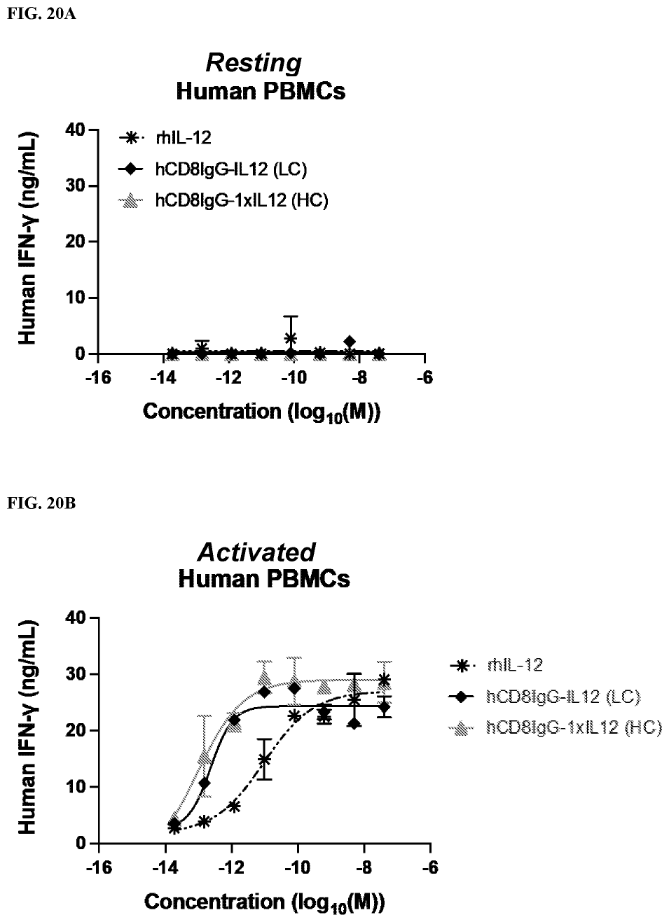

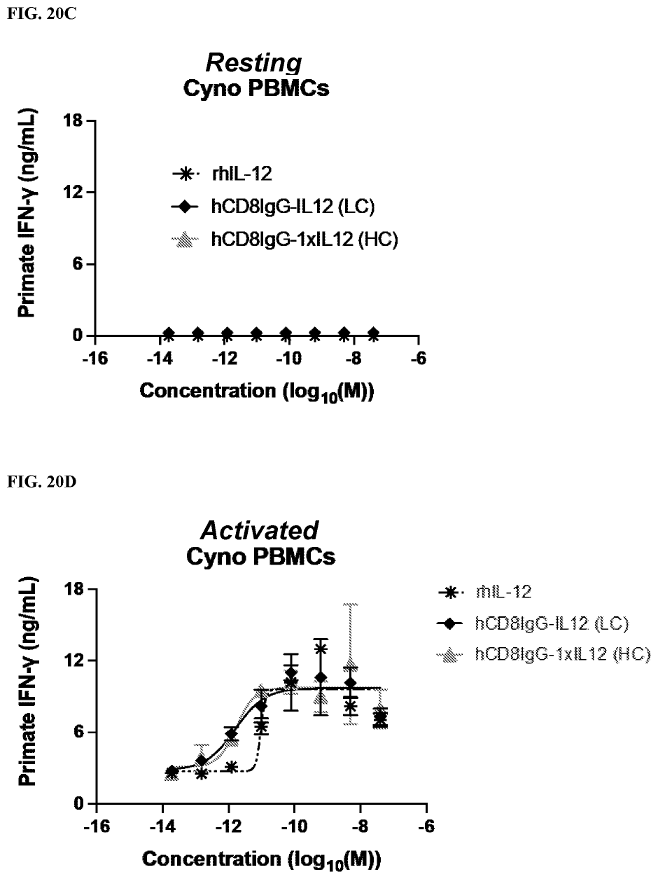

Fusion protein constructs H6K3-IgG-IL12 (LC) and H6K3-IgG-IL12 (1×HC) were also evaluated for their ability to induce dose-dependent IL-12-stimulated IFN-7 release from human and cynomolgus monkey PBMCs in the absence or presence of TCR stimulation. 2×10⁵ human PBMCs (StemCell Technologies) or cynomolgus monkey PBMCs (iQ Biosciences) were incubated with 7 serial 8-fold dilutions of H6K3-IgG-IL12 (LC), H6K3-IgG-IL12 (1×HC), or nontargeted rhIL-12 for 10 minutes at 37° C. In the “rested” groups, PBMCs were resuspended in 200 μL media and incubated for 24 hours at 37° C. In the “activated” groups, PBMCs were resuspended in 200 μL media containing 2 ng/mL phorbol myristate acetate (PMA) and 200 ng/mL phytohemagglutinin-L (PHA-L) for 24 hours at 37° C. The next day, culture supernatants were harvested and the amount of IFN-γ was measured using either human IFN-7 Quantikine ELISA (R&D Systems) or primate IFN-γ DuoSet ELISA (R&D Systems). The complete results for resting and activated human PBMCs are shown in FIG. 20A and FIG. 20B, respectively, and the complete results for resting and activated cynomolgus monkey PBMCs are shown in FIG. 20C and FIG. 20D, respectively. EC₅₀ values for the tested constructs for activated human and cynomolgus monkey PBMCs are reported in Table 18.

TABLE 18
EC₅₀ values (pM) for stimulation of IFN-γ release
in TCR-stimulated human and cynomolgus monkey PBMCs
by rhIL12 and by humanized anti-CD8-hIL12 fusion proteins

| | Activated | Activated |
|---|---|---|
| Construct | Human PBMCs | Cyno. PBMCs |
| rhIL12 | 9.59 | 9.47 |
| H6K3-IgG-IL12 (LC) | 0.23 | 1.60 |
| H6K3-IgG-IL12 (1 × HC) | 0.09 | 1.71 |

As shown in FIG. 20A and FIG. 20C, no IL-12-dependent IFN-γ release was detected in the human or cynomolgus monkey PBMC cultures in the absence of TCR stimulation. In the activated PBMC cultures, an IL-12-dose-dependent increase in IFNγ release was observed from both human PBMCs and cynomolgus monkey PBMCs (FIG. 20B and FIG. 20D). As shown in Table 18, both H6K3-IgG-IL12 (LC) and H6K3-IgG-IL12 (1×HC) were more potent inducers of IFN-γ release than nontargeted rhIL-12.

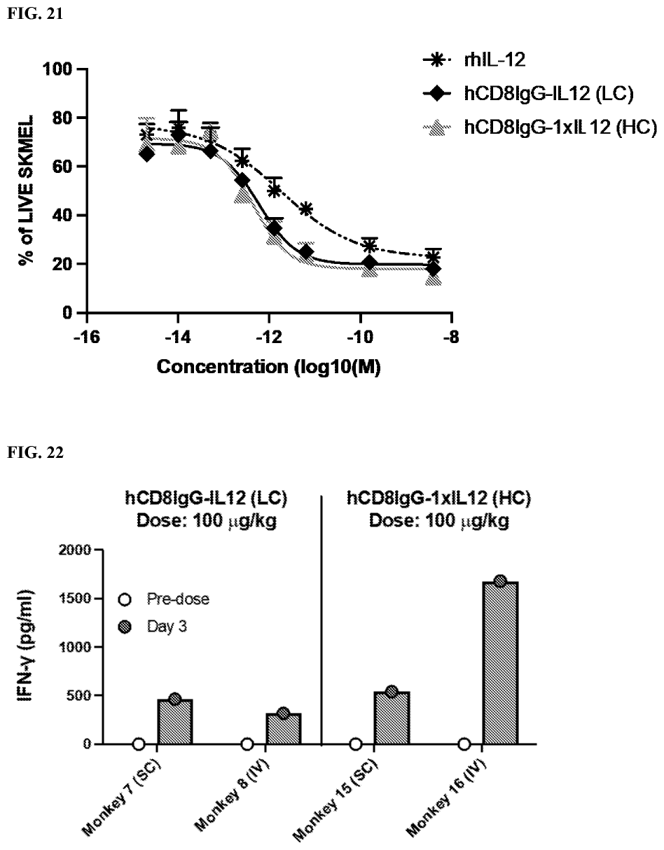

Fusion protein constructs H6K3-IgG-IL12 (LC) and H6K3-IgG-IL12 (1×HC) were also evaluated for their ability to increase CD8+ T cell-mediated killing of a melanoma tumor cell line. Briefly, SK-MEL-5 tumor cells were plated at a density of 5×10³ cells per well on Day −1. On Day 0, 5×10³ MART-1 (melanoma antigen recognized by T cells 1)-specific T cells were incubated with varying concentrations of H6K3-IgG-IL12 (LC), H6K3-IgG-IL12 (1×HC), or rhIL-12 for 15 minutes at 37° C. T cells were then washed twice with media. Tumor cell culture supernatant was aspirated and the SK-MEL-5 tumor cells were combined with MART-1-specific T cells at a 1:1 ratio of tumor cells to T cells. Tumor cells supplemented with untreated T cells were used to establish a baseline of T cell cytotoxicity. On Day 2, the T cells and supernatant were removed from the cultures, the wells were washed with PBS, and tumor cell viability was quantified with Cell Titer Glo. The results were quantified as the number of viable tumor cells in the well relative to a no-T-cell control. As shown in FIG. 21, both H6K3-IgG-IL12 (LC) and H6K3-IgG-IL12 (1×HC) enhanced T cell-mediated killing of SK-MEL-5 tumor cells relative to nontargeted rhIL-12.

### C. In Vivo Studies of Humanized Anti-Human-CD8/rhIL-12 Fusion Protein Constructs.

Two fusion protein constructs were generated that each comprised (1) a humanized anti-human CD8 IgG1 antibody comprising humanized 02A01 VH6 and humanized 02A01 VK3 (D30E, N35Q) (see Table 1), and (2) a human IL-12p70 protein. The constructs were built in accordance with one of the two following scaffold formats described herein: “IgG-immunomodulator (LC)” and “IgG-immunomodulator (1×HC) (see FIG. 1B and FIG. 1E). These fusion proteins are referred to herein as “H6K3EQ-IgG-IL12 (LC)” and “H6K3EQ-IgG-IL12 (1×HC)”, respectively. Both fusion protein constructs utilized a human IgG1 Fc, and comprised the substitutions L234A, L235A, P329G, and N297A in the IgG1 Fc domain that reduce ADCC effector function. The H6K3EQ-IgG-IL12 (1×HC) construct further comprised the substitution T366W in the Fc polypeptide chain linked to IL-12 and the substitutions T366S, L368A, and Y407V in the other Fc polypeptide chain, which promote heterodimerization. Both fusion proteins were tested in vivo in cynomolgus monkeys for their ability to stimulate IFN-γ production and for their ability to selectively bind CD8-expressing immune cells.

The two aforementioned fusion protein constructs were evaluated for their ability to stimulate IFN-γ production following subcutaneous or intravenous administration to cynomolgus monkeys. Briefly, 100 μg/kg of H6K3EQ-IgG-IL12 (LC) or H6K3EQ-IgG-IL12 (1×HC) were administered to cynomolgus monkeys either subcutaneously or intravenously. Serum samples were taken prior to administration to establish a background reference for IFN-γ production, and serum samples were taken again 3 days post-administration of the anti-CD8:IL-12 fusion protein construct. Serum IFN-γ levels were measured using Monkey IFN-γ ELISA Kit (Abcam).

As shown in FIG. 22, subcutaneous and intravenous administration of either anti-CD8:IL-12 fusion protein construct stimulated an increase in IFN-γ production as compared to pre-dose levels. Of all treatments tested, intravenous administration of H6K3EQ-IgG-IL12 (1×HC) elicited the greatest increase in IFN-γ production.

The H6K3EQ-IgG-IL12 (LC) and H6K3EQ-IgG-IL12 (1×HC) fusion protein constructs were also tested for their ability to specifically bind CD8-expressing cynomolgus monkey immune cells (CD8+ T cells and NK cells) in vivo. Briefly, cynomolgus monkeys were intravenously administered varying doses (10 μg/kg, 20 μg/kg, 40 μg/kg, or 100 μg/kg) of either H6K3EQ-IgG-IL12 (LC) or H6K3EQ-IgG-IL12 (1×HC). Blood samples were obtained 24-hours post-administration. Samples were analyzed via flow cytometry with fluorophore-conjugated antibodies against CD45, CD3, CD4, CD8, CD159a, and IL-12. Binding activity for all treatments was quantified and reported as the mean fluorescent intensity values of IL-12 on CD8+ T cells (FIG. 23A). For the 100 μg/kg treatment, results were reported as the frequency of IL-12 binding on the surface of CD4+ T cells, CD8+ T cells, and NK cells (FIG. 23B). As shown in FIG. 23A, higher IL-12 levels were detected on CD8+ T cells at all tested doses following H6K3EQ-IgG-IL12 (1×HC) administration as compared to H6K3EQ-IgG-IL12 (LC) administration, indicating that the (1×HC) construct exhibits better loading on target cells. As shown in FIG. 23B, IL-12 selectively bound to CD8-expressing cynomolgus monkey cells (CD8+ T cells and NK cells), and not CD4+ T cells, 24 hours post-administration of a 100 μg/kg dose of either H6K3EQ-IgG-IL12 (LC) or H6K3EQ-IgG-IL12 (1×HC). IL-12 was detected on CD8⁺ T cells and NK cells at a higher level following H6K3EQ-IgG-IL12 (1×HC) administration as compared to H6K3EQ-IgG-IL12 (LC) administration.

A third fusion construct was generated that was identical to H6K3EQ-IgG-IL12 (1×HC), except that it lacked the P329G IgG1 Fc mutation that reduces ADCC effector function. This construct is referred to herein as 02(VH6/VK3-D30E/N35Q)-IgG-IL12 (1×HC).

The 02(VH6/VK3-D30E/N35Q)-IgG-IL12 (1×HC) fusion construct was tested for its ability to bind circulating CD8-expressing cynomolgus monkey cells in vivo after administration of a range of doses. Briefly, cynomolgus monkeys were given either an intravenous dose of 02(VH6/VK3-D30E/N35Q)-IgG-IL12 (1×HC) at 10 μg/kg, 20 μg/kg, 40 μg/kg, 100 μg/kg, or 500 μg/kg, or a subcutaneous dose at 1000 μg/kg. Blood samples were obtained 24 hours, 72 hours, and 168 hours post-administration, and at an expanded range of timepoints (0 hours, 1 hour, 4 hours, 8 hours, 48 hours, 72 hours, 120 hours, 168 hours, and 240 hours post-administration) for the 100 μg/kg dose. The samples were analyzed by flow cytometry using fluorophore-conjugated antibodies that bind CD45, CD3, CD4, CD8, Ki67, IL-12, and Perforin. Serum concentrations of 02(VH6/VK3-D30E/N35Q)-IgG-IL12 (1×HC) were measured by ELISA, using recombinant human CD8αβ as the capture protein and biotin-conjugated anti-IL12 antibody as the detection reagent.

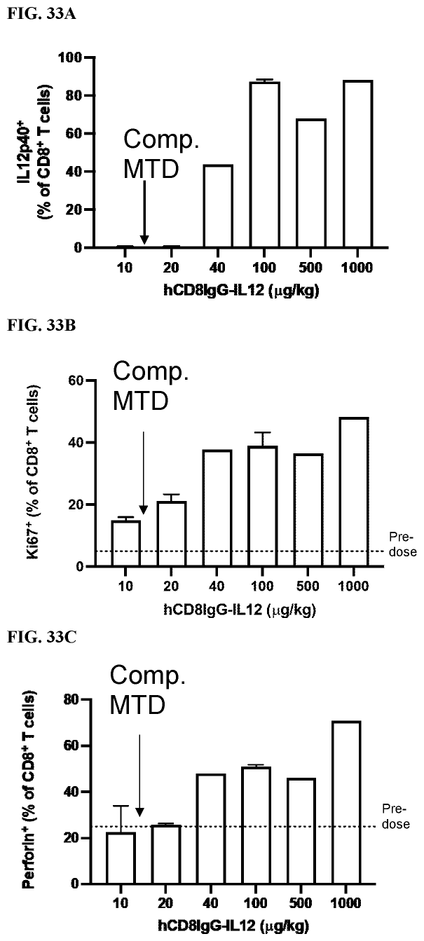

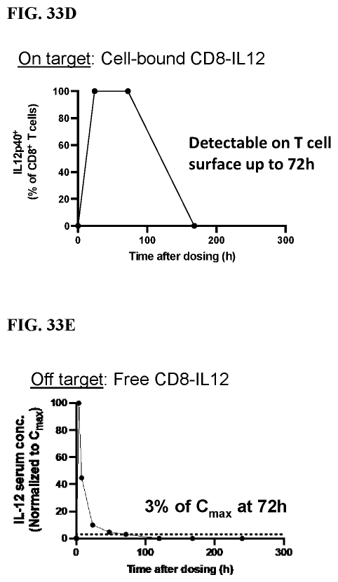

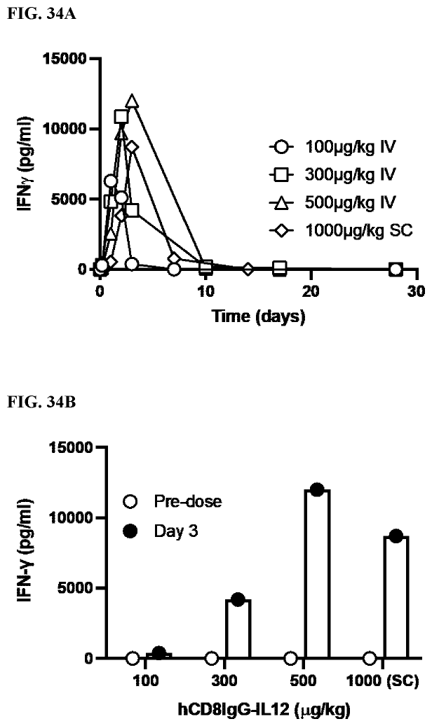

As shown in FIG. 33A, IL-12 was detectable on the surface of CD8+ cynomolgus monkey T cells 24 hours after administration of 40 μg/kg, 100 μg/kg, 500 μg/kg, or 1000 μg/kg of 02(VH6/VK3-D30E/N35Q)-IgG-IL12 (1×HC), indicating construct loading on circulating cynomolgus monkey CD8+ T cells. 02(VH6/VK3-D30E/N35Q)-IgG-IL12 (1×HC) reached saturation levels on the surface of CD8+ T cells at a dose of 100 μg/kg (FIG. 33A). The fusion construct was still detectable on the surface of a majority of CD8+ T cells for up to 72 hours post-administration of a 100 μg/kg dose of the construct (FIG. 33D). Conversely, 72 hours post-administration of a 100 μg/kg dose of the fusion protein, the serum concentration of IL-12 had dropped to 3% of the peak IL-12 concentration (FIG. 33E). These results demonstrate that the construct was sequestered to CD8+ T cells, minimizing systemic exposure and the potential for toxic off-target effects (e.g., from targeting NK cells). By comparison, the maximum tolerated doses of two known IL-12 fusion constructs, NHS-IL12 (Greiner et al., (2021) Immunotargets Ther. 10:155-169) and AS1409 (Rudman et al., (2011) Clin. Cancer Res. 17(7):1998-2005), are 16.8 μg/kg and 15 μg/kg, respectively (indicated by arrows in FIGS. 33A-C).

The administration of 02(VH6/VK3-D30E/N35Q)-IgG-IL12 (1×HC) also increased proliferation and cytotoxicity of CD8+ T cells in vivo. As shown in FIGS. 33B-33C, the percentages of Ki67+(proliferating) and Perforin+ (cytotoxic) CD8+ T cells increased in a dose-dependent manner following administration.

Collectively, the results shown in FIGS. 33A-33E indicate that 02(VH6/VK3-D30E/N35Q)-IgG-IL12 (1×HC) can be safely administered at higher IL-12 equivalent amounts than either NHS-IL12 or AS1409.

The 02(VH6/VK3-D30E/N35Q)-IgG-IL12 (1×HC) fusion construct was further assessed for its ability to increase serum concentration of IFNγ following administration at a range of doses to cynomolgus monkeys. Briefly, cynomolgus monkeys were given either an intravenous dose of 02(VH6/VK3-D30E/N35Q)-IgG-IL12 (1×HC) at 100 μg/kg, 300 μg/kg, or 500 μg/kg, or a subcutaneous dose at 1000 μg/kg. Blood samples were taken at 0 hours, 4 hours, 24 hours, 48 hours, 72 hours, 7 days, 10 days, 14 days, 17 days, and 28 days post-administration. The concentrations of IFNγ in the serum were analyzed by Luminex assay.

As shown in FIGS. 34A-34B, the fusion construct elicited transient, dose-dependent increases in serum concentration of IFNγ. Intravenous administration of the fusion construct at 500 μg/kg elicited higher levels of systemic IFNγ as compared to subcutaneous administration of the construct at 1000 μg/kg.

The 02(VH6/VK3-D30E/N35Q)-IgG-IL12 (1×HC) fusion construct was further assessed for its ability to induce proliferation of antigen-experienced T cells in vivo. Briefly, cynomolgus monkeys were given an intravenous dose of 02(VH6/VK3-D30E/N35Q)-IgG-IL12 (1×HC) at 100 μg/kg. Blood samples were taken at 0 hours, 24 hours, 72 hours, 7 days, 14 days, and 28 days post-administration. The samples from each timepoint were analyzed by flow cytometry using fluorophore-conjugated antibodies that bind CCR7, CD3, CD4, CD8, Ki67, and Perforin.

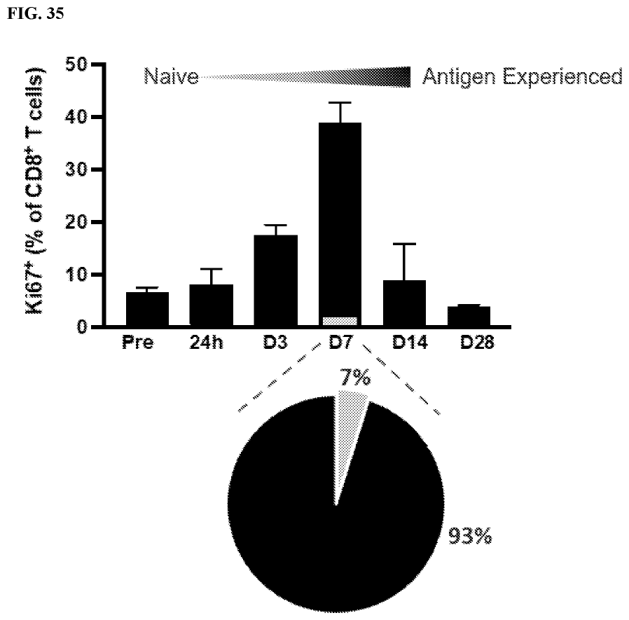

As shown in FIG. 35, administration of the fusion construct induced an increase in the percentage of CD8+ T cells that are Ki67+, indicating that the fusion construct promoted proliferation of CD8+ T cells. About 93% of the Ki67+CD8+ T cells at the 7-day timepoint were negative for CCR7, a marker of naïve T cells. This result indicates that the fusion construct primarily increased proliferation of antigen-experienced T cells.

Cynomolgus monkeys were also monitored for indicators of systemic toxicity following administration of various doses of the 02(VH6/VK3-D30E/N35Q)-IgG-IL12 (1×HC) fusion construct. Briefly, cynomolgus monkeys were given either an intravenous dose of 100 μg/kg, 300 μg/kg, or 500 μg/kg of the fusion construct or a subcutaneous dose of 1000 μg/kg of the fusion construct. Body weights of the subjects were measured at 0, 2, 4, 7, 14, and 21 days post-administration. Blood samples were taken at 0 hours, 4 hours, 24 hours, 3 days, 7 days, 14 days, and 28 days post-administration to measure serum concentration of liver enzymes ALT and AST

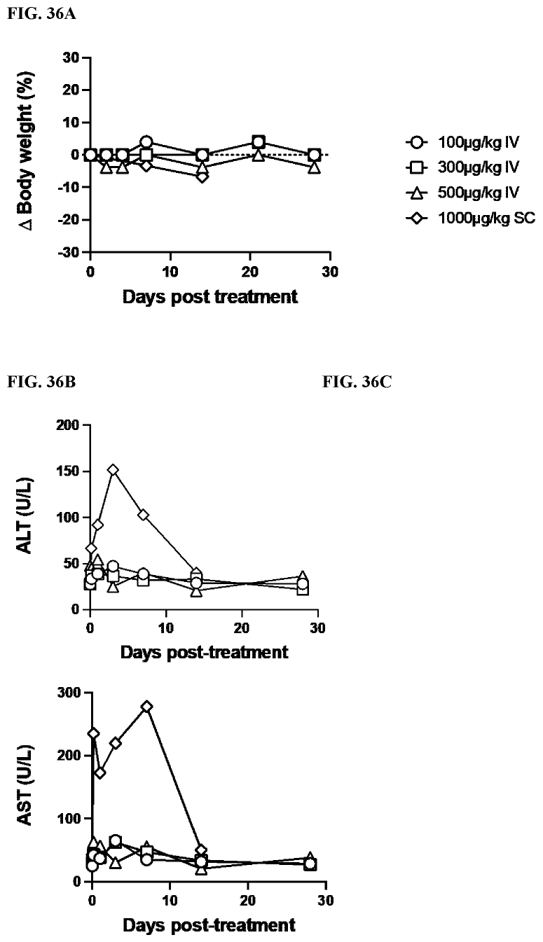

As shown in FIG. 36A, administration of the fusion construct did not cause substantial changes in body weight at any dose. As shown in FIGS. 36B-36C, administration of 1000 μg/kg of the fusion construct resulted in a transient upregulation of serum levels of ALT and AST, which were reversible 2 weeks post-administration. No other dose caused an increase in serum levels of ALT or AST, highlighting the safety of the therapy. A transient decrease in circulating lymphocytes and a transient increase in circulating neutrophils were also observed (data not shown), which may reflect lymphocyte activation. No other clinical signs of systemic toxicity were observed, other than a transient decrease in appetite and food consumption. Collectively, these data demonstrate that no dose-limiting toxicity was observed for 02(VH6/VK3-D30E/N35Q)-IgG-IL12 (1×HC) in cynomolgus monkeys for doses up to 1000 μg/kg..

### INCORPORATION BY REFERENCE

All publications and patents cited throughout the text of this specification (including all patents, patent applications, scientific publications, manufacturer's specifications, instructions, etc.), whether supra or infra, are hereby incorporated by reference in their entirety for all purposes. To the extent the material incorporated by reference contradicts or is inconsistent with this specification, the specification will supersede any such material.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

What is claimed is:

1. A fusion protein comprising an antigen-binding site that binds CD8 and an IL-12 protein, wherein the antigen-binding site does not substantially increase or decrease an activity of CD8, and the fusion protein lacks an antibody Fc domain having ADCC effector function.

2. The fusion protein of claim 1, wherein the fusion protein comprises an antibody Fc domain in which an ADCC effector function is reduced by at least 60% relative to a wild-type human IgG1 Fc domain having the amino acid sequence of SEQ ID NO: 84.

3. The fusion protein of claim 1, wherein the fusion protein lacks an antibody Fc domain.

4. A fusion protein comprising:
(i) (a) two Fab fragments each comprising an identical antigen-binding site that binds CD8;
(b) a single IL-12 protein; and
(c) an antibody Fc domain,
wherein a heavy chain polypeptide of each of the two Fab fragments is linked to an N-terminal amino acid residue of the antibody Fc domain, and the IL-12 protein is linked to a C-terminal amino acid residue of the antibody Fc domain; or
(ii) (a) two Fab fragments each comprising an identical antigen-binding site that binds CD8;
(b) two IL-12 proteins; and
(c) an antibody Fc domain,
wherein a heavy chain polypeptide of each of the two Fab fragments is linked to a N-terminal amino acid residue of the antibody Fc domain, and each of the two IL-12 proteins is linked to a C-terminal amino acid residue of a light chain polypeptide of each of the two Fab fragments.

5. The fusion protein of claim 4, wherein an ADCC effector function of the antibody Fc domain is reduced by at least 60% relative to a wild-type human IgG1 Fc domain having the amino acid sequence of SEQ ID NO: 84.

6. The fusion protein of claim 4 or 5, wherein each of the antigen-binding sites does not substantially increase or decrease an activity of CD8.

7. The fusion protein of any one of claims 1-6, wherein the IL-12 protein comprises an IL-12A subunit comprising an amino acid sequence at least 90% identical to SEQ ID NO: 89 and an IL-12B subunit comprising an amino acid sequence at least 90% identical to SEQ ID NO: 90.

8. The fusion protein of claim 7, wherein the IL-12A subunit and the IL-12B subunit are present in the same polypeptide chain.

9. The fusion protein of claim 7 or 8, wherein the IL-12A subunit is linked to the C-terminus of the IL-12B subunit by a linker comprising the amino acid sequence of SEQ ID NO: 85.

10. The fusion protein of any one of claims 1-9, wherein the IL-12 protein comprises one or more mutations that reduce affinity for an IL-12 receptor, relative to wild-type human IL-12.

11. The fusion protein of any one of claims 1-2 and 4-10, comprising:
(a) two Fab fragments each comprising the antigen-binding site;
(b) a single IL-12 protein; and
(c) an antibody Fc domain,
wherein a heavy chain polypeptide of each of the two Fab fragments is linked to an N-terminal amino acid residue of the antibody Fc domain, and the IL-12 protein is linked to a C-terminal amino acid residue of the antibody Fc domain.

12. The fusion protein of claim 11, wherein the IL-12 protein is linked to the C-terminal amino acid residue of the antibody Fc domain by a linker comprising the amino acid sequence of SEQ ID NO: 87.

13. The fusion protein of claim 12, wherein the IL-12B subunit is linked to the C-terminal amino acid residue of the antibody Fc domain by a linker comprising the amino acid sequence of SEQ ID NO: 87.

14. The fusion protein of any one of claims 11-13, wherein the antibody Fc domain comprises substitutions that promote Fc heterodimerization.

15. The fusion protein of claim 14, wherein the substitutions comprise T366W in a first polypeptide chain of the antibody Fc domain and T366S, L368A, and Y407V in a second polypeptide chain of the antibody Fc domain, the positions numbered under the EU numbering scheme.

16. The fusion protein of any one of claims 1-2 and 4-10, comprising:
(a) two Fab fragments each comprising the antigen-binding site;
(b) two IL-12 proteins; and
(c) an antibody Fc domain,
wherein a heavy chain polypeptide of each of the two Fab fragments is linked to a N-terminal amino acid residue of the antibody Fc domain, and each of the two IL-12 proteins is linked to a C-terminal amino acid residue of a light chain polypeptide of each of the two Fab fragments.

17. The fusion protein of claim 16, wherein each of the two IL-12 proteins is linked to the C-terminal amino acid residue of the light chain polypeptide of one of the two Fab fragments by a linker comprising the amino acid sequence of SEQ ID NO: 86.

18. The fusion protein of any one of claims 1-2 and 4-17, wherein the antibody Fc domain comprises amino acids A at position 234, A at position 235, A at position 297, and optionally G at position 329, the positions numbered under the EU numbering.

19. The fusion protein of any one of claims 1-18, wherein the antigen-binding site comprises a heavy chain variable domain (VH) comprising complementarity determining regions HCDR1, HCDR2, and HCDR3 and a light chain variable domain (VL) comprising complementarity determining regions LCDR1, LCDR2, and LCDR3, wherein the HCDR1, HCDR2, HCDR3, LCDR1, LCDR2, and LCDR3 comprise the amino acid sequences of SEQ ID NOs: 112, 113, 4, 109, 12, and 8, respectively.

20. The fusion protein of claim 19, wherein the LCDR2 comprises the amino acid sequence of SEQ ID NO: 84.

21. The fusion protein of claim 19 or 20, wherein the HCDR1, HCDR2, HCDR3, LCDR1, LCDR2, and LCDR3 comprise the amino acid sequences of SEQ ID NOs: 112, 113, 4, 106, 7, and 8, respectively.

22. The fusion protein of any one of claims 19-21, wherein the VH and the VL comprise amino acid sequences at least 95% identical to SEQ ID NOs: 21 and 105; 14 and 105; 15 and 105; 16 and 105; or 18 and 105, respectively.

23. The fusion protein of claim 19 or claim 20, wherein the HCDR1, HCDR2, HCDR3, LCDR1, LCDR2, and LCDR3 comprise the amino acid sequences of SEQ ID NOs: 112, 113, 4, 6, 7, and 8, respectively.

24. The fusion protein of any one of claims 19-20 or 23, wherein the VH and the VL comprise amino acid sequences at least 95% identical to SEQ ID NOs: 14 and 22; 15 and 22; 16 and 22; 18 and 22; 21 and 22; 14 and 23; 15 and 23; 16 and 23; 18 and 23; 21 and 23; 14 and 24; 15 and 24; 16 and 24; 18 and 24; or 21 and 24, respectively.

25. The fusion protein of any one of claims 19-20 or 23, wherein the VH and the VL comprise amino acid sequences at least 95% identical to SEQ ID NOs: 1 and 5, respectively, or 9 and 5, respectively.

26. The fusion protein of claim 19 or claim 20, wherein the HCDR1, HCDR2, HCDR3, LCDR1, LCDR2, and LCDR3 comprise the amino acid sequences of SEQ ID NOs: 112, 113, 4, 6, 26, and 8, respectively.

27. The fusion protein of any one of claims 19-20 or 26, wherein the VH and the VL comprise amino acid sequences at least 95% identical to SEQ ID NOs: 14 and 25; 15 and 25; 16 and 25; 18 and 25; or 21 and 25, respectively.

28. The fusion protein of any one of claims 1-18, wherein the antigen-binding site comprises a heavy chain variable domain (VH) comprising complementarity determining regions HCDR1, HCDR2, and HCDR3 and a light chain variable domain (VL) comprising complementarity determining regions LCDR1, LCDR2, and LCDR3, wherein the HCDR1, HCDR2, HCDR3, LCDR1, LCDR2, and LCDR3 comprise the amino acid sequences of SEQ ID NOs: 88, 11, 4, 109, 12, and 8, respectively.

29. The fusion protein of claim 28, wherein the HCDR1, HCDR2, and LCDR2 comprise the amino acid sequences of SEQ ID NOs: 2, 13, and 84, respectively.

30. The fusion protein of claim 28 or 29, wherein the HCDR1, HCDR2, HCDR3, LCDR1, LCDR2, and LCDR3 comprise the amino acid sequences of SEQ ID NOs: 2, 17, 4, 106, 7, and 8, respectively.

31. The fusion protein of any one of claims 28-30, wherein the VH and the VL comprise amino acid sequences at least 95% identical to SEQ ID NOs: 21 and 105; 16 and 105; or 18 and 105, respectively.

32. The fusion protein of claim 28 or 29, wherein the HCDR1, HCDR2, HCDR3, LCDR1, LCDR2, and LCDR3 comprise the amino acid sequences of SEQ ID NOs: 2, 17, 4, 6, 7, and 8, respectively.

33. The fusion protein of any one of claims 28-29 and 32, wherein the VH and the VL comprise amino acid sequences at least 95% identical to SEQ ID NOs: 21 and 24; 16 and 24; 18 and 24; 21 and 22; 16 and 22; 18 and 22; 21 and 23; 16 and 23; or 18 and 23, respectively.

34. The fusion protein of claim 28 or 29, wherein the HCDR1, HCDR2, HCDR3, LCDR1, LCDR2, and LCDR3 comprise the amino acid sequences of SEQ ID NOs: 2, 3, 4, 6, 7, and 8, respectively.

35. The fusion protein of any one of claims 28-29 and 34, wherein the VH and the VL comprise amino acid sequences at least 95% identical to SEQ ID NOs: 14 and 24; 15 and 24; 14 and 22; 15 and 22; 14 and 23; or 15 and 23, respectively.

36. The fusion protein of any one of claims 28-29 and 34, wherein the VH and the VL comprise amino acid sequences at least 95% identical to SEQ ID NOs: 1 and 5, respectively.

37. The fusion protein of claim 28 or 29, wherein the HCDR1, HCDR2, HCDR3, LCDR1, LCDR2, and LCDR3 comprise the amino acid sequences of SEQ ID NOs: 2, 17, 4, 6, 26, and 8, respectively.

38. The fusion protein of any one of claims 28-29 and 37, wherein the VH and the VL comprise amino acid sequences at least 95% identical to SEQ ID NOs: 21 and 25; 16 and 25;
or 18 and 25, respectively.

39. The fusion protein of claim 28 or 29, wherein the HCDR1, HCDR2, HCDR3, LCDR1, LCDR2, and LCDR3 comprise the amino acid sequences of SEQ ID NOs: 2, 3, 4, 6, 26, and 8, respectively.

40. The fusion protein of any one of claims 28-29 and 39, wherein the VH and the VL comprise amino acid sequences at least 95% identical to SEQ ID NOs: 14 and 25; or 15 and 25, respectively.

41. The fusion protein of claim 28, wherein the HCDR1, HCDR2, HCDR3, LCDR1, LCDR2, and LCDR3 comprise the amino acid sequences of SEQ ID NOs: 10, 3, 4, 6, 7, and 8, respectively.

42. The fusion protein of claim 41, wherein the VH and the VL comprise amino acid sequences at least 95% identical to SEQ ID NOs: 9 and 5, respectively.

43. The fusion protein of any one of claims 1-18, wherein the antigen-binding site comprises a VH comprising complementarity determining regions HCDR1, HCDR2, and HCDR3 and a VL comprising complementarity determining regions LCDR1, LCDR2, and LCDR3, wherein the HCDR1, HCDR2, HCDR3, LCDR1, LCDR2, and LCDR3 comprise the amino acid sequences of SEQ ID NOs: 115, 118, 30, 32, 33, and 34, respectively.

44. The fusion protein of claim 43, wherein the HCDR1, HCDR2, HCDR3, LCDR1, LCDR2, and LCDR3 comprise the amino acid sequences of SEQ ID NOs: 115, 116, 30, 32, 33, and 34, respectively.

45. The fusion protein of claim 43 or 44, wherein the VH and the VL comprise amino acid sequences at least 95% identical to SEQ ID NOs: 39 and 45; 39 and 46; 39 and 47; 40 and 45; 40 and 46; 40 and 47; 41 and 45; 41 and 46; 41 and 47; 43 and 45; 43 and 46; or 43 and 47, respectively.

46. The fusion protein of claim 43 or 44, wherein the VH and the VL comprise amino acid sequences at least 95% identical to SEQ ID NOs: 27 and 31, respectively.

47. The fusion protein of claim 43, wherein the HCDR1, HCDR2, HCDR3, LCDR1, LCDR2, and LCDR3 comprise the amino acid sequences of SEQ ID NOs: 115, 117, 30, 32, 33, and 34, respectively.

48. The fusion protein of claim 43 or 47, wherein the VH and the VL comprise amino acid sequences at least 95% identical to SEQ ID NOs: 35 and 31, respectively.

49. The fusion protein of any one of claims 1-18, wherein the antigen-binding site comprises a VH comprising complementarity determining regions HCDR1, HCDR2, and HCDR3 and a VL comprising complementarity determining regions LCDR1, LCDR2, and LCDR3, wherein the HCDR1, HCDR2, HCDR3, LCDR1, LCDR2, and LCDR3 comprise the amino acid sequences of SEQ ID NOs: 28, 37, 30, 32, 33, and 34, respectively.

50. The fusion protein of claim 49, wherein the HCDR2 comprises the amino acid sequence of SEQ ID NO: 38.

51. The fusion protein of claim 49 or 50, wherein the HCDR1, HCDR2, HCDR3, LCDR1, LCDR2, and LCDR3 comprise the amino acid sequences of SEQ ID NOs: 28, 29, 30, 32, 33, and 34, respectively.

52. The fusion protein of any one of claims 49-51, wherein the VH and the VL comprise amino acid sequences at least 95% identical to SEQ ID NOs: 39 and 45; 39 and 46; 39 and 47; 40 and 45; 40 and 46; or 40 and 47, respectively.

53. The fusion protein of any one of claims 49-51, wherein the VH and the VL comprise amino acid sequences at least 95% identical to SEQ ID NOs: 27 and 31, respectively.

54. The fusion protein of claim 49 or 50, wherein the HCDR1, HCDR2, HCDR3, LCDR1, LCDR2, and LCDR3 comprise the amino acid sequences of SEQ ID NOs: 28, 42, 30, 32, 33, and 34, respectively.

55. The fusion protein of any one of claims 49-50 and 54, wherein the VH and the VL comprise amino acid sequences at least 95% identical to SEQ ID NOs: 41 and 45; 41 and 46;
or 41 and 47, respectively.

56. The fusion protein of claim 49 or 50, wherein the HCDR1, HCDR2, HCDR3, LCDR1, LCDR2, and LCDR3 comprise the amino acid sequences of SEQ ID NOs: 28, 44, 30, 32, 33, and 34, respectively.

57. The fusion protein of any one of claims 49-50 and 56, wherein the VH and the VL comprise amino acid sequences at least 95% identical to SEQ ID NOs: 43 and 45; 43 and 46; or 43 and 47, respectively.

58. The fusion protein of claim 49, wherein the HCDR1, HCDR2, HCDR3, LCDR1, LCDR2, and LCDR3 comprise the amino acid sequences of SEQ ID NOs: 28, 36, 30, 32, 33, and 34, respectively.

59. The fusion protein of claim 58, wherein the VH and the VL comprise amino acid sequences at least 95% identical to SEQ ID NOs: 35 and 31, respectively.

60. The fusion protein of any one of claims 1-18, wherein the antigen-binding site comprises complementarity determining regions HCDR1, HCDR2, and HCDR3 and a VL comprising complementarity determining regions LCDR1, LCDR2, and LCDR3, wherein the HCDR1, HCDR2, HCDR3, LCDR1, LCDR2, and LCDR3 comprise the amino acid sequences of SEQ ID NOs: 119, 120, 53, 55, 56, and 57, respectively.

61. The fusion protein of any one of claims 1-18, wherein the antigen-binding site comprises a VH comprising complementarity determining regions HCDR1, HCDR2, and HCDR3 and a VL comprising complementarity determining regions LCDR1, LCDR2, and LCDR3, wherein the HCDR1, HCDR2, HCDR3, LCDR1, LCDR2, and LCDR3 comprise the amino acid sequences of SEQ ID NOs: 51, 52, 53, 55, 56, and 57, respectively.

62. The fusion protein of claim 60 or claim 61, wherein the VH and the VL comprise amino acid sequences at least 95% identical to SEQ ID NOs: 50 and 54, respectively.

63. The fusion protein of any one of claims 1-18, wherein the antigen-binding site comprises complementarity determining regions HCDR1, HCDR2, and HCDR3 and a VL comprising complementarity determining regions LCDR1, LCDR2, and LCDR3, wherein the HCDR1, HCDR2, HCDR3, LCDR1, LCDR2, and LCDR3 comprise the amino acid sequences of SEQ ID NOs: 121, 122, 61, 32, 63, and 64, respectively.

64. The fusion protein of any one of claims 1-18, wherein the antigen-binding site comprises a VH comprising complementarity determining regions HCDR1, HCDR2, and HCDR3 and a VL comprising complementarity determining regions LCDR1, LCDR2, and LCDR3, wherein the HCDR1, HCDR2, HCDR3, LCDR1, LCDR2, and LCDR3 comprise the amino acid sequences of SEQ ID NOs: 59, 60, 61, 32, 63, and 64, respectively.

65. The fusion protein of claim 63 or claim 64, wherein the VH and the VL comprise amino acid sequences at least 95% identical to SEQ ID NOs: 58 and 62, respectively.

66. The fusion protein of any one of claims 1-18, wherein the antigen-binding site comprises a VH comprising complementarity determining regions HCDR1, HCDR2, and HCDR3 and a VL comprising complementarity determining regions LCDR1, LCDR2, and LCDR3, wherein the HCDR1, HCDR2, HCDR3, LCDR1, LCDR2, and LCDR3 comprise the amino acid sequences of SEQ ID NOs: 123, 124, 68, 70, 71, and 72, respectively.

67. The fusion protein of any one of claims 1-18, wherein the antigen-binding site comprises a VH comprising complementarity determining regions HCDR1, HCDR2, and HCDR3 and a VL comprising complementarity determining regions LCDR1, LCDR2, and LCDR3, wherein the HCDR1, HCDR2, HCDR3, LCDR1, LCDR2, and LCDR3 comprise the amino acid sequences of SEQ ID NOs: 66, 67, 68, 70, 71, and 72, respectively.

68. The fusion protein of claim 66 or claim 67, wherein the VH and the VL comprise amino acid sequences at least 95% identical to SEQ ID NOs: 65 and 69, respectively.

69. The fusion protein of any one of claims 1-18, wherein the antigen binding-site cross-competes for binding to CD8 with an antigen-binding site comprising a VH and a VL comprising the amino acid sequences of SEQ ID NOs: 1 and 5; or 27 and 31, respectively.

70. The fusion protein of any one of claims 1-18 and 69, wherein the antigen-binding site binds amino acids 28-32, 48, and 51 within a CD8 extracellular region defined by SEQ ID NO: 110.

71. The fusion protein of claim 70, wherein the antigen-binding site binds amino acids 28-32, 34, 48, and 51 within a CD8 extracellular region defined by SEQ ID NO: 110.

72. The fusion protein of claim 70, wherein the antigen-binding site binds amino acids 26, 28-32, 48, 51, 53, 97, and 100 within a CD8 extracellular region defined by SEQ ID NO: 110.

73. The fusion protein of any one of claims 1-72, wherein the antigen-binding site does not increase or decrease the stability of a complex by more than 20%, the complex comprising CD8, a T cell epitope presented by a human class I major histocompatibility complex (MHC) tetramer, and a cognate T cell receptor (TCR), as measured by the amount of the tetramer bound to a cell expressing the TCR.

74. The fusion protein of any one of claims 1-73, wherein the antigen-binding site does not increase or decrease T cell activation by more than 50% as measured by cytotoxicity of cancer cells caused by T cells, wherein the T cells express a TCR that recognize a T cell epitope presented by a human class I MHC on the surface of the cancer cells.

75. The fusion protein of any one of claims 1-2, 4-9, 11-15, 18-24, 28-33, and 69-74, comprising:
(a) a polypeptide comprising the amino acid sequence of SEQ ID NO: 82;
(b) a polypeptide comprising the amino acid sequence of SEQ ID NO: 83; and
(c) two polypeptides each comprising the amino acid sequence of SEQ ID NO: 108 or 78.

76. The fusion protein of any one of claims 1-2, 4-9, 16-24, 28-33, and 69-74, comprising:
(a) two polypeptides each comprising the amino acid sequence of SEQ ID NO: 76; and
(b) two polypeptides each comprising the amino acid sequence of SEQ ID NO: 74 or 107.

77. The fusion protein of any one of claims 1-76, wherein the fusion protein is an immunostimulatory fusion protein.

78. The fusion protein of any one of claims 1-77, wherein the fusion protein is a soluble protein.

79. A pharmaceutical composition comprising the fusion protein of any one of claims 1-78 and a pharmaceutically acceptable carrier or excipient.

80. A method of killing a cancer cell, the method comprising exposing the cancer cell and a CD8⁺ T lymphocyte to the fusion protein of any one of claims 1-78.

81. A method of treating cancer, the method comprising administering to a subject in need thereof an effective amount of the fusion protein of any one of claims 1-78 or the pharmaceutical composition of claim 79.

82. One or more nucleic acids encoding the fusion protein of any one of claims 1-78.

83. A vector comprising the one or more nucleic acids of claim 82.

84. A recombinant cell comprising the one or more nucleic acids of claim 82 or the vector of claim 83.

85. A method of producing a protein, the method comprising culturing the recombinant cell of claim 84 under suitable conditions that allow expression of the fusion protein.

86. The method of claim 85, further comprising isolating the fusion protein.

87. The method of claim 86, further comprising formulating the fusion protein with a pharmaceutically acceptable carrier or excipient.


SEQUENCE LISTING (124 sequences)

SEQ ID NO: 1 - protein, 120 aa, synthetic construct
  source 1..120: note = Murine 02A01 VH
EVQLQQSGPELVKPGASLKISCKASGYTFTDFTMHWVKQSHGKSLEWIGLVNPNNGGNIYNQNFKGKATLTVDTSSSTAYMELRSLTSEDSSVYYCGRVPLYTSHGMDYWGQGTSVTVSS

SEQ ID NO: 2 - protein, 5 aa, synthetic construct
  source 1..5: note = Murine 02A01 VH CDR1
DFTMH

SEQ ID NO: 3 - protein, 17 aa, synthetic construct
  source 1..17: note = Murine 02A01 VH CDR2
LVNPNNGGNIYNQNFKG

SEQ ID NO: 4 - protein, 11 aa, synthetic construct
  source 1..11: note = Murine 02A01 VH CDR3
VPLYTSHGMDY

SEQ ID NO: 5 - protein, 111 aa, synthetic construct
  source 1..111: note = Murine 02A01 VL
DIVLTQSPAFLAVSLGQRATISCRASESVDSYDINSMHWYQQKPGQPPKLLIYRASHLQSGIPARFSGSGSRTDFTLTINFVEADDVATYYCQQSNEDPLTFGAGTKLELK

SEQ ID NO: 6 - protein, 15 aa, synthetic construct
  source 1..15: note = Murine 02A01 VL CDR1
RASESVDSYDINSMH

SEQ ID NO: 7 - protein, 7 aa, synthetic construct
  source 1..7: note = Murine 02A01 VL CDR2
RASHLQS

SEQ ID NO: 8 - protein, 9 aa, synthetic construct
  source 1..9: note = Murine 02A01 VL CDR3
QQSNEDPLT

SEQ ID NO: 9 - protein, 120 aa, synthetic construct
  source 1..120: note = Murine 02A01 VH-M39L
EVQLQQSGPELVKPGASLKISCKASGYTFTDFTLHWVKQSHGKSLEWIGLVNPNNGGNIYNQNFKGKATLTVDTSSSTAYMELRSLTSEDSSVYYCGRVPLYTSHGMDYWGQGTSVTVSS

SEQ ID NO: 10 - protein, 5 aa, synthetic construct
  source 1..5: note = Murine 02A01 VH-M39L CDR1
DFTLH

SEQ ID NO: 11 - protein, 17 aa, synthetic construct
  source 1..17: note = 02A01 Consensus 1 VH CDR2
  VARIANT 12: note = X is N or A
  VARIANT 14: note = X is N or K
  VARIANT 16: note = X is K or Q
LVNPNNGGNIYXZXFXG

SEQ ID NO: 12 - skipped

SEQ ID NO: 13 - protein, 17 aa, synthetic construct
  source 1..17: note = 02A01 Consensus 2 VH CDR2
  VARIANT 12..16: note = AEKFQ or NQNFK
LVNPNNGGNIYXXXFXG

SEQ ID NO: 14 - protein, 120 aa, synthetic construct
  source 1..120: note = Humanized 02A01 VH1
EVQLVQSGPEVVKPGATLKISCKASGYTFTDFTMHWVKQAPGKSLEWIGLVNPNNGGNIYNQNFKGRATLTVDTSTSTAYMELSSLRSEDTSVYYCGRVPLYTSHGMDYWGQGTLVTVSS

SEQ ID NO: 15 - protein, 120 aa, synthetic construct
  source 1..120: note = Humanized 02A01 VH2
EVQLVQSGAEVKKPGATLKISCKASGYTFTDFTMHWVQQAPGKGLEWIGLVNPNNGGNIYNQNFKGRATLTVDTSTSTAYMELSSLRSEDTSVYYCGRVPLYTSHGMDYWGQGTLVTVSS

SEQ ID NO: 16 - protein, 120 aa, synthetic construct
  source 1..120: note = Humanized 02A01 VH3
EVQLVQSGAEVKKPGATLKISCKASGYTFTDFTMHWVQQAPGKGLEWIGLVNPNNGGNIYAEKFQGRATLTVDTSTSTAYMELSSLRSEDTSVYYCGRVPLYTSHGMDYWGQGTLVTVSS

SEQ ID NO: 17 - protein, 17 aa, synthetic construct
  source 1..17: note = Humanized 02A01 VH3 CDR2
LVNPNNGGNIYAEKFQG

SEQ ID NO: 18 - protein, 120 aa, synthetic construct
  source 1..120: note = Humanized 02A01 VH4
EVQLVQSGAEVKKPGATVKISCKASGYTFTDFTMHWVQQAPGKGLEWIGLVNPNNGGNIYAEKFQGRATLTVDTSTSTAYMELSSLRSEDTAVYYCGRVPLYTSHGMDYWGQGTLVTVSS

SEQ ID NO: 19 - protein, 120 aa, synthetic construct
  source 1..120: note = Humanized 02A01 VH5
EVQLVQSGAEVKKPGATLKISCKASGYTFTDYYMHWVQQAPGKGLEWIGLVNPNNGGNIYNQNFKGRATLTVDTSTSTAYMELSSLRSEDTSVYYCGRVPLYTSHGMDYWGQGTLVTVSS

SEQ ID NO: 20 - protein, 5 aa, synthetic construct
  source 1..5: note = Humanized 02A01 VH5 CDR1
DYYMH

SEQ ID NO: 21 - protein, 120 aa, synthetic construct
  source 1..120: note = Humanized 02A01 VH6
EVQLVQSGAEVKKPGATVKISCKASGYTFTDFTMHWVQQAPGKGLEWIGLVNPNNGGNIYAEKFQGRVTLTVDTSTSTAYMELSSLRSEDTAVYYCGRVPLYTSHGMDYWGQGTLVTVSS

SEQ ID NO: 22 - protein, 111 aa, synthetic construct
  source 1..111: note = Humanized 02A01 VK1
EIVLTQSPATLSVSPGERATISCRASESVDSYDINSMHWYQQKPGQPPRLLIYRASHLQSGIPARFSGSGSRTDFTLTISSVEPEDFATYYCQQSNEDPLTFGQGTKLEIK

SEQ ID NO: 23 - protein, 111 aa, synthetic construct
  source 1..111: note = Humanized 02A01 VK2
EIVLTQSPATLSVSPGERATISCRASESVDSYDINSMHWYQQKPGQAPRLLIYRASHLQSGIPARFSGSGSRTDFTLTISSVEPEDFATYYCQQSNEDPLTFGQGTKLEIK

SEQ ID NO: 24 - protein, 111 aa, synthetic construct
  source 1..111: note = Humanized 02A01 VK3
EIVLTQSPATLSLSPGERATISCRASESVDSYDINSMHWYQQKPGQAPRLLIYRASHLQSGIPARFSGSGSRTDFTLTISSLEPEDFATYYCQQSNEDPLTFGQGTKLEIK

SEQ ID NO: 25 - protein, 111 aa, synthetic construct
  source 1..111: note = Humanized 02A01 VK4
EIVLTQSPATLSVSPGERATISCRASESVDSYDINSMHWYQQKPGQAPRLLIYDASNRATGIPARFSGSGSRTDFTLTISSVEPEDFATYYCQQSNEDPLTFGQGTKLEIK

SEQ ID NO: 26 - protein, 7 aa, synthetic construct
  source 1..7: note = Humanized 02A01 VK4 CDR2
DASNRAT

SEQ ID NO: 27 - protein, 120 aa, synthetic construct
  source 1..120: note = 03A01 VH
DVQFQESGPGLVKPSQSLSLTCTVTGYSITSDFAWNWIRQFPGNELEWMGYISYSGDTNYNPSLRSRISITRDTSKNQFFLQLDSVTTEDTATYYCTIRGYYTDSAFVFWGQGTLVTVSA

SEQ ID NO: 28 - protein, 6 aa, synthetic construct
  source 1..6: note = 03A01 VH CDR1
SDFAWN

SEQ ID NO: 29 - protein, 16 aa, synthetic construct
  source 1..16: note = 03A01 VH CDR2
YISYSGDTNYNPSLRS

SEQ ID NO: 30 - protein, 11 aa, synthetic construct
  source 1..11: note = 03A01 VH CDR3
RGYYTDSAFVF

SEQ ID NO: 31 - protein, 107 aa, synthetic construct
  source 1..107: note = 03A01 VL
DIQMTQSSSYLSVSLGGRVTITCKASDHINNWLAWYQQKPGNAPRLLISGATSLETGVPSRFTGSGSGLDYTLSITSLQTEDVATYYCQQYWNSPFTFGSGTKLEIK

SEQ ID NO: 32 - protein, 11 aa, synthetic construct
  source 1..11: note = 03A01 VL CDR1
KASDHINNWLA

SEQ ID NO: 33 - protein, 7 aa, synthetic construct
  source 1..7: note = 03A01 VL CDR2
GATSLET

SEQ ID NO: 34 - protein, 9 aa, synthetic construct
  source 1..9: note = 03A01 VL CDR3
QQYWNSPFT

SEQ ID NO: 35 - protein, 120 aa, synthetic construct
  source 1..120: note = 03A01 VH-D64E
DVQFQESGPGLVKPSQSLSLTCTVTGYSITSDFAWNWIRQFPGNELEWMGYISYSGETNYNPSLRSRISITRDTSKNQFFLQLDSVTTEDTATYYCTIRGYYTDSAFVFWGQGTLVTVSA

SEQ ID NO: 36 - protein, 16 aa, synthetic construct
  source 1..16: note = 03A01 VH-D64E CDR2
YISYSGETNYNPSLRS

SEQ ID NO: 37 - protein, 16 aa, synthetic construct
  source 1..16: note = 03A01 Consensus 1 VH CDR2
  VARIANT 7: note = X is D or E
  VARIANT 9: note = X is N or Y
  VARIANT 15: note = X is R or K
YISYSGXTXYNPSLXS

SEQ ID NO: 38 - protein, 16 aa, synthetic construct
  source 1..16: note = 03A01 Consensus 2 VH CDR2
  VARIANT 9..15: note = NYNPSLR or NYNPSLK or YYNPSLK
YISYSGDTXYNPSLXS

SEQ ID NO: 39 - protein, 120 aa, synthetic construct
  source 1..120: note = Humanized 03A01-VH1
EVQFQESGPGLVKPSQTLSLTCTVTGYSITSDFAWNWIRQPPGKELEWMGYISYSGDTNYNPSLRSRITISRDTSKNQFSLKLSSVTAADTATYYCTIRGYYTDSAFVFWGQGTLVTVSS

SEQ ID NO: 40 - protein, 120 aa, synthetic construct
  source 1..120: note = Humanized 03A01-VH2
EVQFQESGPGLVKPSQTLSLTCTVSGYSITSDFAWNWIRQPPGKGLEWMGYISYSGDTNYNPSLRSRITISRDTSKNQFSLKLSSVTAADTAVYYCTIRGYYTDSAFVFWGQGTLVTVSS

SEQ ID NO: 41 - protein, 120 aa, synthetic construct
  source 1..120: note = Humanized 03A01-VH3
EVQFQESGPGLVKPSQTLSLTCTVSGYSITSDFAWNWIRQPPGKGLEWMGYISYSGDTNYNPSLKSRVTISRDTSKNQFSLKLSSVTAADTAVYYCTIRGYYTDSAFVFWGQGTLVTVSS

SEQ ID NO: 42 - protein, 16 aa, synthetic construct
  source 1..16: note = Humanized 03A01-VH3 CDR2
YISYSGDTNYNPSLKS

SEQ ID NO: 43 - protein, 120 aa, synthetic construct
  source 1..120: note = Humanized 03A01-VH4
EVQFQESGPGLVKPSQTLSLTCTVSGYSITSDFAWNWIRQPPGKGLEWIGYISYSGDTYYNPSLKSRVTISRDTSKNQFSLKLSSVTAADTAVYYCTIRGYYTDSAFVFWGQGTLVTVSS

SEQ ID NO: 44 - protein, 16 aa, synthetic construct
  source 1..16: note = Humanized 03A01-VH4 CDR2
YISYSGDTYYNPSLKS

SEQ ID NO: 45 - protein, 107 aa, synthetic construct
  source 1..107: note = Humanized 03A01-VK1
DIQMTQSSSTLSVSLGDRVTITCKASDHINNWLAWYQQKPGKAPRLLISGATSLETGVPSRFSGSGSGLEYTLTISSLQPDDFATYYCQQYWNSPFTFGQGTKLEIK

SEQ ID NO: 46 - protein, 107 aa, synthetic construct
  source 1..107: note = Humanized 03A01-VK2
DIQMTQSPSTLSVSVGDRVTITCKASDHINNWLAWYQQKPGKAPKLLISGATSLETGVPSRFSGSGSGLEYTLTISSLQPDDFATYYCQQYWNSPFTFGQGTKLEIK

SEQ ID NO: 47 - protein, 107 aa, synthetic construct
  source 1..107: note = Humanized 03A01-VK3
DIQMTQSPSTLSASVGDRVTITCKASDHINNWLAWYQQKPGKAPKLLISGATSLETGVPSRFSGSGSGLEYTLTISSLQPDDFATYYCQQYWNSPFTFGQGTKLEIK

SEQ ID NO: 48 - protein, 107 aa, synthetic construct
  source 1..107: note = Humanized 03A01-VK4
DIQMTQSPSTLSVSVGDRVTITCRASQSISSWLAWYQQKPGKAPKLLISGATSLETGVPSRFSGSGSGLEYTLTISSLQPDDFATYYCQQYWNSPFTFGQGTKLEIK

SEQ ID NO: 49 - protein, 11 aa, synthetic construct
  source 1..11: note = Humanized 03A01-VK4 CDR1
RASQSISSWLA

SEQ ID NO: 50 - protein, 121 aa, synthetic construct
  source 1..121: note = 05F10 VH
EVQLVESGGGLVKPGGSLKLSCAASGFTFSSFAMSWVRQTPAKRLEWVATISSGDLSANYPDTVKGRFTISRDNAKNTLYLQMSSLRSEDTAMYYCTRPPFYFSNDWFAYWGQGTLVTVSA

SEQ ID NO: 51 - protein, 5 aa, synthetic construct
  source 1..5: note = 05F10 VH CDR1
SFAMS

SEQ ID NO: 52 - protein, 17 aa, synthetic construct
  source 1..17: note = 05F10 VH CDR2
TISSGDLSANYPDTVKG

SEQ ID NO: 53 - protein, 12 aa, synthetic construct
  source 1..12: note = 05F10 VH CDR3
PPFYFSNDWFAY

SEQ ID NO: 54 - protein, 112 aa, synthetic construct
  source 1..112: note = 05F10 VL
DVVMTQTPLSLPVSLEDQASISCRSSQSLEHSNGNTYLHWYLQKPGQSPRLLIYKVSNRFSGVPDRFSGSGSGTDFTLKISRVEAEDLGVYFCSQSTHVPFTFGSGTKLEIK

SEQ ID NO: 55 - protein, 16 aa, synthetic construct
  source 1..16: note = 05F10 VL CDR1
RSSQSLEHSNGNTYLH

SEQ ID NO: 56 - protein, 7 aa, synthetic construct
  source 1..7: note = 05F10 VL CDR2
KVSNRFS

SEQ ID NO: 57 - protein, 9 aa, synthetic construct
  source 1..9: note = 05F10 VL CDR3
SQSTHVPFT

SEQ ID NO: 58 - protein, 120 aa, synthetic construct
  source 1..120: note = 11F05 VH
DVQLQESGPGLVKPSQSLSLTCTVTGYSITSDYAWNWIRQFPGNKLEWMGYISYSGSTSYNPSLKSRISITRDTSKNQFFLHLNSVTTEDTATYYCARRGYYSHSAFTYWGQGTLVTVSA

SEQ ID NO: 59 - protein, 6 aa, synthetic construct
  source 1..6: note = 11F05 VH CDR1
SDYAWN

SEQ ID NO: 60 - protein, 16 aa, synthetic construct
  source 1..16: note = 11F05 VH CDR2
YISYSGSTSYNPSLKS

SEQ ID NO: 61 - protein, 11 aa, synthetic construct
  source 1..11: note = 11F05 VH CDR3
RGYYSHSAFTY

SEQ ID NO: 62 - protein, 107 aa, synthetic construct
  source 1..107: note = 11F05 VL
DIQMTQSSSYLSVSLGGRVTITCKASDHINNWLAWYQQKPGNAPRLLISDATSLETGVPSRFSGSVSGMDYTLSITSLQTEDVATYYCQQYWSTPFTFGSGTKLEIE

SEQ ID NO: 63 - protein, 7 aa, synthetic construct
  source 1..7: note = 11F05 VL CDR2
DATSLET

SEQ ID NO: 64 - protein, 9 aa, synthetic construct
  source 1..9: note = 11F05 VL CDR3
QQYWSTPFT

SEQ ID NO: 65 - protein, 122 aa, synthetic construct
  source 1..122: note = 04D08 VH
EVQLQQSGPELEKPGASVKISCKASGYSFTGYSMNWVKQSNGKSLEWIGNIDPYYGGTSYNQNFKGKATLTVDKSSSTAYMQLKSLTSEDSAVYYCARSYFGNYEDTWFAYWGQGTLVTVSA

SEQ ID NO: 66 - protein, 5 aa, synthetic construct
  source 1..5: note = 04D08 VH CDR1
GYSMN

SEQ ID NO: 67 - protein, 17 aa, synthetic construct
  source 1..17: note = 04D08 VH CDR2
NIDPYYGGTSYNQNFKG

SEQ ID NO: 68 - protein, 13 aa, synthetic construct
  source 1..13: note = 04D08 VH CDR3
SYFGNYEDTWFAY

SEQ ID NO: 69 - protein, 113 aa, synthetic construct
  source 1..113: note = 04D08 VL
DIVMTQSPSSLTVTAGEKVTMSCKSSQSLLNIGNQNNYLTWYQQKPGQPPKLLIYWASTRESGVPDRFTGSGSGTDFTLTISSVQAEDLAVYYCQNDYSYPLTFGAGTKLELK

SEQ ID NO: 70 - protein, 17 aa, synthetic construct
  source 1..17: note = 04D08 VL CDR1
KSSQSLLNIGNQNNYLT

SEQ ID NO: 71 - protein, 7 aa, synthetic construct
  source 1..7: note = 04D08 VL CDR2
WASTRES

SEQ ID NO: 72 - protein, 9 aa, synthetic construct
  source 1..9: note = 04D08 VL CDR3
QNDYSYPLT

SEQ ID NO: 73 - protein, 450 aa, synthetic construct
  source 1..450: note = 02(VH3/VK3)-IgG-IL12 (LC) Heavy chain: HC02-VH3 (L234A, L235A, N297A)
EVQLVQSGAEVKKPGATLKISCKASGYTFTDFTMHWVQQAPGKGLEWIGLVNPNNGGNIYAEKFQGRATLTVDTSTSTAYMELSSLRSEDTSVYYCGRVPLYTSHGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 74 - protein, 757 aa, synthetic construct
  source 1..757: note = 02(VH3/VK3)-IgG-IL12 (LC) Light chain: LC02-VK3-IL12
EIVLTQSPATLSLSPGERATISCRASESVDSYDINSMHWYQQKPGQAPRLLIYRASHLQSGIPARFSGSGSRTDFTLTISSLEPEDFATYYCQQSNEDPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGSGGGSGGGSGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 75 - protein, 450 aa, synthetic construct
  source 1..450: note = 02(VH4/VK3)-IgG-IL12 (LC) Heavy chain: HC02-VH4 (L234A, L235A, N297A)
EVQLVQSGAEVKKPGATVKISCKASGYTFTDFTMHWVQQAPGKGLEWIGLVNPNNGGNIYAEKFQGRATLTVDTSTSTAYMELSSLRSEDTAVYYCGRVPLYTSHGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 76 - protein, 450 aa, synthetic construct
  source 1..450: note = 02(VH6/VK3)-IgG-IL12 (LC) Heavy chain: HC02-VH6 (L234A, L235A, N297A)
EVQLVQSGAEVKKPGATVKISCKASGYTFTDFTMHWVQQAPGKGLEWIGLVNPNNGGNIYAEKFQGRVTLTVDTSTSTAYMELSSLRSEDTAVYYCGRVPLYTSHGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 77 - protein, 988 aa, synthetic construct
  source 1..988: note = 02(VH3/VK3)-IgG-IL12 (1x HC) Heavy chain 1: HC02-VH3-IL12 (L234A, L235A, N297A, T366W)
EVQLVQSGAEVKKPGATLKISCKASGYTFTDFTMHWVQQAPGKGLEWIGLVNPNNGGNIYAEKFQGRATLTVDTSTSTAYMELSSLRSEDTSVYYCGRVPLYTSHGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGSGGGGSGGGGSGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGSGGGSGGGSGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 78 - protein, 218 aa, synthetic construct
  source 1..218: note = 02(VH3/VK3)-IgG-IL12 (1x HC) Light chain: LC02-VK3
EIVLTQSPATLSLSPGERATISCRASESVDSYDINSMHWYQQKPGQAPRLLIYRASHLQSGIPARFSGSGSRTDFTLTISSLEPEDFATYYCQQSNEDPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 79 - protein, 450 aa, synthetic construct
  source 1..450: note = 02(VH3/VK3)-IgG-IL12 (1x HC) Heavy chain 2: HC02-VH3 (L234A, L235A, N297A, T366S, L368A, Y407V)
EVQLVQSGAEVKKPGATLKISCKASGYTFTDFTMHWVQQAPGKGLEWIGLVNPNNGGNIYAEKFQGRATLTVDTSTSTAYMELSSLRSEDTSVYYCGRVPLYTSHGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 80 - protein, 988 aa, synthetic construct
  source 1..988: note = 02(VH4/VK3)-IgG-IL12 (1x HC) Heavy chain 1: HC02-VH4-IL12 (L234A, L235A, N297A, T366W)
EVQLVQSGAEVKKPGATVKISCKASGYTFTDFTMHWVQQAPGKGLEWIGLVNPNNGGNIYAEKFQGRATLTVDTSTSTAYMELSSLRSEDTAVYYCGRVPLYTSHGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGSGGGGSGGGGSGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGSGGGSGGGSGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 81 - protein, 450 aa, synthetic construct
  source 1..450: note = 02(VH4/VK3)-IgG-IL12 (1x HC) Heavy chain 2: HC02-VH4 (L234A, L235A, N297A, T366S, L368A, Y407V)
EVQLVQSGAEVKKPGATVKISCKASGYTFTDFTMHWVQQAPGKGLEWIGLVNPNNGGNIYAEKFQGRATLTVDTSTSTAYMELSSLRSEDTAVYYCGRVPLYTSHGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 82 - protein, 988 aa, synthetic construct
  source 1..988: note = 02(VH6/VK3)-IgG-IL12 (1x HC) Heavy chain 1: HC02-VH6- IL12 (L234A, L235A, N297A, T366W)
EVQLVQSGAEVKKPGATVKISCKASGYTFTDFTMHWVQQAPGKGLEWIGLVNPNNGGNIYAEKFQGRVTLTVDTSTSTAYMELSSLRSEDTAVYYCGRVPLYTSHGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGSGGGGSGGGGSGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGSGGGSGGGSGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 83 - protein, 450 aa, synthetic construct
  source 1..450: note = 02(VH6/VK3)-IgG-IL12 (1x HC) Heavy chain 2: HC02-VH6 (L234A, L235A, N297A, T366S, L368A, Y407V)
EVQLVQSGAEVKKPGATVKISCKASGYTFTDFTMHWVQQAPGKGLEWIGLVNPNNGGNIYAEKFQGRVTLTVDTSTSTAYMELSSLRSEDTAVYYCGRVPLYTSHGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 84 - skipped

SEQ ID NO: 85 - protein, 16 aa, synthetic construct
  source 1..16: note = (GGGS)4
GGGSGGGSGGGSGGGS

SEQ ID NO: 86 - protein, 20 aa, synthetic construct
  source 1..20: note = (GGGGS)4
GGGGSGGGGSGGGGSGGGGS

SEQ ID NO: 87 - protein, 19 aa, synthetic construct
  source 1..19: note = GGGS-(GGGGS)3
GGGSGGGGSGGGGSGGGGS

SEQ ID NO: 88 - protein, 5 aa, synthetic construct
  source 1..5: note = 02A01 Consensus 1 VH CDR1
  VARIANT 4: note = X is M or L
DFTXH

SEQ ID NO: 89 - protein, 197 aa, synthetic construct
  source 1..197: note = Human IL-12A (p35)
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 90 - protein, 306 aa, synthetic construct
  source 1..306: note = Human IL-12B (p40)
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 91 - protein, 519 aa, synthetic construct
  source 1..519: note = Human single-chain IL-12 (B-A)
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGSGGGSGGGSGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 92 - protein, 519 aa, synthetic construct
  source 1..519: note = Human single-chain IL-12 (A-B)
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGSGGGSGGGSGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 93-95 - skipped (3 entries)

SEQ ID NO: 96 - protein, 20 aa, synthetic construct
  source 1..20: note = (GS)10
GSGSGSGSGSGSGSGSGSGS

SEQ ID NO: 97 - protein, 30 aa, synthetic construct
  source 1..30: note = (GGS)10
GGSGGSGGSGGSGGSGGSGGSGGSGGSGGS

SEQ ID NO: 98 - protein, 40 aa, synthetic construct
  source 1..40: note = (GGGS)10
GGGSGGGSGGGSGGGSGGGSGGGSGGGSGGGSGGGSGGGS

SEQ ID NO: 99 - protein, 40 aa, synthetic construct
  source 1..40: note = (GGSG)10
GGSGGGSGGGSGGGSGGGSGGGSGGGSGGGSGGGSGGGSG

SEQ ID NO: 100 - protein, 50 aa, synthetic construct
  source 1..50: note = (GGSGG)10
GGSGGGGSGGGGSGGGGSGGGGSGGGGSGGGGSGGGGSGGGGSGGGGSGG

SEQ ID NO: 101 - protein, 50 aa, synthetic construct
  source 1..50: note = (GGGGS)10
GGGGSGGGGSGGGGSGGGGSGGGGSGGGGSGGGGSGGGGSGGGGSGGGGS

SEQ ID NO: 102 - protein, 15 aa, synthetic construct
  source 1..15: note = (GGGGS)3
GGGGSGGGGSGGGGS

SEQ ID NO: 103 - protein, 100 aa, synthetic construct
  source 1..100: note = (GGGGS)20
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 104 - protein, 100 aa, synthetic construct
  source 1..100: note = (GGSGG)20
GGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGG

SEQ ID NO: 105 - protein, 111 aa, synthetic construct
  source 1..111: note = Humanized 02A01 VK3 (D30E, N35Q)
EIVLTQSPATLSLSPGERATISCRASESVESYDIQSMHWYQQKPGQAPRLLIYRASHLQSGIPARFSGSGSRTDFTLTISSLEPEDFATYYCQQSNEDPLTFGQGTKLEIK

SEQ ID NO: 106 - protein, 15 aa, synthetic construct
  source 1..15: note = Humanized 02A01 VK3 (D30E, N35Q) CDR1
RASESVESYDIQSMH

SEQ ID NO: 107 - protein, 757 aa, synthetic construct
  source 1..757: note = "02(VH6/VK3-D30E/N35Q)-IgG-IL12 (LC) Light chain: LC02-VK3(D30E/N35Q)-IL12 "
EIVLTQSPATLSLSPGERATISCRASESVESYDIQSMHWYQQKPGQAPRLLIYRASHLQSGIPARFSGSGSRTDFTLTISSLEPEDFATYYCQQSNEDPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGSGGGSGGGSGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 108 - protein, 218 aa, synthetic construct
  source 1..218: note = 02(VH6/VK3-D30E/N35Q)-IgG-IL12 (1x HC) Light chain: LC02-VK3(D30E/N35Q)
EIVLTQSPATLSLSPGERATISCRASESVESYDIQSMHWYQQKPGQAPRLLIYRASHLQSGIPARFSGSGSRTDFTLTISSLEPEDFATYYCQQSNEDPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 109 - protein, 15 aa, synthetic construct
  source 1..15: note = 02A01 Consensus 1 VL CDR1
  VARIANT 7: note = X is D or E
  VARIANT 12: note = X is N or Q
RASESVXSYDIXSMH

SEQ ID NO: 110 - protein, 161 aa, synthetic construct
  source 1..161: note = Protein 110
SQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACD

SEQ ID NO: 111 - protein, 235 aa, synthetic construct
  source 1..235: note = Protein 111
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 112 - protein, 7 aa, synthetic construct
  source 1..7: note = Humanized 02A01-VH1 CDR1
GYTFTDF

SEQ ID NO: 113 - protein, 6 aa, synthetic construct
  source 1..6: note = Humanized 02A01-VH1 CDR2
NPNNGG

SEQ ID NO: 114 - protein, 7 aa, synthetic construct
  source 1..7: note = Humanized 02A01-VH5 CDR1
GYTFTDY

SEQ ID NO: 115 - protein, 8 aa, synthetic construct
  source 1..8: note = 03A01 VH CDR1
GYSITSDF

SEQ ID NO: 116 - protein, 5 aa, synthetic construct
  source 1..5: note = 03A01 VH CDR2
SYSGD

SEQ ID NO: 117 - protein, 5 aa, synthetic construct
  source 1..5: note = 03A01 VH-D64E CDR2
SYSGE

SEQ ID NO: 118 - protein, 5 aa, synthetic construct
  source 1..5: note = 03A01 Consensus 3 VH CDR1
  VARIANT 5: note = X is D or E
SYSGX

SEQ ID NO: 119 - protein, 7 aa, synthetic construct
  source 1..7: note = 05F10 VH CDR1
GFTFSSF

SEQ ID NO: 120 - protein, 6 aa, synthetic construct
  source 1..6: note = 05F10 VH CDR2
SSGDLS

SEQ ID NO: 121 - protein, 8 aa, synthetic construct
  source 1..8: note = 11F05 VH CDR1
GYSITSDY

SEQ ID NO: 122 - protein, 5 aa, synthetic construct
  source 1..5: note = 11F05 VH CDR2
SYSGS

SEQ ID NO: 123 - protein, 7 aa, synthetic construct
  source 1..7: note = 04D08 VH CDR1
GYSFTGY

SEQ ID NO: 124 - protein, 6 aa, synthetic construct
  source 1..6: note = 04D08 VH CDR2
DPYYGG